(19) United States
(12) Patent Application Publication
(10) Pub. No.: US 2025/0228855 A1
(43) Pub. Date: Jul. 17, 2025

(54) PYRAZINAMIDE COMPOUNDS

(71) Applicants: The General Hospital Corporation, Boston, MA (US); President and Fellows of Harvard College, Cambridge, MA (US)

(72) Inventors: N. Connor Payne, Boston, MA (US); Mark A. Tye, San Ramon, CA (US); Ralph Mazitschek, Weston, MA (US); Dyann F. Wirth, Boston, MA (US)

(21) Appl. No.: 18/850,981

(22) PCT Filed: Apr. 19, 2023
(86) PCT No.: PCT/US2023/019152

§ 371 Date: Sep. 25, 2024

Related U.S. Application Data
(60) Provisional application No. 63/333,080, filed on Apr. 20, 2022.
(60) Provisional application No. 63/353,348, filed on Jun. 17, 2022.
(60) Provisional application No. 63/353,526, filed on Jun. 17, 2022.

(51) Int. Cl. A61K 31/517; A61K 31/496; A61K 31/497; A61K 31/7076; C07D 401/12; C07D 401/14; C07D 403/14; C07H 19/167

(52) U.S. Cl. CPC A61K 31/517; A61K 31/496; A61K 31/497; A61K 31/7076; C07D 401/12; C07D 401/14; C07D 403/14; C07H 19/167

(57) ABSTRACT

The present application provides compounds and methods for treating various diseases such as malaria and cancer.

### CLAIM OF PRIORITY

This application claims priority to U.S. Provisional Patent Application Ser. No. 63/353,348, filed on Jun. 17, 2022, U.S. Provisional Patent Application Ser. No. 63/353,526, filed on Jun. 17, 2022, U.S. Provisional Patent Application Ser. No. 63/333,080, filed on Apr. 20, 2022, the entire contents of which are hereby incorporated by reference.

### TECHNICAL FIELD

The present application provides compounds which are inhibitors of aminoacyl tRNA-synthetase (e.g., prolyl-tRNA-synthetase) and are useful for treating disorders associated with aminoacyl tRNA-synthetase activity and/or expression.

### BACKGROUND

Malaria is an infectious diseases caused by Plasmodium parasites and ranks third among deadly infectious diseases, with over 200 million cases and more than 600,000 deaths per year. The highest burden of disease focused Aon poverty-stricken nations in Asia, South America, and Africa with significant morbidity and mortality. The causative agents of malaria are protozoan parasites of the genus Plasmodium that are transmitted between humans by mosquitoes. In humans, the parasite evolves through a liver stage, a symptomatic intra-erythrocytic asexual stage, and a sexual blood stage (ABS), which is responsible for malaria transmission.

### SUMMARY

Current antimalarial drugs are only relevant for the asexual blood stage (ABS) of the Plasmodium parasite in the infected human, restricting their utility to the treatment of acute malaria. Development of antimalarials that are efficacious against the human liver and sexual blood stages provides advantages over the existing drugs by allowing to prevent the onset of symptoms in the infected individual and well as to stop transmission. Aminoacyl-tRNA synthetase (aaRS) enzymes, including prolyl-tRNA synthetase (ProRS), are protein targets for such a malaria chemotherapy. Accordingly, the present disclosure provides compounds that are high-affinity ProRS inhibitors. The exemplified compounds advantageously are the first triple-site ligands for aaRS enzymes that simultaneously engage all three substrate-binding pockets, exhibit potent dual-stage activity against Plasmodium parasites, and display good cellular host selectivity. Furthermore, emergence and spread of resistance to currently available antimalarials threatens the ability to treat and contain even acute malaria. Importantly here, the compounds of this disclosure advantageously overcome existing resistance mechanisms providing robust and resistance-proof antimalarial therapies.

In one general aspect, the present disclosure provides a compound of Formula (I):

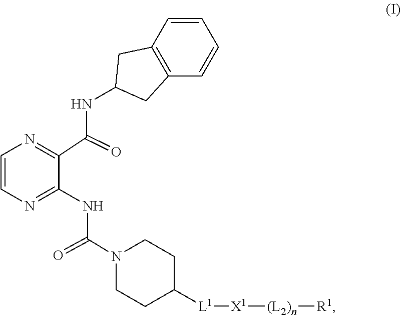

or a pharmaceutically acceptable salt thereof.

In another general aspect, the present disclosure provides a compound of formula:

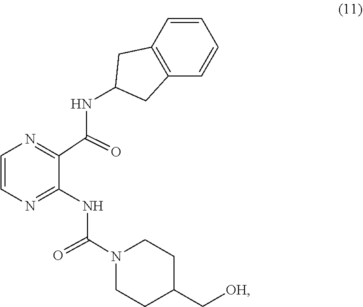 [SMILES: O=C(NC1Cc2ccccc2C1)c1nccnc1NC(=O)N1CCC(CO)CC1]

or a pharmaceutically acceptable salt thereof.

In yet another general aspect, the present disclosure provides a compound of formula:

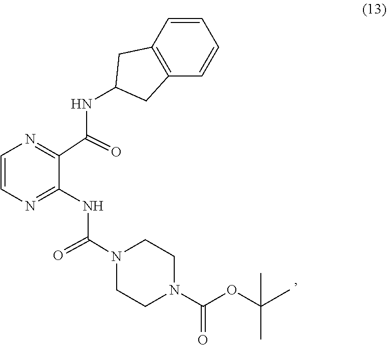 [SMILES: CC(C)(C)OC(=O)N1CCN(C(=O)Nc2nccnc2C(=O)NC2Cc3ccccc3C2)CC1]

or a pharmaceutically acceptable salt thereof.

In yet another general aspect, the present disclosure provides a compound of Formula (II):

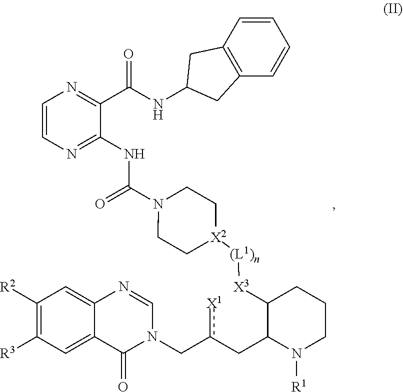

or a pharmaceutically acceptable salt thereof.

In yet another general aspect, the present disclosure provides a compound of formula:

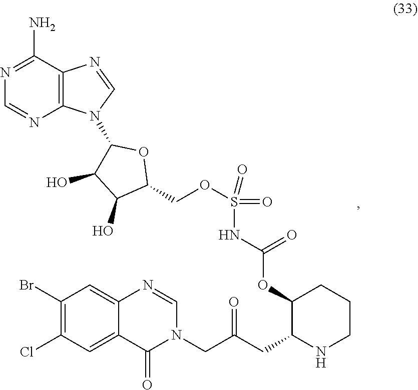 [SMILES: Nc1ncnc2c1ncn2[C@@H]1O[C@H](COS(=O)(=O)NC(=O)O[C@H]2CCCN[C@@H]2CC(=O)Cn2cnc3cc(Br)c(Cl)cc3c2=O)[C@@H](O)[C@H]1O]

or a pharmaceutically acceptable salt thereof.

In yet another general aspect, the present disclosure provides a compound of formula:

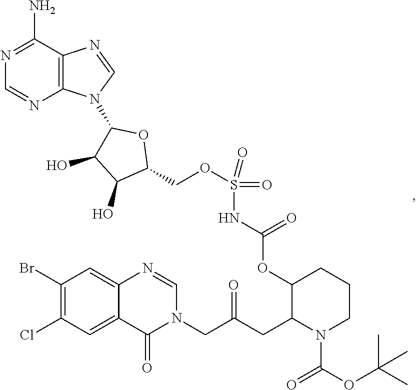 [SMILES: CC(C)(C)OC(=O)N1CCCC(OC(=O)NS(=O)(=O)OC[C@H]2O[C@@H](n3cnc4c(N)ncnc43)[C@H](O)[C@@H]2O)C1CC(=O)Cn1cnc2cc(Br)c(Cl)cc2c1=O]

or a pharmaceutically acceptable salt thereof.

In yet another general aspect, the present disclosure provides a compound of Formula (III):

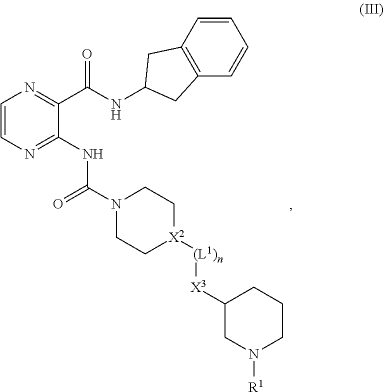

or a pharmaceutically acceptable salt thereof.

In yet another general aspect, the present disclosure provides a compound of Formula (IV):

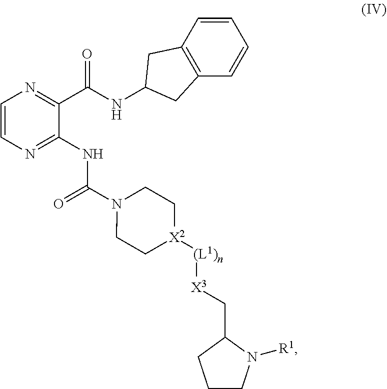

or a pharmaceutically acceptable salt thereof.

In yet another general aspect, the present disclosure provides a compound of formula:

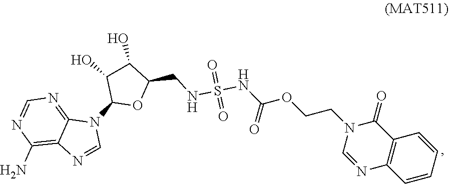 [SMILES: Nc1ncnc2c1ncn2[C@@H]1O[C@H](CNS(=O)(=O)NC(=O)OCCn2cnc3ccccc3c2=O)[C@@H](O)[C@H]1O]

or a pharmaceutically acceptable salt thereof.

In yet another general aspect, the present disclosure provides a compound of Formula (V):

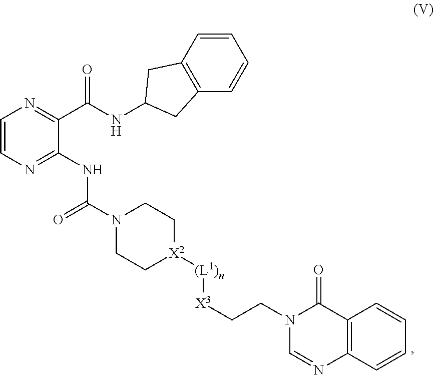

or a pharmaceutically acceptable salt thereof.

In yet another general aspect, the present disclosure provides a pharmaceutical composition comprising a compound as described herein, or a pharmaceutically acceptable salt thereof, and a pharmaceutically acceptable carrier.

In yet another general aspect, the present disclosure provides a method of inhibiting prolyl-tRNA-synthetase in a cell, comprising contacting the cell with a compound as described herein, or a pharmaceutically acceptable salt thereof.

In yet another general aspect, the present disclosure provides a method of inhibiting prolyl-tRNA-synthetase in a subject, comprising administering to the subject a compound as described herein, or a pharmaceutically acceptable salt thereof.

In yet another general aspect, the present disclosure provides a method of treating a disorder associated with glutamyl-prolyl-tRNA synthetase, prolyl-tRNA synthetase, or a combination thereof, the method comprising administering to a subject in need thereof a therapeutically effective amount of a compound as described herein, or a pharmaceutically acceptable salt thereof, and a pharmaceutically acceptable carrier.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1A ProRS inhibitor design and anti-Plasmodium activity. a, Schematic representation of the ProRS active site and binding mode of canonical substrates (proline, ATP, tRNAPro), halofuginone (1), and T-3767758 (2). The active site of ProRS constitutes three distinct substrate pockets, which bind the terminal adenosine (A76) residue of tRNAPro (red), proline (green), and ATP (yellow). Halofuginone binds in the tRNAPro and proline-binding pockets and requires the presence of ATP for tight binding (ATP-uncompetitive), while compound 2 targets the ATP-binding pocket and requires the presence of proline for tight binding (proline-uncompetitive).

FIG. 1B ProRS inhibitor design and anti-Plasmodium activity. In vitro activity of halofuginone (HFG), 2, and NCP26 (3) against wild-type (Dd2-2D4), halofuginone-induced (HFG-induced), and HFGR-I P. falciparum strains. Asexual blood stage (ABS) P. falciparum parasites were treated with test compounds at varying concentrations for 72 h, followed by quantification of parasite growth by SYBR Green staining. Compound 2 and NCP26 are refractory to halofuginone resistance.

FIG. 1C Ribbon representation of HsProRS (blue ribbon/sticks) in complex with proline and compound 2 (silver sticks, PDB: 5VAD) reveals an energetically unfavorable axial confirmation of the cyclohexyl substituent.

FIG. 1D Chemical structures of selected pyrazinamide compounds.

FIG. 1E Co-crystal structure of proline and NCP26 (green sticks) bound to PfcProRS (orange sticks, PDB: 6T7K) shows that the piperidine adapting an energetically more favorable confirmation, with no notable change in the interacting amino acid residues.

FIGS. 2A-2I Saturation binding of fluorescent tracer MAT379 to CoraFluor-1-labeled HT-PfcProRS (1 nM) in the absence or presence of 100 μM Pro or 500 μM ATP. TR-FRET ratios were background-corrected relative to 10 μM ProSA (˜20,000×K_{D}). Dose-response titration of reference compounds using CoraFluor-1-labeled HT-PfcProRS (0.25-1 nM) and MAT379 as tracer at 2.5×K_{D} (250 nM) in the absence or presence of 100 μM Pro or 500 μM ATP. Under all three conditions, ProSA is titrating HT-PfcProRS. Dose-response titration of ProSA using CoraFluor-1-labeled HT-PfcProRS (20 pM), CoraFluor-1-labeled anti-His6 antibody (1 nM), and MAT379 as tracer at 2.5×K_{D} (250 nM). Saturation binding of fluorescent tracer MAT379 to CoraFluor-1-labeled HT-HsProRS (1.5 nM). TR-FRET ratios were background corrected relative to 10 μM ProSA (˜20,000×K_{D}). Dose-response titration of test compounds using CoraFluor-1-labeled HT-HsProRS (1 nM) and MAT379 as tracer at 0.15×K_{D} (250 nM) in the absence or presence of 100 μM Pro. ProSA is titrating HT-HsProRS in both conditions and NCP26 is titrating HT-HsProRS in the presence of 100 μM Pro. Dose-response titration of test compounds using indicated concentrations CoraFluor-1-labeled HT-HsProRS, 1 nM CoraFluor-1-labeled anti-His6 antibody, and MAT379 as tracer at 0.15×K_{D} (250 nM).

FIGS. 3A-3D NCP26-resistance selection and whole genome sequence analysis. A. In vitro activity of NCP26 against ABS P. falciparum Dd2-2D4 (parent) or subclones from three independent resistance selection experiments (S1-3). S1 did not yield resistant parasites and S1—clone A8 was included for comparison. B. In vitro activity of reference compounds against Dd2-2D4 (parent) or subclones from each selection (51-3). Reference compounds include PfcProRS inhibitors (halofuginone and halofuginol) and non-PfcProRS inhibitors dihydroartemisinin (DHA) and borrelidin (P. falciparum threonyl-tRNA synthetase inhibitor). C. Residues T512S(S2) and F405L (S3) were identified by whole genome sequence analysis to mediate NCP26-resistance and are mapped to the co-crystal structure of NCP26 (green) and proline (white) bound to wildtype PfcProRS (PDB: 6T7K). Both amino acid side chains directly interact with the pyrazinamide core. D. Whole genome sequence analysis revealed amplification of the intra-chromosomal region harboring the PfcProRS locus on chromosome 12 that is observed in all S3 clones. No copy number variations were observed in other chromosomes for S3 clones or in any chromosomes for Dd2-2D4 (parent), S1 (no NCP26-resistance observed), or S2 (PfcProRST512S). ABS growth assay data in A is expressed as mean±s.d. (n=3 technical replicates) and are representative of at least 2 independent experiments. ABS growth assay in B is expressed as the mean IC₅₀ values from independent bioreplicates±s.d. of bioreplicates (n=3 technical replicates per bioreplicate) and include at least two independent bioreplicates (experiments), except for DHA in S2—clone B9 which only was only tested once.

FIGS. 4A-4B Comparison of non-conserved ProRS residues adjacent to the active site. A. Comparison of co-crystal structure of T-3767758 (red) and proline (blue) bound to HsProRS (PDB: 5VAD) with PfcProRS (PDB: 6T7K). The surface of PfcProRS is shown in light blue and non-conserved residues are highlighted in orange. B. Sequence alignment between HsProRS and PfcProRS. Active site residues are in bold with non-conserved active site or adjacent residues in orange.

FIGS. 5A-5K Triple-site ProRS inhibitors. A. Interaction of halofuginone with non-hydrolyzable ATP-analog AMP-PNP bound to HsProRS (PDB: 4HVC). Hydrogen-bonds are indicated by yellow dashed lines. B. Chemical structures of triple-site inhibitors based on halofuginone-AMP and pyrazinamide-halofuginone hybrid design, with relative stereochemistry shown for halofuginone moiety. C. Chemical structures of iso-33 (relative stereochemistry for halofuginone moiety) and cyclization byproduct N3,5′-cycloadenosine. D. Dose-response titration of pyrazinamide-halofuginone hybrids. E. Chemical structure of iso-MAT436 (40) with relative stereochemistry for halofuginone moiety. F. Co-crystal structure of MAT436 (cyan sticks, PDB: 7QC1) bound to PfcProRS reveals electrostatic interactions (dashed lines) with conserved residues within the active site (light blue sticks) and occupancy of all three substrate-binding pockets in the active site. G, H. Overlay of co-crystal structures of PfcProRS in complex with MAT436 (cyan sticks, PDB: 7QC1) with either (G) PfcProRS in complex with halofuginone and AMP-PNP (silver sticks, PDB: 4Q15), and (H) PfcProRS in complex with NCP26 (yellow sticks) and proline (orange sticks, PDB: 6T7K). I. In vitro activity of pyrazinamide-halofuginone hybrids and BOC-protected precursors against wild-type ABS P. falciparum Dd2-2D4. J. In vitro activity of pyrazinamide-halofuginone hybrids against halofuginone-induced (HFG-Induced) and HFGR-I ABS P. falciparum. K. In vitro activity of NCP26 and MAT436 against liver stage P. berghei and HuH7 liver cells. TR-FRET assay data in d are expressed as mean±s.d. (n≥2 technical replicates) and are representative of at least 2 independent experiments. ABS growth assay data in I and J are expressed as mean±s.d. (n=3 technical replicates) and are representative of at least 3 independent experiments, except for compound 39 which is only representative of 2 independent experiments for Dd2-2D4 in panel I. Liver stage and HuH7 data in K are expressed as mean±s.d. of 3 biological replicates (total n=9 technical replicates).

FIGS. 6A-6G Asexual blood stage P. falciparum activity of ATP-site targeted pyrazinamide-derived ProRS inhibitors. A-C. In vitro characterization of pyrazinamides 4 (A), 6 (B), and 7 (C) in wildtype (Dd2-2D4; circles and solid lines), halofuginone-induced (squares and dashed lines), and HFGR-I (triangles and dotted lines) ABS P. falciparum parasites. D-G. In vitro characterization of ProRS inhibitors in Dd2-2D4 wildtype (d and g), halofuginone-induced (E), and HFGR-I (F) ABS P. falciparum parasites. Data in panels D and G was split for visualization purposes only. Data are expressed as mean±s.d. (n=3 technical replicates) and are representative of at least 3 independent experiments, except for 8, 13, and 14 which had one bioreplicate each in Dd2-2D4.

FIGS. 7A-7D Structural comparison of free and ligand-bound HsProRS. A. Overlay of HsProRS crystal structures in the apo state (PDB: 4K86), bound to proline alone (PDB: 7OSY), and bound to both proline and 2 (PDB: 5VAD) reveals significant allosteric structural changes upon proline binding, including the ATP-binding pocket and the active site entry. Selected residues in and adjacent to the active site are shown as lines. B. Apo crystal structure of HsProRS (PDB: 4K86). C. Co-crystal structure of HsProRS bound to proline (PDB: 7OSY). D. Co-crystal structure of HsProRS bound to proline and 2 (PDB: 5VAD).

FIG. 8 Data collection and refinement statistics for PfcProRS ligand complexes. Data were collected from one crystal for each structure. Statistics for the highest-resolution shell are shown in parentheses.

### DETAILED DESCRIPTION

Halofuginone is one the most potent known antimalarials and a synthetic derivative of the natural product febrifugine, the curative ingredient of an ancient herbal remedy that has been used in Traditional Chinese Medicine for over 2,000 years for the treatment of fevers and malaria. Structure of halofuginone (1) is shown below:

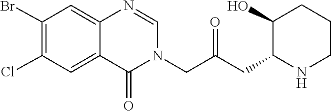 [SMILES: O=C(C[C@H]1NCCC[C@@H]1O)Cn1cnc2cc(Br)c(Cl)cc2c1=O]

However, the therapeutic utility of halofuginone and analogs as antimalarials has been stymied by poor tolerability, and the previously unknown mode of action in the host and parasite has impeded rational development of drugs with improved pharmacological properties.

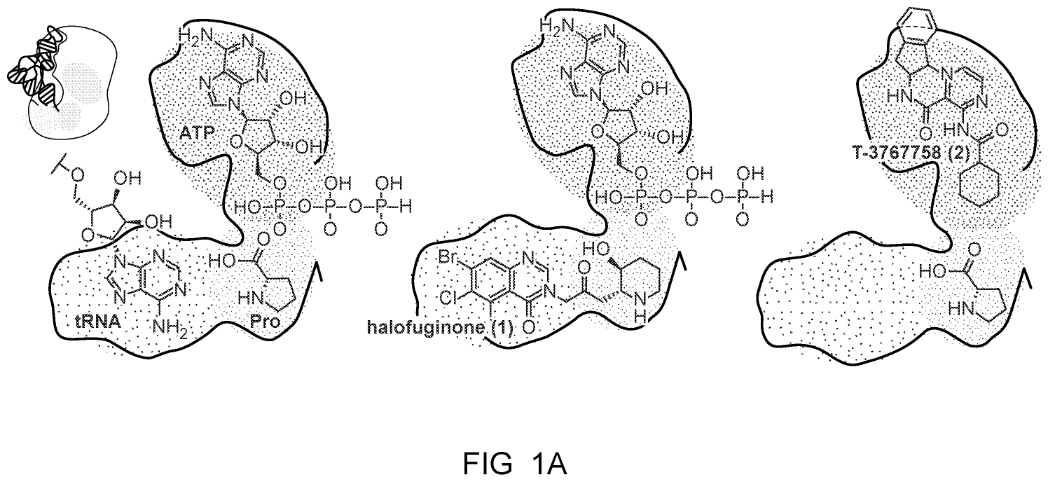

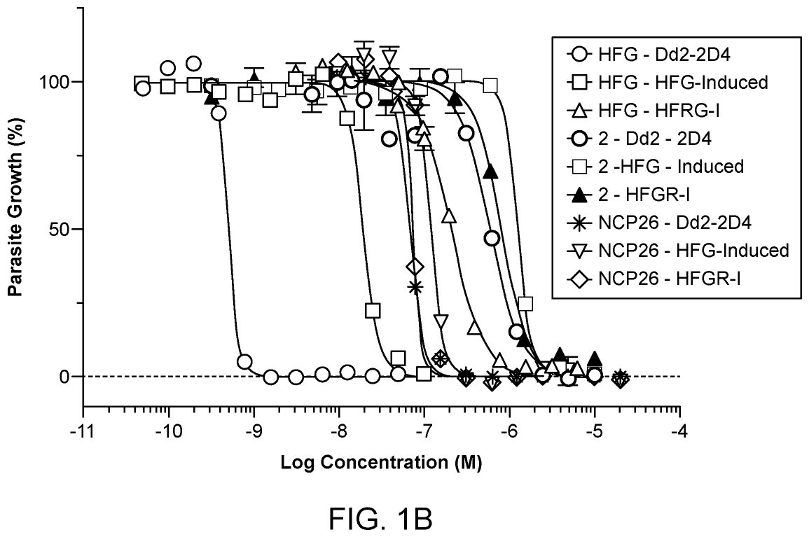

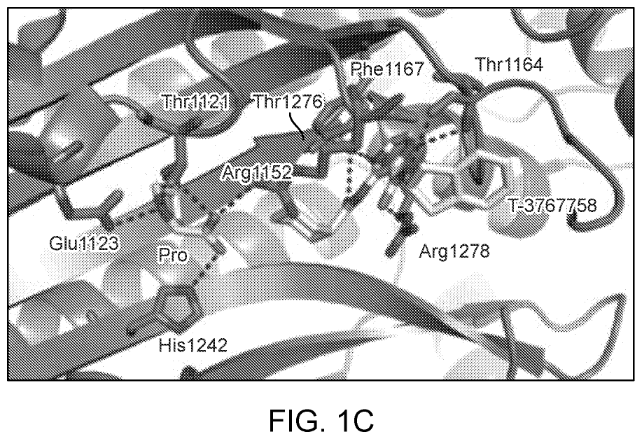

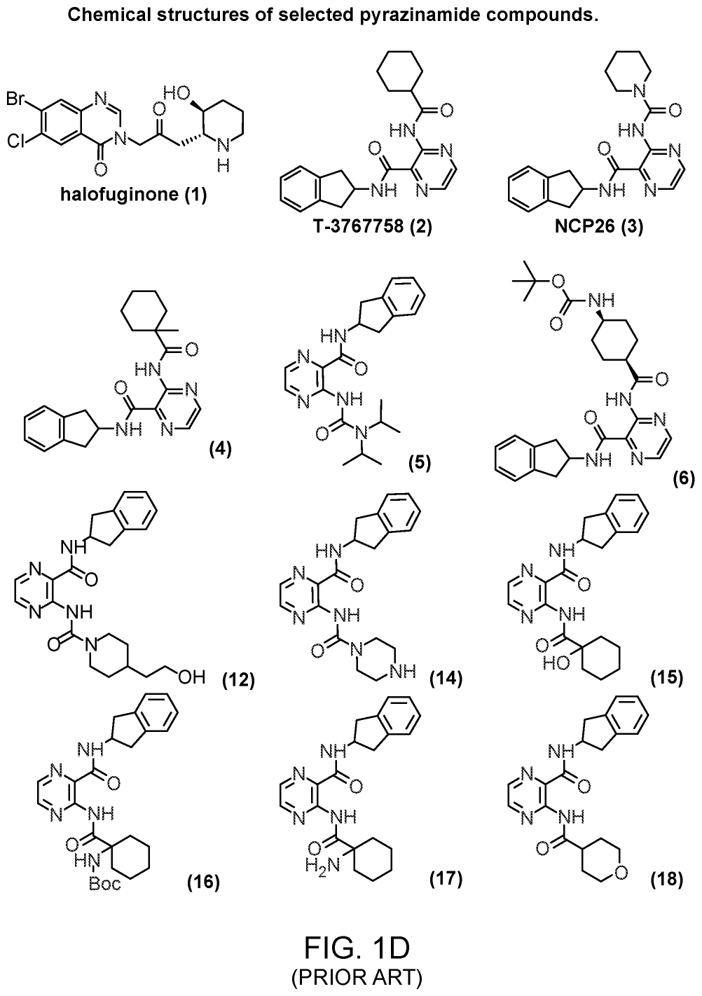

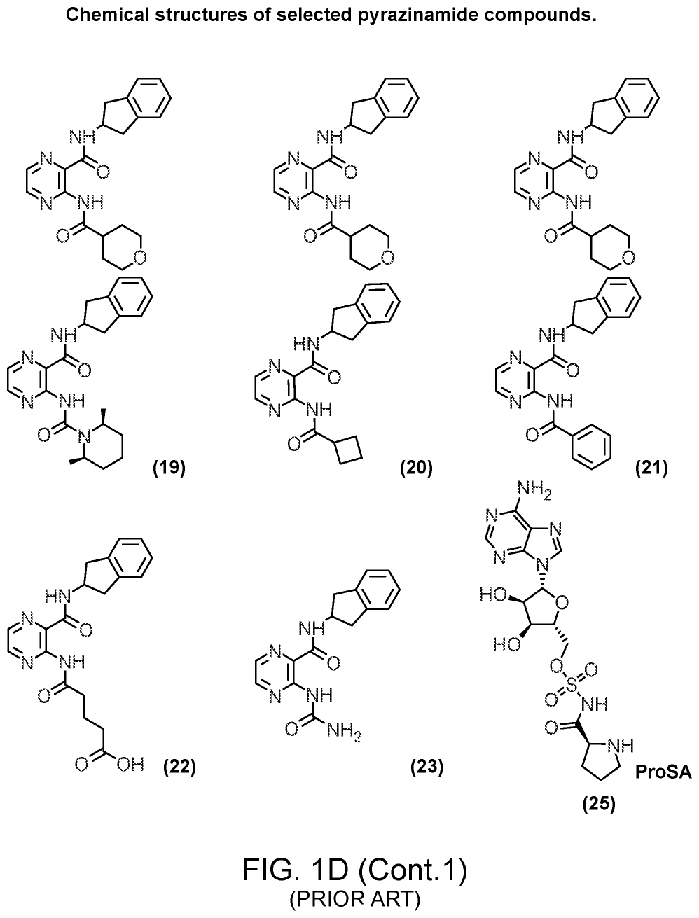

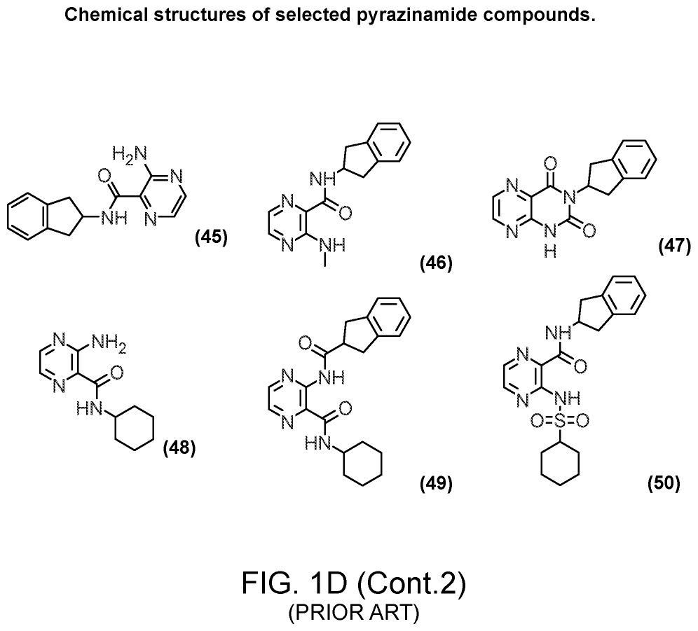

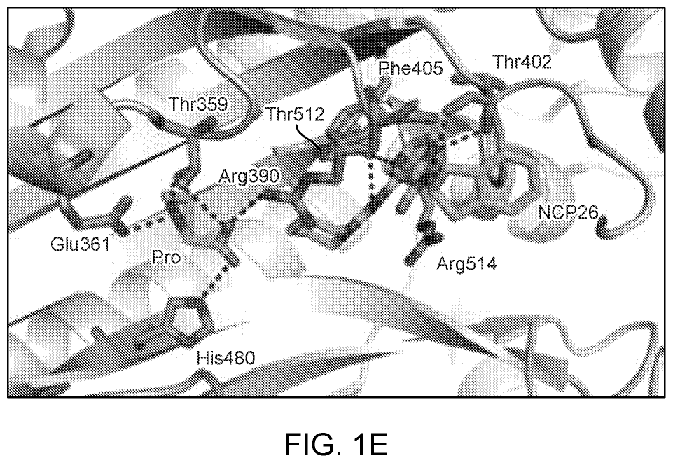

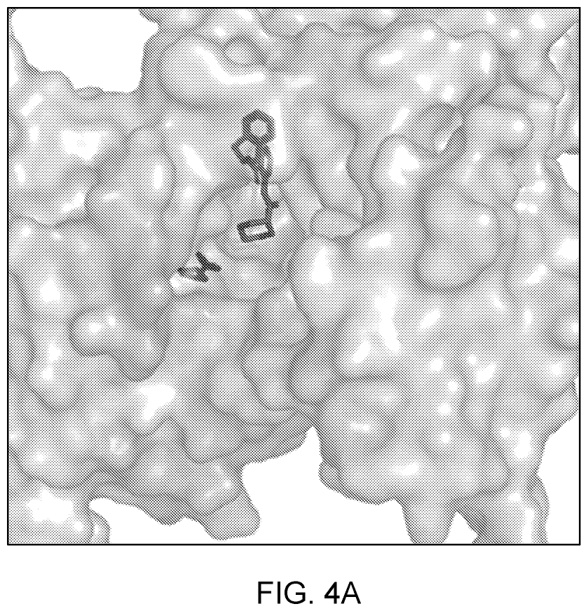

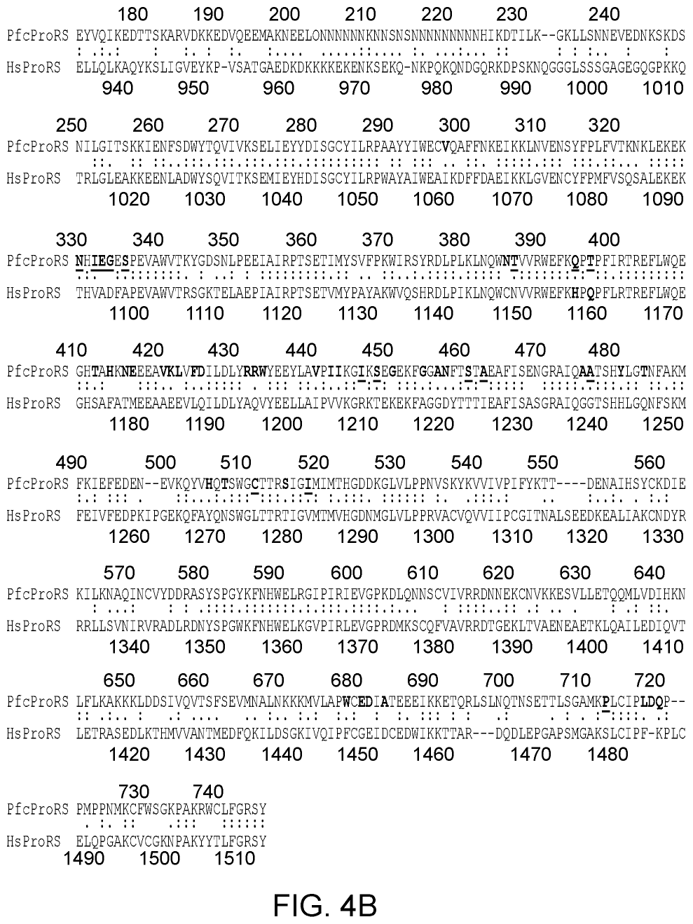

Cytoplasmic prolyl-tRNA synthetase (ProRS) was identified as the molecular target of halofuginone in P. falciparum. ProRS (PfcProRS) is a member of the aaRS enzyme family, which exist in all living cells and catalyze the transfer of amino acids to their cognate tRNAs. However, recent research has also revealed secondary functions of specific aaRS isoforms and tRNAs beyond their canonical role in protein biosynthesis. In addition, halofuginone is also active against liver stage parasites in vitro and in vivo. As to the mode of action of halofuginone in humans, where halofuginone has been studied as chemotherapeutic, antifibrotic, immunomodulatory agent and more recently as antiviral drug, the prolyl-tRNA synthetase activity of the bifunctional glutamyl-prolyl-tRNA synthetase (GluProRS) was identified as the mechanistic target. Crystallographic data of the co-complexes with human and Plasmodium ProRS revealed that halofuginone binds the A76-tRNA^{Pro} and proline-binding pockets of the active site (FIG. 1a), which are highly conserved between both homologs. Despite the high homology between parasite (PfcProRS) and host (HsProRS) enzymes (see FIG. 4) and comparable biochemical potency, halofuginone is significantly more active against asexual blood-stage P. falciparum than mammalian cell lines. As to halofuginone resistance, within five generations (10 days) bulk cultures, termed halofuginone-induced parasites, mounted a 10-20-fold tolerance by upregulation (˜20-fold) of intracellular proline, which is competitive with halofuginone. This mode of resistance explains the failure of febrifugine and halofuginone to control recrudescence in vivo and their narrow therapeutic indices as antimalarials. Without being bound by any particular theory or speculation, it is believed that due to the particular mode of binding to the active site of ProRS for the compounds within the present claims (as discussed below), the parasite advantageously does not develop rapid resistance to these compounds.

The active site of ProRS comprises three distinct pockets that bind ATP, proline, and the 3′-terminal adenosine residue of tRNA^{Pro} (A76), respectively (FIG. 1a). A class of HsProRS inhibitors is represented by T-3767758 (2) (FIG. 1a). Unlike halofuginone and analogs, which span the A76 and proline-binding sites and interact in an ATP-uncompetitive manner (i.e. the inhibitor affinity increases with increasing ATP concentration), this inhibitor class targets the ATP-binding pocket and features adjacent to the active site. For example, T-3767758 (2) (FIG. 1a) displayed proline-uncompetitive steady state kinetics for HsProRS. This property is desirable for halofuginone-tolerant strains with elevated intracellular proline (halofuginone-induced), as it allows to overcome or even select against this resistance mechanism. Sequence analysis of HsProRS and PfcProRS paralogs reveals overall high homology with several nonconserved residues in and adjacent to the binding site occupied by compound 2 (FIG. 4). Without being bound by any particular theory or speculation, it is believed that inhibitors containing the structural core of compound 2 are active against PfcProRS and this core therefore offers potential for the development of selective inhibitors.

Accordingly, the present application provides, inter alia, pyrazine-based compounds that are selective inhibitors of prolyl-tRNA-synthetase (ProRS). As such, the compounds are useful, for example, for treating a disease or disorder in which activity of ProRS is implicated, such as a parasitic infection (e.g., malaria, toxoplasmosis, leishmaniasis, cryptosporidiosis, or coccidiosis), autoimmune disorder, fibrosis, cancer, bacterial infection, fungal infection, or viral infection. Some embodiments of the compounds, methods of their use, methods of their making, as well as the pharmaceutical compositions containing these compounds and combination treatments are described below.

### Compounds

In some embodiments, the present disclosure provides a compound of Formula (I):

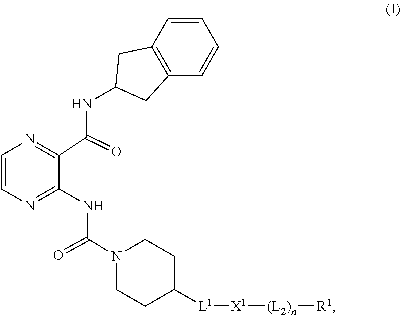

or a pharmaceutically acceptable salt thereof, wherein:
L¹ is C₁₋₃ alkylene, or L¹ is absent;
X¹ is selected from O and NR^{N};
R^{N} is selected from H, C₁-3 alkyl, and C₁-3 haloalkyl;
each L² is independently selected from O, S, S(═O)₂, NR^{N}, C═O, C═S, and C₁₋₆ alkylene;
n is an integer from 0 to 12; and
R¹ is selected from H, C₁₋₆ alkyl, C₃₋₆ cycloalkyl, C₆₋₁₀ aryl.

In some embodiments, L¹ is absent.

In some embodiments, L¹ is C₁₋₃ alkylene.

In some embodiments, L¹ is selected from methylene, 1,2-ethylene, 1,1-ethylene, and propylene.

In some embodiments, L¹ is methylene.

In some embodiments, X¹ is O.

In some embodiments, X¹ is NH.

In some embodiments, the compound of Formula (I) has formula:

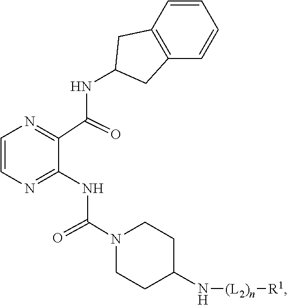

or a pharmaceutically acceptable salt thereof.

In some embodiments, the compound of Formula (I) has formula:

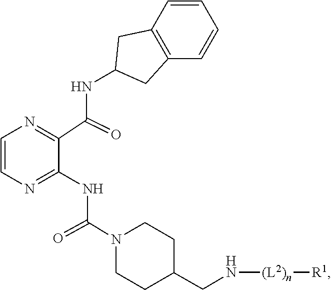

or a pharmaceutically acceptable salt thereof.

In some embodiments, n is 0.

In some embodiments, n is an integer from 1 to 12.

In some embodiments, each L² is independently selected from O, S, NH, C═O, C═S, and C₁₋₆ alkylene.

In some embodiments, the moiety (L²)ₙ comprises (C═O)O.

In some embodiments, the moiety (L²)ₙ comprises NH(C═O)O.

In some embodiments, the moiety (L²)ₙ comprises (C═O)—C₁₋₆ alkylene.

In some embodiments, the moiety (L²)ₙ comprises NH(C═O).

In some embodiments, the moiety (L²)ₙ comprises NH(C═O)NH.

In some embodiments, the moiety (L²)ₙ comprises NH(C═S)NH.

In some embodiments, the moiety (L²)ₙ comprises any one of the following fragments:

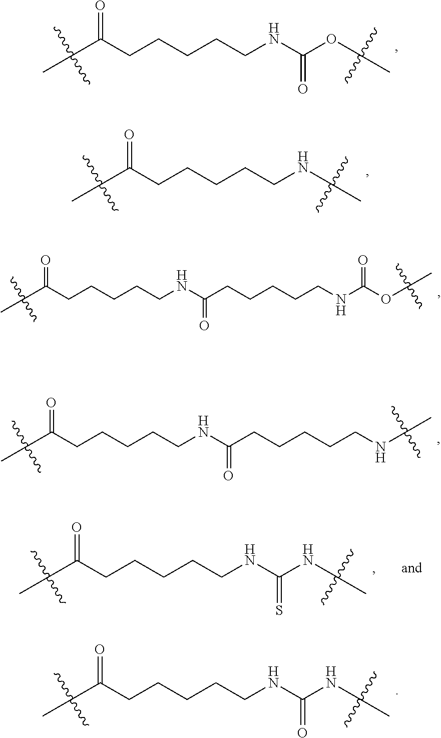 [SMILES: *OC(=O)NCCCCCC(*)=O ; *NCCCCCC(*)=O ; *OC(=O)NCCCCCC(=O)NCCCCCC(*)=O ; *NCCCCCC(=O)NCCCCCC(*)=O ; *NC(=S)NCCCCCC(*)=O ; *NC(=O)NCCCCCC(*)=O]

In some embodiments, R¹ is H.

In some embodiments, R¹ is C₁₋₆ alkyl.

In some embodiments, R¹ is C₃₋₆ cycloalkyl.

In some embodiments, R¹ is C₆₋₁₀ aryl.

In some embodiments, the compound of Formula (I) is selected from any one of the following compounds:

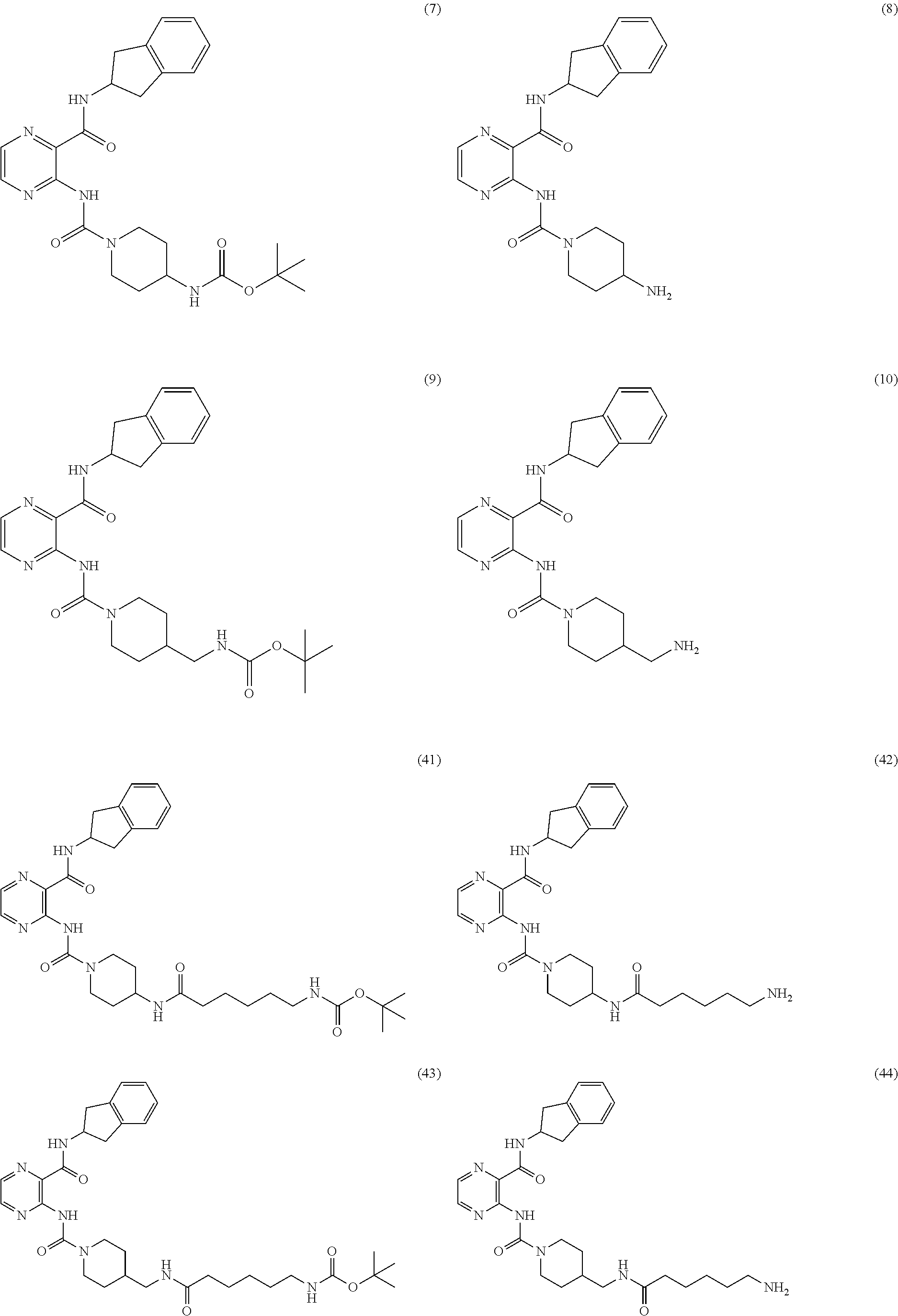

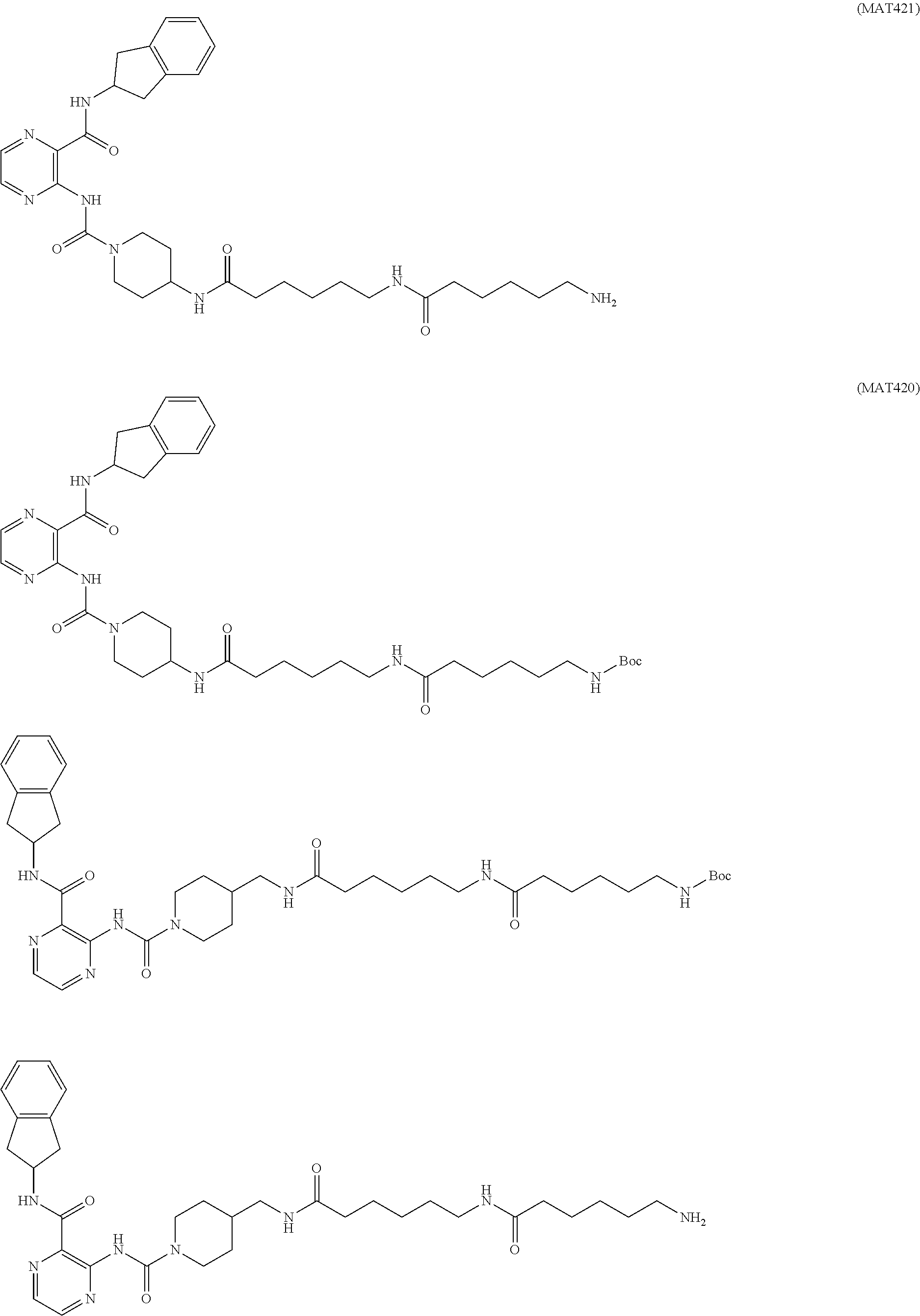 [SMILES: NCCCCCC(=O)NCCCCCC(=O)NC1CCN(C(=O)Nc2nccnc2C(=O)NC2Cc3ccccc3C2)CC1 ; CC(C)(C)OC(=O)NCCCCCC(=O)NCCCCCC(=O)NC1CCN(C(=O)Nc2nccnc2C(=O)NC2Cc3ccccc3C2)CC1 ; CC(C)(C)OC(=O)NCCCCCC(=O)NCCCCCC(=O)NCC1CCN(C(=O)Nc2nccnc2C(=O)NC2Cc3ccccc3C2)CC1 ; NCCCCCC(=O)NCCCCCC(=O)NCC1CCN(C(=O)Nc2nccnc2C(=O)NC2Cc3ccccc3C2)CC1]

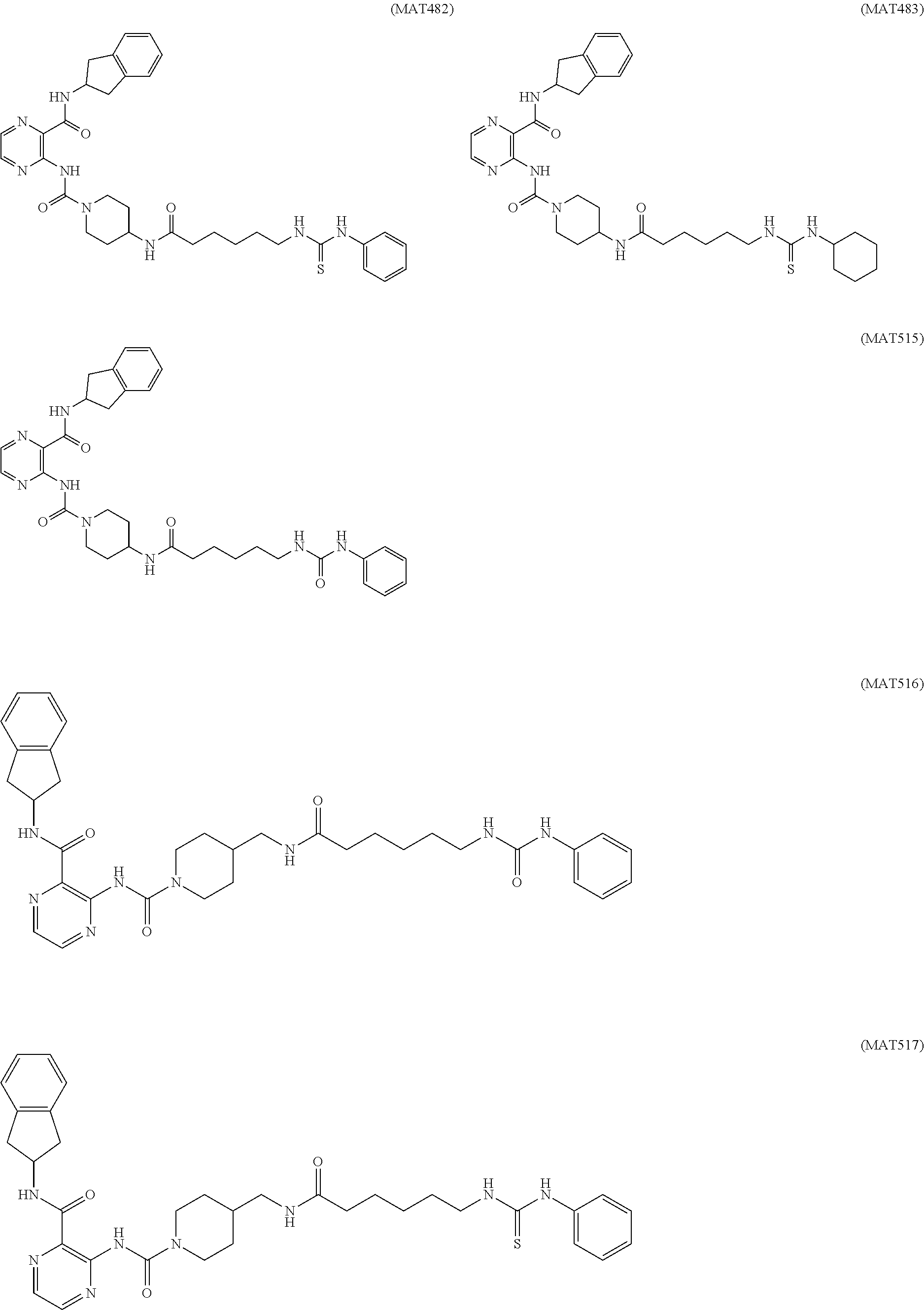 [SMILES: O=C(CCCCCNC(=S)Nc1ccccc1)NC1CCN(C(=O)Nc2nccnc2C(=O)NC2Cc3ccccc3C2)CC1 ; O=C(CCCCCNC(=S)NC1CCCCC1)NC1CCN(C(=O)Nc2nccnc2C(=O)NC2Cc3ccccc3C2)CC1 ; O=C(CCCCCNC(=O)Nc1ccccc1)NC1CCN(C(=O)Nc2nccnc2C(=O)NC2Cc3ccccc3C2)CC1 ; O=C(CCCCCNC(=O)Nc1ccccc1)NCC1CCN(C(=O)Nc2nccnc2C(=O)NC2Cc3ccccc3C2)CC1 ; O=C(CCCCCNC(=S)Nc1ccccc1)NCC1CCN(C(=O)Nc2nccnc2C(=O)NC2Cc3ccccc3C2)CC1]

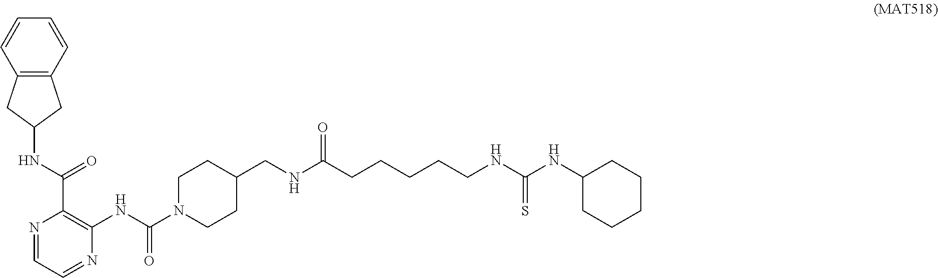 [SMILES: O=C(CCCCCNC(=S)NC1CCCCC1)NCC1CCN(C(=O)Nc2nccnc2C(=O)NC2Cc3ccccc3C2)CC1]

or a pharmaceutically acceptable salt thereof.

In some embodiments, the present disclosure provides a compound of formula:

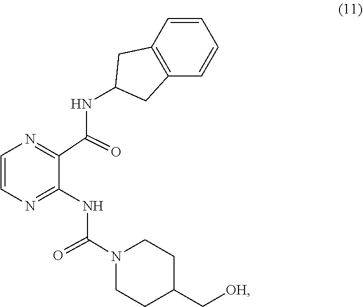 [SMILES: O=C(NC1Cc2ccccc2C1)c1nccnc1NC(=O)N1CCC(CO)CC1]

or a pharmaceutically acceptable salt thereof.

In some embodiments, the present disclosure provides a compound of formula:

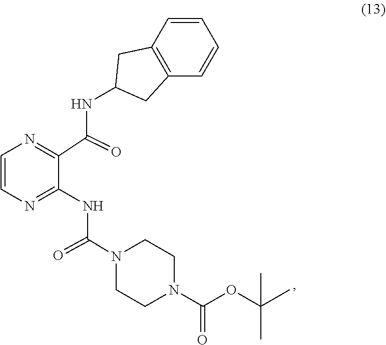 [SMILES: CC(C)(C)OC(=O)N1CCN(C(=O)Nc2nccnc2C(=O)NC2Cc3ccccc3C2)CC1]

or a pharmaceutically acceptable salt thereof.

In some embodiments, the present disclosure provides a compound of Formula (II):

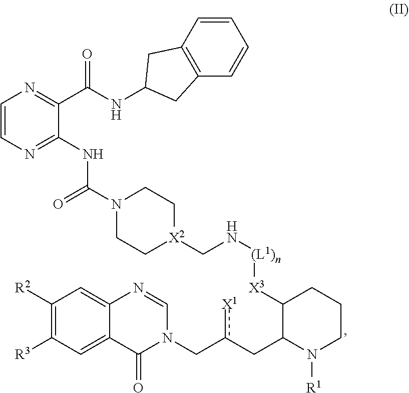

or a pharmaceutically acceptable salt thereof, wherein:
[symbol] is a single bond or a double bond;
when [symbol] is a double bond, X¹ is O or S;
when [symbol] is a single bond, X¹ is OH or SH;
R¹ is selected from H, C₁₋₃ alkyl, C₁₋₃ haloalkyl, —C(═O)R^{A1}, and —C(═O)OR^{A1};
each R^{A1} is selected from H, C₁₋₆ alkyl, and C₁₋₃ haloalkyl;
R² and R³ are each independently a halogen;
X² is selected from N and CH;
X³ is selected from O and NH;
n is an integer from 1 to 6; and
each L¹ is independently selected from O, S, S(═O)₂, NR^{N}, C═O, C═S, and C₁₋₆ alkylene.

In some embodiments, the compound has formula:

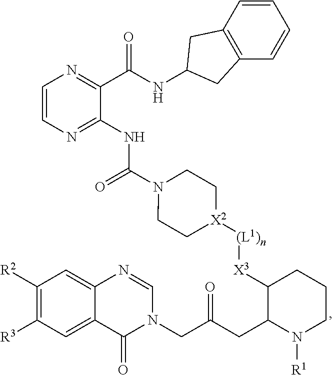

or a pharmaceutically acceptable salt thereof.

In some embodiments, the compound has formula:

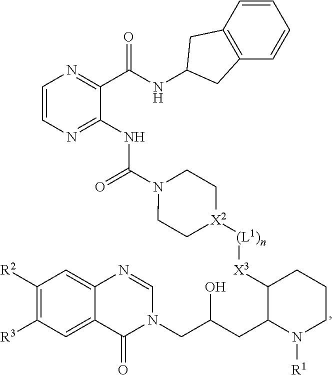

or a pharmaceutically acceptable salt thereof.

In some embodiments, R¹ is H.

In some embodiments, R¹ is C₁₋₃ alkyl or C₁₋₃ haloalkyl.

In some embodiments, R¹ is —C(═O)R^{A1}.

In some embodiments, R¹ is —C(═O)OR^{A1}.

In some embodiments, R^{A1} is H.

In some embodiments, R^{A1} is C₁₋₆ alkyl.

In some embodiments, R² and R³ are each independently Br or Cl.

In some embodiments, X³ is O.

In some embodiments, X³ is NH.

In some embodiments, n is selected from 3, 4, and 5.

In some embodiments, each L¹ is independently selected from S(═O)₂, NH, C═O, and C₁₋₃ alkylene.

In some embodiments, (L¹)ₙ comprises S(═O)₂NH.

In some embodiments, (L¹)ₙ comprises NHC(═O).

In some embodiments, (L¹)ₙ comprises NHS(═O)₂NH.

In some embodiments, (L¹)ₙ comprises CH₂N.

In some embodiments, X² is N.

In some embodiments, (L¹)ₙ comprises:

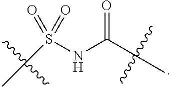 [SMILES: *C(=O)NS(*)(=O)=O]

In some embodiments, X² is CH.

In some embodiments, (L¹)ₙ comprises:

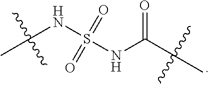 [SMILES: *NS(=O)(=O)NC(*)=O]

In some embodiments, (L¹)ₙ comprises:

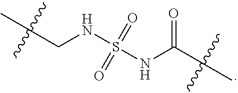 [SMILES: *CNS(=O)(=O)NC(*)=O]

In some embodiments, compound has formula:

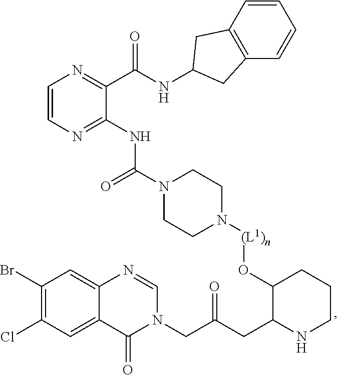

or a pharmaceutically acceptable salt thereof.

In some embodiments, compound has formula:

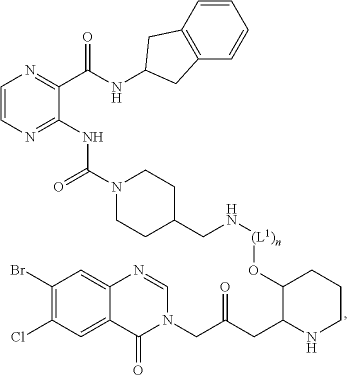

or a pharmaceutically acceptable salt thereof.

In some embodiments, compound has formula:

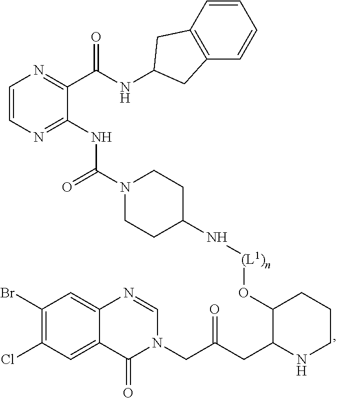

or a pharmaceutically acceptable salt thereof.

In some embodiments, the compound of Formula (II) is selected from any one of the following compounds:

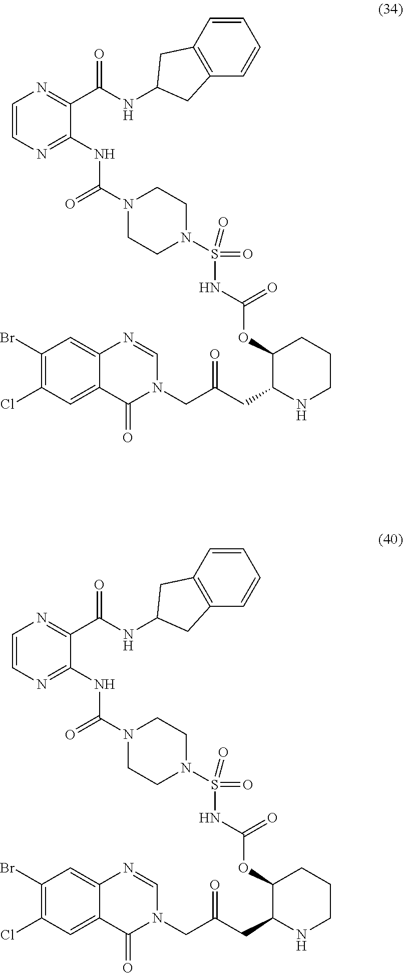 [SMILES: O=C(C[C@H]1NCCC[C@@H]1OC(=O)NS(=O)(=O)N1CCN(C(=O)Nc2nccnc2C(=O)NC2Cc3ccccc3C2)CC1)Cn1cnc2cc(Br)c(Cl)cc2c1=O ; O=C(C[C@@H]1NCCC[C@@H]1OC(=O)NS(=O)(=O)N1CCN(C(=O)Nc2nccnc2C(=O)NC2Cc3ccccc3C2)CC1)Cn1cnc2cc(Br)c(Cl)cc2c1=O]

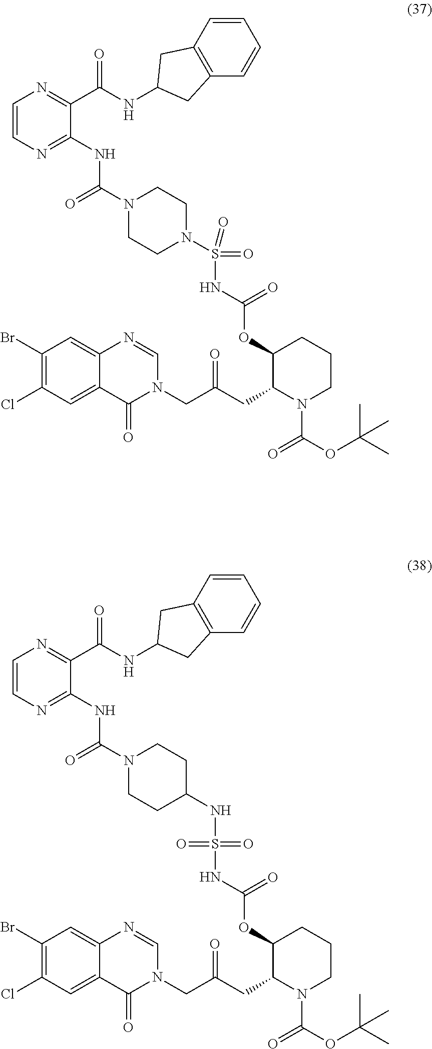 [SMILES: CC(C)(C)OC(=O)N1CCC[C@H](OC(=O)NS(=O)(=O)N2CCN(C(=O)Nc3nccnc3C(=O)NC3Cc4ccccc4C3)CC2)[C@H]1CC(=O)Cn1cnc2cc(Br)c(Cl)cc2c1=O ; CC(C)(C)OC(=O)N1CCC[C@H](OC(=O)NS(=O)(=O)NC2CCN(C(=O)Nc3nccnc3C(=O)NC3Cc4ccccc4C3)CC2)[C@H]1CC(=O)Cn1cnc2cc(Br)c(Cl)cc2c1=O]

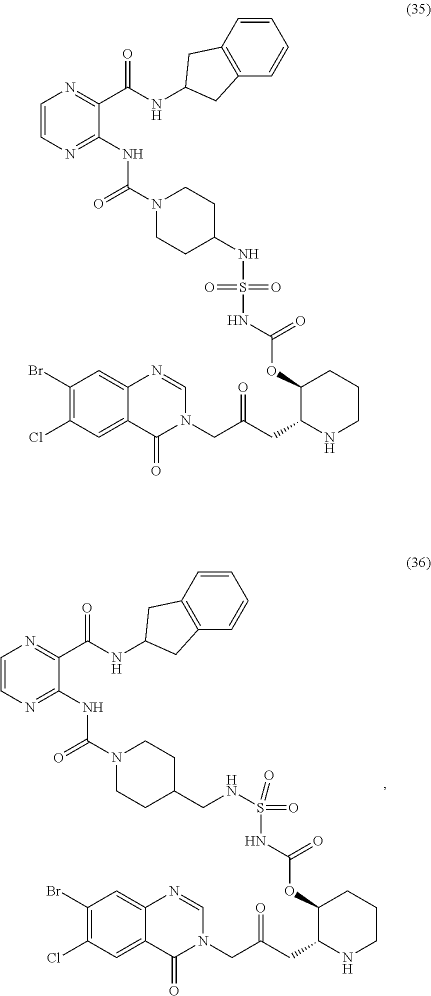 [SMILES: O=C(C[C@H]1NCCC[C@@H]1OC(=O)NS(=O)(=O)NC1CCN(C(=O)Nc2nccnc2C(=O)NC2Cc3ccccc3C2)CC1)Cn1cnc2cc(Br)c(Cl)cc2c1=O ; O=C(C[C@H]1NCCC[C@@H]1OC(=O)NS(=O)(=O)NCC1CCN(C(=O)Nc2nccnc2C(=O)NC2Cc3ccccc3C2)CC1)Cn1cnc2cc(Br)c(Cl)cc2c1=O]

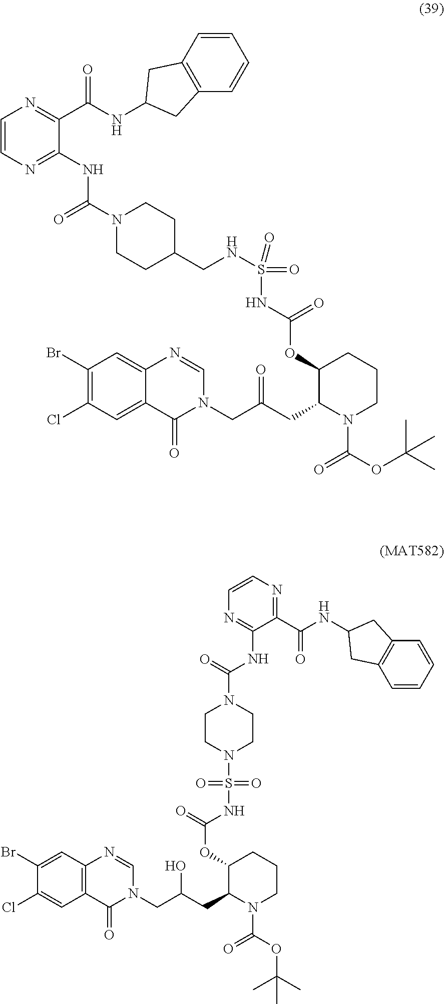 [SMILES: CC(C)(C)OC(=O)N1CCC[C@H](OC(=O)NS(=O)(=O)NCC2CCN(C(=O)Nc3nccnc3C(=O)NC3Cc4ccccc4C3)CC2)[C@H]1CC(=O)Cn1cnc2cc(Br)c(Cl)cc2c1=O ; CC(C)(C)OC(=O)N1CCC[C@@H](OC(=O)NS(=O)(=O)N2CCN(C(=O)Nc3nccnc3C(=O)NC3Cc4ccccc4C3)CC2)[C@@H]1CC(O)Cn1cnc2cc(Br)c(Cl)cc2c1=O]

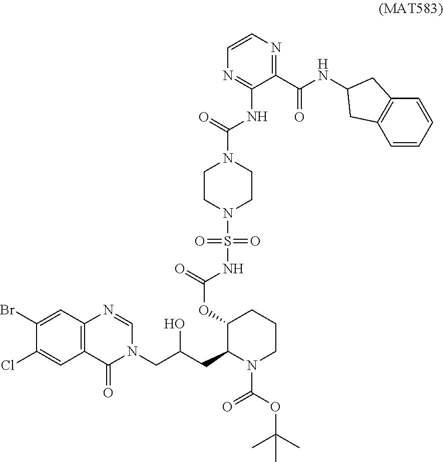 [SMILES: CC(C)(C)OC(=O)N1CCC[C@@H](OC(=O)NS(=O)(=O)N2CCN(C(=O)Nc3nccnc3C(=O)NC3Cc4ccccc4C3)CC2)[C@@H]1CC(O)Cn1cnc2cc(Br)c(Cl)cc2c1=O]

or a pharmaceutically acceptable salt thereof.

In some embodiments, the present disclosure provides a compound of formula:

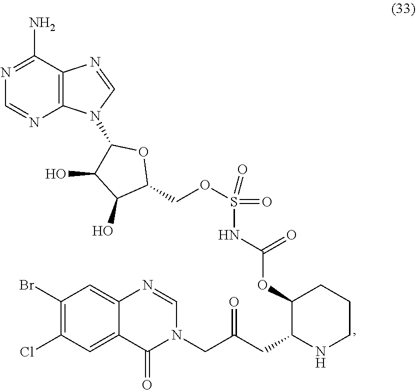 [SMILES: Nc1ncnc2c1ncn2[C@@H]1O[C@H](COS(=O)(=O)NC(=O)O[C@H]2CCCN[C@@H]2CC(=O)Cn2cnc3cc(Br)c(Cl)cc3c2=O)[C@@H](O)[C@H]1O]

or a pharmaceutically acceptable salt thereof.

In some embodiments, the compound is not a compound of formula:

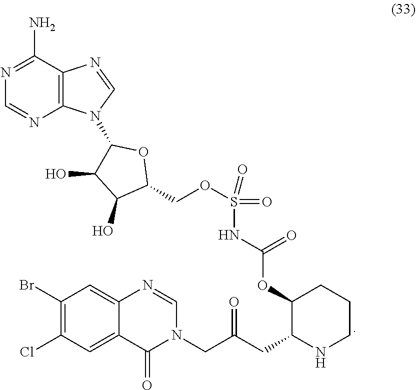 [SMILES: Nc1ncnc2c1ncn2[C@@H]1O[C@H](COS(=O)(=O)NC(=O)O[C@H]2CCCN[C@@H]2CC(=O)Cn2cnc3cc(Br)c(Cl)cc3c2=O)[C@@H](O)[C@H]1O]

In some embodiments, the present disclosure provides a compound of formula:

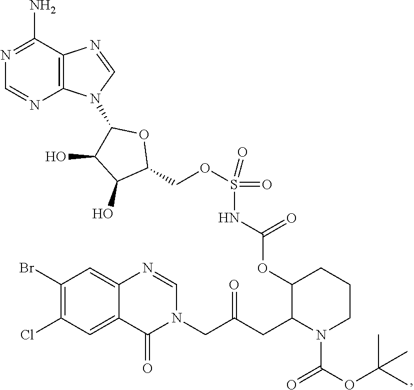 [SMILES: CC(C)(C)OC(=O)N1CCCC(OC(=O)NS(=O)(=O)OC[C@H]2O[C@@H](n3cnc4c(N)ncnc43)[C@H](O)[C@@H]2O)C1CC(=O)Cn1cnc2cc(Br)c(Cl)cc2c1=O]

or a pharmaceutically acceptable salt thereof.

In some embodiments, the present disclosure provides a compound of Formula (III):

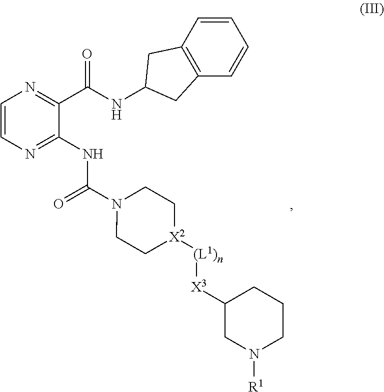

or a pharmaceutically acceptable salt thereof, wherein:
R¹ is selected from H, C₁₋₃ alkyl, C₁₋₃ haloalkyl, —C(═O)R^{A1}, and —C(═O)OR^{A1};
each R^{A1} is selected from H, C₁₋₆ alkyl, and C₁₋₃ haloalkyl;
X² is selected from N and CH;
X³ is selected from O and NH;
n is an integer from 1 to 6; and
each L¹ is independently selected from O, S, S(═O)₂, NR^{N}, C═O, C═S, and C₁₋₆ alkylene.

In some embodiments, R¹ is H.

In some embodiments, R¹ is C₁₋₃ alkyl or C₁₋₃ haloalkyl.

In some embodiments, R¹ is —C(═O)R^{A1}.

In some embodiments, R¹ is —C(═O)OR^{A1}.

In some embodiments, R^{A1} is H.

In some embodiments, R^{A1} is C₁₋₆ alkyl.

In some embodiments, X³ is O.

In some embodiments, X³ is NH.

In some embodiments, n is selected from 3, 4, and 5.

In some embodiments, each L¹ is independently selected from S(═O)₂, NH, C═O, and C₁₋₃ alkylene.

In some embodiments, (L¹)ₙ comprises S(═O)₂NH.

In some embodiments, (L¹)ₙ comprises NHC(═O).

In some embodiments, (L¹)ₙ comprises NHS(═O)₂NH.

In some embodiments, (L¹)ₙ comprises CH₂N.

In some embodiments, X² is N.

In some embodiments, (L¹)ₙ comprises:

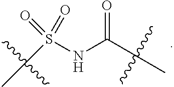 [SMILES: *C(=O)NS(*)(=O)=O]

In some embodiments, X² is CH.

In some embodiments, (L¹)ₙ comprises:

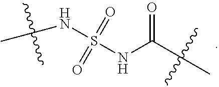 [SMILES: *NS(=O)(=O)NC(*)=O]

In some embodiments, (L¹)ₙ comprises:

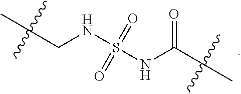 [SMILES: *CNS(=O)(=O)NC(*)=O]

In some embodiments, the compound has formula:

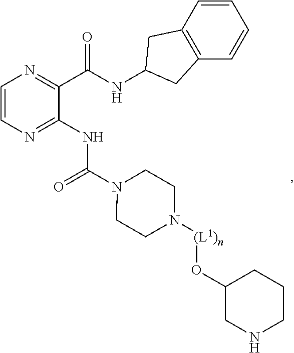

or a pharmaceutically acceptable salt thereof.

In some embodiments, the compound has formula:

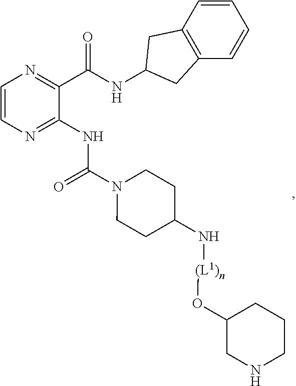

or a pharmaceutically acceptable salt thereof.

In some embodiments, the compound has formula:

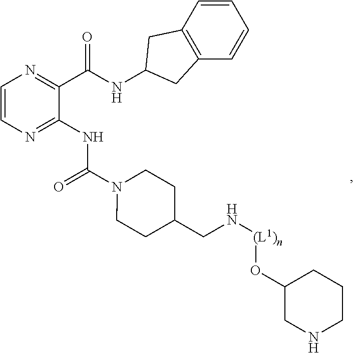

or a pharmaceutically acceptable salt thereof.

In some embodiments, the compound of Formula (III) is selected from any one of the following compounds:

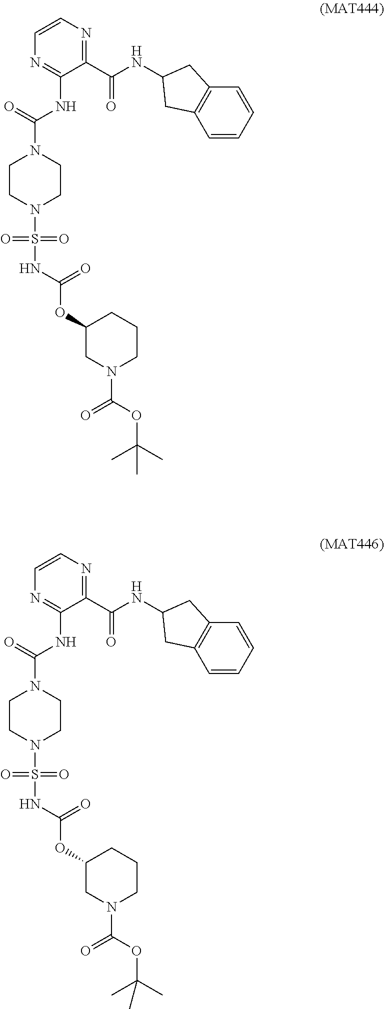 [SMILES: CC(C)(C)OC(=O)N1CCC[C@H](OC(=O)NS(=O)(=O)N2CCN(C(=O)Nc3nccnc3C(=O)NC3Cc4ccccc4C3)CC2)C1 ; CC(C)(C)OC(=O)N1CCC[C@@H](OC(=O)NS(=O)(=O)N2CCN(C(=O)Nc3nccnc3C(=O)NC3Cc4ccccc4C3)CC2)C1]

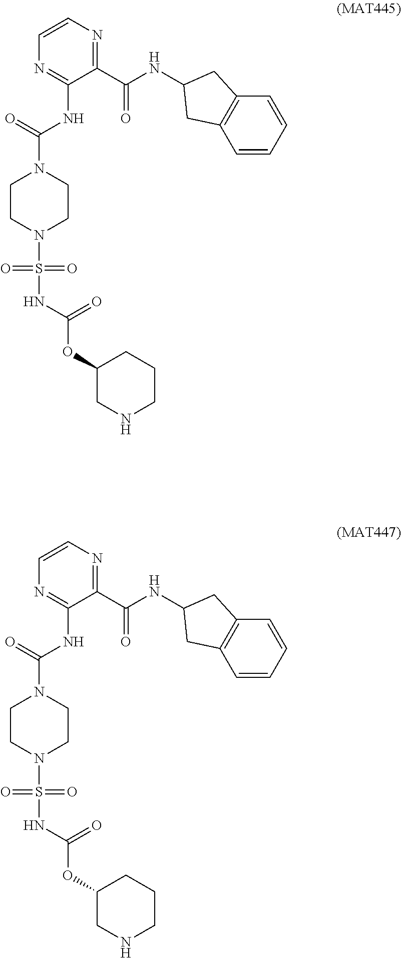 [SMILES: O=C(NS(=O)(=O)N1CCN(C(=O)Nc2nccnc2C(=O)NC2Cc3ccccc3C2)CC1)O[C@H]1CCCNC1 ; O=C(NS(=O)(=O)N1CCN(C(=O)Nc2nccnc2C(=O)NC2Cc3ccccc3C2)CC1)O[C@@H]1CCCNC1]

or a pharmaceutically acceptable salt thereof.

In some embodiments, the present disclosure provides a compound of Formula (IV):

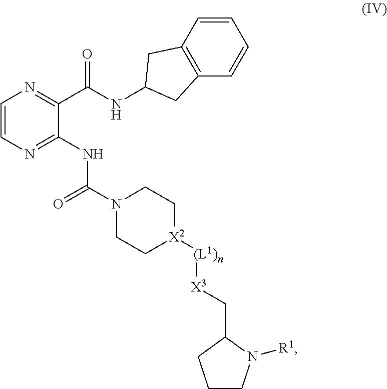

or a pharmaceutically acceptable salt thereof, wherein:
R¹ is selected from H, C₁₋₃ alkyl, C₁₋₃ haloalkyl, —C(═O)R^{A1}, and —C(═O)OR^{A1};
each R^{A1} is selected from H, C₁₋₆ alkyl, and C₁₋₃ haloalkyl;
X² is selected from N and CH;
X³ is selected from O and NH;
n is an integer from 1 to 6; and
each L¹ is independently selected from O, S, S(═O)₂, NR^{N}, C═O, C═S, and C₁₋₆ alkylene.

In some embodiments, R¹ is H.

In some embodiments, R¹ is C₁₋₃ alkyl or C₁₋₃ haloalkyl.

In some embodiments, R¹ is —C(═O)R^{A1}.

In some embodiments, R¹ is —C(═O)OR^{A1}.

In some embodiments, R^{A1} is H.

In some embodiments, R^{A1} is C₁₋₆ alkyl.

In some embodiments, X³ is O.

In some embodiments, X³ is NH.

In some embodiments, n is selected from 3, 4, and 5.

In some embodiments, each L¹ is independently selected from S(═O)₂, NH, C═O, and C₁₋₃ alkylene.

In some embodiments, (L¹)ₙ comprises S(═O)₂NH.

In some embodiments, (L¹)ₙ comprises NHC(═O).

In some embodiments, (L¹)ₙ comprises NHS(═O)₂NH.

In some embodiments, (L¹)ₙ comprises CH₂N.

In some embodiments, X² is N.

In some embodiments, (L¹)ₙ comprises:

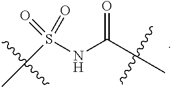 [SMILES: *C(=O)NS(*)(=O)=O]

In some embodiments, X² is CH.

In some embodiments, (L¹)ₙ comprises:

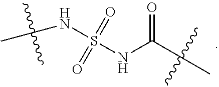 [SMILES: *NS(=O)(=O)NC(*)=O]

In some embodiments, (L¹)ₙ comprises:

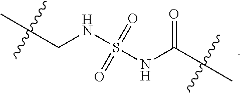 [SMILES: *CNS(=O)(=O)NC(*)=O]

In some embodiments, the compound of Formula (IV) has formula:

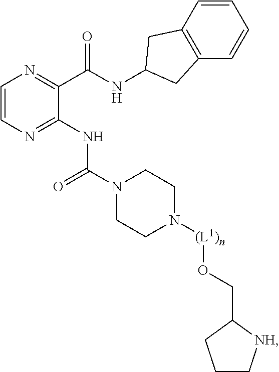

or a pharmaceutically acceptable salt thereof.

In some embodiments, the compound of Formula (IV) has formula:

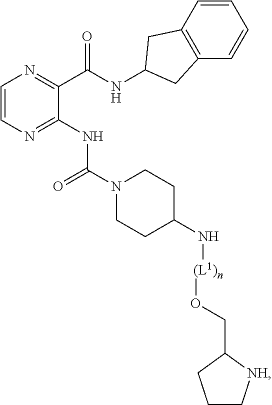

or a pharmaceutically acceptable salt thereof.

In some embodiments, the compound of Formula (IV) has formula:

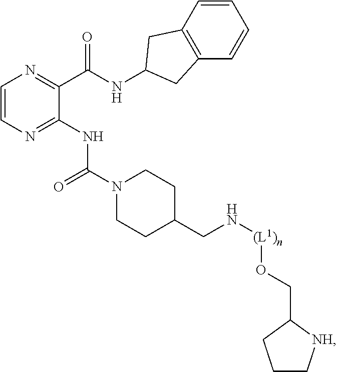

or a pharmaceutically acceptable salt thereof.

In some embodiments, the compound of Formula (IV) is selected from any one of the following compounds:

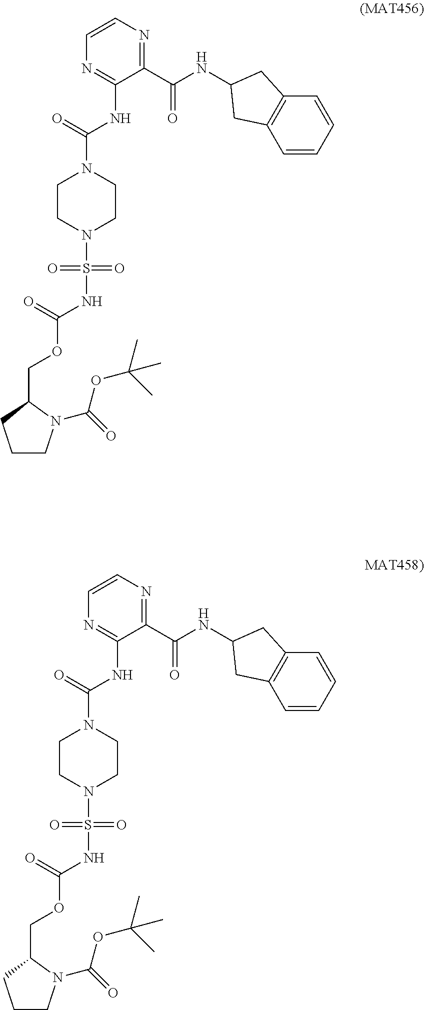 [SMILES: CC(C)(C)OC(=O)N1CCC[C@H]1COC(=O)NS(=O)(=O)N1CCN(C(=O)Nc2nccnc2C(=O)NC2Cc3ccccc3C2)CC1 ; CC(C)(C)OC(=O)N1CCC[C@@H]1COC(=O)NS(=O)(=O)N1CCN(C(=O)Nc2nccnc2C(=O)NC2Cc3ccccc3C2)CC1]

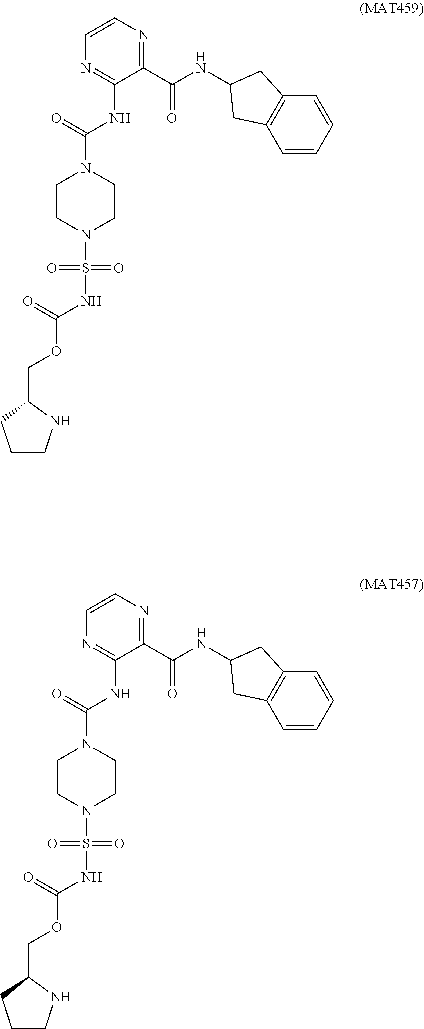 [SMILES: O=C(NS(=O)(=O)N1CCN(C(=O)Nc2nccnc2C(=O)NC2Cc3ccccc3C2)CC1)OC[C@H]1CCCN1 ; O=C(NS(=O)(=O)N1CCN(C(=O)Nc2nccnc2C(=O)NC2Cc3ccccc3C2)CC1)OC[C@@H]1CCCN1]

or a pharmaceutically acceptable salt thereof.

In some embodiments, the present disclosure provides a compound of formula:

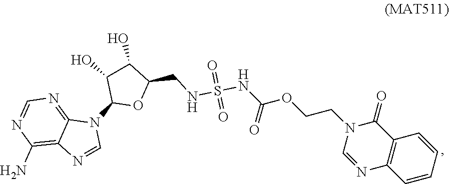 [SMILES: Nc1ncnc2c1ncn2[C@@H]1O[C@H](CNS(=O)(=O)NC(=O)OCCn2cnc3ccccc3c2=O)[C@@H](O)[C@H]1O]

or a pharmaceutically acceptable salt thereof.

In some embodiments, the present disclosure provides a compound of Formula (V):

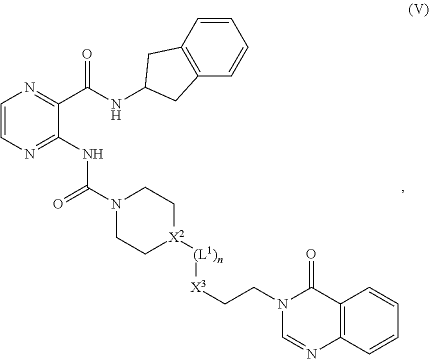

or a pharmaceutically acceptable salt thereof, wherein:
X² is selected from N and CH;
X³ is selected from O and NH;
n is an integer from 1 to 6; and
each L¹ is independently selected from O, S, S(═O)₂, NR^{N}, C═O, C═S, and C₁₋₆ alkylene.

In some embodiments, X³ is O.

In some embodiments, X³ is NH.

In some embodiments, n is selected from 3, 4, and 5.

In some embodiments, each L¹ is independently selected from S(═O)₂, NH, C═O, and C₁₋₃ alkylene.

In some embodiments, (L¹)ₙ comprises S(═O)₂NH.

In some embodiments, (L¹)ₙ comprises NHC(═O).

In some embodiments, (L¹)ₙ comprises NHS(═O)₂NH.

In some embodiments, (L¹)ₙ comprises CH₂N.

In some embodiments, X² is N.

In some embodiments, (L¹)ₙ comprises:

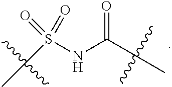 [SMILES: *C(=O)NS(*)(=O)=O]

In some embodiments, X² is CH.

In some embodiments, (L¹)ₙ comprises:

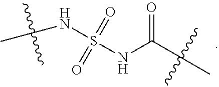 [SMILES: *NS(=O)(=O)NC(*)=O]

In some embodiments, (L¹)ₙ comprises:

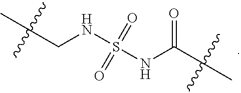 [SMILES: *CNS(=O)(=O)NC(*)=O]

In some embodiments, the compound has formula:

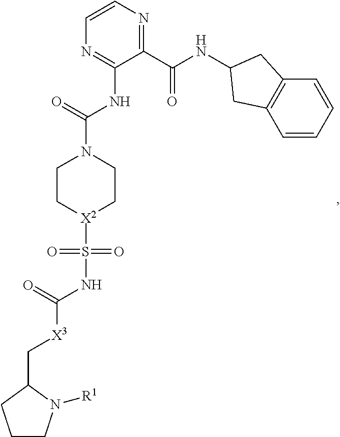

or a pharmaceutically acceptable salt thereof.

In some embodiments, the compound has formula:

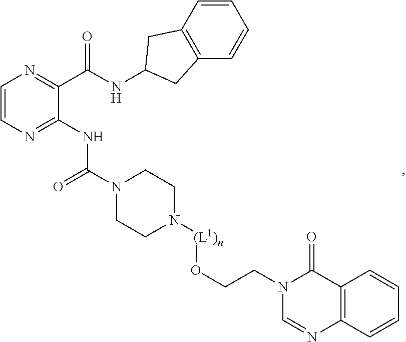

or a pharmaceutically acceptable salt thereof.

In some embodiments, the compound has formula:

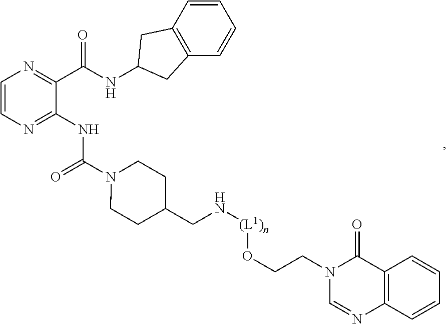

or a pharmaceutically acceptable salt thereof.

In some embodiments, the compound has formula:

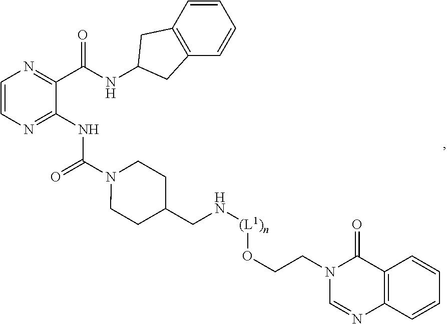

or a pharmaceutically acceptable salt thereof.

In some embodiments, the compound of Formula (V) is selected from any one of the following compounds:

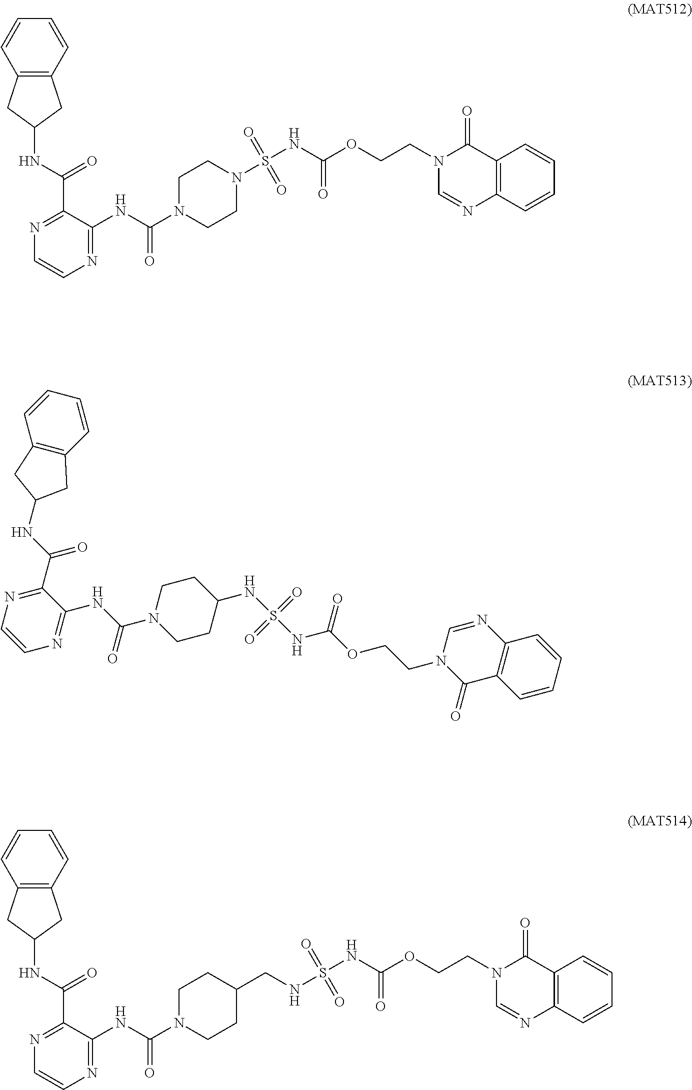 [SMILES: O=C(NS(=O)(=O)N1CCN(C(=O)Nc2nccnc2C(=O)NC2Cc3ccccc3C2)CC1)OCCn1cnc2ccccc2c1=O ; O=C(NS(=O)(=O)NC1CCN(C(=O)Nc2nccnc2C(=O)NC2Cc3ccccc3C2)CC1)OCCn1cnc2ccccc2c1=O ; O=C(NS(=O)(=O)NCC1CCN(C(=O)Nc2nccnc2C(=O)NC2Cc3ccccc3C2)CC1)OCCn1cnc2ccccc2c1=O]

or a pharmaceutically acceptable salt thereof.

### Pharmaceutically Acceptable Salts

In some embodiments, a salt of a compound is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid. In some embodiments, bases commonly employed to form pharmaceutically acceptable salts of the compounds include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. In some embodiments, the compounds or pharmaceutically acceptable salts thereof, are substantially isolated.

### Methods of Use

The present disclosure also provides methods of inhibiting glutamyl-prolyl-tRNA synthetase, prolyl-tRNA synthetase, or a combination thereof. The inhibiting may be carried out in a cell, such as in vitro, in vivo, or ex vivo. In one example, the disclosure provides a method of inhibiting prolyl-tRNA-synthetase in a cell, comprising contacting the cell with a compound of this disclosure, or a pharmaceutically acceptable salt thereof. In some embodiments, the cell is a human cell or a protozoan parasitic cell. In some embodiments, the cell is a human cell (e.g., cancer cell). In some embodiments, the cell is a protozoan parasitic cell. In some embodiments, the protozoan parasitic cell is a Plasmodium parasitic cell. In some embodiments, the protozoan parasitic cell is a Plasmodium falciparum. In some embodiments, the protozoan parasitic cell is selected from the group consisting of a Cryptosporidium, Babesia, Cyclospora, Cystoisospora, Toxoplasma, Giardia, and Plasmodia parasitic cell. In some embodiments, the protozoan parasitic cell is selected a Plasmodia parasitic cell. In some embodiments, the protozoan parasitic cell is selected from the group consisting of Plasmodium vivax, Plasmodium falciparum, Plasmodium malariae, Plasmodium ovale, and Plasmodium knowlesi.

In some embodiments, the present disclosure provides a method of inhibiting prolyl-tRNA-synthetase (e.g., a glutamyl-prolyl-tRNA synthetase) in a subject, comprising administering to the subject an effective amount of a compound as described herein, or a pharmaceutically acceptable salt thereof.

In some embodiments, the human has been infected with protozoan parasite. In some embodiments, the human has been identified as having been infected with protozoan parasite. In some embodiments, the protozoan parasite is selected from the group consisting of Cryptosporidium, Babesia, Cyclospora, Cystoisospora, Toxoplasma, Giardia, and Plasmodium. In some embodiments, the human has been infected with a Plasmodium parasite. In some embodiments, the human has been identified as having been infected with a Plasmodium parasite. In some embodiments, the human has been identified as having been infected with a Plasmodium parasite (e.g., a drug resistant Plasmodium parasite) selected from the group consisting of Plasmodium vivax, Plasmodium falciparum, Plasmodium malariae, Plasmodium ovale, and Plasmodium knowlesi. In some embodiments, the human has been infected with Plasmodium falciparum. In some embodiments, the human has been identified as having been infected with Plasmodium falciparum. In some embodiments, the infected human is diagnosed with malaria.

The present application further provides methods of treating a disorder in a subject (e.g., a subject in need thereof). In some embodiments, the disorder is associated with (e.g., abnormal activity) glutamyl-prolyl-tRNA synthetase, prolyl-tRNA synthetase, or a combination thereof. The method typically includes administering to a subject a therapeutically effective amount of a compound of this disclosure, or a pharmaceutically acceptable salt thereof. In one example, the subject is in need of treatment, for example, the subject may be diagnosed with the disorder by a treating physician.

In some embodiments, the disorder is a parasitic infection. In some embodiments, the parasite is a protozoan parasite. In some embodiments, the parasite is a protozoan parasite selected from the group consisting of Cryptosporidium, Babesia, Cyclospora, Cystoisospora, Toxoplasma, Giardia, and Plasmodium. In some embodiments, the parasite is a Plasmodium parasite. In some embodiments, the parasite is a drug resistant parasite. In some embodiments, the parasite is a drug resistant Plasmodium parasite. In some embodiments, the parasite is Plasmodium falciparum. In some embodiments, the Plasmodium parasite (e.g., a drug resistant Plasmodium parasite) is selected from the group consisting of Plasmodium vivax, Plasmodium falciparum, Plasmodium malariae, Plasmodium ovale, and Plasmodium knowlesi. In some embodiments, the parasite is a drug resistant Plasmodium falciparum.

In some embodiments, the parasitic infection is selected from malaria, toxoplasmosis, leishmaniasis, cryptosporidiosis, coccidiosis, Chagas disease, African sleeping sickness, giardiasis, and babesiosis. In some embodiments, the disorder is malaria. In some embodiments, the infectious disease is malaria, wherein the malaria is associated with a Plasmodium parasite. In some embodiments, the infectious disease is malaria, wherein the malaria is associated with Plasmodium falciparum. In some embodiments, the Plasmodium falciparum is a drug resistant Plasmodium falciparum.

In some embodiments, the disorder is an autoimmune disease. In some embodiments, the autoimmune disease is selected from multiple sclerosis, rheumatoid arthritis, lupus, psoriasis, scleroderma, dry eye syndrome, Crohn's Disease, inflammatory bowel disease, chronic obstructive pulmonary disease (COPD), asthma, fibrosis, scar formation, ischemic damage, and graft versus host disease.

In some embodiments, the disorder is a bacterial infection. In some embodiments, the disorder is a fungal infection. In some embodiments, the disorder is a viral infection. In some embodiments, the viral infection caused by corona virus, dengue virus or chikungunya virus.

In some embodiments, the disorder is selected from neurological disorder (e.g., Alzheimer's, Parkinson's, Huntington's, or ALS), a genetic disorder, a cardiovascular disorder (e.g., ischemia, stroke), a protein aggregation disorder, a metabolic disorder, an inflammatory disorder, and a cosmetic disorder. Compounds of the present disclosure may also be used to promote wound healing and/or prevent scarring and may be useful cosmetically.

In some embodiments, the disorder is amino acid response (AAR)-mediated condition or a Th17-mediated condition. In certain embodiments, compounds of the present invention may be used to inhibit pro-fibrotic behavior in fibroblasts or inhibit the differentiation of Th 17 cells. Therefore, provided compounds may be useful in preventing fibrosis. Provided compounds may also be used as probes of biological pathways. Provided compounds may also be used in studying the differentiation of T cells.

In some embodiments, the genetic disorder is Duchenne muscular dystrophy. In some embodiments, the metabolic disorder is selected from diabetes and obesity. In some embodiments, the cosmetic disorder is selected from the group consisting of cellulite and stretch marks. In some embodiments, the inflammatory disorder is selected from restenosis, macular degeneration, choroidal neovascularization, and chronic inflammation. The disorder may also be a disorder involving angiogenesis, such as cancer. In some embodiments, the disorder is cancer. In some embodiments, the cancer is a T-cell neoplasm selected from mature T-cell leukemia, nodal peripheral T-cell lymphoma (PTCL), extranodal PTCLs, and cutaneous T-cell lymphoma (CTCL). In some embodiments, the cancer is selected from adrenocortical carcinoma, bladder urothelial carcinoma, breast invasive carcinoma, cervical squamous cell carcinoma and endocervical adenocarcinoma, cholangio carcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine corpus endometrial carcinoma, uterine carcinosarcoma, uveal melanoma, multiple myeloma, and chordoma.

The cancer may be any one of cancers described, for example, in Wang et al., Genes 2020, 11, 1384, and Arita et al., Biochemical and Biophysical Research Communications 488 (2017) 648-654, both of which are incorporated here by reference in their entirety.

As used herein, the term “subject,” refers to any animal (e.g., a domesticated animal), including mammals. Exemplary subjects include, but are not limited to, mice, rats, rabbits, dogs, cats, swine, cattle, sheep, horses, primates, and humans. In some embodiments, the subject is a human. In some embodiments, the subject is an animal (e.g., a mammal). In some embodiments, the animal is selected from the group consisting of a rabbit, a dog, a cat, swine, cattle, sheep, a horse, and a primate.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease.

### Compositions, Formulations, and Routes of Administration

The present application also provides pharmaceutical compositions comprising an effective amount of a compound of the present disclosure disclosed herein, or a pharmaceutically acceptable salt thereof, and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

### Dosages and Regimens

In the pharmaceutical compositions of the present application, a compound of the present disclosure is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the proRS with a compound of the invention includes the administration of a compound of the present invention to an individual or patient, such as a human, having ProRS as well as, for example, introducing a compound of the invention into a sample containing a cellular or purified preparation containing the ProRS.

### EXAMPLES

### Example 1—Developments of ProRS Inhibitors

The active site of ProRS comprises three distinct pockets that bind ATP, proline, and the 3′-terminal adenosine residue of tRNA^{Pro} (A76), respectively (FIG. 1a). A class of HsProRS inhibitors is represented by T-3767758 (2) (FIG. 1a). Unlike halofuginone and analogs, which span the A76 and proline-binding sites and interact in an ATP-uncompetitive manner (i.e., the inhibitor affinity increases with increasing ATP concentration), this inhibitor class targets the ATP-binding pocket and features adjacent to the active site. Notably T-3767758 (2) (FIG. 1a) displayed proline-uncompetitive steady state kinetics for HsProRS. This property is desirable for halofuginone-tolerant strains with elevated intracellular proline (halofuginone-induced), as it allows to overcome or even select against this resistance mechanism. Sequence analysis of HsProRS and PfcProRS paralogs reveals overall high homology with several nonconserved residues in and adjacent to the binding site occupied by compound 2, suggesting that inhibitors containing its structural core are active against PfcProRS (FIG. 4).

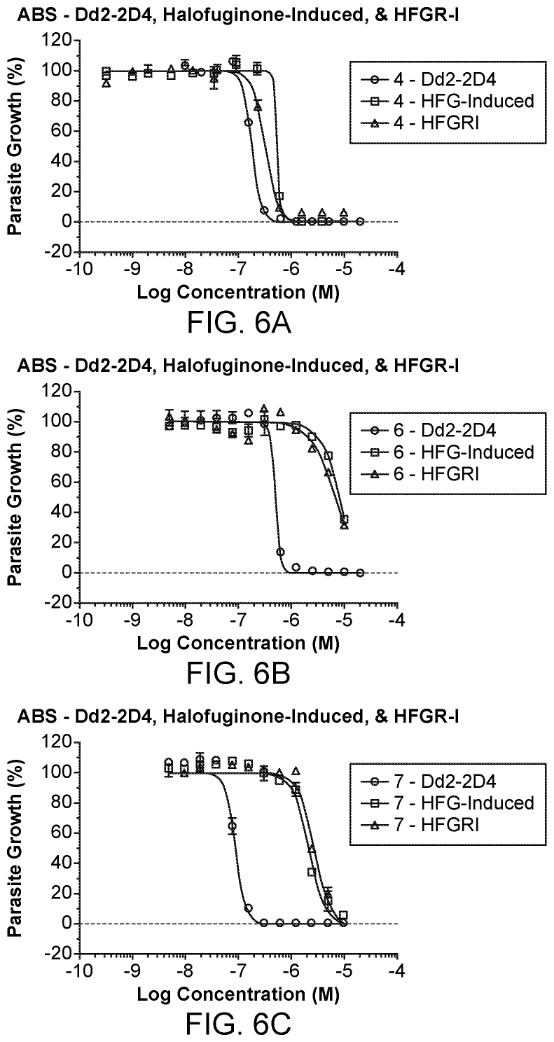

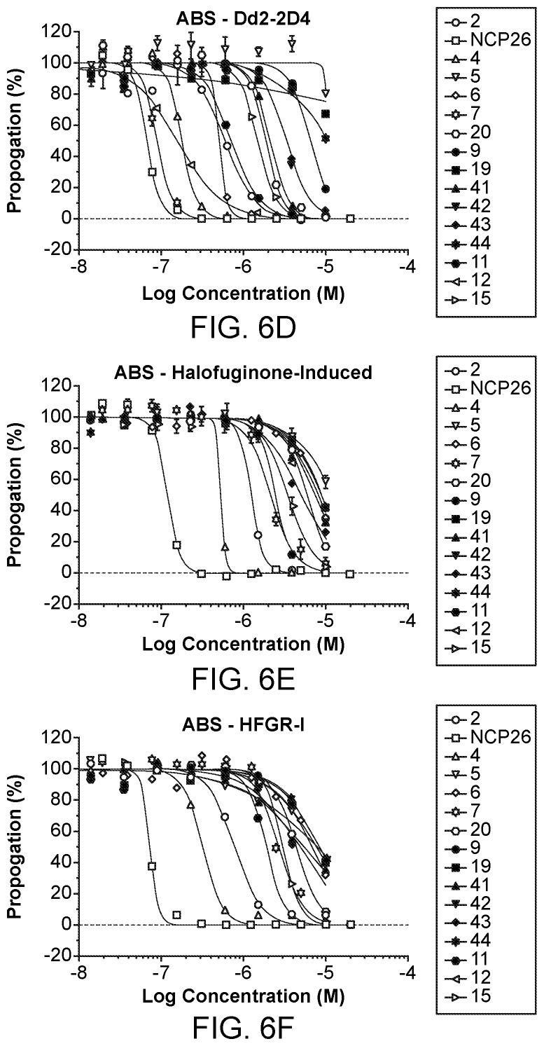

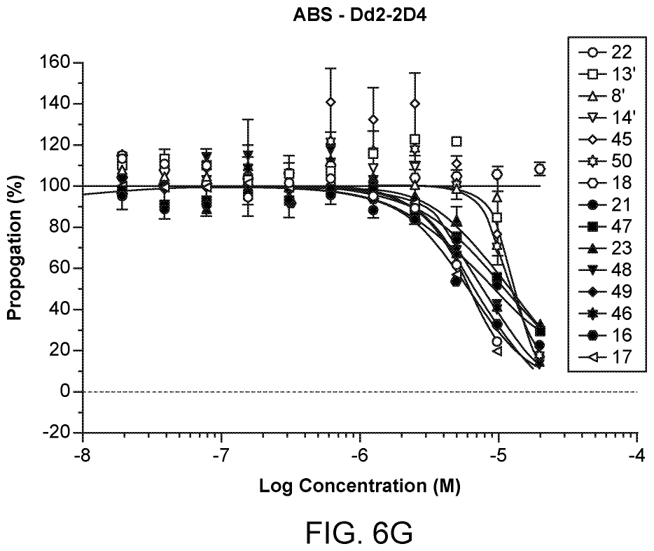

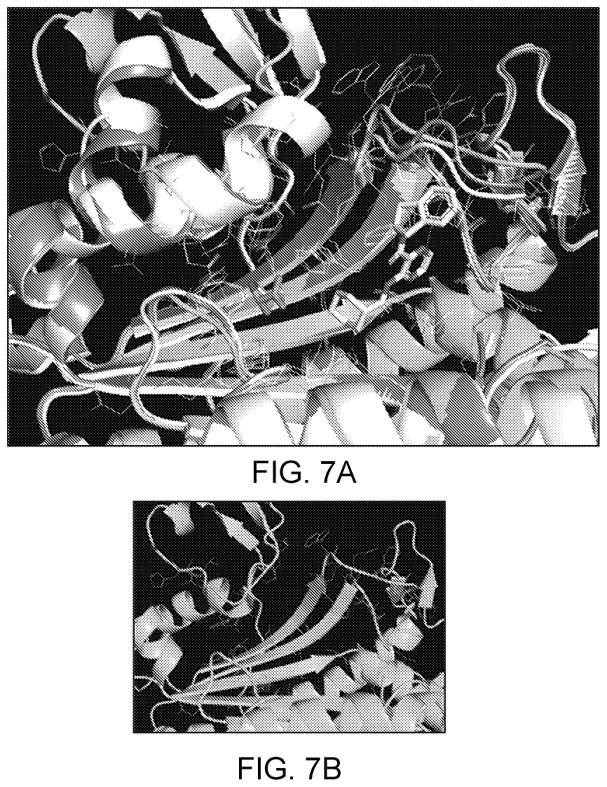

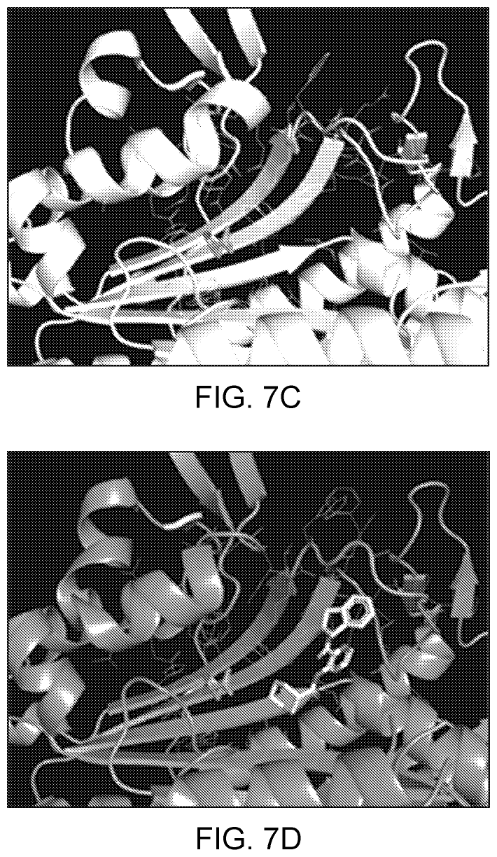

Although no synthetic protocols for compound 2 or analogs have been reported, the compound was prepared via a concise synthetic strategy starting from 3-aminopyrazine-2-carboxylic acid (see Example 2). The activity of compound 2 against asexual blood stage P. falciparum, which displayed good activity (EC₅₀=595 nM) against Dd2-2D4 wildtype parasites. Importantly, compound 2 did not show meaningful cross-resistance in halofuginone-induced parasites (EC₅₀=1.25 μM) and HFGR-I parasites (EC₅₀=736 nM), which, in addition to elevated proline, also feature the PfcProRS^{L482H} mutation that renders them ˜400-fold less sensitive to halofuginone (FIG. 1b). Analysis of reported co-crystal structure of compound 2 bound to HsProRS (PDB: 5VAD) revealed that the cyclohexyl substituent adopts an unfavorable axial geometry (FIG. 1c). Replacement of the cyclohexyl substituent with a piperidyl ring (NCP26, 3) or addition of a geminal methyl group (compound 4) are energetically more favorable and result in increased potency (FIG. 1d). Indeed, both compounds exhibited substantially increased activity against P. falciparum Dd2-2D4 (NCP26 EC₅₀=67.4 nM and compound 4 EC₅₀=180 nM), while retaining comparable potency against halofuginone-induced (NCP26 EC₅₀=120 nM and compound 4 EC₅₀=540 nM) and HFGR-I (NCP26 EC₅₀=68.6 nM and compound 4 EC₅₀=290 nM) parasites (FIG. 1b, FIG. 6a). Moreover, unlike halofuginone, NCP26 did not induce rapid resistance in wild-type parasites that were subjected to intermittent drug pressure.

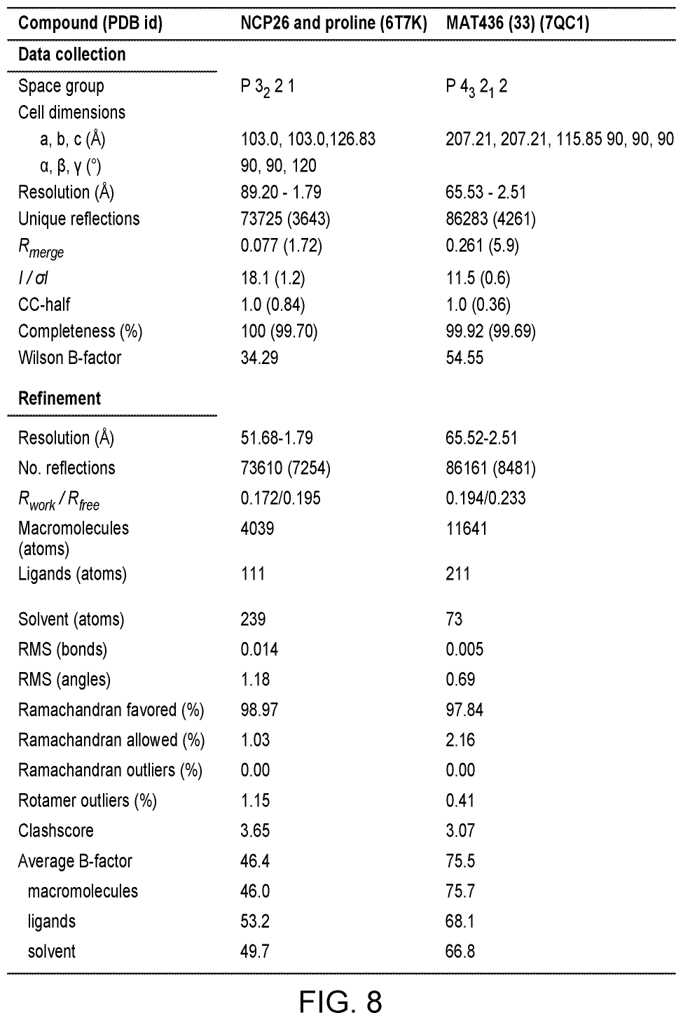

The co-crystal structure of NCP26 (FIG. 1e, PDB: 6T7K, and FIG. 8) in complex with PfcProRS is shown in the figures. Notably, crystallization efforts were only successful in the presence of proline. The piperidyl substituent of NCP26 adopted an energetically more favorable geometry compared to compound 2. Furthermore, the formal cyclohexyl to piperidine substitution eliminates a potential stereocenter. A library of compounds (within the present claims) was prepared, including those modified in the 4-position of the cyclohexyl/piperidinyl moiety (FIG. 1d). Activity profiling of this inhibitor set against wildtype ABS P. falciparum revealed inhibitors with nanomolar potency. For example, 4-BOC-amino group in compound compounds 6 and 7 was well tolerated, yielding inhibitors with comparable activity in wild-type parasites (FIG. 1d, Table 1, and FIG. 6b-d,g). However, unlike the parent compounds, compounds 6 and 7 exhibited significantly decreased activity in halofuginone-induced parasites (FIG. 6e). Interestingly, the activity was not further reduced in the HFGR-I line (FIG. 6f), which is consistent with a proline-competitive binding mode, but no direct interaction within the proline-binding pocket.

Next, the compounds were prepared that are inhibitors (e.g., dual- and triple-site inhibitors) of ProRS that extend to the proline- and tRNA^{Pro} A76-binding pockets, mimicking ProSA (25) or the halofuginone/ATP complex, respectively. These compounds bind to ProSA with high affinity.

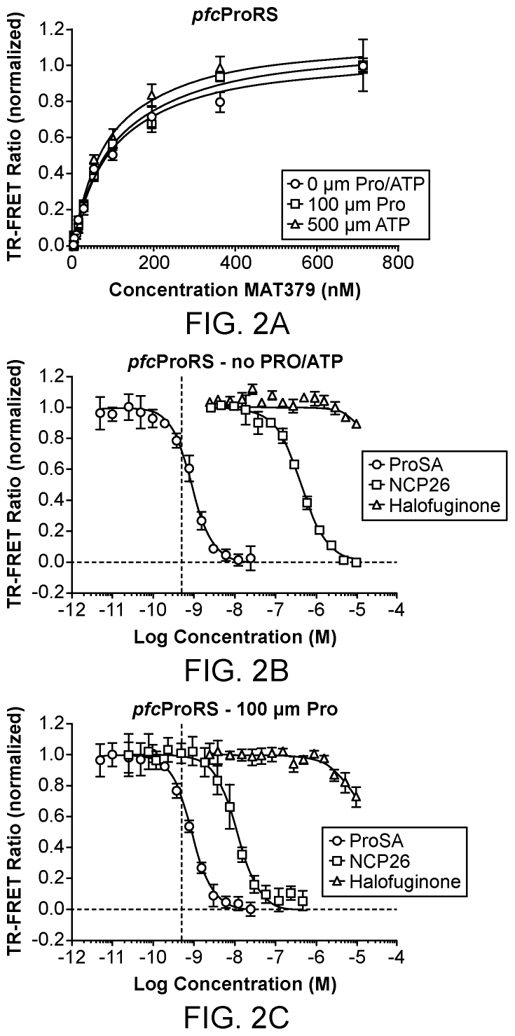

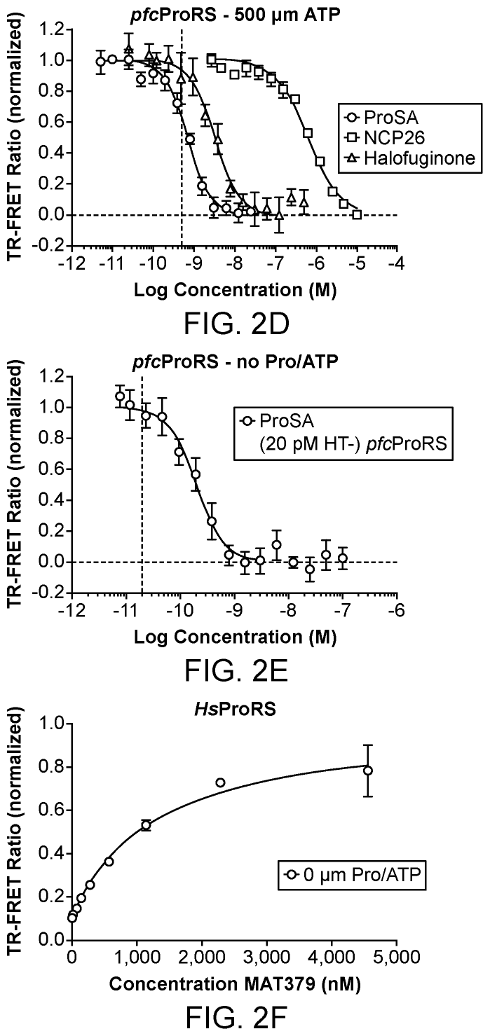

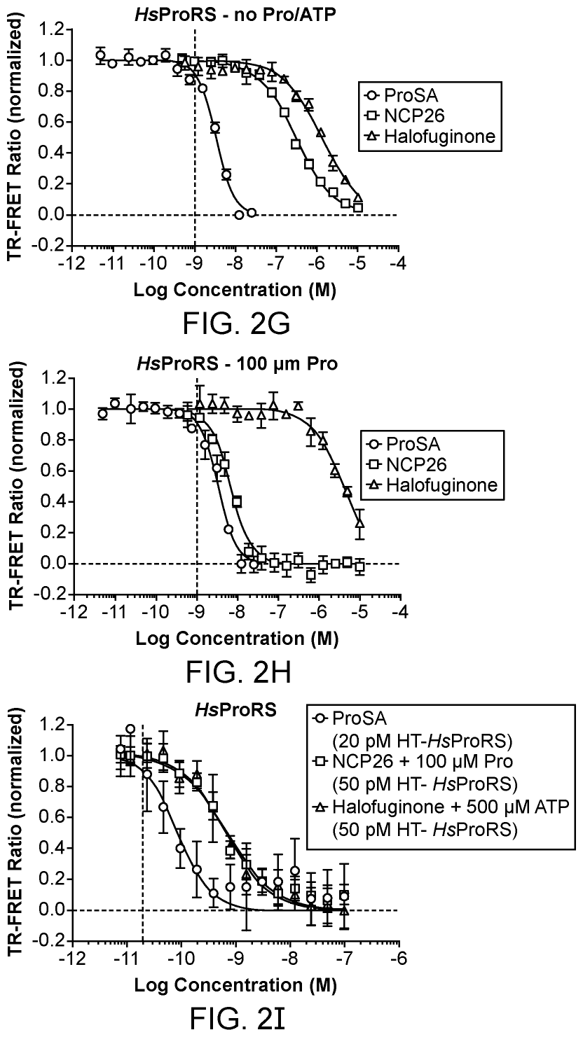

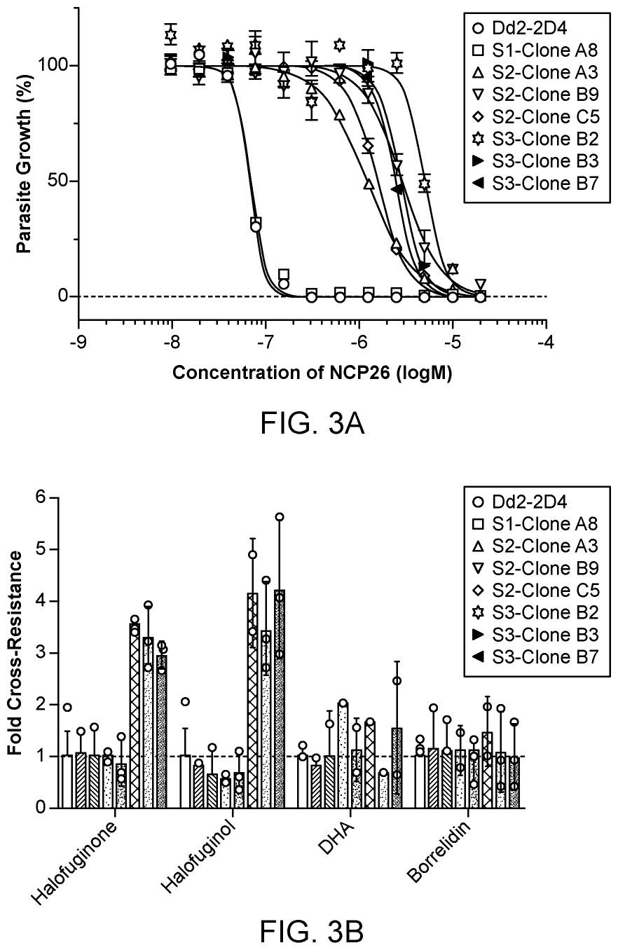

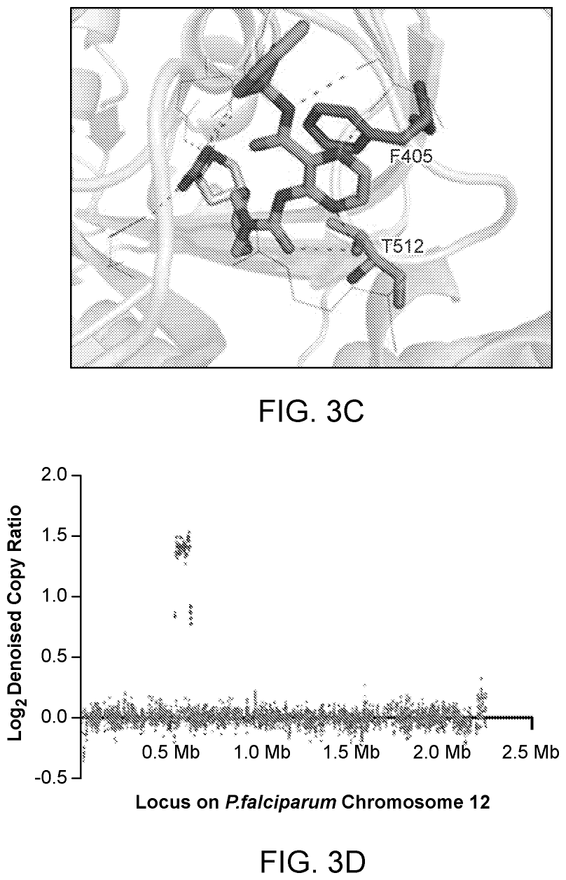

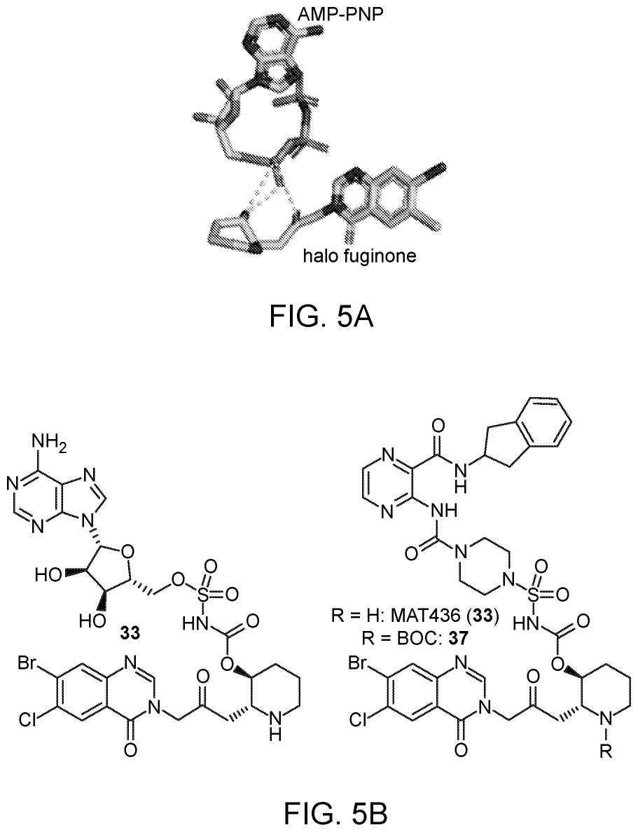

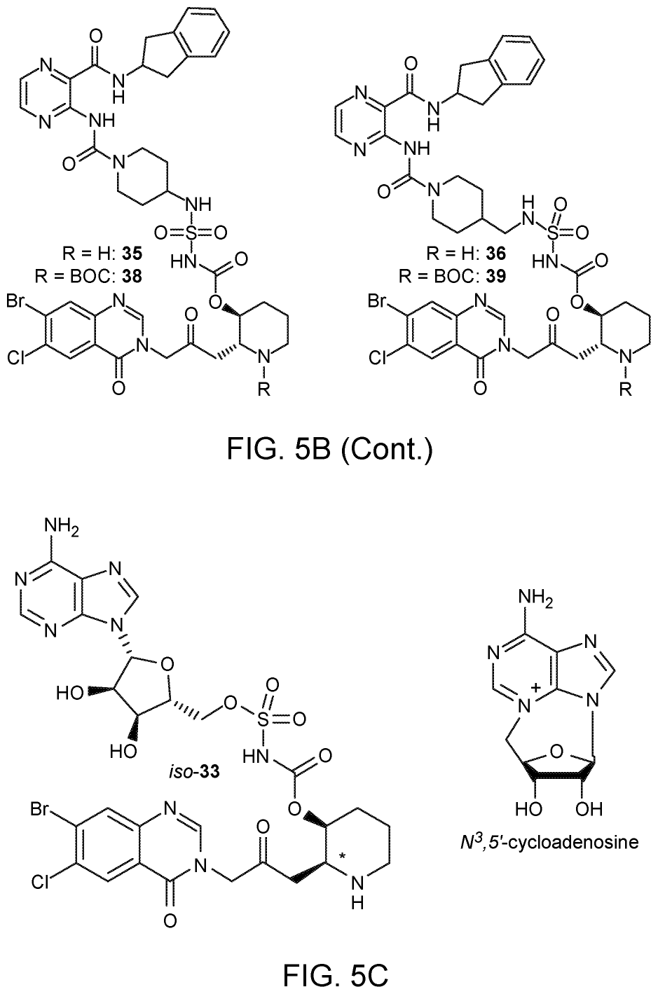

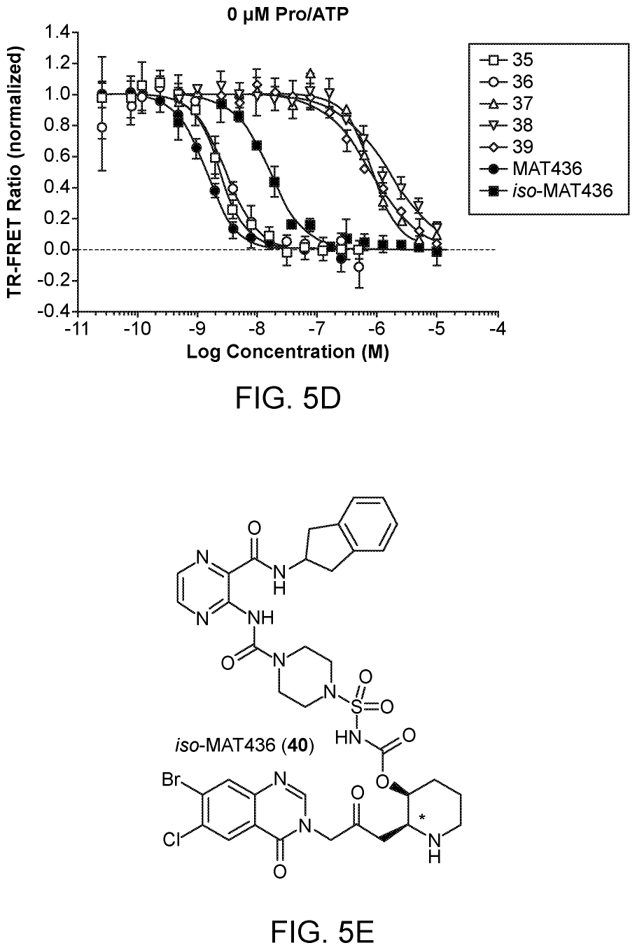

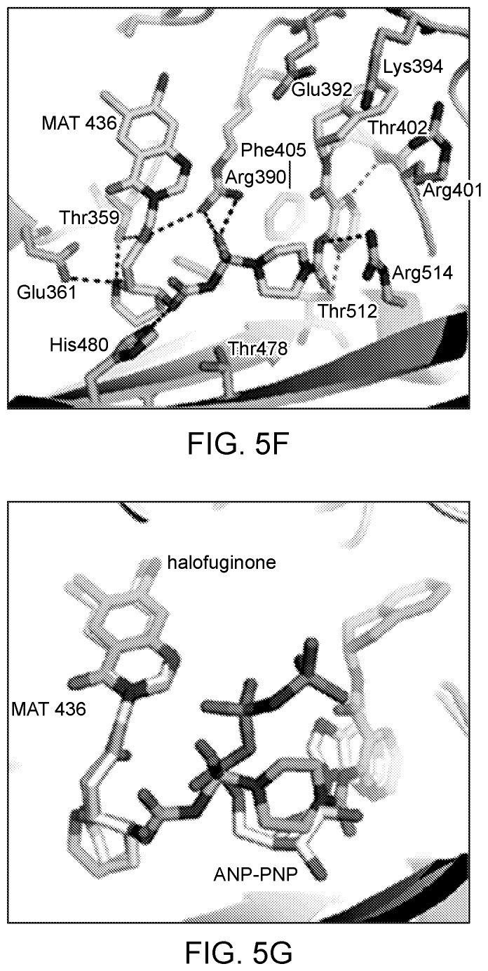

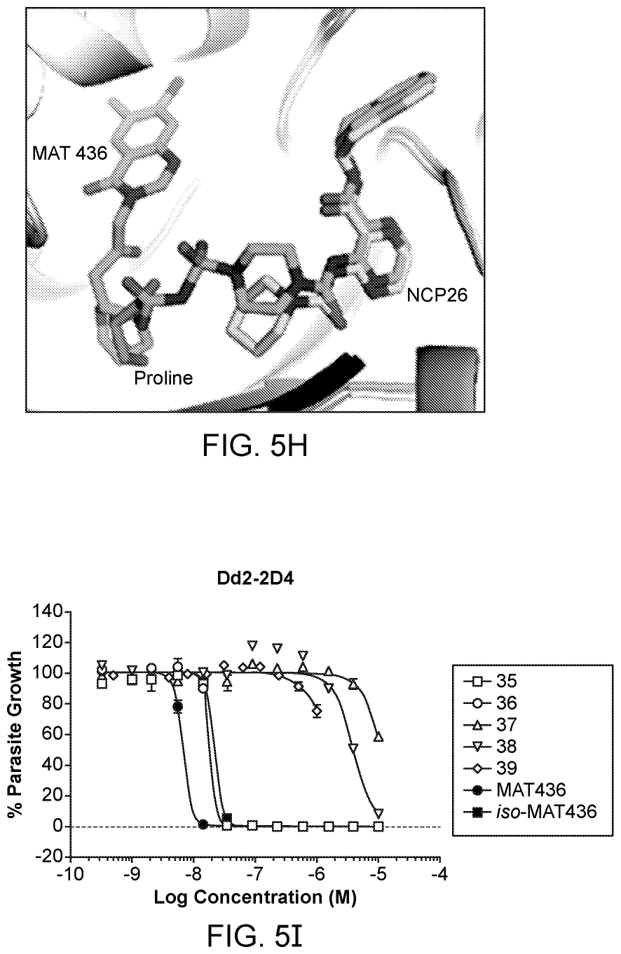

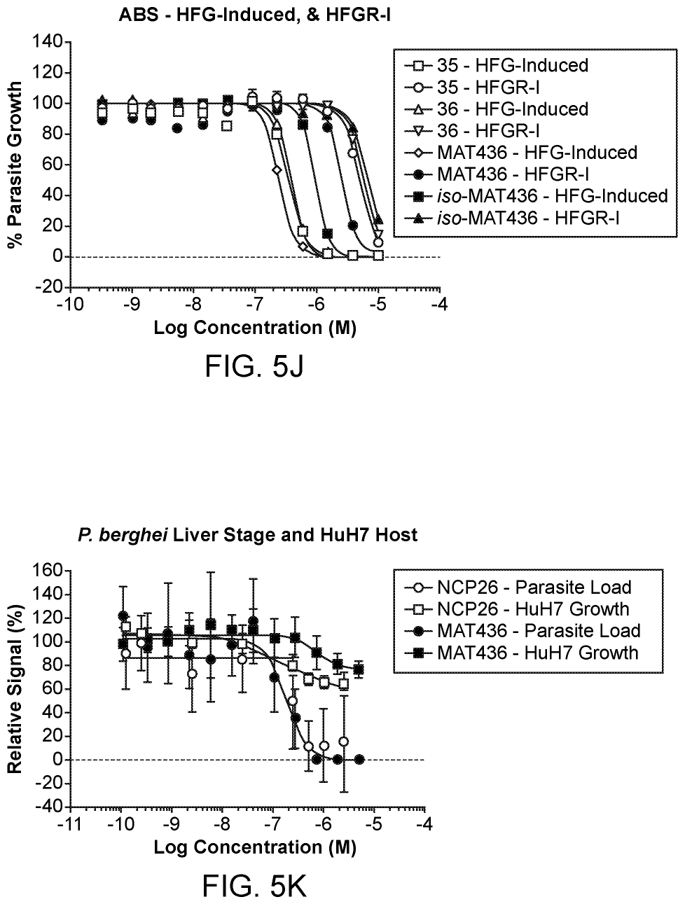

Analysis of the co-crystal structure of halofuginone and ATP with ProRS illustrates the molecular basis of halofuginone's ATP-uncompetitive binding mode. The ketone and hydroxyl-group of halofuginone form two well-defined hydrogen bonds with the α-phosphate of ATP, furnishing the intrinsic inhibitory complex in these ProRS ligand structures (FIG. 5a). The importance of ATP for halofuginone binding to PfcProRS and HsProRS is confirmed by biochemical data that show three orders of magnitude lower affinity in the absence of ATP (see FIG. 2).

A compound was designed where various moieties of the compound occupy all three ProRS binding sites, becoming a true triple-site inhibitor. Compound 33 was prepared as a “hybrid” of halofuginone and adenosine that joined via a sulfamoylcarbamate, analogous to ProSA (FIG. 5b):

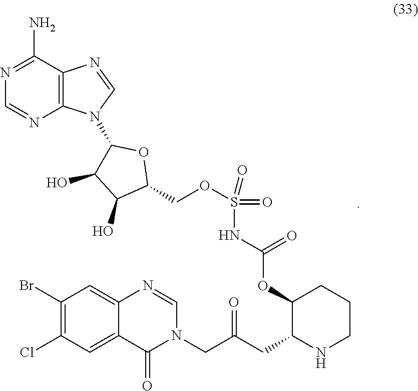 [SMILES: Nc1ncnc2c1ncn2[C@@H]1O[C@H](COS(=O)(=O)NC(=O)O[C@H]2CCCN[C@@H]2CC(=O)Cn2cnc3cc(Br)c(Cl)cc3c2=O)[C@@H](O)[C@H]1O]

Compound 33 was prone to fragmentation by intramolecular cyclization to N³,5′-cycloadenosine, which has been observed previously for aminoacyl-sulfamoyl-adenosine (aaSA) analogs, and the 2-position of the hydroxypiperidine was susceptible to epimerization to iso-33 during purification, which was previously observed with halofuginone (FIG. 5c).

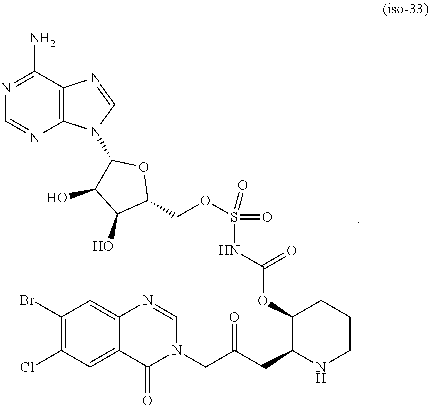 [SMILES: Nc1ncnc2c1ncn2[C@@H]1O[C@H](COS(=O)(=O)NC(=O)O[C@H]2CCCN[C@H]2CC(=O)Cn2cnc3cc(Br)c(Cl)cc3c2=O)[C@@H](O)[C@H]1O]

Pyrazinamide-halofuginone hybrids were prepared exploring three different linker-elements, while retaining the sulfamidylcarbamate as an acylphosphate isostere. Compound MAT436 (34) fitted well in the substrate binding pocket in the modeling studies:

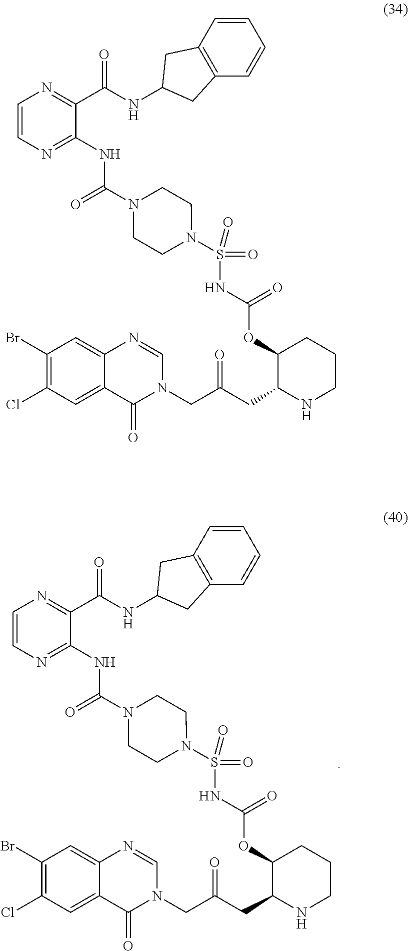 [SMILES: O=C(C[C@H]1NCCC[C@@H]1OC(=O)NS(=O)(=O)N1CCN(C(=O)Nc2nccnc2C(=O)NC2Cc3ccccc3C2)CC1)Cn1cnc2cc(Br)c(Cl)cc2c1=O ; O=C(C[C@@H]1NCCC[C@@H]1OC(=O)NS(=O)(=O)N1CCN(C(=O)Nc2nccnc2C(=O)NC2Cc3ccccc3C2)CC1)Cn1cnc2cc(Br)c(Cl)cc2c1=O]

Linker homologs 35 and 36 were prepared (FIG. 5b):

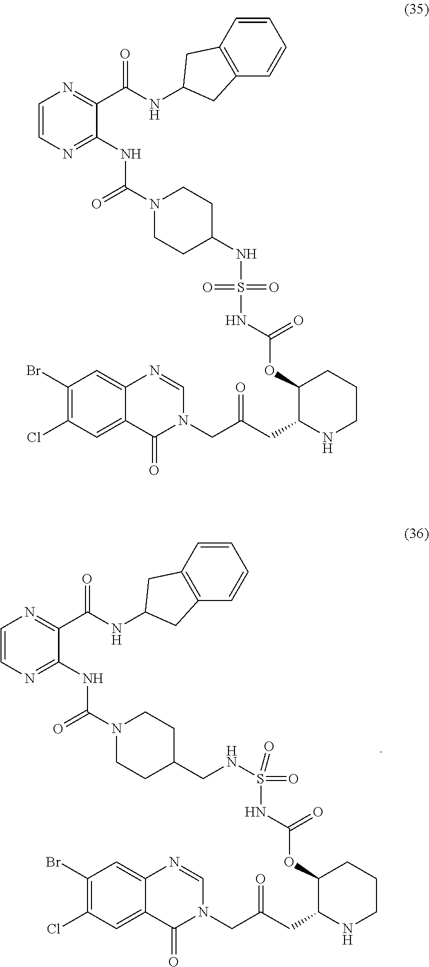 [SMILES: O=C(C[C@H]1NCCC[C@@H]1OC(=O)NS(=O)(=O)NC1CCN(C(=O)Nc2nccnc2C(=O)NC2Cc3ccccc3C2)CC1)Cn1cnc2cc(Br)c(Cl)cc2c1=O ; O=C(C[C@H]1NCCC[C@@H]1OC(=O)NS(=O)(=O)NCC1CCN(C(=O)Nc2nccnc2C(=O)NC2Cc3ccccc3C2)CC1)Cn1cnc2cc(Br)c(Cl)cc2c1=O]

All compounds exhibited very high affinity for PfcProRS and HsProRS in the absence of ATP. As predicted, MAT436 was the highest affinity ligand and displayed exceptional affinity for PfcProRS (K_{D}=375 pM, >10⁴-fold compared to halofuginone in the absence of ATP), while compounds 35 (K_{D}=680 pM) and 36 (K_{D}=821 pM) were only marginally less potent (FIG. 5d). In contrast, the BOC-protected precursors (37, 38, 39), which cannot bind the proline-binding pocket because of steric constraints, were much weaker ligands for PfcProRS, suggesting that these compounds can still bind with the halofuginone moiety pointing outside the active site. Notably, as for the adenosine-halofuginone hybrid 33, MAT436 was susceptible to epimerization of the 2′-position of the piperidine under non-acidic conditions, resulting in the corresponding syn-substituted analog (iso-MAT436, 40), which had only ˜10-fold lower affinity (FIG. 5d-e and Table 1). No epimerization was observed for reduced analogs MAT583, or the BOC-protected precursor MAT582 (See example 2). However, these alcohol compounds had reduced affinity for PfcProRS and were less active in parasites.

To provide further support for the predicted binding modes, MAT436 was co-crystallized with PfcProRS and the structure solved to 2.51 Å resolution (PDB: 7QC1 and FIG. 8). As illustrated in FIG. 5f, MAT436 occupies the ATP-, proline-, and A76-binding pockets, where it forms electrostatic interactions with conserved residues similar to halofuginone, proline, and NCP26. An overlay of MAT436 bound to PfcProRS with the corresponding ternary complex of PfcProRS with halofuginone and AMP-PNP (PDB: 4Q15) revealed a nearly perfect overlap with halofuginone, while the pyrazinamides of MAT436 and NCP26 (PDB: 6T7K) were virtually superimposable (FIG. 5g,h). These results unambiguously validate the pyrazinamide-based compounds of this disclosure as the first ProRS triple-site inhibitor class.

Activity of the pyrazinamide-halofuginone hybrids against ABS P. falciparum was determined. Importantly, the exquisite potency of 35, 36, and MAT436 translated well to cell-based assays.

All three compounds inhibited P. falciparum ABS growth at low nanomolar concentrations with comparable potencies (MAT436, EC₅₀=6.8 nM; 35, EC₅₀=18.7 nM; 36, EC₅₀=18.7 nM).

BOC-protected compounds 37, 38, and 39 were prepared:

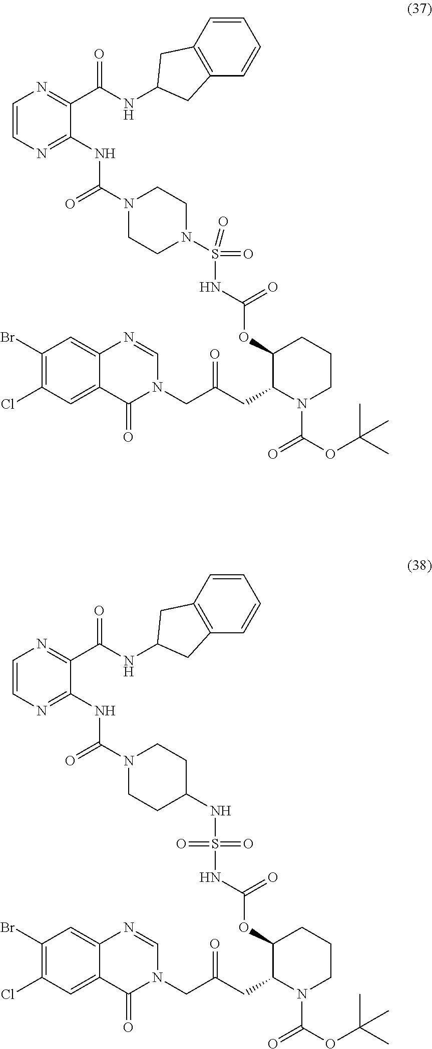 [SMILES: CC(C)(C)OC(=O)N1CCC[C@H](OC(=O)NS(=O)(=O)N2CCN(C(=O)Nc3nccnc3C(=O)NC3Cc4ccccc4C3)CC2)[C@H]1CC(=O)Cn1cnc2cc(Br)c(Cl)cc2c1=O ; CC(C)(C)OC(=O)N1CCC[C@H](OC(=O)NS(=O)(=O)NC2CCN(C(=O)Nc3nccnc3C(=O)NC3Cc4ccccc4C3)CC2)[C@H]1CC(=O)Cn1cnc2cc(Br)c(Cl)cc2c1=O]

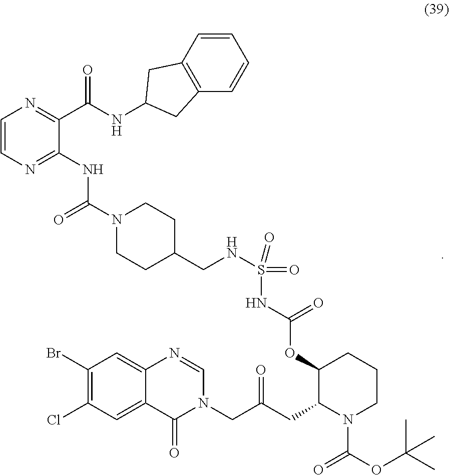 [SMILES: CC(C)(C)OC(=O)N1CCC[C@H](OC(=O)NS(=O)(=O)NCC2CCN(C(=O)Nc3nccnc3C(=O)NC3Cc4ccccc4C3)CC2)[C@H]1CC(=O)Cn1cnc2cc(Br)c(Cl)cc2c1=O]

These compounds, while active, were less active than non-BOC protected counterparts (>1 μM) (FIG. 5i). The epimerized analog of MAT436, 40, was only 3-fold less active, which overall tracked well with its 5-10-fold reduced biochemical potency. MAT436, 35, 36, and 40 exhibited reduced activity towards halofuginone-induced and HFGR-1-mutant parasites, comparable to halofuginone (FIG. 5j). This finding provides further validation for on-target activity.

### Rim Targeting Ligands

Urea and Thiourea-Containing Compounds were Also Prepared (Rim Targeting Ligands):

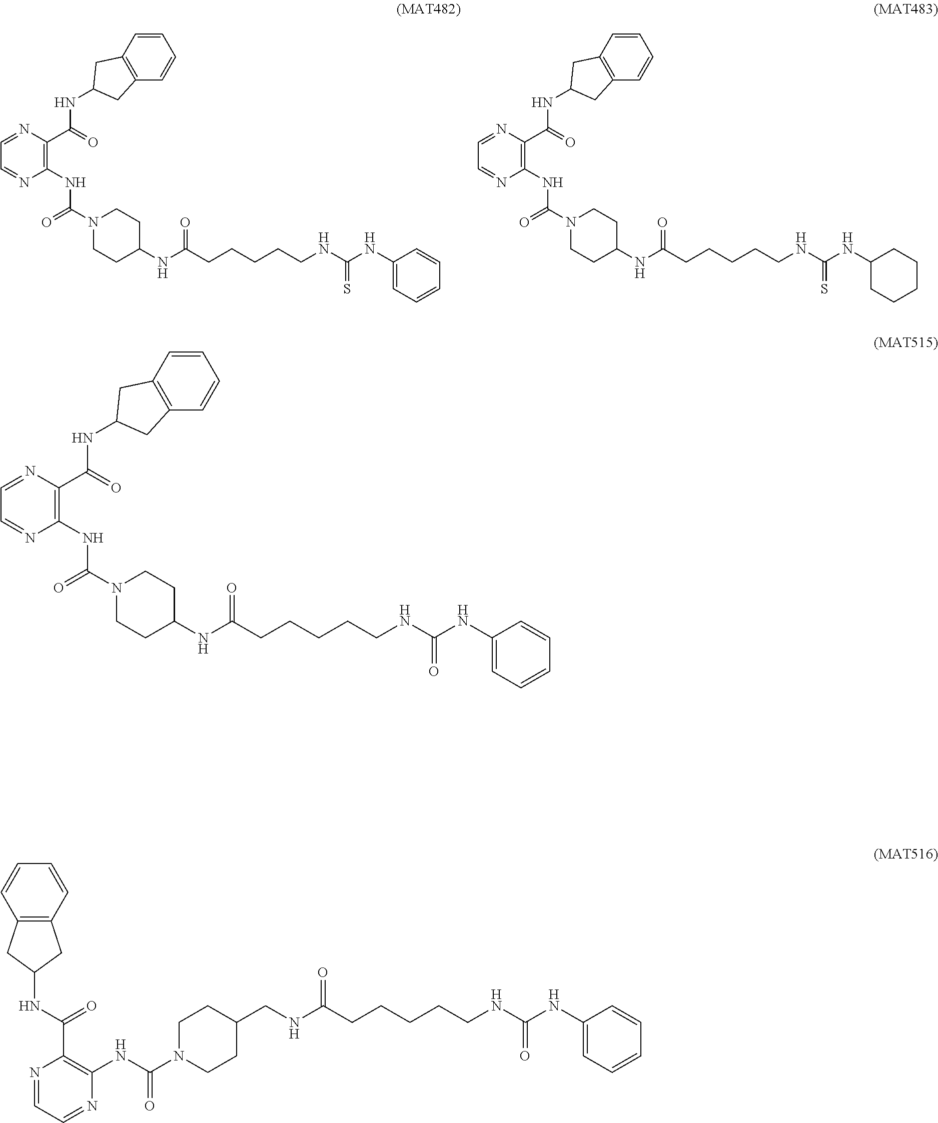 [SMILES: O=C(CCCCCNC(=S)Nc1ccccc1)NC1CCN(C(=O)Nc2nccnc2C(=O)NC2Cc3ccccc3C2)CC1 ; O=C(CCCCCNC(=S)NC1CCCCC1)NC1CCN(C(=O)Nc2nccnc2C(=O)NC2Cc3ccccc3C2)CC1 ; O=C(CCCCCNC(=O)Nc1ccccc1)NC1CCN(C(=O)Nc2nccnc2C(=O)NC2Cc3ccccc3C2)CC1 ; O=C(CCCCCNC(=O)Nc1ccccc1)NCC1CCN(C(=O)Nc2nccnc2C(=O)NC2Cc3ccccc3C2)CC1]

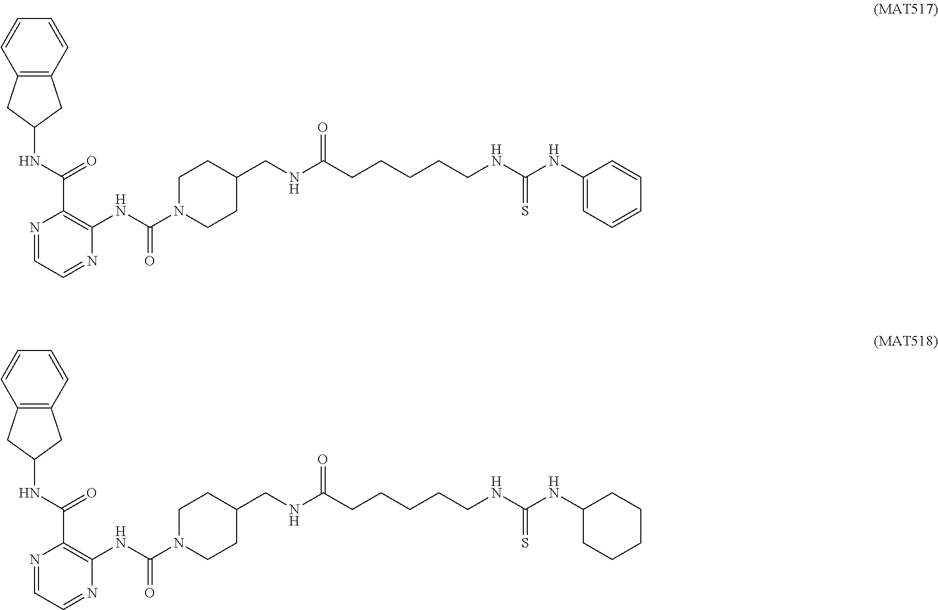 [SMILES: O=C(CCCCCNC(=S)Nc1ccccc1)NCC1CCN(C(=O)Nc2nccnc2C(=O)NC2Cc3ccccc3C2)CC1 ; O=C(CCCCCNC(=S)NC1CCCCC1)NCC1CCN(C(=O)Nc2nccnc2C(=O)NC2Cc3ccccc3C2)CC1]

MAT515 had high affinity for PfcProRS (K_{D}=70.4 nM) at the cost of increased affinity for HsProRS (K_{D}=169 nM) whereas MAT518, had reduced affinity for both paralogs while retaining ˜7-fold selectivity for PfcProRS (K_{D}=372 nM) compared to HsProRS (K_{D}=2.76 μM). The compounds provide evidence that the active site rim is rationally engaged to provide for improved paralog-specificity.

### 3-Hydroxypiperidine Hybrids and Prolinol Hybrids

A series of sulfamoylcarbamate-linked hybrids was prepared connecting the pyrazinamide analog MAT336 with both enantiomers of 3-hydroxypiperidine and both enantiomers of prolinol:

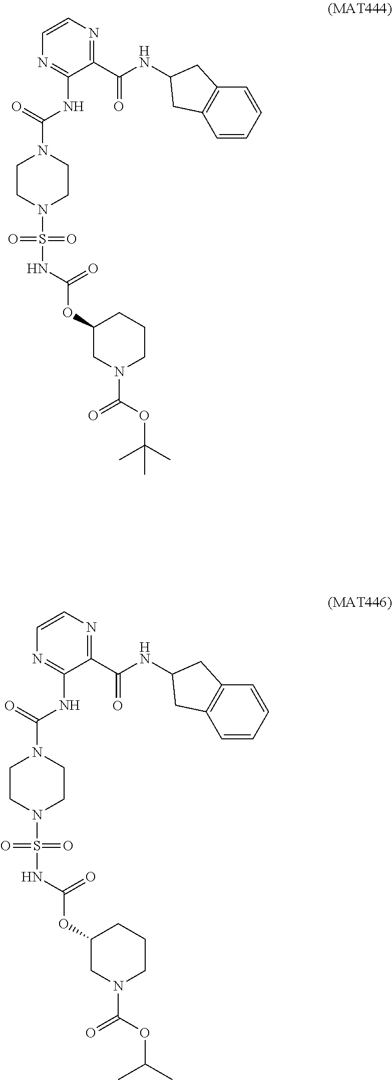 [SMILES: CC(C)(C)OC(=O)N1CCC[C@H](OC(=O)NS(=O)(=O)N2CCN(C(=O)Nc3nccnc3C(=O)NC3Cc4ccccc4C3)CC2)C1 ; CC(C)OC(=O)N1CCC[C@@H](OC(=O)NS(=O)(=O)N2CCN(C(=O)Nc3nccnc3C(=O)NC3Cc4ccccc4C3)CC2)C1]

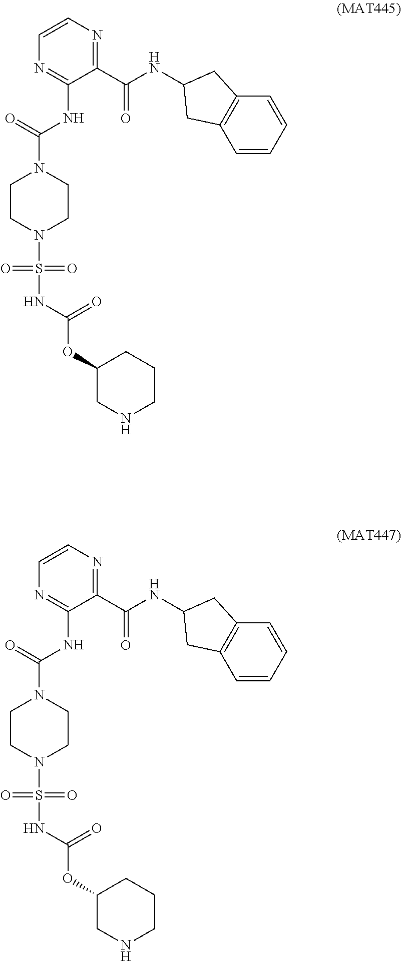 [SMILES: O=C(NS(=O)(=O)N1CCN(C(=O)Nc2nccnc2C(=O)NC2Cc3ccccc3C2)CC1)O[C@H]1CCCNC1 ; O=C(NS(=O)(=O)N1CCN(C(=O)Nc2nccnc2C(=O)NC2Cc3ccccc3C2)CC1)O[C@@H]1CCCNC1]

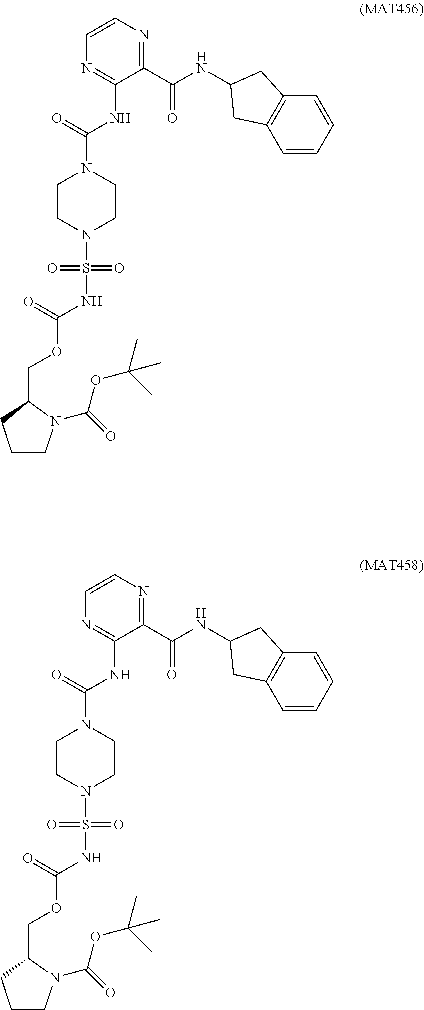 [SMILES: CC(C)(C)OC(=O)N1CCC[C@H]1COC(=O)NS(=O)(=O)N1CCN(C(=O)Nc2nccnc2C(=O)NC2Cc3ccccc3C2)CC1 ; CC(C)(C)OC(=O)N1CCC[C@@H]1COC(=O)NS(=O)(=O)N1CCN(C(=O)Nc2nccnc2C(=O)NC2Cc3ccccc3C2)CC1]

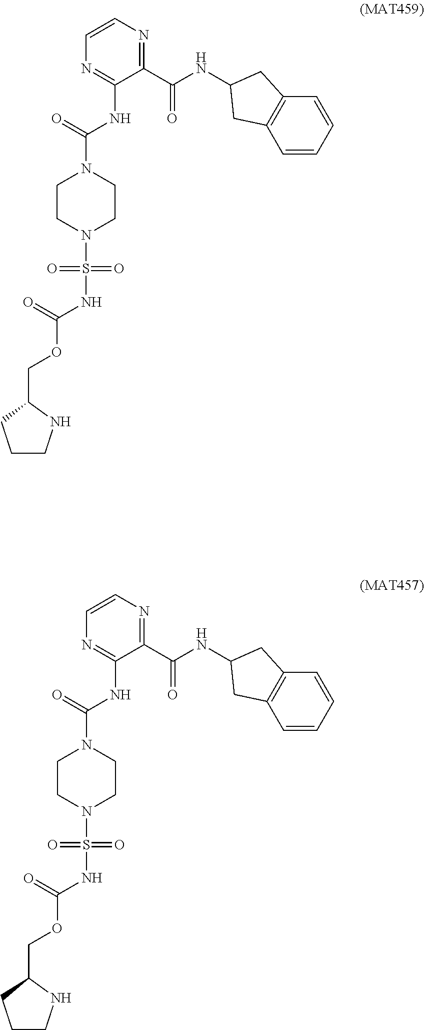 [SMILES: O=C(NS(=O)(=O)N1CCN(C(=O)Nc2nccnc2C(=O)NC2Cc3ccccc3C2)CC1)OC[C@H]1CCCN1 ; O=C(NS(=O)(=O)N1CCN(C(=O)Nc2nccnc2C(=O)NC2Cc3ccccc3C2)CC1)OC[C@@H]1CCCN1]

These compounds were then profiled in the TR-FRET assay and parasite growth. The compounds had affinity for PfcProRS in the absence of proline (MAT445 K_{D}=1.11 μM and MAT447 K_{D}=2.46 μM) or presence of 100 μM proline (MAT445 K_{D}=966 nM and MAT447 K_{D}=2.45 μM). Consistent with the biochemical activity these compounds were active in wildtype ABS P. falciparum Dd2-2D4 (MAT445 EC₅₀=1.16 μM and MAT447 EC₅₀=6.25 μM) and less active (EC₅₀>10 μM) in both halofuginone-induced and HFGR-I parasites. The minimal difference between stereoisomers is consistent with the notion that these compounds bind with the 3-hydroxypiperidine moiety pointed towards the active site rim rather than the proline-binding site but may also reflect the fact the high degree of conformational flexibility of piperidine rings arising from their reduced axial strain relative to analogous cyclohexyl rings. The prolinol hybrids had affinity for PfcProRS in the absence of proline (MAT457 K_{D}=152 nM and MAT459 K_{D}=736 nM) and in the presence of 100 μM proline (MAT445 K_{D}=118 nM and MAT447 K_{D}=552 nM). They were also selective for HsProRS in the absence of proline (MAT457 K_{D}=9.71 nM and MAT459 K_{D}=105 nM) and in the presence of 100 μM proline (MAT457 K_{D}=16.3 nM and MAT459 K_{D}=245 nM). As with the 3-hydroxypiperidine hybrids, the prolinol hybrids were active in wildtype ABS P. falciparum Dd2-2D4 (MAT457 EC₅₀=968 nM and MAT459 EC₅₀=4.62 μM) and both were less active (EC₅₀>10 μM) in both halofuginone-induced and HFGR-I parasites. However, the prolinol hybrids had a much greater difference between the stereoisomers which may indicate they bind in the proline-pocket and/or reflect the increased conformational rigidity of the pyrrolidine ring on prolinol relative to the prior series' piperidine ring.

### Hydroxyethylquinazolinone Hybrids

Inhibitors were prepared that retained the triple-site inhibitor's potency without halofuginone cross-resistance or epimerization. Hydroxyethylquinazolinone hybrids were prepared in attempt to directly link the ATP- and tRNA-binding pockets. The compounds were characterized in the TR-FRET assay and MAT513 and MAT514 selectively inhibited PfcProRS (MAT513 K_{D}=20.3 nM and MAT514 K_{D}=35.2 nM) compared to HsProRS (MAT513 K_{D}=157 nM and MAT514 K_{D}=277 nM). Their cellular activity in wildtype and halofuginone resistant parasites was less than expected (for Dd2-2D4, MAT513 IC₅₀=4.83 μM and MAT514 IC₅₀=4.56 μM). MAT511 was active in parasites (Dd2-2D4 IC₅₀=73.9 nM) despite lesser biochemical activity against PfcProRS and HsProRS (K_{D}>10 μM for both), suggesting a different mechanism of action.

### Liver Stage and Host Toxicity Evaluation

Halofuginone and derivatives are active against liver stage Plasmodium parasites. NCP26 and MAT436 were evaluated in a tissue culture-based P. berghei liver stage model. Specifically, various test compounds were added in dose-response to human hepatocytes (HuH7) followed by infection with luciferase-expressing P. berghei ANKA sporozoites, which enabled quantification of parasite load by luminescence. In parallel, host cell viability was also assessed to provide an estimate of cellular selectivity. Both compounds displayed potent antiparasitic activity resulting in complete inhibition of P. berghei growth at submicromolar concentrations (NCP26 EC₅₀=249 nM and MAT436 EC₅₀=186 nM), while showing less pronounced activity in human hepatocytes that suggested host cytostatic activity rather than the cytotoxic activity that is expected at higher concentrations (FIG. 5k). This is consistent with the results obtained for halofuginone and halofuginol, which, despite comparable biochemical activity, exhibit selectivity for parasite versus host activity in cell-based assays.

### Discussion

Multiple orthogonal classes of PfcProRS inhibitors were developed herein with high affinity for ProRS and potent dual-stage activity against ABS and liver stage Plasmodium parasites. These include a new set of ATP-site directed, proline-uncompetitive inhibitors with low- to sub-nanomolar affinity that overcome halofuginone resistance mechanisms or other forms of rapid drug tolerance. These inhibitors design improve activity of previously reported compound 2, a proline-uncompetitive ligand class for HsProRS. Although most compounds in this series showed moderate biochemical selectivity for HsProRS in the absence and presence of proline, the tested compounds were more selective for Plasmodium in cell-based assays (e.g. NCP26 EC_{50,Dd2-2D4}=67.4 nM and NCP26 EC_{50,HuH7}>2.5 μM).

Ultimately, cell-based selectivity is the more critical metric for drug-development and increased cellular sensitivity of Plasmodium to ProRS inhibition has previously been noted for other ProRS inhibitors including halofuginone. While the higher sensitivity can in part be attributed to the increased sensitivity of rapidly proliferating cells to aaRS inhibition, and potentially favorable differential cellular pharmacokinetics, much of the apparent disconnect between biochemical and cellular selectivity can be explained when considering the differential substrate affinities of the host and parasite ProRS paralogs. The physiological concentration of ATP in human cells (0.5 to 5 mM) and P. falciparum (2.5 mM) are comparable. However, as shown herein, HsProRS binds ATP with much higher affinity and is therefore predicted to exhibit much stronger competition with ATP-site directed ligands, such as NCP26. The relevance of effect has been well-documented for kinase inhibitors, where the disregard for differential K_{M}-values has resulted in misinterpretation of cellular activities.

In addition, PfcProRS inhibition was validated as the primary mode of action of NCP26 by a combination of in vitro resistance selection studies and whole genome sequence analysis. Although the evolution of resistance in response to drug treatment is never desirable, only very few antimalarials fail to induce in vitro resistance and recent guidelines have been developed to assess the risk for new candidates. Compared to other drug-target combinations, NCP26 exhibited a very low propensity to induce resistance, requiring a large population size and long timeframe for resistance to arise (>50 generations). These findings are encouraging and demonstrate that the rapid resistance in response to PfcProRS inhibition, as observed with halofuginone, is likely an inhibitor-class specific issue.

Moreover, by combining features of halofuginone with adenosine or NCP26, the first examples of true triple-site ProRS inhibitors were made herein, i.e., ligands that meaningfully occupy all three substrate-binding pockets of the active site simultaneously. Triple-site aaRS inhibitors have been postulated previously, but have remained elusive. ThrRS ligands, such as borrelidin and analogs, which are triple-competitive and bind the amino acid and tRNA sites of ThrRS in addition to an auxiliary site, have been reported. However, no triple-site aaRS inhibitors that target all substrate pockets have been reported to date. Co-crystal structure of MAT436 and PfcProRS herein confirms the predicted binding mode. The triple-site compounds bound both ProRS paralogs with subnanomolar affinity in the absence of either substrate (proline or ATP). Their high biochemical affinity translated well into antiparasitic activity against both ABS and liver stage Plasmodium parasites. High selectivity for Plasmodium parasites was observed over human cells in the cell-based assays. While the molecular mass of these triple-site inhibitors exceeds the canonical “rule of five” cut-off of 500 Da, consensus based on the analysis of approved drugs agrees that this limit has been overly stringent and suggest that the size of the hybrid ligands to be well within an acceptable range.

### General Methods Used in the Example

Preliminary Docking Studies: While the inhibitor classes were all rationally designed, preliminary docking studies were done to guide in the prioritization of which analogs to synthesize first. These studies were conducted in Spark™ v10.5.0, Forge™ v10.5.0, and Flare™ v4.0.2 (Cresset Biomolecular Discovery Ltd) per the manufacturer's instructions in their respective user guides. Ligands were docked against the ProRS structures reported here (PDB 6T7K, 7QB7, 7QC1, and 7QC2) and previously (for HsProRS, PDB: 5VAD, 4HVC, 4K86, 4K87, 4K88, and 5V58; for PfcProRS, PDB 4Q15, 4NCX, 4YDQ, 40LF, 5IFU, and 4WI1). Protein preparation was accomplished using default settings and the pharmacophore constraints were automatically generated and used without modification. Conformation hunts were done with “very accurate but slow” setting modified to allow rotation about acyclic secondary amide bonds. Alignments were performed using both “normal” (unbiased) and “substructure” (guided by ligands from crystal structures) settings. No model building was used to guide chemical synthesis.

Crystallization, data collection and structure determination: PfcProRS was co-crystallized with NCP26 (3) and MAT436 (34) at 20° C. using the sitting drop vapor diffusion method.

For crystals of PfcProRS in complex with NCP26 and proline (PDB: 6T7K), 2 mM NCP26 was added to 39 mg/mL PfcProRS together with 5 mM L-Proline, and crystals were obtained in a drop containing 75 nL of protein-compound mixture and 75 nL precipitant composed of 0.1 M HEPES, pH 7.5, and 20% PEG 10000.

For crystals of PfcProRS in complex with MAT436 (PDB: 7QC1), 1 mM MAT436 was added to 22.4 mg/mL PfcProRS, and crystals obtained in a drop containing 100 nL of the protein compound mixture and 50 nL of 1.5 M malic acid.

The crystals were cryo-protected in precipitant solution supplemented with 25-30% ethylene glycol and then flash cooled in liquid nitrogen. Data was collected on beamlines 103 and 104 at the Diamond Light Source UK, and the dataset processed, scaled, and merged at the Diamond Light Source using Xia2. Electron density maps were obtained by molecular replacement using PHASER with previously determined structures of PfcProRS as a search model.

The complex structure of PfcProRS with NCP26 (PDB 6T7K) was solved to 1.79 Å resolution using PDB 4Q15 as a search model. The complex structure of PfcProRS with MAT436 was solved to 2.51 Å resolution (PDB 7QC1) using PDB 6T7K as search model. The structures were refined in an iterative process using PHENIX with electron density map inspections and model improvement in WinCOOT and terminated when there were no substantial changes in the R_{work} and R_{free} values and inspection of the electron density map suggested that no further corrections or additions were justified. Structural analysis and figures were performed with PyMOL.

Crystallographic data and refinement statistics are available in FIG. 8.

P. falciparum Cell Lines and Culture Conditions: Parasites were maintained under standard culture conditions as described previously. The P. falciparum Dd2-2D4 clone was derived from Malaria Research and Reagent Resource Repository line MRA-156 (BEI Resources). The P. falciparum HFG-induced (elevated proline homeostasis) and HFGR1 (elevated proline homeostasis and PfcProRS^{L482H}) were previously reported previously.

P. falciparum Asexual Blood Stage Growth Assay: This assay was performed as previously described. In short, P. falciparum erythrocytic-stage parasites at 1% parasitemia and 1% hematocrit in RPMI+0.5% Albumax were seeded at 40 L/well in 384-well plates with test compounds in triplicate, dose-response format with 10 μM dihydro-artemisinin as a kill-control and blank (no compound) wells as a growth-control. DMSO concentration did not exceed 1% (v/v). After 72 h, growth was quantified by measuring fluorescence following SYBR Green staining. Data was analyzed in Excel and plotted in GraphPad PRISM.

P. falciparum Asexual Blood Stage Short-Term Resistance Susceptibility Assay: Using the robust procedure previously used to generate HFG-induced parasites (HFG-tolerant with elevated proline homeostasis), unsuccessful attempts were made to generate NCP26-tolerant/resistant parasites. In short, three independent flasks of P. falciparum Dd2-2D4 parasites were treated with 4×EC₅₀ NCP26 until no parasites were detected by Giemsa staining microscopy. Following recrudescence, sensitivity to NCP26 and halofuginone was assayed using the ABS growth assay.

NCP26 Resistance Selection: Three independent selections for NCP26-resistant mutants of P. falciparum Dd2-2D4 parasites were conducted in vitro as previously reported. In short, parasites were treated with 4×EC₅₀ NCP26 until no parasites were detected by giemsa staining microscopy. Following recrudescence, the asexual blood stage growth assay was used to determine sensitivity to NCP26 and control compounds including ProRS inhibitors halofuginone (1) and ProSA (25); threonyl-tRNA synthetase (ThrRS) inhibitor borrelidin; and dihydroartemisinin (DHA). This cycle was repeated for ˜50 generations (˜100 days), corresponding to 5-6 cycles of drug pressure. Selections were initially made with ˜3×10⁸ parasites per flask (i.e. per independent selection), but did not observe any resistance after 2 cycles of drug pressure (38 days; ˜19 generations) so selection cultures were expanded to ˜1×10⁹ parasites per flask and maintained this for the remainder of the selection.

Subcloning: Clonal parasites were isolated from each selection flask by limiting dilution of ring stage parasites in 96-well plates to an average of 0.8 and 0.2 parasites per well. Following recrudescence, these clonal parasites were assayed in the asexual blood stage blood stage viability assay to ensure no phenotypic differences from the corresponding bulk population (all isolated clones had EC₅₀ values for all inhibitors tested within 2-fold of corresponding bulk population).

Library preparation and whole genome sequencing: Infected RBCs were washed with 0.05% saponin and genomic DNA was isolated from the parasites using a DNeasy Blood and Tissue Kit (Qiagen) according to the standard protocols. Sequencing libraries were prepared with the Nextera XT kit (Cat. No FC-131-1024, Illumina) via the standard dual index protocol and sequenced on the Illumina NovaSeq 6000 S4 flow cell to generate paired-end reads 100 bp in length. Sequence data is available under BioProject Accession number: PRJNA811614 in the NCBI Sequence Read Archive. Reads were aligned to the P. falciparum 3D7 reference genome (PlasmoDB v13.0) using the previously described pipeline. A total of 8 samples were sequenced to an average whole genome coverage of 157×, with an average of 89% of reads mapping to the reference genome. Following alignment, SNVs and INDELs were called using GATK HaplotypeCaller and filtered according to GATK's best practice recommendations. Variants were annotated using a custom SnpEff database and further filtered by comparing those from resistant clones to the parent clone, such that only a mutation present in the resistant clone but not the sensitive parent clone would be retained. CNVs were identified by differential Log 2 copy ratio as described in the GATK 4 workflow. Briefly, read counts were collected across genic intervals for each sample. Copy ratios were calculated after denoising read counts against a strain-matched Panel of Normals composed of non-drug-selected Dd2 parasite samples.

P. berghei Liver Stage and HuH7 Host Hepatocyte Growth Assay: HuH7 cells (Sigma) were cultured in DMEM+L-Glutamine (Gibco) supplemented with 10% (v/v) heat-inactivated FBS (Sigma) and 1% (v/v) antibiotic/antimycotic (Sigma). Hepatocyte cultures were maintained in a standard tissue culture incubator at 37° C. Anopheles mosquitoes infected with luciferase-expressing P. berghei ANKA sporozoites were obtained from the Sporocore at the University of Georgia. Liver stage P. berghei assays were completed as previously described. Briefly, 4,000 HuH7 cells were seeded into 384-well plates (Corning) one day prior to infection. Compounds (0-50 μM) were added in triplicate to wells before infection with 4,000 P. berghei sporozoites. At ˜44 hpi, HuH7 cell viability and P. berghei parasite load was assessed using CellTiter-Fluor (Promega) and Bright-Glo (Promega), respectively, using an Envision plate reader. Relative fluorescence and luminescence signal intensities were normalized to the negative control, 1% DMSO. EC₅₀ values were determined using GraphPad Prism through fitting data to a dose response curve. Reported EC₅₀ values are averages of three independent experiments.

### Example 2—Synthetic Preparation of ProRS Inhibitors

General Methods: Unless otherwise noted, all reagents were used as received from vendors.

Column purifications were performed on a Biotage Isolera 4 Purification System equipped with a 200-400 nm diode array detector. For normal phase flash column chromatography purifications, Sorbtech Purity Flash Cartridges were used (CFC-52300-012-18 and CFC-52500-025-12). For reverse phase flash column chromatography purifications, Biotage SNAP KP-C18-HS (FSLO-1118-0012 and FSLO-1118-0030) and Biotage Sfar Bio C18 Duo 300 Å, 20 m cartridges were used (FSBD-0411-0010 and FSBD-0411-0025).

Analytical LC-MS was performed on a Waters 2545 HPLC equipped with a 2998 diode array detector, a 2424 evaporative light scattering detector, a 2475 multichannel fluorescence detector, and a Waters 3100 ESI-MS module, using a XTerraMS C18, 5 μm, 4.6×50 mm column at a flow rate of 5 mL/min with a linear gradient (95% A: 5% B to 100% B over 90 sec and 30 sec hold at 100% B; solvent A=water+0.1% formic acid, solvent B=acetonitrile+0.1% formic acid). LC-MS data analysis was performed using Waters Masslynx V4.1 SCN 846 software.

Proton and carbon nuclear magnetic resonance (¹H and ¹³C NMR spectra) were recorded on a Bruker Avance III 400 spectrometer using Topspin 3.2 software and data were analyzed using MestreNova (version 12.0.1-20560, Mestrelab Research). Chemical shifts for NMR spectra are reported in parts per million (ppm) and are referenced to residual solvent peaks (except for ¹³C NMR in D₂O). Data is reported as follows: chemical shift, multiplicity (s=singlet, br s, =broad singlet, d=doublet, t=triplet, q=quartet, p=pentet, m=multiplet), proton coupling constants (J, Hz), and integration.

Abbreviations: DCC (N,N′-Dicyclohexylcarbodiimide), DCM (dichloromethane), DIPEA (ethyldiisopropylamine), DMAc (N,N-dimethylacetamide), DMF (N,N-dimethylformamide), DMSO (dimethylsulfoxide), EtOAc (ethyl acetate), LC-MS (Liquid Chromatography-Mass Spectrometry), MeCN (acetonitrile), MeOH (methanol), MTBE (methyl tert-butyl ether), NHS (N-hydroxysuccinimide), THE (tetrahydrofuran).

### 3-amino-N-(2,3-dihydro-1H-inden-2-yl)pyrazine-2-carboxamide (45)

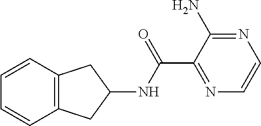 [SMILES: Nc1nccnc1C(=O)NC1Cc2ccccc2C1]

Under an argon atmosphere, mixed 3-aminopyrazine-2-carboxylic acid (7.5 g, 54 mmol, 1 eq), DCM (600 mL), and DIPEA (28 mL, 21 g, 160 mmol, 3 eq). Added isobutyl chloroformate (7.8 mL, 8.1 g, 59 mmol, 1.1 eq) and stirred 16 h. Added 2,3-dihydro-1H-inden-2-amine (9.2 g, 54 mmol, 1 eq) and stirred for 24 h. Diluted with 150 mL DCM and 5 mL MeOH. Washed sequentially with 1:1 saturated NaHCO_{3(aq)}/H₂O (2×300 mL) and 1:1 saturated NaCl_{(aq)}/H₂O (1×300 mL), keeping the emulsion with the organic layer each time. Filtered insoluble (largely product, but not pure) and set filtrand aside, dried filtrate over anhydrous Na₂SO₄, filtered, and concentrated under reduced pressure. Recombined the concentrated material with the filtrand from before. Triturated with 50 mL MeCN (note: sonicated vigorously until the solid was visually homogeneous beige, the MeCN was orange, and no black/brown spots were observed; typically ˜10-15 min) and filtered. Yield: 11.1 g, 80.7%. Beige solid. ¹H NMR (400 MHz, DMSO-d₆) δ 8.81 (d, J=7.9 Hz, 1H), 8.20 (d, J=2.1 Hz, 1H), 7.80 (d, J=2.1 Hz, 1H), 7.55 (s, 2H), 7.22 (dd, J=5.5, 3.3 Hz, 2H), 7.15 (dd, J=5.4, 3.2 Hz, 2H), 4.70 (h, J=7.5 Hz, 1H), 3.17 (dd, J=15.7, 7.6 Hz, 2H), 3.01 (dd, J=15.7, 7.3 Hz, 2H). ¹³C NMR (101 MHz, DMSO-d₆) δ 165.83, 155.19, 146.83, 141.18, 130.81, 126.46, 125.65, 124.47, 50.23, 38.59. LC-MS (C₁₄H₁₄N₄O): Calculated [M+H]⁺ m/z=255.12. Observed [M+H]⁺ m/z 255.33.

### 3-(cyclohexanecarboxamido)-N-(2,3-dihydro-1H-inden-2-yl)pyrazine-2-carboxamide (T-3767758, 2)

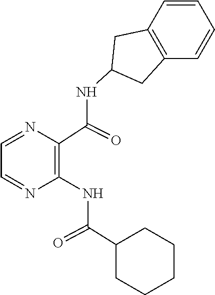 [SMILES: O=C(NC1Cc2ccccc2C1)c1nccnc1NC(=O)C1CCCCC1]

Under an argon atmosphere, mixed 45 (117 mg, 462 μmol, 1 eq), DCM (5 mL), cyclohexanecarbonyl chloride (92.6 μL, 101 mg, 693 μmol, 1.5 eq), and DIPEA (0.24 mL, 1.4 mmol, 3.0 eq). After reaction was complete by LC-MS, diluted with DCM (25 mL) and quenched with H₂O (20 mL). Washed with 1N HCl_{(aq)} (2×20 mL), 1:1 saturated NaHCO_{3(aq)}/H₂O (2×20 mL), and 1:1 saturated NaCl_{(aq)}/H₂O (2×20 mL). Dried over Na₂SO₄, filtered, and concentrated in vacuo. Purified by normal phase flash column chromatography (20%-60% EtOAc in hexanes gradient). Yield: 45.5 mg, 27%. ¹H NMR (400 MHz, Chloroform-d) δ 11.98 (s, 1H), 8.57 (d, J=2.3 Hz, 1H), 8.33 (d, J=8.2 Hz, 1H), 8.11 (s, 1H), 7.29-7.25 (m, 2H), 7.24-7.19 (m, 2H), 4.88 (tt, J=12.3, 8.1, 4.8 Hz, 1H), 3.43 (dd, J=16.2, 7.2 Hz, 2H), 2.97 (dd, J=16.2, 4.8 Hz, 2H), 2.44 (tt, J=11.7, 3.6 Hz, 1H), 2.05 (d, J=12.9 Hz, 2H), 1.91-1.80 (m, 2H), 1.71 (d, J=10.0 Hz, 1H), 1.59 (q, J=12.2 Hz, 2H), 1.42-1.24 (m, 3H). ¹³C NMR (101 MHz, CDCl₃) δ 174.69, 165.32, 149.72, 146.58, 140.58, 136.30, 129.16, 127.11, 124.93, 50.71, 47.51, 40.06, 29.49, 25.82, 25.76. LC-MS (C₂₁H₂₄N₄O₂): Calculated [M+H]⁺ m/z=365.19, [M−H]⁻ m/z=363.19. Observed [M+H]⁺ m/z=365.28, [M−H]⁻ m/z=363.09.

### N-(2,3-dihydro-1H-inden-2-yl)-3-(piperidine-1-carboxamido)pyrazine-2-carboxamide (NCP26, 3)

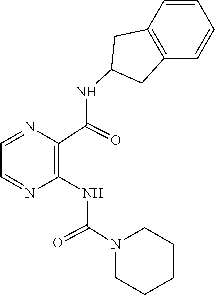 [SMILES: O=C(NC1Cc2ccccc2C1)c1nccnc1NC(=O)N1CCCCC1]

To a solution of 45 (3.00 g, 11.8 mmol, 1 eq) in anhydrous DCM (300 mL), added anhydrous lutidine (5.5 mL, 47 mmol, 4 eq) and cooled to 0° C. Over the course of 10 min, added 15% w/v phosgene₍ₜₒₗᵤₑₙₑ₎ (11.8 mL, 16.5 mmol, 1.4 eq) slowly along the walls and then stirred 40 min at 0° C. While waiting, charged a second flask with piperidine (2.33 mL, 23.6 mmol, 2 eq) and DCM (150 mL). Over the course of 3 min, added the phosgene solution to the piperidine solution and stirred at room temperature overnight. Quenched by addition of MeOH (30 mL) and stirred at room temperature for 30 min. Concentrated reaction mixture directly onto silica (˜20 g) in vacuo and purified by normal phase flash column chromatography eluting with 20-100% EtOAc in hexanes. Yield: 2.08 g, 48.2%. White solid. ¹H NMR (400 MHz, Chloroform-d) δ 11.37 (s, 1H), 8.47 (s, 1H), 8.28 (d, J=8.1 Hz, 1H), 7.96 (s, 1H), 7.26-7.22 (m, 2H), 7.20-7.16 (m, 2H), 4.84 (h, J=7.5 Hz, 1H), 3.57 (s, 4H), 3.39 (dd, J=16.1, 7.2 Hz, 2H), 2.94 (dd, J=16.1, 4.8 Hz, 2H), 1.63 (s, 6H). ¹³C NMR (101 MHz, CDCl₃) δ 165.80, 152.49, 151.25, 146.59, 140.53, 134.61, 128.47, 126.95, 124.82, 50.52, 45.18, 39.96, 25.80, 24.52. LC-MS (C₂₀H₂₃N₅O₂): Calculated [M+H]⁺ m/z=366.19, [M−H]⁻ m/z=364.19. Observed [M+H]⁺ m/z=366.40, [M−H]⁻ m/z=364.13.

### N-(2,3-dihydro-1H-inden-2-yl)-3-(1-methylcyclohexane-1-carboxamido)pyrazine-2-carboxamide (4)

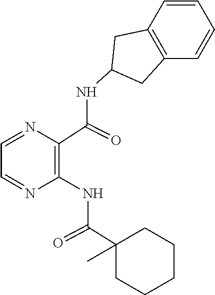 [SMILES: CC1(C(=O)Nc2nccnc2C(=O)NC2Cc3ccccc3C2)CCCCC1]

Dissolved 1-methylcyclohexane-1-carboxylic acid (55.2 mg, 388 μmol, 2 eq) in THE (2 mL). Successively added a drop of DMF and then oxalyl chloride (25.8 μL, 37.4 mg, 295 mol, 1.5 eq). Stirred for 15 min. During this time, separately dissolved 45 (50.1 mg, 197 mol, 1 eq) in THE (4.6 mL) and added 1.0M NaHMDS_{(THF)} (982 μL, 982 μmol, 5 eq) to produce a cloudy, yellow suspension. This suspension was added dropwise to the first solution dropwise. The mixture was a cloudy orange suspension. Checked by LC-MS after 15 min and saw reaction was complete. Concentrated to remove THE and diluted with DCM (30 mL). Washed twice with 1M HCl_{(aq)} (30 mL), twice with water (30 mL), and once with saturated NaCl_{(aq)} (30 mL). Dried over Na₂SO₄, filtered, and concentrated. Purified by normal phase FCC, eluting with hexanes/EtOAc. Yield: 15.1 mg, 20.3%. ¹H NMR (400 MHz, Chloroform-d) δ 12.18 (s, 1H), 8.57 (s, 1H), 8.33 (d, J=6.8 Hz, 1H), 8.10 (s, 1H), 7.28-7.24 (m, 2H), 7.23-7.19 (m, 2H), 4.97-4.85 (m, 1H), 3.42 (dd, J=16.1, 7.1 Hz, 2H), 2.96 (dd, J=16.1, 4.3 Hz, 2H), 2.16 (d, J=9.7 Hz, 2H), 1.66-1.42 (m, 8H), 1.32 (s, 3H). ¹³C NMR (101 MHz, CDCl₃) δ 176.63, 165.32, 150.14, 146.57, 140.63, 136.11, 129.25, 127.09, 124.96, 50.65, 45.02, 40.13, 35.62, 26.49, 25.94, 23.07. LC-MS (C₂₂H₂₆N₄O₂): Calculated [M+H]⁺ m/z=379.21, [M−H]⁻ m/z=377.21. Observed [M+H]⁺ m/z=379.41, [M−H]⁻ m/z=377.25.

### N-(2,3-dihydro-1H-inden-2-yl)-3-(3,3-diisopropylureido)pyrazine-2-carboxamide (MAT107, 5)

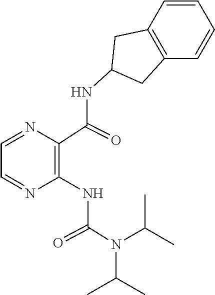 [SMILES: CC(C)N(C(=O)Nc1nccnc1C(=O)NC1Cc2ccccc2C1)C(C)C]

To a solution of 45 (50 mg, 197 μmol, 1 eq) in anhydrous DCM (12 mL), added anhydrous lutidine (114 μL, 983 μmol, 5 eq). Cooled to 0° C. Added 15% w/v phosgene₍ₜₒₗᵤₑₙₑ₎ (196 μL, 275 μmol, 1.4 eq), removed from 0° C. bath, and stirred for 1 h. While waiting, charged a second vial with diisopropylamine (200 μL, 143 mg, 1.42 mmol, 7.2 eq) and DCM (2 mL). Added the contents of the phosgene solution to this vial and stirred at room temperature overnight. Concentrated reaction mixture to dryness. Purified by reverse phase flash column chromatography (water/MeCN; both with 0.1% formic acid). Yield: 38.4 mg, 51.2%. White solid. ¹H NMR (400 MHz, DMSO-d₆) δ 10.79 (s, 1H), 9.20 (d, J=7.9 Hz, 1H), 8.49 (d, J=2.3 Hz, 1H), 8.18 (d, J=2.5 Hz, 1H), 7.25-7.18 (m, 3H), 7.18-7.11 (m, 3H), 4.73 (hept, J=7.3 Hz, 1H), 3.91 (hept, J=6.7 Hz, 2H), 3.25-3.12 (m, 3H), 3.05 (dd, J=15.9, 7.3 Hz, 2H), 1.98 (s, 1H), 1.28 (d, J=6.7 Hz, 12H). ¹³C NMR (101 MHz, DMSO-d₆) δ 166.13, 152.14, 150.89, 146.33, 141.52, 135.53, 130.08, 126.89 (d, J=2.7 Hz), 124.88, 50.86, 46.57, 38.91, 21.26. LC-MS (C₂₁H₂₇N₅O₂): Calculated [M+H]⁺ m/z=382.22, [M−H]⁻ m/z=380.22. Observed [M+H]⁺ m/z=382.45, [M−H]⁻ m/z=380.25.

### tert-butyl ((1s,4s)-4-((3-((2,3-dihydro-1H-inden-2-yl)carbamoyl)pyrazin-2-yl)carbamoyl)cyclohexyl)carbamate (6)

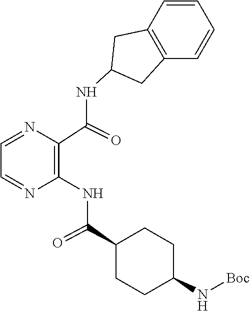 [SMILES: CC(C)(C)OC(=O)N[C@H]1CC[C@@H](C(=O)Nc2nccnc2C(=O)NC2Cc3ccccc3C2)CC1]

Mixed (1s,4s)-4-((tert-butoxycarbonyl)amino)cyclohexane-1-carboxylic acid (232 mg, 952 μmol, 1.18 eq), N-hydroxysuccinimide (111 mg, 966 μmol, 1.20 eq), MeCN (10 mL), and DCM (5 mL) to produce a cloudy, white suspension. Stirred 5 min. Added DCC (197 mg, 955 μmol, 1.18 eq) and stirred overnight. Filtered off insoluble and concentrated to obtain a white solid containing 2,5-dioxopyrrolidin-1-yl (1s,4s)-4-((tert-butoxycarbonyl)amino)cyclohexane-1-carboxylate that was used without further purification or characterization. Dissolved in THE (4 mL). Separately dissolved 45 (205 mg, 806 μmol, 1 eq) in THE (10.0 mL) and added 1.0 M NaHMDS_{(THF)} (1.6 mL, 1.60 mmol, 1.98 eq) dropwise to produce a cloudy, yellow suspension that was stirred 2.67 h. Added this solution dropwise to the N-hydroxysuccinimide solution and stirred for 2 h. Added more 1.0 M NaHMDS_{(THF)} (1.0 mL, 1.00 mmol, 1.25 eq) and stirred 5 min. Added more 1.0 M NaHMDS_{(THF)} (1.0 mL, 1.00 mmol, 1.25 eq) and stirred 5 min. Diluted with DCM (100 mL), washed twice with 3:1 water/saturated NH₄Cl_{(aq)} (100 mL), dried over Na₂SO₄, filtered, and concentrated. Purified by normal phase flash column chromatography (hexanes/EtOAc). Yield: 217.0 mg, 56.1%. Off-white solid. ¹H NMR (400 MHz, Chloroform-d) δ 12.09 (s, 1H), 8.57 (d, J=2.3 Hz, 1H), 8.32 (d, J=8.2 Hz, 1H), 8.13 (d, J=2.4 Hz, 1H), 7.30-7.25 (m, 2H), 7.24-7.19 (m, 2H), 4.88 (dt, J=12.1, 6.1 Hz, 1H), 4.68 (d, J=7.2 Hz, 1H), 3.43 (dd, J=16.2, 7.2 Hz, 2H), 2.97 (dd, J=16.4, 4.4 Hz, 2H), 2.59 (tt, J=8.6, 4.2 Hz, 1H), 1.94 (dt, J=9.0, 4.7 Hz, 2H), 1.88 (p, J=5.1, 4.6 Hz, 2H), 1.81-1.68 (m, 5H), 1.44 (s, 9H). ¹³C NMR (101 MHz, CDCl₃) δ 173.79, 165.33, 149.63, 146.58, 140.56, 136.48, 129.17, 127.15, 124.99, 79.29, 77.36, 50.78, 46.49, 44.39, 40.10, 29.80, 28.58, 25.02. LC-MS (C₂₆H₃₃N₅O₄): Calculated [M+H]⁺ m/z=480.25, [M−H]⁻ m/z=478.25. Observed [M+H]⁺ m/z=480.38, [M−H]⁻ m/z=478.26.

### tert-butyl (1-((3-((2,3-dihydro-1H-inden-2-yl)carbamoyl)pyrazin-2-yl)carbamoyl)piperidin-4-yl)carbamate (7)

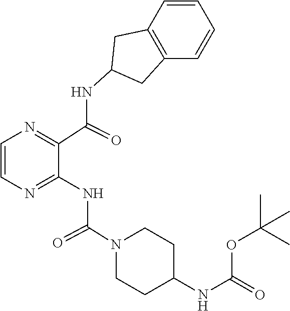 [SMILES: CC(C)(C)OC(=O)NC1CCN(C(=O)Nc2nccnc2C(=O)NC2Cc3ccccc3C2)CC1]

To a solution of 45 (2.00 g, 7.87 mmol, 1 eq) in anhydrous DCM (300 mL), added anhydrous lutidine (3.64 mL, 3.37 g, 31.5 mmol, 4 eq). Cooled to 0° C. Added 15% w/v phosgene₍ₜₒₗᵤₑₙₑ₎ (7.86 mL, 11.0 mmol, 1.4 eq) slowly along the walls over 5 min, removed from 0° C. bath, and stirred for 1 h. Added a solution of tert-butyl piperidin-4-ylcarbamate (3.15 g, 15.7 mmol, 2 eq) in DCM (125 mL) and stirred at room temperature overnight. Quenched reaction mixture with MeOH and concentrated reaction mixture to dryness. Purified by normal phase flash column chromatography (hexanes/EtOAc). Yield: 776 mg, 20.5%. White solid. ¹H NMR (400 MHz, CDCl₃) δ 11.49 (s, 1H), 8.52 (d, J=2.2 Hz, 1H), 8.28 (d, J=8.2 Hz, 1H), 8.01 (d, J=2.3 Hz, 1H), 7.31-7.24 (m, 2H), 7.24-7.18 (m, 2H), 4.93-4.79 (m, 1H), 4.46 (s, 1H), 4.23 (d, J=13.6 Hz, 2H), 3.70 (s, 1H), 3.42 (dd, J=16.2, 7.2 Hz, 2H), 3.08 (t, J=12.6 Hz, 2H), 2.96 (dd, J=16.1, 4.9 Hz, 2H), 2.04 (d, J=11.3 Hz, 2H), 1.45 (d, J=7.6 Hz, 9H), 1.39 (dd, J=12.1, 4.2 Hz, 2H). ¹³C NMR (101 MHz, CDCl₃) δ 165.89, 155.20, 152.59, 151.20, 146.78, 140.63, 135.06, 128.69, 127.13, 124.99, 79.74, 50.68, 48.00, 43.27, 40.12, 32.59, 28.55. LC-MS (C₂₅H₃₂N₆O₄): Calculated [M+H]⁺ m/z=481.26, [M−H]⁻ m/z=479.24. Observed [M+H]⁺ m/z=481.39, [M−H]⁻ m/z=479.40.

### 3-(4-aminopiperidine-1-carboxamido)-N-(2,3-dihydro-1H-inden-2-yl)pyrazine-2-carboxamide dihydrochloride (8·2HCl)

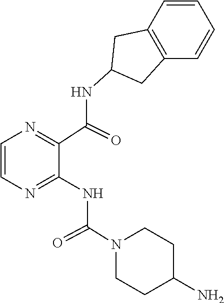 [SMILES: NC1CCN(C(=O)Nc2nccnc2C(=O)NC2Cc3ccccc3C2)CC1]

Charged a flask with 7 (2.00 g, 4.16 mmol, 1 eq) and methanol (10 mL). Added 4.0 M HCl_{(1,4-dioxane)} (10 mL) and stirred vigorously for 1.75 h. Concentrated in vacuo. Yield: 1.85 g, >95%. White solid. ¹H NMR (400 MHz, DMSO) δ 11.04 (s, 1H), 9.21 (d, J=7.8 Hz, 1H), 8.51 (d, J=2.4 Hz, 1H), 8.43 (d, J=5.2 Hz, 3H), 8.23 (d, J=2.4 Hz, 1H), 7.27-7.16 (m, 2H), 7.18-7.05 (m, 2H), 4.71 (h, J=7.6 Hz, 1H), 4.08 (d, J=13.8 Hz, 2H), 3.28 (tt, J=10.6, 5.0 Hz, 1H), 3.19 (dd, J=15.8, 7.8 Hz, 2H), 3.09-2.94 (m, 4H), 2.08-1.93 (m, 2H), 1.55 (qd, J=12.3, 4.2 Hz, 2H). ¹³C NMR (101 MHz, DMSO) δ 165.66, 152.29, 149.82, 145.63, 141.13, 135.71, 130.75, 126.51, 124.50, 50.48, 47.47, 42.11, 38.48, 29.56. LC-MS (C₂₀H₂₄N₆O₂): Calculated [M+H]⁺ m/z=381.20, [M−H]⁻ m/z=379.19. Observed [M+H]⁺ m/z=381.44, [M−H]⁻ m/z=379.43.

### tert-butyl ((1-((3-((2,3-dihydro-1H-inden-2-yl)carbamoyl)pyrazin-2-yl)carbamoyl)piperidin-4-yl)methyl)carbamate (9)

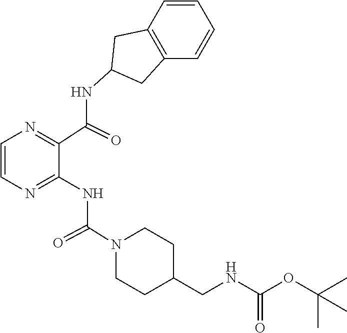 [SMILES: CC(C)(C)OC(=O)NCC1CCN(C(=O)Nc2nccnc2C(=O)NC2Cc3ccccc3C2)CC1]

Charged a flask with 45 (2.99 g, 11.8 mmol, 1 eq), DCM (400 mL), and 2,6-lutidine (6.83 mL, 6.32 g, 59.0 mmol, 5 eq). Cooled reaction to 0° C. Added 15% w/v phosgene₍ₜₒₗᵤₑₙₑ₎ (12 mL, 16.8 mmol, 1.4 eq), removed reaction from 0° C. bath, and let stir at room temperature for 1 h. During this time, charged a second flask with tert-butyl (piperidin-4-ylmethyl)carbamate (4.00 g, 18.7 mmol, 1.59 eq) and DCM (100 mL). Transferred the contents of the first flask to the second flask and stirred vigorously for 20 h. Quenched with methanol, added granular silica (˜15 g), and concentrated to dryness. Purified by normal phase flash column chromatography (hexanes/EtOAc). Yield: 1.14 g, 19.7%. White solid. ¹H NMR (400 MHz, CDCl₃) δ 11.46 (s, 1H), 8.52 (d, J=2.3 Hz, 1H), 8.28 (d, J=8.1 Hz, 1H), 8.00 (d, J=2.3 Hz, 1H), 7.30-7.24 (m, 2H), 7.24-7.15 (m, 2H), 4.92-4.80 (m, 1H), 4.65 (t, J=6.1 Hz, 1H), 4.32 (d, J=13.3 Hz, 2H), 3.42 (dd, J=16.2, 7.2 Hz, 2H), 3.05 (t, J=6.3 Hz, 2H), 3.01-2.86 (m, 4H), 1.79 (d, J=13.3 Hz, 2H), 1.75-1.64 (m, 1H), 1.44 (s, 9H), 1.25 (qd, J=12.2, 4.3 Hz, 2H). ¹³C NMR (101 MHz, CDCl₃) δ 165.89, 156.18, 152.59, 151.24, 146.75, 140.65, 134.92, 128.61, 127.10, 124.97, 79.48, 53.57, 51.02, 50.65, 46.05, 44.29, 41.07, 40.11, 36.91, 29.78, 28.54. LC-MS (C₂₆H₃₄N₆O₄): Calculated [M+H]⁺ m/z=495.27, [M−H]⁻ m/z=493.26. Observed [M+H]⁺ m/z=495.67, [M−H]⁻ m/z=493.45.

### 3-(4-(aminomethyl)piperidine-1-carboxamido)-N-(2,3-dihydro-1H-inden-2-yl)pyrazine-2-carboxamide dihydrochloride (10-2HCl)

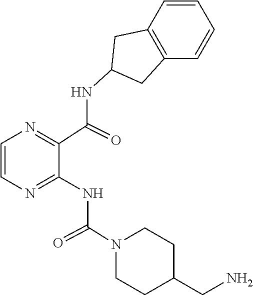 [SMILES: NCC1CCN(C(=O)Nc2nccnc2C(=O)NC2Cc3ccccc3C2)CC1]

Diluted 4.0 M HCl_{(1,4-dioxane)} (4 mL) with methanol (12 mL) and stirred until mixture cooled to room temperature. Added this to a vial containing 9 (799 mg, 1.62 mmol, 1 eq) and stirred vigorously for 2 h. Concentrated in vacuo. Yield: 797.9 mg, 93.2%. White solid. 1H NMR (400 MHz, DMSO) δ 11.01 (s, 1H), 9.22 (d, J=7.8 Hz, 1H), 8.51 (d, J=2.4 Hz, 1H), 8.22 (d, J=2.3 Hz, 1H), 8.03 (s, 3H), 7.27-7.19 (m, 2H), 7.19-7.09 (m, 2H), 4.71 (h, J=7.5 Hz, 1H), 4.07 (d, J=13.4 Hz, 2H), 3.24-3.14 (m, 2H), 3.06 (dd, J=15.8, 7.3 Hz, 2H), 2.91 (t, J=12.7 Hz, 2H), 2.73 (h, J=6.0 Hz, 2H), 1.92-1.83 (m, 1H), 1.83-1.73 (m, 2H), 1.19 (qd, J=12.3, 4.0 Hz, 2H). 13C NMR (101 MHz, DMSO) δ 165.68, 152.16, 149.96, 145.72, 141.11, 135.52, 130.50, 126.49, 124.47, 50.45, 43.48, 38.43, 33.86, 28.91. LC-MS (C₂₁H₂₆N₆O₂): Calculated [M+H]⁺ m/z=395.22, [M−H]⁻ m/z=393.20. Observed [M+H]⁺ m/z=395.04, [M−H]⁻ m/z=393.36.

### N-(2,3-dihydro-1H-inden-2-yl)-3-(4-(hydroxymethyl)piperidine-1-carboxamido)pyrazine-2-carboxamide (11)

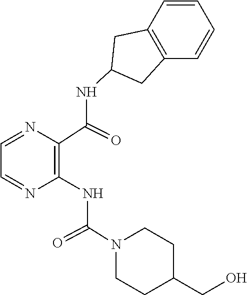 [SMILES: O=C(NC1Cc2ccccc2C1)c1nccnc1NC(=O)N1CCC(CO)CC1]

Cooled a mixture of 45 (500 mg, 1.97 mmol, 1 eq), DCM (80 mL), and 2,6-lutidine (1.14 mL, 1.05 g, 9.83 mmol, 5 eq) to 0° C. Added 15% w/v phosgene₍ₜₒₗᵤₑₙₑ₎ (1.96 mL, 2.75 mmol, 1.4 eq) dropwise and then stirred 15 min at 0° C. Added this solution to a flask piperidin-4-ylmethanol (454 mg, 3.94 mmol, 2 eq) and DCM (20 mL). After stirring reaction mixture overnight, it was diluted with DCM, washed twice with 1M HCl_{(aq)}, and washed once with saturated NaCl_{(aq)}. Dried over Na₂SO₄, filtered, and concentrated in vacuo. Purified by reverse phase flash column chromatography (water+0.1% formic acid/MeCN+0.1% formic acid). Product fractions were concentrated in vacuo. The solid was extracted with MeOH, centrifuged to pellet the insoluble, and decanted. The MeOH-soluble material was concentrated in vacuo and then purified by normal phase flash column chromatography (loaded with DCM and eluted with EtOAc→3:1 EtOAc/EtOH). Yield: 138 mg, 17.7%. White solid. ¹H NMR (400 MHz, CDCl₃) δ 11.84 (d, 1H), 8.69 (s, 1H), 8.29 (d, J=8.1 Hz, 1H), 8.12 (d, J=2.7 Hz, 1H), 7.30-7.26 (m, 1H), 7.26-7.19 (m, 3H), 4.87 (dtd, J=12.4, 7.4, 4.8 Hz, 1H), 4.40-4.26 (m, 2H), 3.55 (d, J=6.1 Hz, 2H), 3.42 (dd, J=16.1, 7.3 Hz, 4H), 2.98 (td, J=10.3, 4.4 Hz, 4H), 1.86 (d, J=13.7 Hz, 2H), 1.82-1.73 (m, 1H), 1.45-1.22 (m, 2H). ¹³C NMR (101 MHz, CDCl₃) δ 165.85, 152.60, 151.13, 146.62, 146.48, 140.64, 140.62, 135.06, 134.90, 128.78, 127.11, 124.99, 75.54, 67.60, 50.70, 50.68, 44.34, 43.93, 40.12, 38.94, 35.60, 28.76, 28.43. LC-MS (C₂₁H₂₅N₅O₃): Calculated [M+H]⁺ m/z=396.20, [M−H]⁻ m/z=394.19. Observed [M+H]⁺ m/z=396.38, [M−H]⁻ m/z=394.37.

### N-(2,3-dihydro-1H-inden-2-yl)-3-(4-(2-hydroxyethyl)piperidine-1-carboxamido)pyrazine-2-carboxamide (12)

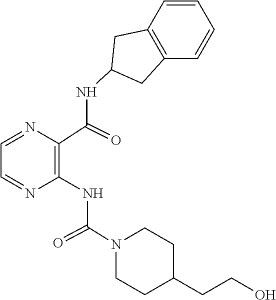 [SMILES: O=C(NC1Cc2ccccc2C1)c1nccnc1NC(=O)N1CCC(CCO)CC1]

Cooled a mixture of 45 (500 mg, 1.97 mmol, 1 eq), DCM (80 mL), and 2,6-lutidine (1.14 mL, 1.05 g, 9.83 mmol, 5 eq) to 0° C. Added 15% w/v phosgene₍ₜₒₗᵤₑₙₑ₎ (1.96 mL, 2.75 mmol, 1.4 eq) dropwise and then stirred 15 min at 0° C. Added this solution to a flask containing 2-(piperidin-4-yl)ethan-1-ol (508 mg, 3.93 mmol, 2 eq) and DCM (20 mL). After stirring reaction mixture overnight, it was diluted with DCM, washed twice with 1M HCl_{(aq)}, and washed once with saturated NaCl_{(aq)}. Dried over Na₂SO₄, filtered, and concentrated in vacuo. Purified by reverse phase flash column chromatography (water+0.1% formic acid/MeCN+0.1% formic acid). Product fractions were concentrated in vacuo. The solid was extracted with MeOH, centrifuged to pellet the insoluble, and decanted. The MeOH-soluble material was concentrated in vacuo and then purified by normal phase flash column chromatography (loaded with DCM and eluted with EtOAc→3:1 EtOAc/EtOH). Yield: 160 mg, 19.9%. White solid. ¹H NMR (400 MHz, CDCl₃) δ 11.74 (d, 1H), 8.72-8.58 (m, 1H), 8.29 (d, J=8.1 Hz, 1H), 8.12-8.06 (m, 1H), 7.30-7.26 (m, 1H), 7.26-7.24 (m, 1H), 7.24-7.17 (m, 2H), 4.93-4.81 (m, 1H), 4.27 (d, J=13.4 Hz, 2H), 3.81 (s, 2H), 3.74 (t, J=6.5 Hz, 2H), 3.42 (dd, J=16.2, 7.2 Hz, 2H), 2.96 (dt, J=14.9, 5.3 Hz, 4H), 1.82 (d, J=13.1 Hz, 2H), 1.78-1.68 (m, 1H), 1.56 (q, J=6.6 Hz, 2H), 1.29 (qd, J=12.5, 4.2 Hz, 2H). ¹³C NMR (101 MHz, CDCl₃) δ 165.77, 152.52, 150.98, 146.22, 140.61, 134.83, 128.84, 127.10, 124.97, 60.31, 50.67, 44.65, 40.10, 39.22, 32.71, 32.22. LC-MS (C₂₂H₂₇N₅O₃): Calculated [M+H]⁺ m/z=410.22, [M−H]⁻ m/z=408.20. Observed [M+H]⁺ m/z=410.41, [M−H]⁻ m/z=408.48.

### tert-butyl 4-((3-((2,3-dihydro-1H-inden-2-yl)carbamoyl)pyrazin-2-yl)carbamoyl)piperazine-1-carboxylate (13)

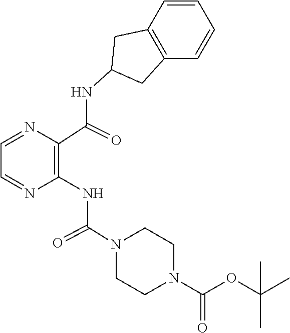 [SMILES: CC(C)(C)OC(=O)N1CCN(C(=O)Nc2nccnc2C(=O)NC2Cc3ccccc3C2)CC1]

Charged a flask with 45 (500 mg, 197 mmol, 1 eq), DCM (75 mL), and 2,6-lutidine (911 μL, 843 mg, 7.87 mmol, 4 eq). Cooled reaction to 0° C. Added 15% w/v phosgene₍ₜₒₗᵤₑₙₑ₎ (2.00 mL, 2.75 mmol, 1.4 eq), removed reaction from 0° C. bath, and let stir at room temperature for 1 h. Added tert-butyl piperazine-1-carboxylate (394 mg, 3.93 mmol, 2 eq) and stirred 24 h. Quenched with methanol and concentrated to dryness. Purified by normal phase flash column chromatography (hexanes/EtOAc). Yield: 156.2 mg, 17.0%. White solid. ¹H NMR (400 MHz, CDCl₃) δ 11.57 (s, 1H), 8.52 (d, J=2.3 Hz, 1H), 8.30 (d, J=8.1 Hz, 1H), 8.06-8.01 (m, 1H), 7.28 (d, J=4.3 Hz, 2H), 7.26-7.18 (m, 5H), 4.86 (tq, J=12.6, 6.0 Hz, 1H), 3.67-3.60 (m, 4H), 3.53 (t, J=5.1 Hz, 4H), 3.43 (dd, J=16.2, 7.3 Hz, 2H), 2.97 (dd, J=16.2, 4.8 Hz, 2H), 1.48 (s, 9H). ¹³C NMR (101 MHz, CDCl₃) δ 165.88, 154.74, 152.71, 151.05, 146.78, 140.60, 135.25, 128.69, 127.14, 125.00, 80.39, 50.72, 43.95, 40.11, 28.55. LC-MS (C₂₄H₃₀N₆O₄): Calculated [M+H]⁺ m/z=467.24, [M−H]⁻ m/z=465.23. Observed [M+H]⁺ m/z=467.50, [M−H]⁻ m/z=465.50.

### N-(2,3-dihydro-1H-inden-2-yl)-3-(piperazine-1-carboxamido)pyrazine-2-carboxamide dihydrochloride (14·2HCl)

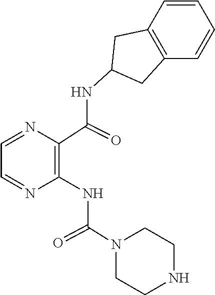 [SMILES: O=C(NC1Cc2ccccc2C1)c1nccnc1NC(=O)N1CCNCC1]

Diluted 4.0 M HCl_{(1,4-dioxane)} (1.50 mL) with methanol (1.50 mL) and stirred until mixture cooled to room temperature. Added this to a vial of 13 (93.0 mg, 199 μmol, 1 eq) and stirred vigorously for 3 h. Concentrated in vacuo. Yield: >95%. White solid. ¹H NMR (400 MHz, DMSO) δ 11.04 (s, 1H), 9.21 (d, J=7.8 Hz, 1H), 8.50 (d, J=2.4 Hz, 1H), 8.21 (d, J=2.4 Hz, 1H), 7.21 (dt, J=7.3, 3.6 Hz, 2H), 7.19-7.10 (m, 2H), 4.72 (h, J=7.5 Hz, 1H), 3.68-3.45 (m, 2H), 3.41 (t, J=5.0 Hz, 4H), 3.19 (dd, J=15.8, 7.7 Hz, 2H), 3.05 (dd, J=15.8, 7.2 Hz, 2H), 2.74 (t, J=5.0 Hz, 4H). ¹³C NMR (101 MHz, DMSO) δ 165.65, 152.34, 149.98, 145.74, 141.07, 135.43, 130.23, 126.44, 124.43, 50.45, 45.36, 44.84, 38.42. LC-MS (C₁₉H₂₂N₆O₂): Calculated [M+H]⁺ m/z=367.19, [M−H]⁻ m/z=365.17. Observed [M+H]⁺ m/z=367.38, [M−H]⁻ m/z=365.41.

### N-(2,3-dihydro-1H-inden-2-yl)-3-(1-hydroxycyclohexane-1-carboxamido)pyrazine-2-carboxamide (15)

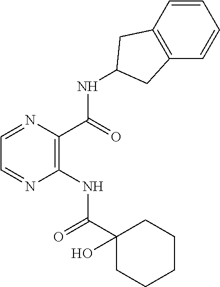 [SMILES: O=C(NC1Cc2ccccc2C1)c1nccnc1NC(=O)C1(O)CCCCC1]

Mixed 1-hydroxycyclohexane-1-carboxylic acid (100 mg, 0.694 mmol), N-hydroxysuccinimide (95.4 mg, 829 μmol), and MeCN (3.5 mL). Added solution of N,N-dicyclohexylcarbodiimide (147 mg, 0.713 mmol) in DCM (1 mL) to produce a white precipitate. Stirred 1.25 h, filtered with cotton to remove the insoluble material, dried over Na₂SO₄, filtered with cotton, and concentrated to obtain crude 1-hydroxycyclohexane-1-carboxylate N-hydroxysuccinimide ester which was used without further purification or characterization. To a solution of 1-hydroxycyclohexane-1-carboxylate N-hydroxysuccinimide ester (50 mg, 0.21 mmol) in DCM (500 μL), successively added chlorotrimethylsilane (TMSCl; 53 μL, 45 mg, 0.42 mmol) and imidazole (57 mg, 0.84 mmol). After 1 h, reaction was complete by LC-MS and a white precipitate was observed. Filtered with cotton and concentrated the filtrate to obtain crude 2,5-dioxopyrrolidin-1-yl 1-((trimethylsilyl)oxy)cyclohexane-1-carboxylate which was used without further purification or characterization. Resuspended this in anhydrous THE (1.0 mL). In a fresh vial, dissolved 45 (30.0 mg, 0.118 mmol, 1 eq) in THF (3.0 mL) and added 1.0 M NaHMDS_{(THF)} (590 μL, 0.590 mmol) to produce a cloudy, orange suspension. Added the slurry of 2,5-dioxopyrrolidin-1-yl 1-((trimethylsilyl)oxy)cyclohexane-1-carboxylate in THF. After 5 min, reaction was complete by LC-MS. Diluted with DCM (30 mL) and quenched with 1.0 M HCl_{(aq)} (30 mL). Stirred overnight. Discarded HCl_{(aq)} quench and washed organic layer with 1.0 M HCl_{(aq)} (30 mL). Dried over Na₂SO₄, filtered, and concentrated. To remove the TMS protecting group, dissolved in mixture of MeOH (2.5 mL), DCM (2.5 mL), and acetic acid (20 L). After stirring for 4 h, LC-MS showed reaction was complete. Diluted with DCM (30 mL), washed twice with saturated NaHCO_{3(aq)} (30 mL), and washed once with saturated NaCl_{(aq)} (30 mL). Dried over Na₂SO₄, filtered, and concentrated. Purified by normal phase flash column chromatography (hexanes/EtOAc). Yield: 5.1 mg, 11.3%. White solid. ¹H NMR (400 MHz, Chloroform-d) δ 12.85 (s, 1H), 8.60 (s, 1H), 8.31 (d, J=7.9 Hz, 1H), 8.15 (s, 1H), 7.28-7.24 (m, 2H), 7.24-7.18 (m, 2H), 4.93 (h, J=7.7 Hz, 1H), 3.42 (dd, J=16.2, 7.2 Hz, 2H), 2.96 (dd, J=16.2, 4.6 Hz, 2H), 2.74 (s, 1H), 2.02 (td, J=13.7, 3.8 Hz, 2H), 1.81-1.69 (m, 5H), 1.67-1.58 (m, 2H), 1.44-1.35 (m, 1H). ¹³C NMR (101 MHz, CDCl₃) δ 175.49, 164.94, 149.34, 146.50, 140.67, 136.70, 129.92, 127.10, 124.98, 75.93, 50.65, 40.19, 34.62, 25.08, 21.36. LC-MS (C₂₁H₂₄N₄O₃): Calculated [M−H]⁻ m/z=379.18. Observed [M−H]⁻ m/z=379.21.

### tert-butyl (1-((3-((2,3-dihydro-1H-inden-2-yl)carbamoyl)pyrazin-2-yl)carbamoyl)cyclohexyl)carbamate (16)

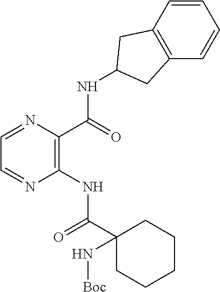 [SMILES: CC(C)(C)OC(=O)NC1(C(=O)Nc2nccnc2C(=O)NC2Cc3ccccc3C2)CCCCC1]

To a solution of 1-((tert-butoxycarbonyl)amino)cyclohexane-1-carboxylic acid (86.1 mg, 354 μmol, 0.9 eq) in 1:1 MeCN/DCM, added N-hydroxysuccinimide (40.7 mg, 354 mol, 0.9 eq) and then a solution of N,N′-dicyclohexylcarbodiimide (73.0 mg, 354 μmol, 0.9 eq) in MeCN (0.71 mL). Stirred 15 min, filtered, concentrated in vacuo, and resuspended in DMF (2 mL). Separately combined 45 (100 mg, 393 μmol, 1 eq), DMF (5 mL), and 1.0M NaHMDS_{(THF)} (0.79 mL, 787 μmol, 2 eq) and added this dropwise to the reaction mixture. After 15 min, diluted with EtOAc (100 mL) and washed twice with saturated NH₄Cl_{(aq)}. Dried over Na₂SO₄, filtered, and concentrated in vacuo. Purified by normal phase flash column chromatography (hexanes/EtOAc). Yield: 50.0 mg, 26.5%. ¹H NMR (400 MHz, Chloroform-d) δ 12.58 (s, 1H), 8.58 (d, J=2.4 Hz, 1H), 8.29 (d, J=8.3 Hz, 1H), 8.09 (s, 1H), 7.27 (s, 2H), 7.24-7.19 (m, 2H), 5.10 (s, 1H), 4.96-4.84 (m, 1H), 3.40 (dd, J=16.2, 7.3 Hz, 2H), 2.95 (dd, J=16.0, 5.1 Hz, 2H), 2.07 (s, 2H), 1.99 (td, J=13.4, 12.9, 3.8 Hz, 2H), 1.79-1.64 (m, 3H), 1.44 (d, J=14.0 Hz, 12H). ¹³C NMR (101 MHz, CDCl₃) δ 173.29, 164.96, 164.72, 154.27, 149.80, 146.46, 145.50, 140.53, 136.02, 129.27, 126.98, 124.85, 50.40, 40.06, 31.95, 28.38, 25.15, 21.37. LC-MS (C₂₆H₃₃N₅O₄): Calculated [M+H]⁺ m/z=480.25, [M−H]⁻ m/z=478.25. Observed [M+H]⁺ m/z=480.44, [M−H]⁻ m/z=478.33.

### 3-(1-aminocyclohexane-1-carboxamido)-N-(2,3-dihydro-1H-inden-2-yl)pyrazine-2-carboxamide (17)

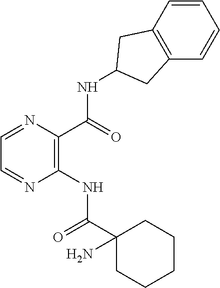 [SMILES: NC1(C(=O)Nc2nccnc2C(=O)NC2Cc3ccccc3C2)CCCCC1]

To a solution of 16 (10.0 mg, 20.9 μmol, 1 eq) in DCM (1 mL), added TFA (1 mL) and stirred vigorously for 30 min. Concentrated in vacuo. Purified by reverse phase flash column chromatography (water+0.1% formic acid/MeCN+0.1% formic acid). Yield: 8.5 mg, >95%. ¹H NMR (400 MHz, DMF-d₇) δ 9.15 (d, J=7.8 Hz, 1H), 8.60 (d, J=2.3 Hz, 1H), 8.32 (d, J=2.3 Hz, 1H), 7.35-7.22 (m, 2H), 7.19 (dd, J=5.5, 3.2 Hz, 2H), 4.86 (h, J=7.4 Hz, 1H), 3.32 (dd, J=15.8, 7.6 Hz, 2H), 3.17 (dd, J=15.7, 6.9 Hz, 2H), 1.94 (dd, J=12.2, 9.3 Hz, 2H), 1.77-1.54 (m, 7H), 1.36-1.18 (m, 1H). ¹³C NMR (101 MHz, DMF-d₇) δ 177.42, 166.19, 163.87, 149.98, 146.85, 142.44, 137.99, 132.39, 127.63, 125.62, 59.56, 52.00, 39.94, 26.54, 22.11. ¹³C NMR (101 MHz, CDCl₃) δ 172.80, 165.33, 149.58, 146.60, 140.54, 136.61, 129.24, 127.16, 124.99, 67.34, 50.79, 44.02, 40.08, 29.02. LC-MS (C₂₁H₂₅N₅O₂): Calculated [M+H]⁺ m/z=380.20, [M−H]⁻ m/z=378.20. Observed [M+H]⁺ m/z=380.40, [M−H]⁻ m/z=378.20.

### N-(2,3-dihydro-1H-inden-2-yl)-3-(tetrahydro-2H-pyran-4-carboxamido)pyrazine-2-carboxamide (18)

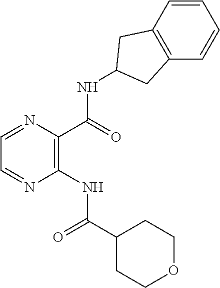 [SMILES: O=C(NC1Cc2ccccc2C1)c1nccnc1NC(=O)C1CCOCC1]

Dissolved 45 (50.0 mg, 197 μmol, 1 eq) in THE (5.6 mL) and added 1.0M NaHMDS_{(THF)} (983 μL, 983 μmol, 5 eq) to produce a cloudy, yellow suspension. In a second vial, dissolved tetrahydro-2H-pyran-4-carboxylic acid (39.0 mg, 300 μmol, 1.5 eq) in THE (1.0 mL) and successively added a drop of DMF and then oxalyl chloride (20.0 μL, 29.0 mg, 228 μmol, 1.15 eq) (note: this mixture is defined as being 1.5 equivalents of tetrahydro-2H-pyran-4-carbonyl chloride solution). Added the oxalyl chloride solution dropwise to the 45 vial to produce a cloudy, orange solution. LC-MS analysis showed only partial conversion so after 5 min, so another one equivalent of tetrahydro-2H-pyran-4-carbonyl chloride solution was prepared as described above and was added to the reaction 40 min after it started. LC-MS analysis showed only a minor increase in yield so after 1.67 h, added 1.0M NaHMDS_{(THF)} (383 μL, 0.383 mmol, 2 eq) and an additional two equivalents of tetrahydro-2H-pyran-4-carbonyl chloride solution. Stirred reaction 15 h. Concentrated to remove THF, diluted with EtOAc (30 mL), washed twice with 3:1 water/saturated NH₄Cl_{(aq)} (30 mL), twice with water (30 mL), and once with saturated NaCl_{(aq)} (30 mL). Dried over Na₂SO₄, filtered, and concentrated. Purified by normal phase FCC, eluting with 1% EtOH in EtOAc. Product fractions were concentrated and repurified by normal phase flash column chromatography (hexanes/EtOAc). Yield: 9.3 mg, 12.9%. ¹H NMR (400 MHz, Chloroform-d) δ 12.11 (s, 1H), 8.58 (s, 1H), 8.33 (d, J=8.2 Hz, 1H), 8.14 (s, 1H), 7.29-7.26 (m, 2H), 7.24-7.19 (m, 2H), 4.94-4.82 (m, 1H), 4.07 (d, J=11.5 Hz, 2H), 3.58-3.47 (m, 2H), 3.43 (dd, J=16.2, 7.3 Hz, 2H), 2.97 (dd, J=16.2, 4.8 Hz, 2H), 2.70 (p, J=7.8 Hz, 1H), 2.01-1.92 (m, 4H). ¹³C NMR (101 MHz, CDCl₃) δ 172.80, 165.33, 149.58, 146.60, 140.54, 136.61, 129.24, 127.16, 124.99, 67.34, 50.79, 44.02, 40.08, 29.02. LC-MS (C₂₀H₂₂N₄O₃): Calculated [M+H]⁺ m/z=367.17, [M−H]⁻ m/z=365.17. Observed [M+H]⁺ m/z=367.38, [M−H]⁻ m/z=365.20.

### N-(2,3-dihydro-1H-inden-2-yl)-3-(cis-2,6-dimethylpiperidine-1-carboxamido)pyrazine-2-carboxamide (19)

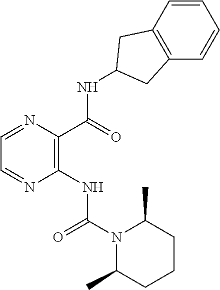 [SMILES: C[C@@H]1CCC[C@H](C)N1C(=O)Nc1nccnc1C(=O)NC1Cc2ccccc2C1]

To a solution of 45 (100 mg, 0.393 mmol, 1 eq) in anhydrous DCM (15 mL), added anhydrous lutidine (184 μL, 170 mg, 1.57 mmol, 4 eq). Cooled to 0° C. Added 15% w/v phosgene₍ₜₒₗᵤₑₙₑ₎ (7.9 mL, 11 mmol, 1.4 eq) and stirred 20 min at 0° C. Added cis-2,6-dimethylpiperidine (178 mg, 1.57 mmol, 4 eq), warmed to room temperature, and stirred overnight. Concentrated, resuspended in DCM (3 mL), filtered off insoluble, and concentrated the DCM soluble. Purified by normal phase flash column chromatography (hexanes/EtOAc). Yield: 80.6 mg, 52.0%. White solid. ¹H NMR (400 MHz, DMSO-d₆) δ 11.00 (s, 1H), 9.21 (d, J=7.9 Hz, 1H), 8.51 (d, J=2.0 Hz, 1H), 8.21 (d, J=2.2 Hz, 1H), 7.22 (d, J=4.5 Hz, 2H), 7.15 (t, J=4.5 Hz, 2H), 4.72 (q, J=7.6 Hz, 1H), 4.35 (s, 2H), 3.17 (d, J=7.7 Hz, 2H), 3.05 (dd, J=15.7, 7.3 Hz, 2H), 1.79 (dq, J=13.5, 7.0 Hz, 1H), 1.68-1.57 (m, 4H), 1.46 (d, J=13.0 Hz, 1H), 1.26 (d, J=6.9 Hz, 6H). ¹H NMR (400 MHz, Chloroform-d) δ 11.38 (s, 1H), 8.50 (t, J=1.7 Hz, 1H), 8.28 (d, J=8.3 Hz, 1H), 7.97 (t, J=1.7 Hz, 1H), 7.25 (q, J=4.1 Hz, 2H), 7.22-7.16 (m, 2H), 4.87 (ddt, J=12.8, 7.9, 4.2 Hz, 1H), 4.53 (p, J=6.7 Hz, 2H), 3.40 (dd, J=16.1, 7.3 Hz, 2H), 2.94 (dd, J=16.2, 5.0 Hz, 2H), 1.91-1.75 (m, 1H), 1.77-1.61 (m, 4H), 1.52 (dq, J=12.3, 3.7 Hz, 1H), 1.36 (d, J=7.0 Hz, 6H). ¹³C NMR (101 MHz, CDCl₃) δ 165.80, 153.02, 151.42, 146.59, 140.66, 134.60, 128.76, 126.99, 124.89, 50.49, 46.06, 40.07, 30.41, 21.05, 13.89. LC-MS (C₂₂H₂₇N₅O₂): Calculated [M+H]⁺ m/z=394.22, [M−H]⁻ m/z=392.22. Observed [M+H]⁺ m/z=394.48, [M−H]⁻ m/z=392.35.

### 3-(cyclobutanecarboxamido)-N-(2,3-dihydro-1H-inden-2-yl)pyrazine-2-carboxamide (20)

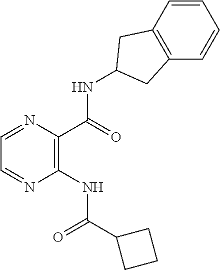 [SMILES: O=C(NC1Cc2ccccc2C1)c1nccnc1NC(=O)C1CCC1]

Combined 45 (100 mg, 0.20 mmol, 1 eq), THE (10 mL), and 1.0M NaHMDS_{(THF)} (1.97 mL, 1.97 mmol, 5 eq) and stirred vigorously for 5 min. Added this to a solution of cyclobutanecarbonyl chloride (93.2 mg, 0.787 mmol, 2 eq) in THE (5 mL) and stirred vigorously. After 2 h, diluted with EtOAc and washed twice with 1:1 water/saturated NH₄Cl_{(aq)}. Dried over Na₂SO₄, filtered, and concentrated in vacuo. Purified by reverse phase flash column chromatography (water+0.1% formic acid/MeCN+0.1% formic acid) and then further purified by normal phase flash column chromatography (hexanes/EtOAc). Yield: 35.7 mg, 27.0%. ¹H NMR (400 MHz, Chloroform-d) δ 11.92 (s, 1H), 8.60 (s, 1H), 8.35 (d, J=8.1 Hz, 1H), 8.15 (s, 1H), 7.30 (q, J=4.1 Hz, 2H), 7.28-7.22 (m, 2H), 4.91 (tq, J=7.5, 4.8, 3.8 Hz, 1H), 3.48 (d, J=7.3 Hz, 1H), 3.46-3.37 (m, 2H), 3.01 (dd, J=16.2, 4.8 Hz, 2H), 2.49 (dq, J=11.7, 9.1 Hz, 2H), 2.35 (qd, J=8.8, 4.4 Hz, 2H), 2.03 (dddd, J=25.1, 20.5, 12.4, 7.9 Hz, 2H). ¹³C NMR (101 MHz, CDCl₃) δ 173.63, 165.20, 149.49, 146.45, 140.53, 136.21, 129.01, 127.03, 124.89, 50.66, 41.90, 40.00, 25.29, 18.09. LC-MS (C₁₉H₂₀N₄O₂): Calculated [M+H]⁺ m/z=337.16, [M−H]⁻ m/z=335.16. Observed [M+H]⁺ m/z=337.39, [M−H]⁻ m/z=335.20.

### 3-benzamido-N-(2,3-dihydro-1H-inden-2-yl)pyrazine-2-carboxamide (21)

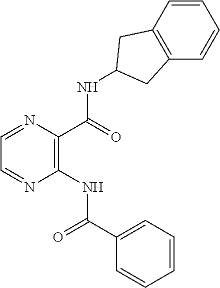 [SMILES: O=C(Nc1nccnc1C(=O)NC1Cc2ccccc2C1)c1ccccc1]

Under argon atmosphere, dissolved 45 (20 mg, 78.7 μmol; 1 eq) in anhydrous THE (2.0 mL) and added 1M NaHMDS_{(THF)} (0.20 mL, 197 μmol, 2.5 eq). Separately combined benzoyl chloride (18.3 μL, 22.2 mg, 157 μmol, 2 eq) and THE (0.6 mL). Added the benzoyl chloride solution to the 45 suspension dropwise and stirred overnight. Concentrated to dryness, diluted with EtOAc (30 mL), washed twice with 3:1 water/saturated NH₄Cl_{(aq)} (30 mL), twice with water (30 mL), and once with saturated NaCl_{(aq)}. Dried over Na₂SO₄, filtered, and concentrated in vacuo. Purified by normal phase flash column chromatography (1% EtOH in EtOAc). Repurified by normal phase flash column chromatography (hexanes/EtOAc). Yield: 12.9 mg, 45.7%. ¹H NMR (400 MHz, Chloroform-d) δ 12.94 (s, 1H), 8.64 (s, 1H), 8.37 (d, J=8.0 Hz, 1H), 8.16 (s, 1H), 8.12 (d, J=7.2 Hz, 2H), 7.60-7.50 (m, 3H), 7.28-7.24 (m, 2H), 7.23-7.19 (m, 2H), 4.99-4.84 (m, 1H), 3.44 (dd, J=16.3, 7.0 Hz, 2H), 2.99 (dd, J=16.3, 4.7 Hz, 2H). ¹³C NMR (101 MHz, CDCl₃) δ 165.47, 164.83, 149.97, 146.73, 140.59, 136.63, 134.48, 132.53, 129.54, 129.01, 127.89, 127.13, 124.99, 50.79, 40.11. LC-MS (C₂₁H₁₈N₄O₂): Calculated [M+H]⁺ m/z=359.14, [M−H]⁻ m/z=357.14. Observed [M+H]⁺ m/z=359.34, [M−H]⁻ m/z=357.14.

### 5-((3-((2,3-dihydro-1H-inden-2-yl)carbamoyl)pyrazin-2-yl)amino)-5-oxopentanoic acid (22)

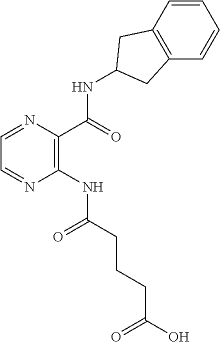 [SMILES: O=C(O)CCCC(=O)Nc1nccnc1C(=O)NC1Cc2ccccc2C1]

Combined 45 (50 mg, 0.20 mmol, 1 eq), glutaric anhydride (22 mg, 0.20 mmol, 1 eq), DCM (5 mL), and 1.0M NaHMDS_{(THF)} (0.6 mL, 0.59 mmol, 3 eq). After 2 h, diluted with DCM, washed with 1M HCl_{(aq)}, and washed with saturated NaCl_{(aq)}. Dried over Na₂SO₄, filtered, and concentrated in vacuo. Purified by ion exchange chromatography on a 500 mg Biotage Evolute AX column (successively pre-washed with MeOH, water, and then 5% NH₄OH_{(aq)}; loaded in 5% NH₄OH_{(aq)}; successively washed with water, MeOH, DCM, and then again with MeOH; and eluted with 0.25→0.5% formic acid_{(MeOH)}). Concentrated in vacuo. Yield: 5.8 mg, 8%. Brown solid. ¹H NMR (400 MHz, Methanol-d₄) δ 8.47 (s, 1H), 8.30 (s, 1H), 7.26-7.19 (m, 2H), 7.15 (dd, J=5.5, 3.3 Hz, 2H), 4.81 (q, J=7.0 Hz, 1H), 3.66 (s, 1H), 3.35 (d, J=7.6 Hz, 1H), 3.04 (dd, J=15.8, 6.6 Hz, 2H), 2.69 (t, J=7.3 Hz, 2H), 2.44 (t, J=7.3 Hz, 2H), 2.39 (t, J=7.3 Hz, 1H), 2.34 (t, J=7.2 Hz, 1H), 2.03 (p, J=7.4 Hz, 2H), 1.88 (p, J=7.3 Hz, 1H). ¹³C NMR (101 MHz, MeOD) δ 175.28, 173.66, 166.87, 149.51, 146.57, 142.07, 138.70, 132.09, 127.81, 125.58, 52.23, 52.01, 40.07, 38.12, 34.10, 33.85, 21.46, 21.37. LC-MS (C₁₉H₂₀N₄O₄): Calculated [M+H]⁺ m/z=369.15, [M−H]⁻ m/z=367.15. Observed [M+H]⁺ m/z=369.30, [M−H]⁻ m/z=367.15.

### N-(2,3-dihydro-1H-inden-2-yl)-3-ureidopyrazine-2-carboxamide (23)

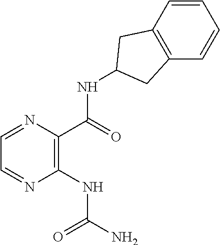 [SMILES: NC(=O)Nc1nccnc1C(=O)NC1Cc2ccccc2C1]

To a solution of 45 (20.0 mg, 78.7 μmol, 1 eq) in anhydrous DCM (2 mL), added anhydrous lutidine (37 μL, 34 mg, 0.32 mmol, 4 eq) and cooled to 0° C. Added 15% w/v phosgene₍ₜₒₗᵤₑₙₑ₎ (63 μL, 0.94 mmol, 1.2 eq) dropwise and then stirred 10 min at 0° C. Added N-hydroxysuccinimide (30 mg, 0.26 mmol) and stirred overnight. Diluted with DCM (30 mL), washed twice with 1:1 water/saturated NH₄Cl_{(aq)}, washed twice with 1:1 water/saturated NaHCO_{3(aq)}, and washed once with saturated NaCl_{(aq)}. Dried over Na₂SO₄, filtered, and concentrated in vacuo. Triturated with MeCN, filtered, and successively washed with MeCN and then DCM. Yield: 6.1 mg, 26%. White solid. ¹H NMR (400 MHz, DMSO-d₆) δ 10.89 (s, 1H), 9.27 (d, J=7.9 Hz, 1H), 8.48 (s, 1H), 8.29 (s, 1H), 8.25 (s, 1H), 7.33 (s, 1H), 7.22 (d, J=4.4 Hz, 2H), 7.16 (d, J=4.4 Hz, 2H), 4.73 (h, J=7.6 Hz, 1H), 3.20 (dd, J=15.7, 7.7 Hz, 2H), 3.05 (dd, J=15.7, 7.4 Hz, 2H). ¹³C NMR (101 MHz, DMSO-d₆) δ 164.99, 153.93, 149.10, 144.44, 141.04, 134.87, 128.49, 126.47, 124.44, 50.46, 38.33. LC-MS (C₁₅H₁₅N₅O₂): Calculated [M+H]⁺ m/z=298.12, [M−H]⁻ m/z=296.12. Observed [M+H]⁺ m/z=298.38, [M−H]⁻ m/z=296.19.

### 5′-O—[N-(D-Prolyl)-sulfamoyl]adenosine (D-ProSA, 27)

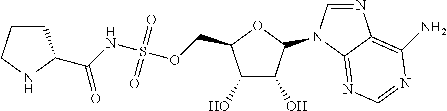

Synthesized as described previously from 53. ¹H NMR (D₂O) and LC-MS data matched what was reported Heacock et al. The ¹³C NMR data was not reported by Heacock et al.

¹³C NMR (101 MHz, D20) δ 175.31, 155.51, 152.85, 148.89, 139.72, 118.58, 87.42, 82.17, 73.95, 70.14, 68.52, 62.19, 46.23, 29.49, 23.55.

### Perfluorophenyl 6-((tert-butoxycarbonyl)amino)hexanoate (51)

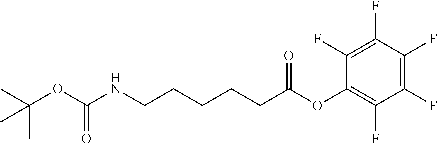 [SMILES: CC(C)(C)OC(=O)NCCCCCC(=O)Oc1c(F)c(F)c(F)c(F)c1F]

Dissolved 6-((tert-butoxycarbonyl)amino)hexanoic acid (500 mg, 2.16 mmol, 1 eq) in DMF (4 mL) and added bis(perfluorophenyl) carbonate (1.02 g, 2.59 mmol, 1.2 eq) and DIPEA (1.51 mL, 1.12 g, 8.65 mmol, 4 eq). Stirred 25 min. Diluted with EtOAc (150 mL), washed twice with 0.2M HCl_{(aq)} (100 mL), washed three times with 4:1 mixture of water and saturated NaHCO_{3(aq)} (100 mL), and washed once with 3:1 mixture of water and saturated NaCl_{(aq)} (100 mL). Dried over Na₂SO₄, filtered, and concentrated in vacuo. Yield: 424 mg, 49.3%. White solid. MS and NMR data matched that previously described.

### tert-butyl (6-((1-((3-((2,3-dihydro-1H-inden-2-yl)carbamoyl)pyrazin-2-yl)carbamoyl)piperidin-4-yl)amino)-6-oxohexyl)carbamate (41)

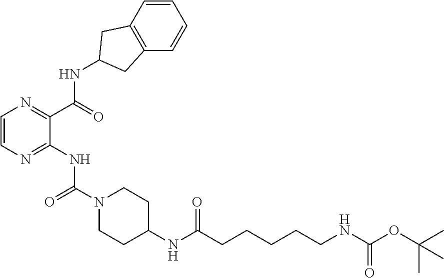 [SMILES: CC(C)(C)OC(=O)NCCCCCC(=O)NC1CCN(C(=O)Nc2nccnc2C(=O)NC2Cc3ccccc3C2)CC1]

To a stirred solution of 8·2HCl (213 mg, 470 μmol, 1 eq), DCM (5 mL), and DIPEA (307 μL, 228 mg, 1.76 mmol, 3.47 eq), added a solution of 51 (214.5 mg, 540 μmol, 1.15 eq) in DCM (5 mL) and stirred 4 h. Diluted with EtOAc (300 mL)+MeOH (3 mL), washed twice with 1:1 mixture of water and saturated NaHCO_{3(aq)} (200 mL), washed twice with 0.2M HCl_{(aq)} (50 mL), and washed once with 3:1 mixture of water and saturated NaCl_{(aq)} (100 mL). Dried over Na₂SO₄, filtered, and concentrated in vacuo. Purified by reverse phase flash column chromatography (water+0.1% formic acid/MeCN+0.1% formic acid). Yield: 254 mg, 88.9%. White solid. ¹H NMR (400 MHz, CDCl₃) δ 11.51 (s, 1H), 8.49 (d, J=2.4 Hz, 1H), 8.28 (d, J=8.2 Hz, 1H), 8.02 (d, J=2.4 Hz, 1H), 7.29-7.16 (m, 4H), 5.63 (d, J=8.0 Hz, 1H), 4.85 (dtd, J=12.3, 7.4, 4.9 Hz, 1H), 4.56 (s, 1H), 4.25 (d, J=13.6 Hz, 2H), 4.03 (dtt, J=11.6, 7.9, 3.9 Hz, 1H), 3.41 (dd, J=16.1, 7.2 Hz, 2H), 3.14-3.02 (m, 4H), 2.95 (dd, J=16.1, 4.8 Hz, 2H), 2.17 (t, J=7.6 Hz, 2H), 2.02 (dd, J=13.2, 3.9 Hz, 2H), 1.65 (p, J=7.6 Hz, 2H), 1.55-1.25 (m, 15H). ¹³C NMR (101 MHz, CDCl₃) δ 172.73, 165.72, 156.21, 152.57, 150.85, 146.15, 140.56, 135.12, 128.93, 127.12, 124.96, 79.34, 50.71, 46.73, 43.37, 40.58, 40.07, 36.53, 32.13, 29.83, 28.55, 26.41, 25.42. LC-MS (C₃₁H₄₃N₇O₅): Calculated [M+H]⁺ m/z=594.73, [M−H]⁻ m/z=592.73. Observed [M+H]⁺ m/z=594.66, [M−H]⁻ m/z=592.56.

### 3-(4-(6-aminohexanamido)piperidine-1-carboxamido)-N-(2,3-dihydro-1H-inden-2-yl)pyrazine-2-carboxamide dihydrochloride (42-2HCl)

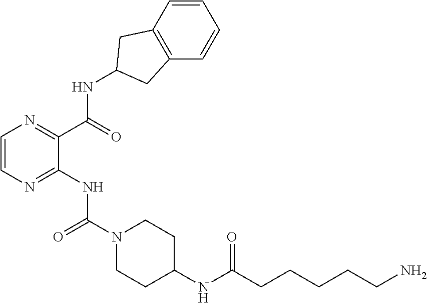 [SMILES: NCCCCCC(=O)NC1CCN(C(=O)Nc2nccnc2C(=O)NC2Cc3ccccc3C2)CC1]

To a vigorously stirred solution of MeOH (6 mL), added SOCl₂ (500 μL, 815 mg, 6.85 mmol, 35.5 eq) dropwise over 5 minutes and stirred for 20 minutes. Added the entire MeOH/SOCl₂ solution to dry 41 (115 mg, 193 μmol, 1 eq) and stirred for 45 min. Concentrated to a dry white solid. Yield: 108.3 mg, >95%. White solid. ¹H NMR (400 MHz, MeOD) δ 8.43 (s, 2H), 7.29-7.11 (m, 4H), 4.85 (t, J=6.6 Hz, 1H), 4.18 (d, J=13.0 Hz, 2H), 3.99 (s, 1H), 3.39-3.32 (m, 2H), 3.22 (t, J=12.1 Hz, 2H), 3.07 (dd, J=15.8, 6.4 Hz, 2H), 2.93 (t, J=7.2 Hz, 2H), 2.25 (t, J=7.1 Hz, 2H), 1.99 (d, J=11.4 Hz, 2H), 1.78-1.62 (m, 4H), 1.62-1.48 (m, 2H), 1.43 (q, J=7.5 Hz, 2H). ¹³C NMR (101 MHz, MeOD) δ 175.26, 166.17, 154.03, 148.65, 141.91, 139.39, 136.96, 134.29, 127.86, 125.56, 52.36, 47.58, 44.25, 40.56, 39.87, 36.57, 32.42, 28.22, 26.92, 26.31. LC-MS (C₂₆H₃₅N₇O₃): Calculated [M+H]⁺ m/z=494.29, [M−H]⁻ m/z=492.27. Observed [M+H]⁺ m/z=494.48, [M−H]⁻ m/z=492.44.

### tert-butyl (6-(((1-((3-((2,3-dihydro-1H-inden-2-yl)carbamoyl)pyrazin-2-yl)carbamoyl)piperidin-4-yl)methyl)amino)-6-oxohexyl)carbamate (43)

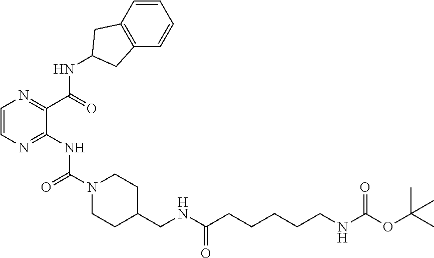 [SMILES: CC(C)(C)OC(=O)NCCCCCC(=O)NCC1CCN(C(=O)Nc2nccnc2C(=O)NC2Cc3ccccc3C2)CC1]

Charged a vial with 10·2HCl (204 mg, 437 μmol, 1 eq), DCM (5 mL), and DIPEA (298 μL, 221 mg, 1.71 mmol, 3.92 eq) and stirred vigorously. Over the course of 1 min, slowly added solution of 51 (215 mg, 540 μmol, 1.24 eq) and DCM (5 mL). Stirred 3 h. Diluted with EtOAc (300 mL) and MeOH (3 mL). Washed twice with 1:1 water/saturated NaHCO_{3(aq)} (200 mL), washed twice with 0.2M HCl_{(aq)} (50 mL), and washed once with 3:1 water/saturated NaCl_{(aq)} (100 mL). Dried over Na₂SO₄, filtered, and concentrated in vacuo. Purified by reverse phase flash column chromatography (water+0.1% formic acid/MeCN+0.1% formic acid). Yield: 247 mg, 93%. Light yellow solid. ¹H NMR (400 MHz, CDCl₃) δ 11.45 (s, 1H), 8.49 (d, J=2.3 Hz, 1H), 8.28 (d, J=8.1 Hz, 1H), 7.99 (d, J=2.3 Hz, 1H), 7.29-7.16 (m, 4H), 5.79 (s, 1H), 4.85 (dtd, J=12.3, 7.5, 4.9 Hz, 1H), 4.58 (s, 1H), 4.30 (d, J=13.3 Hz, 2H), 3.41 (dd, J=16.2, 7.2 Hz, 2H), 3.17 (t, J=6.1 Hz, 2H), 3.09 (t, J=7.1 Hz, 2H), 3.01-2.81 (m, 4H), 2.18 (t, J=7.6 Hz, 2H), 1.85-1.72 (m, 3H), 1.65 (p, J=7.6 Hz, 2H), 1.53-1.18 (m, 15H). ¹³C NMR (101 MHz, CDCl₃) δ 173.43, 165.80, 156.18, 152.55, 151.09, 146.47, 140.60, 134.93, 128.70, 127.08, 124.95, 79.27, 50.66, 44.89, 44.25, 40.50, 40.06, 36.55, 36.41, 29.87, 28.54, 26.49, 25.46.

LC-MS (C₃₂H₄₅N₇O₅): Calculated [M+H]⁺ m/z=608.76, [M−H]⁻ m/z=606.76. Observed [M+H]⁺ m/z=608.37, [M−H]⁻ m/z=606.49.

### 3-(4-((6-aminohexanamido)methyl)piperidine-1-carboxamido)-N-(2,3-dihydro-1H-inden-2-yl)pyrazine-2-carboxamide dihydrochloride (44-2HCl)

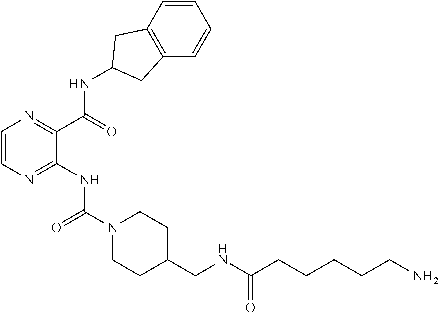 [SMILES: NCCCCCC(=O)NCC1CCN(C(=O)Nc2nccnc2C(=O)NC2Cc3ccccc3C2)CC1]

To a vigorously stirred solution of MeOH (6 mL), added SOCl₂ (500 μL, 815 mg, 6.85 mmol, 49.2 eq) dropwise over 5 minutes and stirred for 20 minutes. Added the entire MeOH/SOCl₂ solution to dry 43 (84.6 mg, 139 μmol, 1 eq) and stirred for 45 min. Concentrated to a dry white solid. Yield: 69.1 mg, 85.5%. Light yellow solid. ¹H NMR (400 MHz, MeOD) δ 8.50 (s, 1H), 8.41 (s, 1H), 7.28-7.10 (m, 4H), 4.86 (d, J=9.1 Hz, 1H), 4.23 (s, 2H), 3.40-3.32 (m, 2H), 3.19-3.04 (m, 6H), 2.92 (d, J=7.4 Hz, 2H), 2.27 (d, J=7.1 Hz, 2H), 1.87 (d, J=11.1 Hz, 3H), 1.68 (q, J=7.8 Hz, 4H), 1.43 (t, J=7.4 Hz, 2H), 1.37-1.18 (m, 2H). ¹³C NMR (101 MHz, MeOD) δ 176.15, 165.79, 153.92, 147.86, 141.89, 136.83, 135.40, 127.88, 127.82, 125.57, 52.42, 45.45, 40.57, 39.82 (s, 2C), 37.22, 36.56, 30.74, 28.22, 27.00, 26.39. LC-MS (C₂₇H₃₉Cl₂N₇O₃): Calculated [M+H]⁺ m/z=581.56, [M−H]⁻ m/z=579.56. Observed [M+H]⁺ m/z=508.47, [M−H]⁻ m/z=506.46.

### N-(2,3-dihydro-1H-inden-2-yl)-3-(methylamino)pyrazine-2-carboxamide (46)

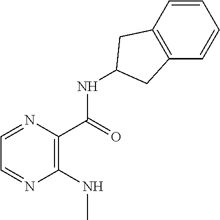 [SMILES: CNc1nccnc1C(=O)NC1Cc2ccccc2C1]

To a solution of 45 (25.0 mg, 98.3 μmol, 1 eq) in DMF (3 mL), successively added 1.0 M NaHMDS_{(THF)} (200 μL, 197 μmol, 2 eq) and then a solution of methyl iodide (61.5 μL, 140 mg, 983 μmol, 10 eq) in DMF (0.5 mL). After 5 minutes, diluted with DCM (100 mL), washed once with saturated NH₄Cl_{(aq)} (100 mL), and washed twice with 1M HCl_{(aq)} (100 mL). Dried over Na₂SO₄, filtered, and concentrated in vacuo. Purified by normal phase flash column chromatography (hexanes/EtOAc). Yield: 8.6 mg, 32.6%. Yellow solid. ¹H NMR (400 MHz, Chloroform-d) δ 8.68 (s, 1H), 8.22 (d, J=2.4 Hz, 1H), 8.14 (d, J=8.0 Hz, 1H), 7.65 (d, J=2.5 Hz, 1H), 7.31 (dd, J=5.9, 3.1 Hz, 2H), 7.25 (dd, J=5.4, 3.3 Hz, 2H), 4.89 (dtd, J=12.7, 7.5, 5.2 Hz, 1H), 3.45 (dd, J=16.1, 7.3 Hz, 2H), 3.10 (d, J=5.0 Hz, 3H), 3.00 (dd, J=16.1, 5.2 Hz, 2H). ¹³C NMR (101 MHz, CDCl₃) δ 166.49, 155.41, 146.41, 140.97, 129.05, 126.95, 126.92, 124.93, 50.43, 40.17, 27.47. LC-MS (C₁₅H₁₆N₄O): Calculated [M+H]⁺ m/z=269.13. Observed [M+H]⁺ m/z=269.37.

### 3-(2,3-dihydro-1H-inden-2-yl)pteridine-2,4(1H,3H)-dione (47)

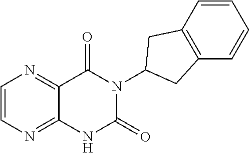 [SMILES: O=c1[nH]c2nccnc2c(=O)n1C1Cc2ccccc2C1]

Synthesized using the general procedure described for synthesizing NCP26 modified to replace piperidine with water and all quantities scaled to 20 mg 45. Reaction was allowed to proceed overnight before it was quenched with MeOH, concentrated in vacuo, and purified by reverse phase flash column chromatography (water+0.1% formic acid/MeCN+0.1% formic acid). Yield: 18 mg, 83%. ¹H NMR (400 MHz, DMSO-d₆) δ 12.19 (s, 1H), 8.66 (s, 1H), 8.55 (d, J=2.3 Hz, 1H), 7.26-7.19 (m, 2H), 7.19-7.13 (m, 2H), 5.76 (dd, J=11.6, 6.5 Hz, 1H), 3.51 (dd, J=15.9, 8.2 Hz, 2H), 3.12 (dd, J=15.9, 9.8 Hz, 2H). ¹³C NMR (101 MHz, DMSO-d₆) δ 160.72, 149.93, 148.26, 148.10, 141.42, 140.25, 127.60, 126.24, 124.31, 50.78, 35.01. LC-MS (C₁₅H₁₂N₄O₂): Calculated [M−H]⁻ m/z=279.10. Observed [M−H]⁻ m/z=279.21.

### 3-amino-N-cyclohexylpyrazine-2-carboxamide (48)

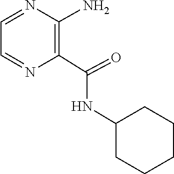 [SMILES: Nc1nccnc1C(=O)NC1CCCCC1]

Suspended 3-aminopyrazine-2-carboxylic acid (205 mg, 1.44 mmol, 1 eq) in DCM (60 mL) and successively added cyclohexylamine (200 μL, 173 mg, 1.74 mmol, 1.2 eq), PyBOP (830 mg, 1.59 mmol, 1.1 eq), and DIPEA (750 μL, 4.31 mmol, 3 eq). After 72 h, diluted reaction with DCM (200 mL), washed twice with 1 M HCl_{(aq)} (200 mL), washed twice with saturated NaHCO_{3(aq)} (200 mL), washed once with saturated NaCl_{(aq)} (200 mL), dried over Na₂SO₄, filtered, and concentrated in vacuo. Purified by normal phase flash column chromatography (1:1 hexanes/EtOAc). Yield: 262 mg, 82.8%. Off-white solid. ¹H NMR (400 MHz, Chloroform-d) δ 8.11 (s, 1H), 7.78 (s, 2H), 3.98-3.81 (m, 1H), 1.98 (d, J=12.0 Hz, 2H), 1.85-1.71 (m, 2H), 1.70-1.60 (m, 1H), 1.43 (q, J=12.0 Hz, 2H), 1.36-1.17 (m, 3H). ¹³C NMR (101 MHz, CDCl₃) δ 165.13, 155.10, 146.17, 131.58, 127.26, 48.19, 33.19, 25.70, 24.99. LC-MS (C₁₁H₁₆N₄O): Calculated [M+H]⁺ m/z=221.13. Observed [M+H]⁺ m/z=221.37.

### N-cyclohexyl-3-(2,3-dihydro-1H-indene-2-carboxamido)pyrazine-2-carboxamide (49)

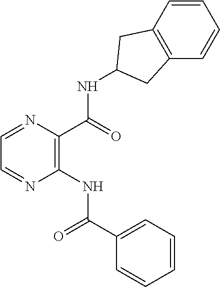 [SMILES: O=C(Nc1nccnc1C(=O)NC1Cc2ccccc2C1)c1ccccc1]

Suspended 2,3-dihydro-1H-indene-2-carboxylic acid (30 mg, 185 μmol, 1 eq) in THE (1 mL). Successively added a drop of DMF and then oxalyl chloride (16.2 μL, 23.5 mg, 185 mol, 1 eq). Stirred 15 min. While waiting, charged a second vial with 48 (50 mg, 220 μmol, 1.2 eq), THE (3 mL), and 1.0 M NaHMDS_{(THF)} (0.55 mL, 0.56 mmol, 3 eq) to obtain a cloudy yellow suspension. After 5 min, added the NaHMDS suspension to the first solution and stirred for 1 h. Diluted reaction with DCM (100 mL), washed twice with 1 M HCl_{(aq)} (100 mL), washed once with saturated NaCl_{(aq)} (100 mL), dried over Na₂SO₄, filtered, and concentrated in vacuo. Purified by normal phase flash column chromatography (hexanes/EtOAc). Yield: 4.6 mg, 6.8%. ¹H NMR (400 MHz, Chloroform-d) δ 12.25 (s, 1H), 8.59 (d, J=2.4 Hz, 1H), 8.19 (d, J=2.4 Hz, 1H), 8.08 (d, J=8.7 Hz, 1H), 7.23 (dd, J=5.4, 3.4 Hz, 2H), 7.19-7.13 (m, 2H), 3.93 (tdd, J=10.1, 7.2, 4.0 Hz, 1H), 3.56 (q, J=8.7 Hz, 1H), 3.43 (dd, J=15.7, 8.7 Hz, 2H), 3.32 (dd, J=15.6, 8.8 Hz, 2H), 2.07-1.97 (m, 2H), 1.80 (dt, J=13.2, 3.9 Hz, 2H), 1.72-1.63 (m, 1H), 1.51-1.21 (m, 4H). ¹³C NMR (101 MHz, CDCl₃) δ 173.06, 164.54, 149.58, 146.40, 141.74, 136.51, 129.55, 126.72, 124.51, 48.63, 47.89, 36.34, 33.00, 25.59, 24.90. LC-MS (C₂₁H₂₄N₄O₂): Calculated [M+H]⁺ m/z=365.19, [M−H]⁻ m/z=363.19. Observed [M+H]⁺ m/z=365.38, [M−H]⁻ m/z=363.22.

### 3-(cyclohexanesulfonamido)-N-(2,3-dihydro-1H-inden-2-yl)pyrazine-2-carboxamide (50)

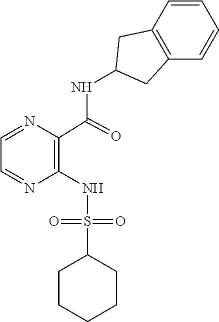 [SMILES: O=C(NC1Cc2ccccc2C1)c1nccnc1NS(=O)(=O)C1CCCCC1]

Dissolved 45 (50.5 mg, 199 μmol, 1 eq) in THE (4.6 mL) under Ar_{(g)} atmosphere and added 1.0M NaHMDS_{(THF)} (600 μL, 600 μmol, 3 eq). Stirred 15 min before adding this mixture dropwise over 5 min to a stirred solution of cyclohexanesulfonyl chloride (34.5 μL, 43.4 mg, 238 μmol, 1.2 eq) in THF (2 mL) under Ar_{(g)} atmosphere. After 1 h, added more THF (2 mL) as some had evaporated through a hole in the septum. Replaced septum and stirred reaction overnight under Ar_{(g)} atmosphere. In morning, reaction was a cloudy, yellow suspension and LC-MS analysis indicated incomplete conversion (˜10-15%). Added more 1.0M NaHMDS_{(THF)} (400 μL, 400 μmol, 2 eq) dropwise. Reaction mixture turned a cloudy orange but showed minimal additional conversion after 5 min by LC-MS analysis. Added a solution of cyclohexanesulfonyl chloride (34.5 μL, 43.4 mg, 238 μmol, 1.2 eq) in THE (0.5 mL) dropwise and stirred for 3 h. Concentrated to remove THF, dissolved in DCM (30 mL), washed twice with 1M HCl_{(aq)} (30 mL), twice with water (30 mL), and once with saturated NaCl_{(aq)} (30 mL). Dried over Na₂SO₄, filtered, and concentrated. Purified by normal phase flash column chromatography, eluting with DCM/MeOH. Yield: 12.1 mg, 15%. ¹H NMR (400 MHz, Chloroform-d) δ 11.36 (s, 1H), 8.43 (s, 1H), 8.27-8.02 (m, 2H), 7.29-7.24 (m, 2H), 7.24-7.16 (m, 2H), 4.87 (dq, J=12.1, 7.6, 6.1 Hz, 1H), 3.73 (ddd, J=12.1, 9.0, 3.2 Hz, 1H), 3.42 (dd, J=16.2, 7.2 Hz, 2H), 2.96 (dd, J=16.2, 4.7 Hz, 2H), 2.25 (d, J=12.6 Hz, 2H), 1.91 (d, J=10.9 Hz, 2H), 1.70 (q, J=12.9, 12.4 Hz, 2H), 1.34-1.22 (m, 4H). ¹³C NMR (101 MHz, CDCl₃) δ 164.76, 149.52, 145.79, 140.49, 135.99, 128.83, 126.99, 124.87, 62.13, 50.64, 39.95, 25.86, 25.15, 25.10. LC-MS (C₂₀H₂₄N₄O₃S): Calculated [M+H]⁺ m/z=401.16, [M−H]⁻ m/z=399.16. Observed [M+H]⁺ m/z=401.33, [M−H]⁻ m/z=399.17.

### Sulfamoyl chloride (52)

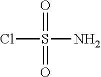

Synthesized as previously described, except that the benzene used for precipitation was replaced by toluene. In short, a vial was charged with chlorosulfonyl isocyanate (3.00 mL, 4.89 g, 34.6 mmol, 1.02 eq), flushed with argon, and cooled to 0° C. Added formic acid (1.27 mL, 1.55 g, 33.7 mmol, 1.00 eq) dropwise. Reaction bubbled vigorously before solidifying within 1 minute. After 1 h, added toluene (12 mL) and stirred at room temperature overnight. Removed insoluble by filtration and concentrated in vacuo. Yield: 3.54 g, 88.6%. White solid. ¹H NMR (400 MHz, CDCl₃) δ 6.07 (s, 2H). ¹³C NMR—does not contain any carbons. LC-MS (ClH2NO2S): Calculated [M+H]⁺ m/z=116.53, [M+H]⁻ m/z=114.53. Did not observe by LC-MS. Product of the hydrolysis of the S—Cl bond would be below our LC-MS limit of detection. Note: when the reaction solidified, the stir bar ceased functioning, but adding solvent (DCM or toluene) during the initial reaction step significantly reduced yields in subsequent couplings.

### 2′,3′-O-Isopropylidene-5′-O-sulfamoyladenosine (53)

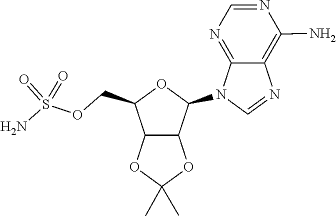 [SMILES: CC1(C)OC2C(O1)[C@@H](COS(N)(=O)=O)O[C@H]2n1cnc2c(N)ncnc21]

Synthesized as described previously. In short, dissolved ((3aR,4R,6R,6aR)-6-(6-amino-9H-purin-9-yl)-2,2-dimethyltetrahydrofuro[3,4-d][1,3]dioxol-4-yl)methanol (880 mg, 2.86 mmol, 1 eq) in DMAc (4.4 mL) and cooled to 0° C. Separately dissolved sulfamoyl chloride (761 mg, 6.59 mmol, 2.3 eq) in MeCN (3 mL) and cooled to 0° C. Slowly added the MeCN solution to the DMAc solution. After 45 min, quenched reaction by successive addition of triethylamine (3 mL) and MeOH (6 mL). Concentrated to ˜4-5 mL. Diluted with EtOAc (100 mL), washed three times with 5% LiCl_{(aq)} (50 mL), washed twice with half saturated NaHCO_{3(aq)} (50 mL), washed twice with quarter saturated NaCl_{(aq)} (50 mL), dried over sodium sulfate, and concentrated in vacuo without heating. Yield: 522 mg, 47.2%. Flaky yellow-white solid. ¹H NMR, ¹³C NMR, and LC-MS data all matched those from made previously. Notes: adding DMAc or DMF to the sulfamoyl chloride solution in the absence of the alcohol will cause it to rapidly decompose.

### 5′-O—[N-(L-Prolyl)-sulfamoyl]adenosine (ProSA, 25)

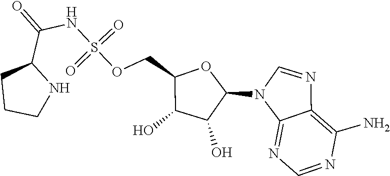 [SMILES: Nc1ncnc2c1ncn2[C@@H]1O[C@H](COS(=O)(=O)NC(=O)[C@@H]2CCCN2)[C@@H](O)[C@H]1O]

Synthesized as described previously for similar molecules with minor modifications. In short, a flask was charged with 53 (522 mg, 1.35 mmol, 1 eq), L-Boc-Pro-OSu (507 mg, 1.62 mmol, 1.2 eq), and DMF (13.5 mL). To this, added DBU (489 μL, 494 mg, 3.25 mmol, 2.4 eq) and stirred 25 minutes. The reaction was successively diluted with water (27 mL) and then quarter saturated aqueous sodium citrate until pH=9. Extracted five times with EtOAc. Washed pooled EtOAc twice with 5% LiCl_{(aq)}, twice with saturated NH₄Cl_{(aq)}, twice with half saturated NaHCO_{3(aq)}, twice with quarter saturated NaCl_{(aq)}, and dried over sodium sulfate. LC-MS analysis indicated that much of the product remained in the sodium citrate and LiCl washes so these were pooled and spiked with Na₂SO₄(s). Unsuccessfully tried to extract three times with EtOAc. To the pooled sodium citrate/LiCl fraction, added NaCl(s) until saturated. Extracted three times with iPrOH. Pooled iPrOH with both sets of EtOAc fractions and concentrated in vacuo. Purified by normal phase flash column chromatography and eluted with EtOAc/EtOH to obtain crude 2′,3′-O-Isopropylidene-5′-O—[N-(Boc-L-prolyl)-sulfamoyl]adenosine (160 mg, 20.3%) which was used without further purification or characterization. To crude 2′,3′-O-Isopropylidene-5′-O—[N-(Boc-L-prolyl)-sulfamoyl]adenosine (160 mg, 0.275 mmol), added 5:1 TFA/water (2.4 mL) and stirred 30 minutes. Concentrated in vacuo and azeotroped three times with EtOH. Purified by reverse phase flash column chromatography and eluted with water/MeCN (no additives) to obtain 25 as a white solid. ¹H NMR (d₆-DMSO) and MS data all matched those from those described previously. ¹³C NMR (101 MHz, DMSO) δ 172.08*, 156.04, 152.66, 149.57, 139.39, 118.95, 87.07, 82.40, 73.41, 70.70, 67.69, 61.98, 45.41, 29.20, 23.43. *Resonance assigned based upon ¹H-¹³C HMBC. Note: Our ¹³C NMR spectra (d₆-DMSO) matched the spectra of ProSA·Et₃N reported previously, except our spectra did not include the Et₃N ¹³C resonances (10.2 ppm and 44 ppm).

### tert-butyl trans-2-(3-(7-bromo-6-chloro-4-oxoquinazolin-3(4H)-yl)-2-oxopropyl)-3-hydroxypiperidine-1-carboxylate (54, MAT427)

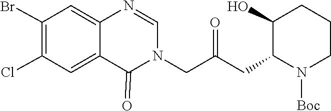 [SMILES: CC(C)(C)OC(=O)N1CCC[C@H](O)[C@H]1CC(=O)Cn1cnc2cc(Br)c(Cl)cc2c1=O]

To a suspension of trans-halofuginone (2.06 g, 4.96 mmol, 1 eq) in DMF (90 mL), successively added Boc₂O (1.44 g, 6.59 mmol, 1.32 eq) and DIPEA (2.50 mL, 1.86 g, 14.4 mmol, 2.90 eq). After 2 h, concentrated in vacuo to ˜0.5 mL. Diluted with EtOAc (600 mL), washed twice with 0.2 M HCl_{(aq)} (300 mL), twice with 5% LiCl_{(aq)} (150 mL), and once with saturated NaCl_{(aq)} (100 mL). Dried over sodium sulfate, filtered, and concentrated in vacuo. Resuspended in methyl tert-butyl ether (MTBE; 5 mL), sonicated vigorously, and filtered. Yield: 2.48 g, >95%. White solid. ¹H NMR, ¹³C NMR, and LC-MS data match the previously reported data.

### tert-butyl trans-2-(3-(7-bromo-6-chloro-4-oxoquinazolin-3(4H)-yl)-2-oxopropyl)-3-((((4-((3-((2,3-dihydro-1H-inden-2-yl)carbamoyl)pyrazin-2-yl)carbamoyl)piperazin-1-yl)sulfonyl)carbamoyl)oxy)piperidine-1-carboxylate (37)

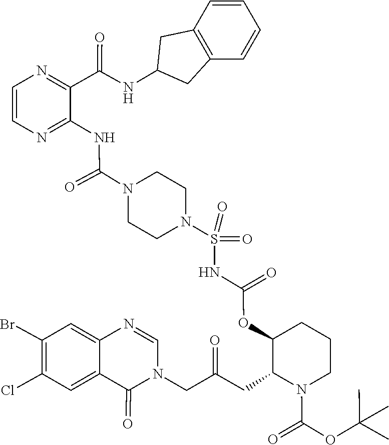 [SMILES: CC(C)(C)OC(=O)N1CCC[C@H](OC(=O)NS(=O)(=O)N2CCN(C(=O)Nc3nccnc3C(=O)NC3Cc4ccccc4C3)CC2)[C@H]1CC(=O)Cn1cnc2cc(Br)c(Cl)cc2c1=O]

To a stirred solution of 54 (206 mg, 401 μmol, 1 eq) in DCM (5 mL), added chlorosulfonyl isocyanate (37.1 μL, 60.5 mg, 427 μmol, 1.1 eq). After 10 min, added solution of 14·2HCl (269 mg, 613 μmol, 1.53 eq), DIPEA (540 μL, 401 mg, 3.10 mmol, 7.74 eq), and DCM (5 mL). After reaction was complete by LC-MS (typically ˜5 min), diluted with 0.2 M HCl_{(aq)} (200 mL) and extracted twice with DCM (200 mL). Pooled DCM fractions were dried with sodium sulfate, filtered, and concentrated in vacuo. Purified by normal phase flash column chromatography (DCM/MeOH). Yield: 244 mg, 61.6%. White solid. ¹H NMR (400 MHz, DMSO) δ 11.47 (s, 1H), 11.09-10.84 (m, 1H), 9.15 (d, J=7.6 Hz, 1H), 8.56-8.40 (m, 1H), 8.34-8.04 (m, 4H), 7.29-7.06 (m, 4H), 5.20-4.88 (m, 2H), 4.88-4.52 (m, 3H), 3.97-3.72 (m, 1H), 3.66-3.49 (m, 4H), 3.34-3.25 (m, 4H), 3.17 (dd, J=15.8, 7.8 Hz, 2H), 3.12-2.65 (m, 5H), 1.90-1.55 (m, 3H), 1.48-1.27 (m, 10H). ¹³C NMR (101 MHz, DMSO) δ 200.67, 165.52, 158.65, 154.59, 154.04, 152.59, 151.11, 149.59, 147.32, 147.28, 145.49, 141.10, 135.73, 132.38, 131.73, 131.41, 131.06, 128.36, 126.93, 126.45, 124.45, 121.81, 121.76, 79.27, 79.02, 71.53, 54.32, 53.99, 50.39, 49.56, 46.04, 43.53, 38.91*, 38.47, 38.44, 37.34, 28.05, 27.91, 23.15, 21.32, 19.15. *Assigned based upon DEPT-135 and ¹H-¹³C HSQC. LC-MS (C₄₁H₄₆BrClN₁₀O₁₀S): Calculated [M+H]⁺ m/z=985.20, [M−H]⁻ m/z=983.20. Observed [M+H]⁺ m/z=985.43, [M−H]⁻ m/z=983.45. Note: ¹H and ¹³C NMR spectra for this compound shows rotomers. All resonances observed are reported.

### trans-2-(3-(7-bromo-6-chloro-4-oxoquinazolin-3(4H)-yl)-2-oxopropyl)piperidin-3-yl ((4-((3-((2,3-dihydro-1H-inden-2-yl)carbamoyl)pyrazin-2-yl)carbamoyl)piperazin-1-yl)sulfonyl)carbamate hydrobromide (MAT436·HBr, 34·HBr)

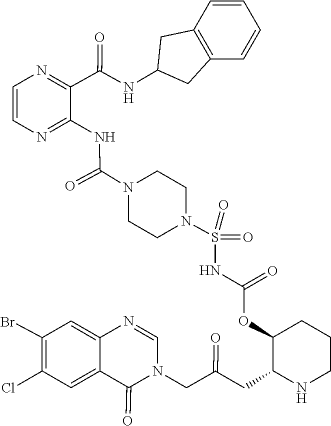 [SMILES: O=C(C[C@H]1NCCC[C@@H]1OC(=O)NS(=O)(=O)N1CCN(C(=O)Nc2nccnc2C(=O)NC2Cc3ccccc3C2)CC1)Cn1cnc2cc(Br)c(Cl)cc2c1=O]

To EtOH (792 L), added 48% HBr_{(aq)} (208 μL, 1.84 mmol, 160 eq) and stirred for 5 min. Added this to a vial of 37 (11.3 mg, 11.5 μmol, 1 eq) and stirred vigorously for 20 h. Concentrated in vacuo and then azeotroped once with EtOH (2 mL), once with 1:1 DCM/EtOH (4 mL), and then twice with EtOH (2 mL). Triturated with EtOH (1 mL), sonicated vigorously, centrifuged to pellet the product, and decanted supernatant. Dried the insoluble pellet in vacuo. Yield: 7.2 mg, 71%. White solid. ¹H NMR (400 MHz, DMSO) δ 11.65 (s, 1H), 10.97 (s, 1H), 9.17 (d, J=7.8 Hz, 1H), 8.97-8.71 (m, 2H), 8.47 (d, J=2.4 Hz, 1H), 8.29 (s, 1H), 8.21 (d, J=2.4 Hz, 1H), 8.18 (s, 1H), 8.14 (s, 1H), 7.28-7.09 (m, 4H), 5.10-4.94 (m, 2H), 4.76-4.60 (m, 2H), 3.75 (s, 1H), 3.64-3.54 (m, 4H), 3.34-3.29 (m, 4H), 3.24-3.11 (m, 4H), 3.11-2.95 (m, 4H), 2.15-2.02 (m, 1H), 1.89-1.77 (m, 1H), 1.75-1.57 (m, 2H). ¹³C NMR (101 MHz, DMSO) δ 200.36, 165.51, 158.76, 152.47, 150.80, 149.57, 149.53, 147.23, 145.54, 141.08, 135.78, 132.44, 131.92, 131.00, 128.59, 126.86, 126.48, 124.45, 121.64, 70.97, 54.40, 52.45, 50.36, 46.04, 43.46, 42.79, 38.92*, 38.47, 38.43, 27.09, 19.79. *Assigned based upon DEPT-135 and ¹H-¹³C HSQC. LC-MS (C₃₆H₃₈BrClN₁₀O₈S): Calculated [M+H]⁺ m/z=885.15, [M−H]⁻ m/z=883.15. Observed [M+H]⁺ m/z=885.56, [M−H]⁻ m/z=883.54. Note: neat MAT436·HBr appears to be stable indefinitely at 4° C., but 10 mM stock solutions of MAT436·HBr in DMSO are not stable indefinitely at room temperature (LC-MS analysis after 8 days at room temperature indicated ˜36% hydrolyzed at carbamate carbonyl) so long-term storage of MAT436·HBr stock solutions at −80° C. is recommended.

### cis-2-(3-(7-bromo-6-chloro-4-oxoquinazolin-3(4H)-yl)-2-oxopropyl)piperidin-3-yl ((4-((3-((2,3-dihydro-1H-inden-2-yl)carbamoyl)pyrazin-2-yl)carbamoyl)piperazin-1-yl)sulfonyl)carbamate (iso-MAT436 (40))

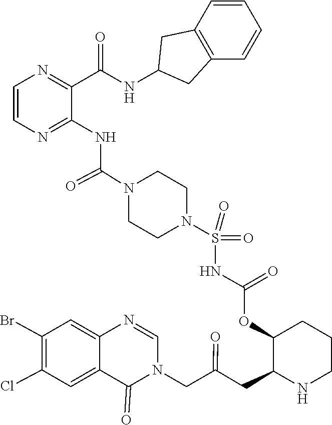 [SMILES: O=C(C[C@@H]1NCCC[C@@H]1OC(=O)NS(=O)(=O)N1CCN(C(=O)Nc2nccnc2C(=O)NC2Cc3ccccc3C2)CC1)Cn1cnc2cc(Br)c(Cl)cc2c1=O]

Dissolved 37 (29.5 mg, 29.9 μmol) in a premixed solution of 5:1 TFA/water (1.2 mL) and stirred vigorously until reaction was complete by LC-MS (typically −5 min). Concentrated in vacuo and then azeotroped three times with 2 mL EtOH to obtain MAT436·3TFA. Purified by reverse phase flash column chromatography (water/MeCN, no buffering agent present). Yield: 7.5 mg, 28.0%. White solid. ¹H NMR (400 MHz, DMSO) δ 11.01 (s, 1H), 9.20 (dd, J=13.8, 7.8 Hz, 1H), 8.55-8.40 (m, 2H), 8.20 (d, J=2.3 Hz, 1H), 8.16 (s, 1H), 8.09 (s, 1H), 7.31-7.08 (m, 4H), 5.30-4.85 (m, 2H), 4.77-4.61 (m, 1H), 4.51-4.35 (m, 1H), 3.61-3.47 (m, 4H), 3.18 (dt, J=16.2, 8.3 Hz, 4H), 3.06 (dp, J=16.7, 5.4 Hz, 7H), 2.89 (t, J=12.1 Hz, 1H), 2.79-2.69 (m, 1H), 2.03 (q, J=13.8 Hz, 1H), 1.79 (d, J=13.5 Hz, 1H), 1.68-1.54 (m, 1H), 1.51-1.39 (m, 1H), 1.10 (s, 1H). ¹³C NMR (101 MHz, DMSO) δ 200.54, 165.54, 158.72, 152.07, 150.00, 149.86, 149.72, 147.31, 145.80, 141.12, 141.08, 135.56, 132.52, 131.68, 130.09, 128.33, 126.82, 126.46, 124.45, 121.68, 70.25*, 55.54, 54.57, 50.38, 48.58*, 46.25, 45.76, 43.63, 43.40*, 40.17*, 38.46, 38.39, 28.85k*, 26.86, 21.41*. *Assigned based upon DEPT-135 and ¹H-¹³C HSQC. LC-MS (C₃₆H₃₈BrClN₁₀O₈S): Calculated [M+H]⁺ m/z=885.15, [M−H]⁻ m/z=883.15. Observed [M+H]⁺ m/z=885.39, [M−H]⁻ m/z=883.31. Note: stereochemistry of the epimerizing carbon was assigned by comparison to NMR spectra of halofuginone and iso-halofuginone. Based upon these spectra, the possibility was ruled out that the isoMAT436 (40) was isolated from deprotection of the cis-epimer of 37.

### tert-butyl trans-2-(3-(7-bromo-6-chloro-4-oxoquinazolin-3(4H)-yl)-2-oxopropyl)-3-(((N-((1-((3-((2,3-dihydro-1H-inden-2-yl)carbamoyl)pyrazin-2-yl)carbamoyl)piperidin-4-yl)methyl)sulfamoyl)carbamoyl)oxy)piperidine-1-carboxylate (38)

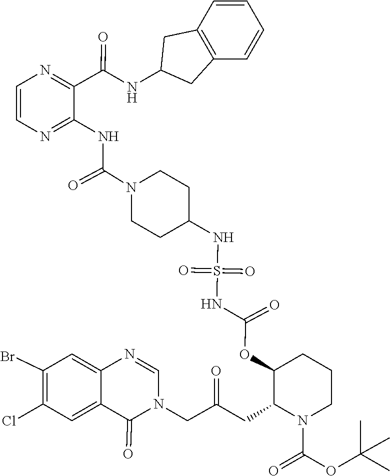 [SMILES: CC(C)(C)OC(=O)N1CCC[C@H](OC(=O)NS(=O)(=O)NC2CCN(C(=O)Nc3nccnc3C(=O)NC3Cc4ccccc4C3)CC2)[C@H]1CC(=O)Cn1cnc2cc(Br)c(Cl)cc2c1=O]

To a stirred solution of 54 (100 mg, 195 μmol, 1 eq) in DCM (2.5 mL), added chlorosulfonyl isocyanate (18.6 μL, 30.3 mg, 214 μmol, 1.1 eq). After 10 min, added solution of 10 (120 mg, 303 μmol, 1.56 eq), DIPEA (540 μL, 401 mg, 3.10 mmol, 15.9 eq), and DCM (2.5 mL). After reaction was complete by LC-MS (typically ˜5 min), diluted with 0.2 M HCl_{(aq)} (100 mL) and extracted twice with DCM (100 mL). Pooled DCM fractions were dried with sodium sulfate, filtered, and concentrated in vacuo. Purified by normal phase flash column chromatography (DCM/MeOH). Yield: 69.9 mg, 35.4%. White solid. ¹H NMR (400 MHz, DMSO) δ 11.16 (s, 1H), 11.00 (s, 1H), 9.19 (d, J=7.8 Hz, 1H), 8.48 (s, 1H), 8.31-8.16 (m, 3H), 8.11 (d, J=8.7 Hz, 1H), 7.92-7.44 (m, 1H), 7.29-7.18 (m, 2H), 7.18-7.07 (m, 2H), 5.18-4.87 (m, 2H), 4.87-4.51 (m, 3H), 4.04 (d, J=13.0 Hz, 2H), 3.96-3.69 (m, 1H), 3.26-2.70 (m, 11H), 1.95-1.81 (m, 1H), 1.81-1.66 (m, 4H), 1.63 (d, J=13.3 Hz, 1H), 1.42 (d, J=12.3 Hz, 1H), 1.36 (s, 9H), 1.11 (s, 2H). ¹³C NMR (101 MHz, DMSO) δ 200.75, 165.66, 158.67, 154.77, 152.07, 150.04, 149.63, 147.29, 145.73, 141.09, 135.37, 132.40, 131.74, 130.20, 128.36, 126.92, 126.46, 124.45, 121.78, 79.36, 70.82, 54.15, 50.44, 49.71, 48.63, 48.08, 43.70, 39.06*, 38.43, 37.52, 35.40, 29.34, 28.09, 23.29, 19.22. *Assigned based upon DEPT-135 and ¹H-¹³C HSQC. LC-MS (C₄₃H₅₀BrClN₁₀O₁₀S): Calculated [M+H]⁺ m/z=1013.23, [M−H]⁻ m/z=1011.23. Observed [M+H]⁺ m/z=1013.41, [M−H]⁻ m/z=1011.51.

### trans-2-(3-(7-bromo-6-chloro-4-oxoquinazolin-3(4H)-yl)-2-oxopropyl)piperidin-3-yl (N-((1-((3-((2,3-dihydro-1H-inden-2-yl)carbamoyl)pyrazin-2-yl)carbamoyl)piperidin-4-yl)methyl)sulfamoyl)carbamate hydrobromide (35·HBr)

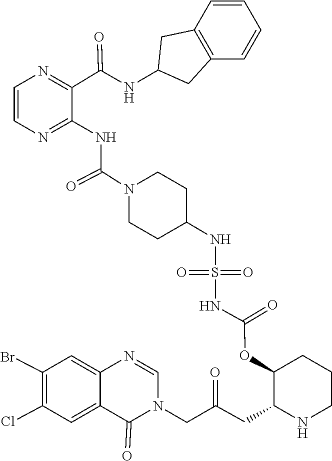 [SMILES: O=C(C[C@H]1NCCC[C@@H]1OC(=O)NS(=O)(=O)NC1CCN(C(=O)Nc2nccnc2C(=O)NC2Cc3ccccc3C2)CC1)Cn1cnc2cc(Br)c(Cl)cc2c1=O]

To EtOH (1.2 mL), added 48% HBr_{(aq)} (312 μL, 2.76 mmol, 62.4 eq) and stirred for 5 min. Added this to a vial of 38 (44.8 mg, 44.2 μmol, 1 eq) and stirred vigorously for 24 h. Concentrated in vacuo and then azeotroped once with EtOH (2 mL), once with 1:1 DCM/EtOH (4 mL), and then twice with EtOH (2 mL). Triturated with EtOH (1 mL), sonicated vigorously, centrifuged to pellet the product, and decanted supernatant. Dried the insoluble pellet in vacuo. Yield: 36.7 mg, 83.5%. White solid. ¹H NMR (400 MHz, DMSO) δ 11.25 (s, 1H), 11.05 (s, 1H), 9.20 (d, J=7.8 Hz, 1H), 9.13-8.90 (m, 2H), 8.48 (s, 1H), 8.35 (s, 1H), 8.24-8.17 (m, 2H), 8.15 (s, 1H), 8.00 (t, J=5.9 Hz, 1H), 7.28-7.07 (m, 4H), 5.14-5.01 (m, 2H), 4.78 (td, J=9.5, 4.2 Hz, 1H), 4.74-4.64 (m, 1H), 4.14-3.98 (m, 2H), 3.91-3.76 (m, 1H), 3.31-3.10 (m, 5H), 3.10-2.95 (m, 3H), 2.95-2.72 (m, 4H), 2.09 (dd, J=10.7, 5.3 Hz, 1H), 1.96-1.83 (m, 1H), 1.83-1.60 (m, 5H), 1.19-1.02 (m, 2H). ¹³C NMR (101 MHz, DMSO) δ 200.23, 165.64, 158.80, 152.10, 150.50, 149.95, 149.59, 147.26, 145.60, 141.08, 135.42, 132.46, 131.91, 130.24, 128.61, 126.90, 126.52, 126.48, 124.46, 121.67, 70.60, 54.42, 52.61, 50.44, 48.01, 43.70, 42.83, 39.08*, 38.45, 35.36, 29.28, 27.18, 19.71. *Assigned based upon DEPT-135 and ¹H-¹³C HSQC. LC-MS (C₃₈H₄₂BrClN₁₀O₈S): Calculated [M+H]⁺ m/z=913.18, [M−H]⁻ m/z=911.18. Observed [M+H]⁺ m/z=913.58, [M−H]⁻ m/z=911.59. Note: based upon studies with the analogous MAT436, this compound might be labile to epimerization if it is purified by reverse phase flash column chromatography (water/MeCN).

### tert-butyl trans-2-(3-(7-bromo-6-chloro-4-oxoquinazolin-3(4H)-yl)-2-oxopropyl)-3-(((N-(1-((3-((2,3-dihydro-1H-inden-2-yl)carbamoyl)pyrazin-2-yl)carbamoyl)piperidin-4-yl)sulfamoyl)carbamoyl)oxy)piperidine-1-carboxylate (39, M4T435)

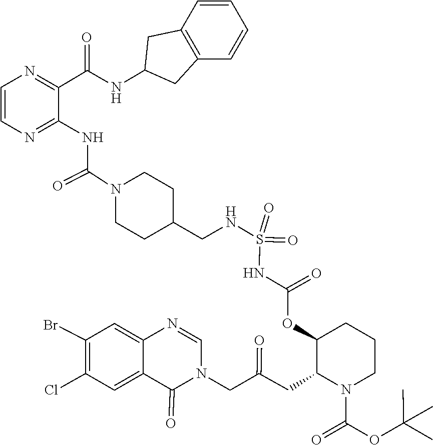 [SMILES: CC(C)(C)OC(=O)N1CCC[C@H](OC(=O)NS(=O)(=O)NCC2CCN(C(=O)Nc3nccnc3C(=O)NC3Cc4ccccc4C3)CC2)[C@H]1CC(=O)Cn1cnc2cc(Br)c(Cl)cc2c1=O]

To a stirred solution of 54 (103 mg, 200 μmol, 1 eq) in DCM (2.5 mL), added chlorosulfonyl isocyanate (18.6 μL, 30.3 mg, 214 μmol, 1.1 eq). After 10 min, added solution of 8·2HCl (134 mg, 296 μmol, 1.48 eq), DIPEA (540 μL, 401 mg, 3.10 mmol, 15.5 eq), and DCM (2.5 mL). After reaction was complete by LC-MS (typically ˜5 min), diluted with 0.2 M HCl_{(aq)} (100 mL) and extracted twice with DCM (100 mL). Pooled DCM fractions were dried with sodium sulfate, filtered, and concentrated in vacuo. Purified by normal phase flash column chromatography (DCM/MeOH). Yield: 82.0 mg, 41.1%. White solid. ¹H NMR (400 MHz, DMSO) δ 11.30 (s, 1H), 11.00 (s, 1H), 9.18 (d, J=7.9 Hz, 1H), 9.13-8.89 (m, 2H), 8.48 (s, 1H), 8.36 (s, 1H), 8.23-8.18 (m, 1H), 8.18-8.15 (m, 1H), 8.15-8.09 (m, 2H), 7.25-7.08 (m, 4H), 5.15-4.99 (m, 2H), 4.79 (td, J=9.4, 4.0 Hz, 1H), 4.70-4.64 (m, 1H), 3.96 (d, J=13.2 Hz, 2H), 3.89-3.76 (m, 1H), 3.45-3.32 (m, 1H), 3.27-3.10 (m, 5H), 3.09-2.91 (m, 5H), 2.15-2.01 (m, 1H), 1.97-1.60 (m, 5H), 1.47 (h, J=9.1 Hz, 2H). ¹³C NMR (101 MHz, DMSO) δ 200.69, 165.61, 158.67, 154.82, 152.09, 149.95, 149.61, 147.28, 145.72, 141.09, 135.50, 132.38, 131.74, 130.34, 128.36, 126.92, 126.46, 124.44, 121.78, 79.43, 70.80, 54.08, 50.44, 49.77, 42.55, 42.40, 39.13*, 38.42, 38.39, 31.79, 31.32, 29.05, 28.11, 23.30, 22.13, 19.22, 13.99. *Assigned based upon DEPT-135 and ¹H-¹³C HSQC. LC-MS (C₄₂H₄₈BrClN₁₀O₁₀S): Calculated [M+H]⁺ m/z=999.21, [M−H]⁻ m/z=997.21. Observed [M+H]⁺ m/z=999.44, [M−H]⁻ m/z=997.27.

### trans-2-(3-(7-bromo-6-chloro-4-oxoquinazolin-3(4H)-yl)-2-oxopropyl)piperidin-3-yl (N-(1-((3-((2,3-dihydro-1H-inden-2-yl)carbamoyl)pyrazin-2-yl)carbamoyl)piperidin-4-yl)sulfamoyl)carbamate hydrobromide (36·HBr)

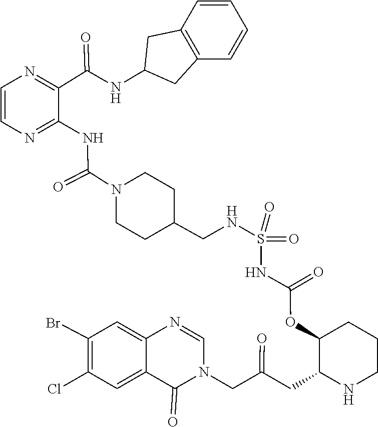 [SMILES: O=C(C[C@H]1NCCC[C@@H]1OC(=O)NS(=O)(=O)NCC1CCN(C(=O)Nc2nccnc2C(=O)NC2Cc3ccccc3C2)CC1)Cn1cnc2cc(Br)c(Cl)cc2c1=O]

To EtOH (1.2 mL), added 48% HBr_{(aq)} (312 μL, 2.76 mmol, 47.0 eq) and stirred for 5 min. Added this to a vial of 39 (58.7 mg, 58.7 μmol, 1 eq) and stirred vigorously for 24 h. Concentrated in vacuo and then azeotroped once with EtOH (2 mL), once with 1:1 DCM/EtOH (4 mL), and then twice with EtOH (2 mL). Triturated with EtOH (1 mL), sonicated vigorously, centrifuged to pellet the product, and decanted supernatant. Dried the insoluble pellet in vacuo. Yield: 54.2 mg, 94.1%. White solid. ¹H NMR (400 MHz, DMSO) δ 11.30 (s, 1H), 11.00 (s, 1H), 9.18 (d, J=7.9 Hz, 1H), 9.13-8.89 (m, 2H), 8.48 (s, 1H), 8.36 (s, 1H), 8.23-8.18 (m, 1H), 8.18-8.15 (m, 1H), 8.15-8.09 (m, 2H), 7.25-7.08 (m, 4H), 5.15-4.99 (m, 2H), 4.79 (td, J=9.4, 4.0 Hz, 1H), 4.70-4.64 (m, 1H), 3.96 (d, J=13.2 Hz, 2H), 3.89-3.76 (m, 1H), 3.45-3.32 (m, 1H), 3.27-3.10 (m, 5H), 3.09-2.91 (m, 5H), 2.15-2.01 (m, 1H), 1.97-1.60 (m, 5H), 1.47 (h, J=9.1 Hz, 2H). ¹³C NMR (101 MHz, DMSO) δ 200.28, 165.59, 158.79, 152.13, 150.50, 149.83, 149.57, 147.23, 145.56, 141.06, 135.56, 132.44, 131.90, 130.45, 128.61, 126.89, 126.47, 124.45, 121.65, 70.57, 54.41, 52.66, 50.59, 50.40, 42.88, 42.60, 42.49, 39.00, 38.43, 31.93, 31.86, 27.21, 19.71. LC-MS (C₃₇H₄₀BrClN₁₀O₈S): Calculated [M+H]⁺ m/z=899.16, [M−H]⁻ m/z=897.16. Observed [M+H]⁺ m/z=899.56, [M−H]⁻ m/z=897.56. Note: based upon studies with the analogous MAT436, this compound might be labile to epimerization if it is purified by reverse phase flash column chromatography (water/MeCN).

### tert-butyl (2S,3R)-2-(3-(7-bromo-6-chloro-4-oxoquinazolin-3(4H)-yl)-2-hydroxypropyl)-3-((((4-((3-((2,3-dihydro-1H-inden-2-yl)carbamoyl)pyrazin-2-yl)carbamoyl)piperazin-1-yl)sulfonyl)carbamoyl)oxy)piperidine-1-carboxylate (MAT582)

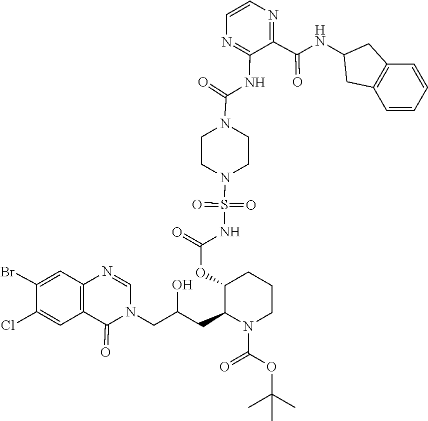 [SMILES: CC(C)(C)OC(=O)N1CCC[C@@H](OC(=O)NS(=O)(=O)N2CCN(C(=O)Nc3nccnc3C(=O)NC3Cc4ccccc4C3)CC2)[C@@H]1CC(O)Cn1cnc2cc(Br)c(Cl)cc2c1=O]

Procedure is based upon protocol for synthesis of BOC-halofuginol with minor modifications.¹ Charged a vial with MAT435 (37.2 mg, 37.7 μmol, 1 eq), MeOH (1.60 mL), and THE (480 L). Stirred vigorously and flushed with argon. Added NaBH₄ (10.4 mg, 275 mol, 7.3 eq) and stirred for 30 min. Diluted with saturated NH₄Cl_{(aq)} (40 mL) and water (40 mL). Extracted twice with DCM (40 mL). Washed pooled DCM fractions with a mixture of 20 mL saturated NaCl_{(aq)} (20 mL) and water (40 mL). Dried over sodium sulfate, filtered, and concentrated in vacuo. Yield: Quantitative. White solid. ¹H NMR (400 MHz, DMSO) δ 11.45 (s, 1H), 10.96 (d, J=17.5 Hz, 1H), 9.17 (t, J=7.3 Hz, 1H), 8.49 (d, J=5.4 Hz, 1H), 8.29-8.02 (m, 3H), 7.27-7.12 (m, 4H), 7.12-6.99 (m, 1H), 5.15-4.84 (m, 1H), 4.83-4.57 (m, 3H), 4.47 (s, 1H), 4.15 (d, J=13.0 Hz, 1H), 3.99-3.62 (m, 3H), 3.57 (t, J=5.1 Hz, 4H), 3.28 (s, 4H), 3.22-3.12 (m, 2H), 3.03 (dd, J=16.1, 7.1 Hz, 2H), 2.96-2.69 (m, 1H), 1.89-1.50 (m, 5H), 1.50-1.26 (m, 11H). LC-MS (C₄₁H₄₈BrClN₁₀O₁₀S): Calculated [M+H]⁺ m/z=987.21, [M−H]⁻ m/z=985.21. Observed [M+H]⁺ m/z=987.67, [M−H]⁻ m/z=985.58.

### (2R,3S)-2-(3-(7-bromo-6-chloro-4-oxoquinazolin-3(4H)-yl)-2-hydroxypropyl)piperidin-3-yl ((4-((3-((2,3-dihydro-1H-inden-2-yl)carbamoyl)pyrazin-2-yl)carbamoyl)piperazin-1-yl)sulfonyl)carbamate 2,2,2-trifluoroacetate (MAT583·0.8TFA)

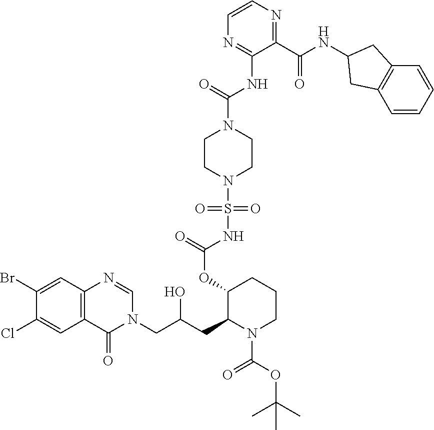 [SMILES: CC(C)(C)OC(=O)N1CCC[C@@H](OC(=O)NS(=O)(=O)N2CCN(C(=O)Nc3nccnc3C(=O)NC3Cc4ccccc4C3)CC2)[C@@H]1CC(O)Cn1cnc2cc(Br)c(Cl)cc2c1=O]

Dissolved MAT582 (4.8 mg, 4.9 μmol) in a premixed solution of 5:2 TFA/water (1.4 mL) and stirred vigorously until reaction was complete by LCMS (typically ˜5 min). Concentrated in vacuo and then azeotroped three times with 2 mL EtOH. Purified by reverse phase flash column chromatography (water/MeCN, no buffering agent present) and concentrated in vacuo to obtain MAT583·0.8TFA. Yield: 2.9 mg, 61%. White solid. ¹H NMR (400 MHz, DMSO) δ 11.05 (s, 1H), 9.18 (t, J=7.3 Hz, 1H), 8.46 (d, J=2.4 Hz, 1H), 8.27 (d, J=11.9 Hz, 1H), 8.18 (h, J=7.1 Hz, 2H), 8.08 (d, J=5.5 Hz, 1H), 7.21 (t, J=4.2 Hz, 2H), 7.17-7.11 (m, 2H), 4.68 (dt, J=15.6, 7.9 Hz, 1H), 4.39 (s, 1H), 4.04 (d, J=11.8 Hz, 2H), 3.84-3.68 (m, 1H), 3.50 (t, J=5.2 Hz, 4H), 3.17 (dd, J=15.7, 7.7 Hz, 2H), 3.03 (p, J=8.0 Hz, 7H), 2.74 (s, 1H), 1.93 (s, 2H), 1.74 (s, 1H), 1.55 (s, 2H), 1.42 (s, 2H), 1.23 (s, 2H). LC-MS (C₃₆H₄₀BrClN₁₀O₈S): Calculated [M+H]⁺ m/z=887.16, [M−H]⁻ m/z=885.16. Observed [M+H]⁺ m/z=887.58, [M−H]⁻ m/z=885.55.

### tert-butyl (2R,3S)-2-(3-(7-bromo-6-chloro-4-oxoquinazolin-3(4H)-yl)-2-oxopropyl)-3-(carbamoyloxy)piperidine-1-carboxylate (M4T468)

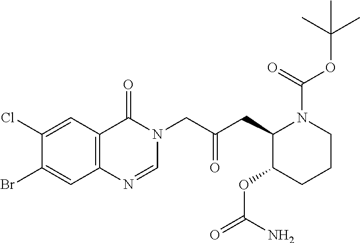 [SMILES: CC(C)(C)OC(=O)N1CCC[C@H](OC(N)=O)[C@H]1CC(=O)Cn1cnc2cc(Br)c(Cl)cc2c1=O]

Accidentally isolated when attempting to synthesize MAT429 (above) using a different procedure.

To a vigorously stirred solution of MAT427 (53.0 mg, 103 μmol, 1 eq) in MeCN (3.5 mL), added chlorosulfonyl isocyanate (10.1 μL, 116 μmol, 1.1 eq) and stirred vigorously. After 15 minutes, added solution of 2′,3′-O-isopropylideneadenosine (45.9.0 mg, 149 μmol, 1.45 eq), DIPEA (102 μL, 586 μmol, 5.7 eq), MeCN (3.5 mL), and DMAc (2 mL). After 2 h, diluted with DCM (300 mL). Washed with 0.2 M NaOAc, pH 4 (140 mL). Observed emulsion that took several minutes to separate. Dried DCM fraction over sodium sulfate, filtered, and concentrated. Purified by reverse phase flash column chromatography (water/MeCN) to obtain MAT468. Yield: 27.5 mg, 48%. White solid. ¹H NMR (400 MHz, CDCl₃) δ 8.30 (s, 1H), 8.11 (s, 1H), 8.05 (s, 1H), 4.96 (td, J=63.5, 43.8 Hz, 6H), 3.94 (s, 1H), 2.97 (d, J=26.9 Hz, 3H), 2.81 (p, J=7.1 Hz, 2H), 2.08 (s, 1H), 1.85 (s, 1H), 1.76 (t, J=11.5 Hz, 2H), 1.56-1.46 (m, 1H), 1.42 (s, 9H). ¹³C NMR (101 MHz, CDCl₃) δ 199.18, 170.90, 159.42, 156.22, 148.60, 146.26, 133.88, 132.19, 129.80, 127.84, 121.80, 80.79, 70.15, 53.87, 51.56, 40.69, 39.30, 28.51, 28.48, 24.29, 24.12, 21.65, 19.63. LC-MS (C₂₂H₂₆BrClN₄O₆): Calculated [M+H]⁺ m/z=557.08, [M−H]⁻ m/z=555.07. Observed [M+H]⁺ m/z=557.25, [M−H]⁻ m/z=555.24.

### (2R,3S)-2-(3-(7-bromo-6-chloro-4-oxoquinazolin-3(4H)-yl)-2-oxopropyl)piperidin-3-yl carbamate (MA T469)

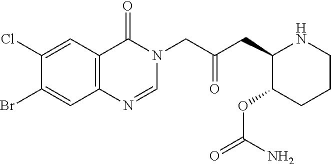 [SMILES: NC(=O)O[C@H]1CCCN[C@@H]1CC(=O)Cn1cnc2cc(Br)c(Cl)cc2c1=O]

To MAT468 (29.7 mg, 53.2 μmol, 1 eq), added 5:1 TFA/water (1.8 mL) and stirred vigorously for 30 minutes. Concentrated in vacuo. Azeotroped three times with EtOH (2 mL). Yield: Quantitative. White solid. ¹H NMR (400 MHz, DMSO) δ 8.93 (d, J=24.9 Hz, 2H), 8.31-8.16 (m, 3H), 6.73 (s, 2H), 5.11-4.95 (m, 2H), 4.56 (td, J=9.8, 4.0 Hz, 1H), 3.65 (s, 1H), 3.22 (d, J=12.5 Hz, 1H), 3.10 (dd, J=18.2, 5.2 Hz, 1H), 3.05-2.92 (m, 2H), 2.07-1.97 (m, 1H), 1.90-1.79 (m, 1H), 1.67 (q, J=12.7 Hz, 1H), 1.61-1.49 (m, 1H). ¹³C NMR (101 MHz, DMSO) δ 200.19, 158.83, 155.39, 149.66, 147.32, 132.52, 131.94, 128.62, 126.93, 121.73, 68.91, 54.46, 53.31, 42.99, 27.80, 20.11. LC-MS (C₁₇H₁₈BrClN₄O₄): Calculated [M+H]⁺ m/z=457.03, [M−H]⁻ m/z=455.01. Observed [M+H]⁺ m/z=457.16, [M−H]⁻ m/z=455.23.

### tert-butyl (2S,3R)-3-((((((3aR,4R,6R,6aR)-6-(6-amino-9H-purin-9-yl)-2,2-dimethyltetrahydrofuro[3,4-d][1,3]dioxol-4-yl)methoxy)sulfonyl)carbamoyl)oxy)-2-(3-(7-bromo-6-chloro-4-oxoquinazolin-3(4H)-yl)-2-oxopropyl)piperidine-1-carboxylate (33, MAT429)

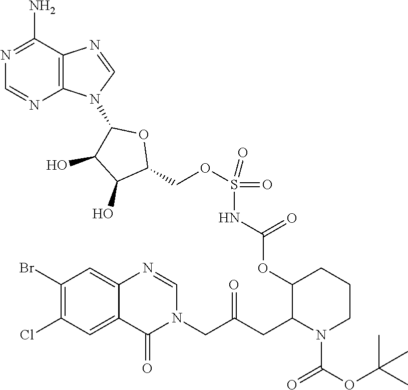 [SMILES: CC(C)(C)OC(=O)N1CCCC(OC(=O)NS(=O)(=O)OC[C@H]2O[C@@H](n3cnc4c(N)ncnc43)[C@H](O)[C@@H]2O)C1CC(=O)Cn1cnc2cc(Br)c(Cl)cc2c1=O]

To a vigorously stirred solution of MAT427 (253 mg, 492 μmol, 1 eq) in DCM (5 mL), added chlorosulfonyl isocyanate (84.3 μL, 971 μmol, 2 eq) and stirred vigorously. Charged a second flask with 2′,3′-O-isopropylideneadenosine (778 mg, 2.53 mmol, 5.1 eq), DIPEA (1.3 mL, 7.5 mmol, 15 eq), and DMF (20 mL). After 20 minutes, added the DCM solution to the DMF solution. Monitored by LCMS and worked up when reaction was complete (˜20 minutes). Quenched with 1 M HCl (200 mL) and observed that a large amount of material (including product) precipitated as a mixture of a solid (trace quantity) and an extremely viscous oil (majority of precipitate). Discarded the mother liquor. Dried the precipitated material in vacuo to remove residual solvent and obtain a mixture of MAT429 and MAT468. Yield: 185 mg, 41% yield (not adjusted for MAT468 impurity). White solid. ¹H NMR (400 MHz, DMSO) δ 8.55 (d, J=3.0 Hz, 1H), 8.43 (s, 1H), 8.30-8.18 (m, 4H), 8.18-8.13 (m, 2H), 6.50 (s, 1H), 6.29 (t, J=1.9 Hz, 1H), 5.75 (s, 1H), 5.43-5.35 (m, 1H), 5.15-4.92 (m, 6H), 4.84-4.31 (m, 9H), 3.84 (d, J=22.3 Hz, 2H), 1.94-1.72 (m, 3H), 1.63 (d, J=14.0 Hz, 5H), 1.52 (s, 4H), 1.47 (d, J=2.2 Hz, 1H), 1.36 (s, 15H), 1.33 (s, 9H), 1.30 (s, 3H), 1.28-1.09 (m, 3H). ¹³C NMR (101 MHz, DMSO) δ 162.32, 149.69, 147.32, 132.42, 131.75, 126.96, 121.80, 35.80, 30.78, 28.02, 26.82, 25.09. Note: ¹³C NMR spectra had insufficient signal to background to see all expected resonances but we have listed what was observed. LC-MS (C₃₅H₄₁BrClN₉O₁₂S): Calculated [M+H]⁺ m/z=926.15, [M−H]⁻ m/z=924.15. Observed [M+H]⁺ m/z=926.37, [M−H]⁻ m/z=924.32.

### ((2R,3S,4R,5R)-5-(6-amino-9H-purin-9-yl)-3,4-dihydroxytetrahydrofuran-2-yl)methy ((((2S,3R)-2-(3-(7-bromo-6-chloro-4-oxoquinazolin-3(4H)-yl)-2-oxopropyl)piperidin-3-yl)oxy)carbonyl)sulfamate (MAT430)

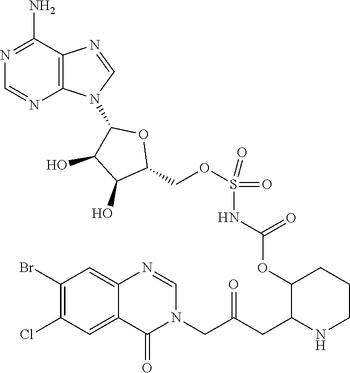 [SMILES: Nc1ncnc2c1ncn2[C@@H]1O[C@H](COS(=O)(=O)NC(=O)OC2CCCNC2CC(=O)Cn2cnc3cc(Br)c(Cl)cc3c2=O)[C@@H](O)[C@H]1O]

Charged vial with 4 M HCl_{(1,4-dioxane)} (0.5 mL) and MeOH (1.5 mL). Stirred vigorously until it cooled to room temperature. Added this solution to a vial containing vial containing MAT429 (55.5 mg, 59.9 μmol, 1 eq) and stirred vigorously. After 1 h, concentrated in vacuo to ˜100 μL of liquid while keeping the temperature below 20° C. Continuing to keep temperature below 20° C., azeotroped once with 3 mL 10% HBr(EtOH/water) (prepared from 0.21 parts 48% HBr_{(aq)} and 0.79 parts EtOH), azeotroped 2× with EtOH (3 mL), azeotroped once with MeOH (5 mL), and azeotroped twice more with EtOH (3 mL). After last azeotrope, concentrated under strong vacuum to a solid. Triturated twice with EtOH and kept the EtOH insoluble material (note: there is substantial product in EtOH soluble, but it is less clean). Dried the EtOH insoluble material under strong vacuum to obtain MAT429+MAT469 (74:26 by LCMS). Yield: 25.0 mg, 53.0% (not adjusted for impurity). White solid. ¹H NMR (400 MHz, DMSO) δ 9.62 (s, 1H), 9.10-8.87 (m, 3H), 8.65 (d, J=3.5 Hz, 1H), 8.50 (d, J=2.6 Hz, 1H), 8.33 (s, 2H), 8.24-8.12 (m, 4H), 6.72 (s, 1H), 5.99 (q, J=5.3 Hz, 1H), 5.10-5.02 (m, 3H), 4.73 (ddq, J=10.3, 7.6, 4.7 Hz, 1H), 4.54 (tdd, J=20.4, 10.5, 5.8 Hz, 4H), 4.23 (tq, J=6.7, 3.5 Hz, 2H), 3.82-3.72 (m, 1H), 3.69 (d, J=12.9 Hz, 1H), 3.43 (q, J=6.8 Hz, 1H), 3.21 (t, J=7.3 Hz, 3H), 3.13 (dd, J=6.2, 3.4 Hz, 1H), 3.08 (d, J=4.9 Hz, 1H), 3.02 (t, J=11.1 Hz, 2H), 2.10-1.96 (m, 2H), 1.90-1.78 (m, 2H), 1.78-1.66 (m, 2H), 1.66-1.49 (m, 2H), 1.04 (t, J=7.0 Hz, 1H). ¹³C NMR (101 MHz, DMSO) δ 200.08, 158.75, 155.37, 150.19, 149.66, 148.26, 147.30, 132.48, 131.90, 128.60, 126.92, 121.70, 118.79, 69.87, 68.91, 54.48, 53.34, 27.80, 20.03, 18.59. LC-MS (C₂₇H₂₉BrClN₉O₁₀S): Calculated [M+H]⁺ m/z=786.06, [M−H]⁻ m/z=784.06. Observed [M+H]⁺ m/z=786.32, [M−H]⁻ m/z=784.29.

### tert-butyl (S)-3-((((4-((3-((2,3-dihydro-1H-inden-2-yl)carbamoyl)pyrazin-2-yl)carbamoyl)piperazin-1-yl)sulfonyl)carbamoyl)oxy)piperidine-1-carboxylate (MAT444)

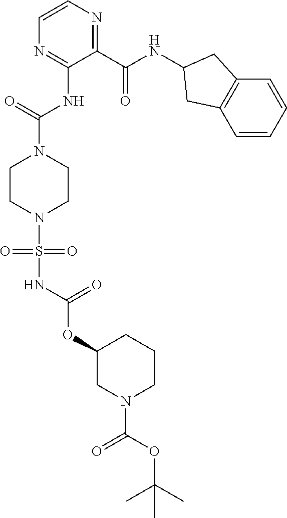 [SMILES: CC(C)(C)OC(=O)N1CCC[C@H](OC(=O)NS(=O)(=O)N2CCN(C(=O)Nc3nccnc3C(=O)NC3Cc4ccccc4C3)CC2)C1]

Combined (S)-N-BOC-3-hydroxypiperidine (24.1 mg, 120 μmol, 1 eq) and DCM (2 mL). Added chlorosulfonyl isocyanate (9.5 μL, 15 mg, 110 μmol, 0.91 eq) and stirred vigorously. To this, added premixed solution of cmpd 14·2HCl (65.5 mg, 149 μmol, 1.25 eq), DCM (2 mL), and DIPEA (86.5 μL, 497 μmol, 4.15 eq). After reaction was complete by LCMS, diluted with DCM (100 mL), washed with 0.2 M HCl, dried over Na₂SO₄, filtered, and concentrated to dryness. Purified by reverse phase flash column chromatography (water/MeCN, both with 0.1% formic acid). Yield: 73.2 mg, 91%. White solid. ¹H NMR (400 MHz, CDCl₃) δ 11.69 (s, 1H), 9.13 (d, J=7.2 Hz, 1H), 8.98 (s, 1H), 8.30 (d, J=5.5 Hz, 1H), 8.15-8.00 (m, 2H), 7.30-7.12 (m, 4H), 4.79-4.67 (m, 2H), 3.99-3.81 (m, 2H), 3.79-3.68 (m, 3H), 3.64-3.34 (m, 11H), 3.32-3.20 (m, 1H), 2.95 (ddd, J=16.1, 11.6, 5.9 Hz, 3H), 1.88 (ddt, J=14.0, 9.0, 4.3 Hz, 1H), 1.80-1.67 (m, 2H), 1.54-1.38 (m, 12H). ¹³C NMR (101 MHz, CDCl₃) δ 165.72, 164.77, 155.01, 153.88, 150.77, 150.71, 149.11, 141.43, 141.10, 140.54, 135.47, 134.27, 133.52, 127.11, 126.81, 124.96, 124.80, 80.16, 71.17, 71.15, 51.45, 50.75, 47.26, 47.18, 46.51, 46.23, 43.85, 42.22, 40.34, 40.04, 29.13, 28.53, 28.48, 22.50, 21.68. LC-MS (C₃₀H₄₀N₈O₈S): Calculated [M+H]⁺ m/z=673.28, [M−H]⁻ m/z=671.26. Observed [M+H]⁺ m/z=673.44, [M−H]⁻ m/z=671.31.

### (S)-piperidin-3-yl ((4-((3-((2,3-dihydro-1H-inden-2-yl)carbamoyl)pyrazin-2-yl)carbamoyl)piperazin-1-yl)sulfonyl)carbamate (MAT445)

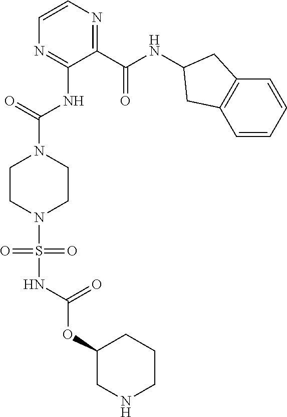 [SMILES: O=C(NS(=O)(=O)N1CCN(C(=O)Nc2nccnc2C(=O)NC2Cc3ccccc3C2)CC1)O[C@H]1CCCNC1]

To MAT444 (46.7 mg, 69.4 μmol, 1 eq), added 2 mL 5:1 TFA/water. Stirred vigorously and monitored by LCMS. After 40 minutes, concentrated to dryness. Azeotroped with ethanol three times. Purified by reverse phase flash column chromatography (water/MeCN).

Yield: Quantitative. White solid. ¹H NMR (400 MHz, DMSO) δ 11.61 (s, 1H), 9.07 (s, 1H), 8.74 (d, J=28.4 Hz, 2H), 8.35 (d, J=2.6 Hz, 1H), 8.25 (dd, J=6.2, 2.5 Hz, 1H), 7.66 (d, J=7.1 Hz, 1H), 7.29-7.12 (m, 4H), 4.89-4.74 (m, 1H), 4.39 (h, J=6.4 Hz, 1H), 3.75-3.65 (m, 4H), 3.41-3.14 (m, 7H), 3.11-2.91 (m, 3H), 2.85-2.76 (m, 2H), 1.99-1.71 (m, 2H), 1.71-1.44 (m, 2H). ¹³C NMR (101 MHz, DMSO) δ 165.59, 158.29, 150.83, 146.89, 142.41, 141.12, 141.04, 136.08, 126.56, 126.50, 124.64, 124.48, 67.68, 61.84, 50.92, 48.61, 46.24, 45.78, 44.92, 43.13, 42.63, 38.48, 26.77, 18.87. LC-MS (C₂₅H₃₂N₈O₆S): Calculated [M+H]⁺ m/z=573.22, [M−H]⁻ m/z=571.21. Observed [M+H]⁺ m/z=572.98, [M−H]⁻ m/z=571.35.

### tert-butyl (R)-3-((((4-((3-((2,3-dihydro-1H-inden-2-yl)carbamoyl)pyrazin-2-yl)carbamoyl)piperazin-1-yl)sulfonyl)carbamoyl)oxy)piperidine-1-carboxylate (MAT446)

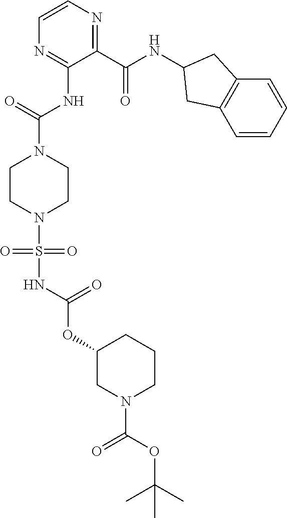 [SMILES: CC(C)(C)OC(=O)N1CCC[C@@H](OC(=O)NS(=O)(=O)N2CCN(C(=O)Nc3nccnc3C(=O)NC3Cc4ccccc4C3)CC2)C1]

Combined (R)-N-BOC-3-hydroxypiperidine (23.5 mg, 117 μmol, 1 eq) and DCM (2 mL). Added chlorosulfonyl isocyanate (9.5 μL, 15 mg, 110 μmol, 0.94 eq) and stirred vigorously. To this, added premixed solution of 14·2HCl (65.5 mg, 149 μmol, 1.28 eq), DCM (2 mL), and DIPEA (86.5 μL, 497 μmol, 4.25 eq). After reaction was complete by LCMS, diluted with DCM (100 mL), washed with 0.2 M HCl, dried over Na₂SO₄, filtered, and concentrated to dryness. Purified by reverse phase flash column chromatography (water/MeCN, both with 0.1% formic acid). Yield: 64.9 mg, 83%. White solid. ¹H NMR (400 MHz, CDCl₃) δ 11.70 (s, 1H), 9.13 (d, J=7.2 Hz, 1H), 9.01 (s, 1H), 8.19 (s, 1H), 8.13-8.01 (m, 2H), 7.30-7.13 (m, 4H), 4.80-4.68 (m, 2H), 4.02-3.82 (m, 2H), 3.82-3.66 (m, 3H), 3.66-3.48 (m, 6H), 3.48-3.33 (m, 5H), 3.33-3.19 (m, 1H), 3.04-2.89 (m, 3H), 1.94-1.82 (m, 1H), 1.82-1.68 (m, 2H), 1.57-1.35 (m, 12H). ¹³C NMR (101 MHz, CDCl₃) δ 164.78, 155.01, 153.86, 152.52, 150.73, 150.68, 149.15, 141.48, 141.11, 140.54, 134.27, 133.48, 127.12, 126.81, 124.97, 124.81, 80.16, 71.21, 71.18, 66.38, 51.45, 50.76, 47.28, 47.20, 46.54, 46.25, 43.87, 42.23, 40.35, 40.06, 29.13, 28.54, 28.49, 21.68. LC-MS (C₃₀H₄₀N₈O₈S): Calculated [M+H]⁺ m/z=673.28, [M−H]⁻ m/z=671.26. Observed [M+H]⁺ m/z=673.51, [M−H]⁻ m/z=671.45.

### (R)-piperidin-3-yl ((4-((3-((2,3-dihydro-1H-inden-2-yl)carbamoyl)pyrazin-2-yl)carbamoyl)piperazin-1-yl)sulfonyl)carbamate (MAT447)

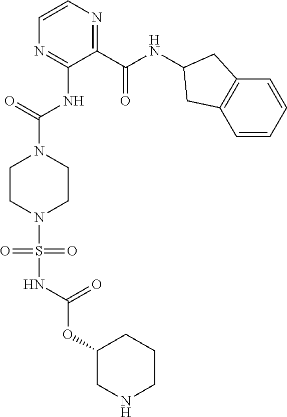 [SMILES: O=C(NS(=O)(=O)N1CCN(C(=O)Nc2nccnc2C(=O)NC2Cc3ccccc3C2)CC1)O[C@@H]1CCCNC1]

To MAT446 (32.7 mg, 55.3 μmol, 1 eq), added 2 mL 5:1 TFA/water. Stirred vigorously and monitored by LCMS. After 20 minutes, concentrated to dryness. Azeotroped with ethanol three times. Purified by reverse phase flash column chromatography (water/MeCN). Yield: Quantitative. White solid. ¹H NMR (400 MHz, DMSO) δ 11.61 (s, 1H), 9.08 (s, 1H), 8.75 (d, J=28.8 Hz, 2H), 8.35 (d, J=2.6 Hz, 1H), 8.25 (dd, J=6.2, 2.5 Hz, 1H), 7.66 (d, J=7.0 Hz, 1H), 7.31-7.20 (m, 2H), 7.20-7.10 (m, 2H), 4.87-4.74 (m, 1H), 4.39 (h, J=6.4 Hz, 1H), 3.76-3.65 (m, 3H), 3.42-3.12 (m, 9H), 3.12-2.89 (m, 4H), 2.82 (dd, J=16.0, 5.4 Hz, 2H), 1.94-1.72 (m, 2H), 1.72-1.52 (m, 2H). ¹³C NMR (101 MHz, DMSO) δ 165.59, 158.27, 157.94, 153.63, 150.84, 146.89, 142.41, 141.12, 141.04, 136.08, 126.56, 126.49, 124.63, 124.48, 67.68, 61.85, 50.92, 50.41, 48.60, 46.23, 45.78, 44.92, 43.12, 42.62, 40.89, 38.48, 29.66, 26.77, 18.86, 18.51. LC-MS (C₂₅H₃₂N₈O₆S): Calculated [M+H]⁺ m/z=573.22, [M−H]⁻ m/z=571.21. Observed [M+H]⁺ m/z=572.97, [M−H]⁻ m/z=571.35.

### tert-butyl (S)-2-(((((4-((3-((2,3-dihydro-1H-inden-2-yl)carbamoyl)pyrazin-2-yl)carbamoyl)piperazin-1-yl)sulfonyl)carbamoyl)oxy)methyl)pyrrolidine-1-carboxylate (MAT456)

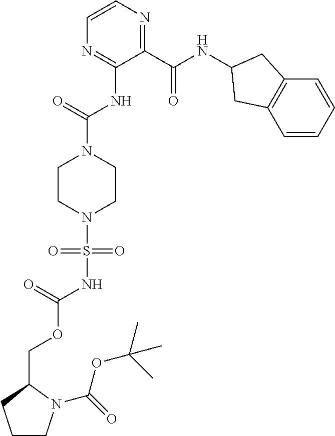 [SMILES: CC(C)(C)OC(=O)N1CCC[C@H]1COC(=O)NS(=O)(=O)N1CCN(C(=O)Nc2nccnc2C(=O)NC2Cc3ccccc3C2)CC1]

Combined BOC-L-prolinol (50 mg, 250 μmol, 1 eq) and DCM (2 mL). Added chlorosulfonyl isocyanate (23.7 μL, 38.7 mg, 273 μmol, 1.1 eq) and stirred vigorously. To this, added premixed solution of MAT336·2HCl (162 mg, 370 μmol, 1.5 eq), DCM (2 mL), and DIPEA (216 μL). After reaction was complete by LCMS, quenched by addition of MeOH (1 mL). Concentrated to an oil. Purified by reverse phase flash column chromatography (water/MeCN, both with 0.1% formic acid). Yield: Quantitative. White solid. ¹H NMR (400 MHz, CDCl₃) δ 11.69 (s, 1H), 8.53 (s, 1H), 8.29 (d, J=8.1 Hz, 1H), 8.05 (d, J=2.3 Hz, 1H), 7.86-7.58 (m, 1H), 7.30-7.24 (m, 3H), 7.24-7.19 (m, 2H), 4.85 (dtd, J=12.2, 7.4, 4.8 Hz, 1H), 4.33-3.87 (m, 3H), 3.76 (dd, J=10.8, 5.9 Hz, 4H), 3.54-3.26 (m, 8H), 2.97 (dd, J=16.2, 4.8 Hz, 2H), 2.22-1.72 (m, 5H), 1.46 (d, J=6.6 Hz, 9H). ¹³C NMR (101 MHz, CDCl₃) δ 165.79, 152.52, 150.83, 146.67, 140.56, 135.48, 128.77, 127.14, 124.99, 77.36, 66.97, 55.45, 53.77, 50.75, 46.53, 46.21, 43.86, 40.07, 39.92, 28.60, 28.55. LC-MS (C₃₀H₄₀N₈O₈S): Calculated [M+H]⁺ m/z=673.28, [M−H]⁻ m/z=671.26. Observed [M+H]⁺ m/z=673.46, [M−H]⁻ m/z=671.40.

### (S)-pyrrolidin-2-ylmethyl ((4-((3-((2,3-dihydro-1H-inden-2-yl)carbamoyl)pyrazin-2-yl)carbamoyl)piperazin-1-yl)sulfonyl)carbamate hydrochloride (MAT457 HCl)

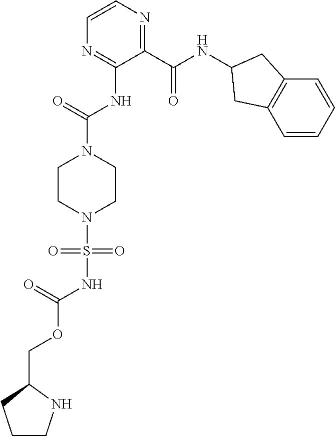 [SMILES: O=C(NS(=O)(=O)N1CCN(C(=O)Nc2nccnc2C(=O)NC2Cc3ccccc3C2)CC1)OC[C@@H]1CCCN1]

To MAT456 (46.4 mg, 69.0 μmol, 1 eq), added 1.2 mL 5:1 TFA/water. Stirred vigorously and monitored by LCMS. After 40 minutes, concentrated to dryness. Azeotroped with methanol once. Purified by reverse phase flash column chromatography (water/MeCN).

Yield: 22.3 mg, 56.5%. White solid. ¹H NMR (400 MHz, DMSO) δ 10.99 (s, 1H), 9.23 (d, J=7.8 Hz, 1H), 9.02 (s, 1H), 8.67 (s, 1H), 8.52 (d, J=2.4 Hz, 1H), 8.26 (d, J=2.4 Hz, 1H), 7.23 (dt, J=7.5, 3.7 Hz, 2H), 7.20-7.13 (m, 2H), 4.72 (h, J=7.5 Hz, 1H), 4.14 (dd, J=12.1, 3.5 Hz, 1H), 4.02-3.90 (m, 1H), 3.77-3.66 (m, 1H), 3.55 (t, J=5.1 Hz, 4H), 3.24-3.10 (m, 8H), 3.06 (dd, J=15.7, 7.3 Hz, 2H), 2.02 (dtt, J=11.4, 8.2, 4.2 Hz, 1H), 1.92 (dq, J=11.6, 5.7 Hz, 1H), 1.82 (dq, J=12.6, 7.8 Hz, 1H), 1.61 (dq, J=12.5, 8.1 Hz, 1H). ¹³C NMR (101 MHz, DMSO) δ 165.60, 152.44, 149.75, 145.66, 141.10, 135.77, 130.92, 126.48, 124.47, 63.57, 58.10, 50.42, 46.28, 45.31, 43.62, 40.43, 38.44, 26.27, 23.37. *Note this is missing one of the carbonyl carbons due to insufficient signal to noise. However, in the AMR for the enantiomer MAT459 (below), it was observed at 157.6 ppm. LC-MS (C₂₅H₃₂N₈O₆S): Calculated [M+H]⁺ m/z=573.22, [M−H]⁻ m/z=571.21. Observed [M+H]⁺ m/z=572.86, [M−H]⁻ m/z=571.41.

### tert-butyl (R)-2-(((((4-((3-((2,3-dihydro-1H-inden-2-yl)carbamoyl)pyrazin-2-yl)carbamoyl)piperazin-1-yl)sulfonyl)carbamoyl)oxy)methyl)pyrrolidine-1-carboxylate (MAT458)

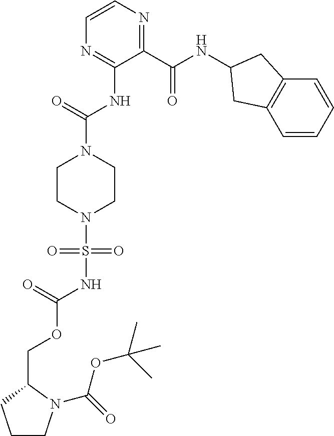 [SMILES: CC(C)(C)OC(=O)N1CCC[C@@H]1COC(=O)NS(=O)(=O)N1CCN(C(=O)Nc2nccnc2C(=O)NC2Cc3ccccc3C2)CC1]

Combined BOC-D-prolinol (50 mg, 250 μmol, 1 eq) and DCM (2 mL). Added chlorosulfonyl isocyanate (23.7 μL, 38.7 mg, 273 μmol, 1.1 eq) and stirred vigorously. To this, added premixed solution of MAT336·2HCl (162 mg, 370 μmol, 1.5 eq), DCM (2 mL), and DIPEA (216 μL). After reaction was complete by LCMS, quenched by addition of MeOH (1 mL). Concentrated to an oil. Purified by reverse phase flash column chromatography (water/MeCN, both with 0.1% formic acid). Yield: Quantitative. White solid. ¹H NMR (400 MHz, CDCl₃) δ 11.77 (s, 1H), 8.56 (s, 1H), 8.29 (d, J=8.1 Hz, 1H), 8.10-8.06 (m, 1H), 7.30-7.24 (m, 2H), 7.24-7.20 (m, 2H), 4.84 (qd, J=7.4, 3.7 Hz, 1H), 4.24-3.97 (m, 3H), 3.75 (t, J=5.0 Hz, 4H), 3.51 (d, J=5.2 Hz, 3H), 3.42 (dd, J=16.2, 7.2 Hz, 3H), 3.38-3.29 (m, 2H), 2.97 (dd, J=16.2, 4.8 Hz, 2H), 1.91 (dp, J=40.4, 7.8 Hz, 8H), 1.45 (s, 9H). ¹³C NMR (101 MHz, CDCl₃) δ 165.65, 152.41, 150.66, 146.45, 140.44, 135.36, 128.70, 127.02, 124.87, 55.32, 53.64, 50.85, 50.64, 46.68, 46.43, 46.40, 43.76, 39.95, 28.48. LC-MS (C₃₀H₄₀N₈O₈S): Calculated [M+H]⁺ m/z=673.28, [M−H]⁻ m/z=671.26. Observed [M+H]⁺ m/z=673.51, [M−H]⁻ m/z=671.43.

### (R)-pyrrolidin-2-ylmethyl ((4-((3-((2,3-dihydro-1H-inden-2-yl)carbamoyl)pyrazin-2-yl)carbamoyl)piperazin-1-yl)sulfonyl)carbamate (MAT459 HCl)

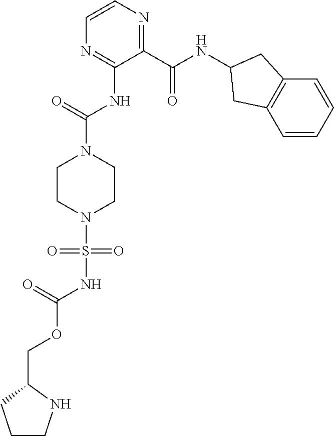 [SMILES: O=C(NS(=O)(=O)N1CCN(C(=O)Nc2nccnc2C(=O)NC2Cc3ccccc3C2)CC1)OC[C@H]1CCCN1]

To MAT458 (46.3 mg, 68.8 μmol, 1 eq), added 1.2 mL 5:1 TFA/water. Stirred vigorously and monitored by LCMS. After 40 minutes, concentrated to dryness. Azeotroped with methanol once. Purified by reverse phase flash column chromatography (water/MeCN). Yield: 22.4 mg, 56.8%. White solid. ¹H NMR (400 MHz, DMSO) δ 11.00 (s, 1H), 9.23 (d, J=7.8 Hz, 1H), 8.85 (s, 2H), 8.51 (d, J=2.4 Hz, 1H), 8.24 (d, J=2.4 Hz, 1H), 7.22 (dt, J=7.5, 3.8 Hz, 2H), 7.19-7.10 (m, 2H), 4.72 (h, J=7.6 Hz, 1H), 4.03 (dd, J=12.1, 3.4 Hz, 1H), 3.84 (dd, J=12.1, 8.7 Hz, 1H), 3.65 (qd, J=8.4, 3.4 Hz, 1H), 3.50 (t, J=5.1 Hz, 4H), 3.24-2.97 (m, 11H), 2.06-1.75 (m, 3H), 1.58 (dq, J=12.5, 8.2 Hz, 1H). ¹³C NMR (101 MHz, DMSO) δ 165.62, 157.67, 152.31, 149.86, 145.73, 141.10, 135.69, 130.59, 126.48, 124.46, 62.95, 58.60, 50.44, 46.41, 45.13, 43.65, 40.43, 38.43, 26.25, 23.36. LC-MS (C₂₅H₃₂N₈O₆S): Calculated [M+H]⁺ m/z=573.22, [M−H]⁻ m/z=571.22. Observed [M+H]⁺ m/z=572.90, [M−H]⁻ m/z=571.31.

### 3-(2-hydroxyethyl)quinazolin-4(3H)-one (MAT509)

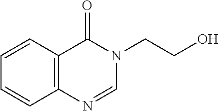 [SMILES: O=c1c2ccccc2ncn1CCO]

Synthesized based upon the method reported by He et al. with minor modifications.⁵² In short, charged a flask with 3-methyl-1H-indole (658 mg, 5.02 mmol, 1 eq), acetonitrile (30 mL), water (15 mL), ethanolamine (600 μL, 606 mg, 9.92 mmol, 2 eq), 70% tert-butyl hydroperoxide_{(aq)} (4.00 mL, 29.1 mmol, 5.8 eq), and tetrabutylammonium iodide (315 mg, 852 μmol, 017 eq). Heated to 100° C. and left stirring for 36 h. Washed with water (100 mL) and extracted 3× with EtOAc (100 mL). Dried over Na₂SO₄, filtered, and concentrated. Purified by reverse phase flash column chromatography (water/MeCN). Yield: 371 mg, 39%. ¹H NMR (400 MHz, DMSO) δ 8.26 (s, 1H), 8.16 (dd, J=8.0, 1.6 Hz, 1H), 7.82 (ddd, J=8.5, 7.2, 1.6 Hz, 1H), 7.67 (d, J=8.1 Hz, 1H), 7.54 (ddd, J=8.2, 7.2, 1.2 Hz, 1H), 4.96 (t, J=5.6 Hz, 1H), 4.04 (t, J=5.3 Hz, 2H), 3.67 (dt, J=5.4, 5.4 Hz, 2H). ¹³C NMR (101 MHz, DMSO) δ 160.30, 148.63, 148.08, 134.21, 127.12, 126.86, 126.01, 121.68, 58.32, 48.63. LC-MS (C₁₀H₁₀N₂O₂): Calculated [M+H]⁺ m/z=191.08. Observed [M+H]⁺ m/z=191.09.

### 2-(4-oxoquinazolin-3(4H)-yl)ethyl (N-(((3aR,4R,6R,6aR)-6-(6-amino-9H-purin-9-yl)-2,2-dimethyltetrahydrofuro[3,4-d][1,3]dioxol-4-yl)methyl)sulfamoyl)carbamate (MAT510)

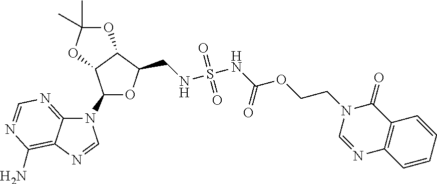 [SMILES: CC1(C)O[C@@H]2[C@H](O1)[C@@H](CNS(=O)(=O)NC(=O)OCCn1cnc3ccccc3c1=O)O[C@H]2n1cnc2c(N)ncnc21]

Synthesis of 5′-Amino-5′-deoxy-2′,3′-O-isopropylidene-adenosine (MAT521) was reported previously and is described herein. Combined MAT509 (100 mg, 526 μmol, 1 eq) and MeCN (3.5 mL). Added chlorosulfonyl isocyanate (50.2 μL, 81.9 mg, 578 μmol, 1.1 eq) and stirred vigorously. To this, added premixed solution of MAT521 (65.5 mg, 149 μmol, 1.25 eq), MeCN (3.5 mL), dimethylacetamide (2 mL) and DIPEA (458 μL, 2.63 mmol, 5 eq). After reaction was complete by LCMS, quenched with MeOH (1 mL) and concentrated to an oil. Purified by reverse phase flash column chromatography (water/MeCN, both with 0.1% formic acid). Yield: 149.4 mg, 47%. ¹H NMR (400 MHz, CDCl₃) δ 8.58 (t, J=5.8 Hz, 1H), 8.31 (s, 1H), 8.25 (s, 1H), 8.16 (s, 1H), 8.12 (d, 1H), 7.83-7.75 (m, 1H), 7.64 (d, J=8.1 Hz, 1H), 7.51 (t, J=7.6 Hz, 1H), 7.42 (s, 2H), 6.10 (d, J=3.2 Hz, 1H), 5.33 (dd, J=6.2, 3.3 Hz, 1H), 4.95 (dd, J=6.3, 2.6 Hz, 1H), 4.33 (t, J=5.1 Hz, 2H), 4.28 (td, J=5.5, 2.4 Hz, 1H), 4.23 (t, J=5.0 Hz, 2H), 3.23 (dt, J=13.4, 5.2 Hz, 1H), 3.13 (td, J=11.1, 4.5 Hz, 1H), 1.53 (s, 3H), 1.30 (s, 3H). ¹³C NMR (101 MHz, CDCl₃) δ 160.31, 156.20, 151.22, 148.36, 148.10, 147.85, 134.35, 127.17, 127.01, 121.45, 119.38, 113.44, 89.85, 83.51, 82.74, 81.66, 62.86, 44.93, 44.64, 27.04, 25.20. LC-MS (C₂₄H₂₇N₉O₈S): Calculated [M+H]⁺ m/z=602.18, [M−H]⁻ m/z=600.16. Observed [M+H]⁺ m/z=602.34, [M−H]⁻ m/z=600.30.

### 2-(4-oxoquinazolin-3(4H)-yl)ethyl (N-(((2R,3S,4R,5R)-5-(6-amino-9H-purin-9-yl)-3,4-dihydroxytetrahydrofuran-2-yl)methyl)sulfamoyl)carbamate (MAT511)

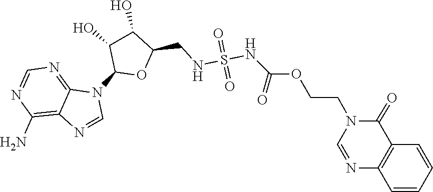 [SMILES: Nc1ncnc2c1ncn2[C@@H]1O[C@H](CNS(=O)(=O)NC(=O)OCCn2cnc3ccccc3c2=O)[C@@H](O)[C@H]1O]

To MAT510 (50.4 mg, 83.8 μmol, 1 eq), added 1.2 mL 5:1 TFA/water. Stirred vigorously and monitored by LCMS. After 30 minutes, concentrated to dryness. Azeotroped with methanol once. Purified by reverse phase flash column chromatography (water/MeCN). Yield: 22.5 mg, 47.8%. ¹H NMR (400 MHz, DMSO) δ 11.37 (s, 1H), 9.19 (dd, J=8.2, 3.9 Hz, 1H), 8.31 (s, 1H), 8.27 (s, 1H), 8.16 (s, 1H), 8.13 (dd, J=7.9, 1.5 Hz, 1H), 7.82-7.74 (m, 1H), 7.65 (d, J=8.1 Hz, 1H), 7.50 (dd, J=13.1, 5.5 Hz, 3H), 5.82 (d, J=7.0 Hz, 1H), 5.59-5.42 (m, 1H), 5.32 (s, 1H), 4.70 (t, J=5.9 Hz, 1H), 4.45-4.31 (m, J=5.4, 4.9 Hz, 2H), 4.24 (dd, J=6.5, 4.1 Hz, 2H), 4.08 (d, J=4.6 Hz, 2H), 3.26-3.10 (m, 3H). ¹³C NMR (101 MHz, DMSO) δ 160.31, 156.12, 152.15, 151.31, 148.64, 148.43, 148.03, 147.83, 140.75, 134.30, 127.15, 126.99, 126.07, 121.42, 119.69, 88.61, 83.69, 72.32, 71.40, 62.76, 48.62, 45.02, 44.98, 40.43. LC-MS (C₂₁H₂₃N₉O₈S): Calculated [M+H]⁺ m/z=562.15, [M−H]⁻ m/z=560.13. Observed [M+H]⁺ m/z=562.29, [M−H]⁻ m/z=560.23.

### 2-(4-oxoquinazolin-3(4H)-yl)ethyl ((4-((3-((2,3-dihydro-1H-inden-2-yl)carbamoyl)pyrazin-2-yl)carbamoyl)piperazin-1-yl)sulfonyl)carbamate (MAT512)

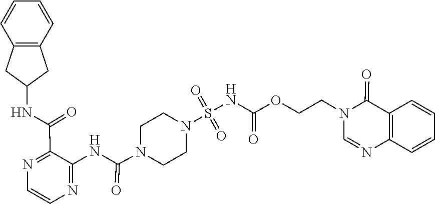 [SMILES: O=C(NS(=O)(=O)N1CCN(C(=O)Nc2nccnc2C(=O)NC2Cc3ccccc3C2)CC1)OCCn1cnc2ccccc2c1=O]

Combined MAT509 (24.1 mg, 127 μmol, 1 eq) and DCM (2 mL). Added chlorosulfonyl isocyanate (12.6 μL, 20.5 mg, 145 μmol, 1.15 eq) and stirred vigorously. To this, added premixed solution of 14 (MAT336) 2HCl (86.5 mg, 197 μmol, 1.25 eq), DCM (2 mL), and DIPEA (114 μL, 654 μmol, 5.2 eq). After reaction was complete by LCMS, quenched with MeOH (1 mL) and concentrated. Purified by reverse phase flash column chromatography (water/MeCN, both with 0.1% formic acid). Yield: 68.5 mg, 82%. White solid. ¹H NMR (400 MHz, DMSO) δ 11.55 (s, 1H), 10.96 (s, 1H), 9.21 (d, J=7.8 Hz, 1H), 8.52 (d, J=2.4 Hz, 1H), 8.29 (s, 1H), 8.25 (d, J=2.4 Hz, 1H), 8.14 (d, J=7.9 Hz, 1H), 7.79-7.71 (m, 1H), 7.65 (d, J=8.1 Hz, 1H), 7.49 (t, J=7.6 Hz, 1H), 7.25-7.18 (m, 2H), 7.18-7.11 (m, 2H), 4.72 (h, J=7.6 Hz, 1H), 4.38 (t, J=5.0 Hz, 2H), 4.25 (t, J=5.0 Hz, 2H), 3.49 (t, J=5.2 Hz, 4H), 3.19 (q, J=8.1 Hz, 6H), 3.05 (dd, J=15.7, 7.3 Hz, 2H). ¹³C NMR (101 MHz, DMSO) δ 165.56, 160.30, 152.51, 149.67, 148.63, 148.05, 147.82, 145.62, 141.11, 135.80, 134.38, 134.22, 131.13, 127.15, 127.04, 126.87, 126.45, 126.08, 126.01, 124.46, 121.42, 58.29, 50.42, 48.61, 45.85, 45.07, 43.43, 38.45. LC-MS (C₃₀H₃₁N₉O₇S): Calculated [M+H]⁺ m/z=662.21, [M−H]⁻ m/z=660.20. Observed [M+H]⁺ m/z=662.43, [M−H]⁻ m/z=660.38.

### 2-(4-oxoquinazolin-3(4H)-yl)ethyl (N-(1-((3-((2,3-dihydro-1H-inden-2-yl)carbamoyl)pyrazin-2-yl)carbamoyl)piperidin-4-yl)sulfamoyl)carbamate (MAT513)

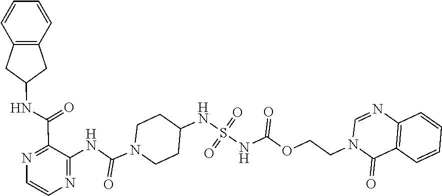 [SMILES: O=C(NS(=O)(=O)NC1CCN(C(=O)Nc2nccnc2C(=O)NC2Cc3ccccc3C2)CC1)OCCn1cnc2ccccc2c1=O]

Combined MAT509 (24.4 mg, 187 μmol, 1 eq) and DCM (2 mL). Added chlorosulfonyl isocyanate (12.6 μL, 20.5 mg, 145 μmol, 1.13 eq) and stirred vigorously. To this, added premixed solution of cmpd 8 (MAT332) 2HCl (91.3 mg, 201 μmol, 1.57 eq), DCM (2 mL), and DIPEA (114 μL, 654 μmol, 5.1 eq). After reaction was complete by LCMS, quenched with MeOH (1 mL) and concentrated. Purified by reverse phase flash column chromatography (water/MeCN, both with 0.1% formic acid). Yield: 55.8 mg, 64.4%. White solid. ¹H NMR (400 MHz, DMSO) δ 11.30 (s, 1H), 10.98 (s, 1H), 9.23 (d, J=7.8 Hz, 1H), 8.51 (d, J=2.4 Hz, 1H), 8.29 (s, 1H), 8.23 (d, J=2.4 Hz, 1H), 8.16 (dd, J=8.0, 1.5 Hz, 1H), 7.94 (s, 1H), 7.82-7.75 (m, 1H), 7.68 (d, J=8.2 Hz, 1H), 7.52 (t, J=7.6 Hz, 1H), 7.21 (dt, J=7.6, 3.8 Hz, 2H), 7.18-7.11 (m, 2H), 4.73 (h, J=7.5 Hz, 1H), 4.41 (t, J=5.0 Hz, 2H), 4.27 (t, J=5.0 Hz, 2H), 3.89 (d, J=12.9 Hz, 2H), 3.19 (dd, J=15.7, 7.7 Hz, 2H), 3.05 (dd, J=15.8, 7.3 Hz, 2H), 2.97 (t, J=12.0 Hz, 2H), 1.80-1.67 (m, 2H), 1.44-1.30 (m, 2H). ¹³C NMR (101 MHz, DMSO) δ 165.66, 160.32, 152.09, 149.97, 148.09, 147.89, 145.75, 141.10, 135.54, 134.41, 130.42, 127.21, 127.08, 126.47, 126.10, 124.46, 121.45, 62.69, 50.44, 45.12, 42.46, 38.43, 31.80. LC-MS (C₃₁H₃₃N₉O₇S): Calculated [M+H]⁺ m/z=676.23, [M−H]⁻ m/z=674.22. Observed [M+H]⁺ m/z=676.41, [M−H]⁻ m/z=674.32.

### 2-(4-oxoquinazolin-3(4H)-yl)ethyl (N-((1-((3-((2,3-dihydro-1H-inden-2-yl)carbamoyl)pyrazin-2-yl)carbamoyl)piperidin-4-yl)methyl)sulfamoyl)carbamate (MAT514)

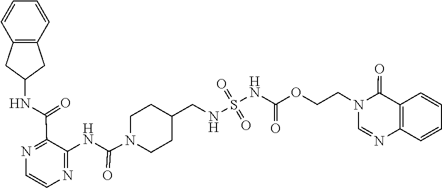 [SMILES: O=C(NS(=O)(=O)NCC1CCN(C(=O)Nc2nccnc2C(=O)NC2Cc3ccccc3C2)CC1)OCCn1cnc2ccccc2c1=O]

Combined MAT509 (25.4 mg, 134 μmol, 1 eq) and DCM (2 mL). Added chlorosulfonyl isocyanate (12.6 μL, 20.5 mg, 145 μmol, 1.09 eq) and stirred vigorously. To this, added premixed solution of cmpd 10·2HCl (96.9 mg, 207 μmol, 1.55 eq), DCM (2 mL), and DIPEA (114 μL, 654 μmol, 4.9 eq). After reaction was complete by LCMS, quenched with MeOH (1 mL) and concentrated. Purified by reverse phase flash column chromatography (water/MeCN, both with 0.1% formic acid). Yield: 60.3 mg, 66%. Light orange solid. ¹H NMR (400 MHz, DMSO) δ 11.24 (s, 1H), 10.99 (s, 1H), 9.21 (d, J=7.8 Hz, 1H), 8.50 (d, J=2.3 Hz, 1H), 8.28 (s, 1H), 8.23-8.21 (m, 1H), 8.15 (dd, J=8.0, 1.5 Hz, 1H), 7.86-7.77 (m, 2H), 7.67 (d, J=8.2 Hz, 1H), 7.53 (t, J=7.6 Hz, 1H), 7.22 (dt, J=7.5, 3.8 Hz, 2H), 7.18-7.11 (m, 2H), 4.80-4.63 (m, 1H), 4.40 (t, J=5.0 Hz, 2H), 4.27 (t, J=5.0 Hz, 2H), 4.10 (t, J=5.3 Hz, 3H), 4.01 (d, J=13.1 Hz, 2H), 3.24-3.18 (m, 2H), 3.17 (d, J=3.4 Hz, 7H), 3.05 (dd, J=15.8, 7.2 Hz, 2H), 2.80 (t, J=12.6 Hz, 2H), 2.70 (t, J=6.2 Hz, 2H), 1.65 (t, J=10.2 Hz, 3H), 1.10-0.95 (m, 2H). ¹³C NMR (101 MHz, DMSO) δ 165.69, 160.32, 152.06, 150.06, 148.12, 147.89, 145.76, 141.19, 141.11, 135.42, 134.44, 130.29, 127.22, 127.09, 126.47, 126.10, 124.46, 121.46, 62.68, 50.44, 48.62, 47.87, 45.13, 43.64, 38.60, 38.44, 35.34, 29.25. LC-MS (C₃₂H₃₅N₉O₇S): Calculated [M+H]⁺ m/z=690.25, [M−H]⁻ m/z=688.23. Observed [M+H]⁺ m/z=690.48, [M−H]⁻ m/z=688.51.

### Example 3—Biochemical and In Vitro Activity of Selected Compounds

| | Dd2 (EC₅₀ [nM]) | | | | | | | | |
|---|---|---|---|---|---|---|---|---|---|
| | | Halofuginone- | HFGR- | PfcProRS (K_{D} [nM]) | | | HsProRS (K_{D} [nM]) | | |
| Compound | wt | induced | I | − | +proline | +ATP | − | +proline | +ATP |
| halofuginone(1) | 0.519 | 19.2 | 196 | >3,000 | >3,000 | 0.503 | 1,160 | 2,044 | 0.225 |
| 2 | 595 | 1,250 | 736 | 437 | 7.17 | 291 | 371 | 2.16 | 954 |
| NCP26 (3) | 67.4 | 120 | 68.6 | 130 | 2.52 | 103 | 271 | 0.351 | 678 |
| 4 | 180 | 540 | 290 | 275 | 3.42 | — | 221 | 2.55 | — |
| 5 | >10000 | | | >2860 | | | >8,720 | | |
| 6 | 508 | 7,930 | 6,870 | 31.5 | 26.3 | — | 56.8 | 82.7 | — |
| 7 | 90.6 | 2,170 | 2,750 | 13.1 | 13.2 | — | 22.1 | 34.8 | — |
| ProSA (25) | 151 | 137 | 170 | 0.0559 | <0.5 | <0.5 | 0.0702 | <1 | <1 |
| MAT436 (34) | 6.76 | 251 | 2630 | 0.375 | 0.722 | — | 0.465 | 6.65 | — |
| 35 | 18.7 | 355 | 4990 | 0.680 | 0.729 | — | 0.35 | 3.49 | — |
| 36 | 18.7 | 383 | 5730 | 0.821 | 0.772 | — | 0.367 | 6.63 | — |
| 37 | 59% † | 7150 | 6950 | 246 | 175 | — | 502 | 570 | — |
| 38 | 3950 | 6710 | 5590 | 455 | 279 | — | 720 | 609 | — |
| 39 | 72% †† | 98% †† | 95% †† | 227 | 179 | — | 216 | 658 | — |
| iso- | 21.9 | 979 | 6,820 | 4.57 | 3.91 | — | 18.6 | 20.8 | — |
| MAT436(40) | | | | | | | | | |
| 8 | | | | >2,860 | >2,180 | | >8,720 | >5,090 | |
| 9 | 6570 | 7530 | 8210 | 382 | 353 | | 2,510 | >5,090 | |
| 10 | | | | >2,860 | >2,180 | | >8,720 | 1,360 | |
| 11 | 712 | | | 299 | | | 265 | | |
| 12 | 153 | 6350 | 7970 | 515 | 339 | — | 687 | 150 | |
| 13 | | | | >2,860 | >2,180 | | >8,720 | >5,090 | |
| 14 | | | | >2,860 | 1,630 | | >8,720 | 347 | |
| 15 | 1500 | 3510 | 3040 | 737 | 127 | — | 2,700 | 21.8 | |
| 16 | 6,110 | | | >2,860 | >2,180 | | >8,720 | >5,090 | |
| 17 | 5790 | | | 1,390 | 131 | | 4,430 | 25.5 | |
| 18 | >20000 | | | >2,860 | 1,590 | | 7,400 | 225 | |
| 19 | >10,000 | 8590 | 7890 | >2,860 | 1,180 | | >8,720 | 1,320 | |
| 20 | 3740 | 6010 | 4400 | 1,260 | 100 | | 738 | 13.2 | |
| 21 | 9600 | | | >2,860 | 1,430 | | 7,830 | 140 | |
| 22 | 12,300 | | | >2,860 | >2,180 | | >8,720 | >5,090 | |
| 23 | 12,100 | | | >2,860 | >2,180 | | >8,720 | >5,090 | |
| 24 | | | | 100 | | | 1,700 | | |
| 41 | 2110 | 6910 | 4520 | 227 | 182 | | 139 | 561 | |
| 42 | 3120 | 9080 | 8250 | 433 | 295 | | 674 | 780 | |
| 43 | 3330 | 5090 | 4810 | 375 | 320 | | 611 | 2,280 | |
| 44 | >10,000 | 8910 | 8430 | 321 | 270 | | 1,570 | 330 | |
| 45 | 13,600 | | | >2,860 | >2,180 | | >8,720 | >5,090 | |
| 46 | 8,110 | | | >2,860 | >2,180 | | >8,720 | >5,090 | |
| 47 | 11,100 | | | >2,860 | >2,180 | | >8,720 | | |
| 48 | 8,740 | | | >2,860 | >2,180 | | >8,720 | >5,090 | |
| 49 | 6,800 | | | >2,860 | >2,180 | | >8,720 | >5,090 | |
| 50 | 13,000 | | | >2,860 | >2,180 | | >8,720 | >5,090 | |
| MAT420 | | | | 292 | 332 | | 415 | 781 | |
| MAT421 | | | | 2280 | 1960 | | 2340 | 2260 | |
| MAT482 | 2,720 | 5,540 | 5,910 | 78.3 | 61.6 | | 516 | 1,360 | |
| MAT483 | | | | 198 | 171 | | 1,000 | 3,020 | |
| MAT515 | 381 | 3,300 | 4,580 | 70.4 | 46.6 | | 169 | 504 | |
| MAT516 | 636 | 4,760 | 4,040 | 94.1 | 60.8 | | 402 | 159 | |
| MAT517 | 2,680 | 6,840 | 5,670 | 95.6 | 80.2 | | 865 | 1,680 | |
| MAT518 | 1,820 | 1,500 | 1,740 | 372 | 288 | | 2,760 | >5,090 | |
| MAT430 | | | | 5.01 | | | 18.4 | | |
| MAT582 | 8,660 | >10,000 | >10,000 | 2,490 | 2,740 | | 265 | 8.42 | |
| MAT583 | 2,270 | 7,750 | >10,000 | 187 | 19 | | 687 | 150 | |
| MAT445 | 1,160 | 7,620 | 7,120 | 1,110 | 966 | | | | |
| MAT447 | 6,250 | 6,990 | 7,320 | 2,460 | 2,450 | | | | |
| MAT457 | 968 | 6,570 | >10,000 | 152 | 118 | | 9.71 | 16.3 | |
| MAT459 | 4,620 | 7,080 | >10,000 | 736 | 552 | | 105 | 245 | |
| MAT511 | 76.5 | 52.2 | 69.0 | >2,860 | 1,810 | | >8,720 | >5,090 | |
| MAT512 | >10,000 | | | | | | | | |
| MAT513 | 4,830 | | | | | | | | |
| MAT514 | 4,560 | 7,040 | 7,840 | 35.2 | 32.6 | | 277 | 372 | |

Data are presented as the mean of n ≥ 2 technical replicates and are representative of n ≥ 3 biologically independent experiments.
† Data shown is percent growth at 10 μM because compound had <50% growth inhibition (>50% growth).
†† Data shown is percent growth at 1 μM because compound had <50% growth inhibition (>50% growth).

### NUMBERED PARAGRAPHS

The invention of the present disclosure can be described by reference to the following numbered paragraphs:

Paragraph 1. A compound of Formula (I):

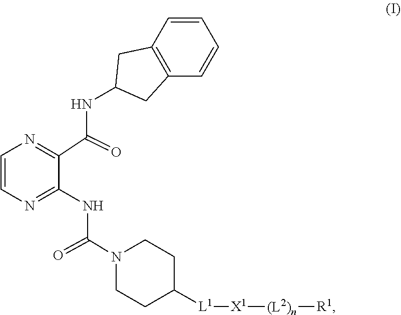

or a pharmaceutically acceptable salt thereof, wherein:
L¹ is C₁₋₃ alkylene, or L¹ is absent;
X¹ is selected from O and NR^{N};
R^{N} is selected from H, C₁₋₃ alkyl, and C₁₋₃ haloalkyl;
each L² is independently selected from O, S, S(═O)₂, NR^{N}, C═O, C═S, and C₁₋₆ alkylene;
n is an integer from 0 to 12; and
R¹ is selected from H, C₁₋₆ alkyl, C₃₋₆ cycloalkyl, C₆₋₁₀ aryl.

Paragraph 2. The compound of paragraph 1, wherein L¹ is absent.

Paragraph 3. The compound of paragraph 1, wherein L¹ is C₁₋₃ alkylene.

Paragraph 4. The compound of paragraph 3, wherein L¹ is selected from methylene, 1,2-ethylene, 1,1-ethylene, and propylene.

Paragraph 5. The compound of paragraph 3, wherein L¹ is methylene.

Paragraph 6. The compound of any one of paragraphs 1-5, wherein X¹ is O.

Paragraph 7. The compound of any one of paragraphs 1-5, wherein X¹ is NH.

Paragraph 8. The compound of paragraph 1, wherein the compound of Formula (I) has formula:

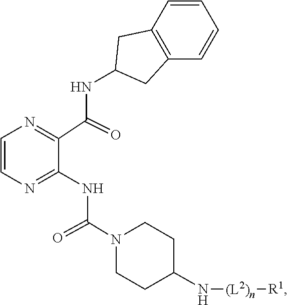

or a pharmaceutically acceptable salt thereof.

Paragraph 9. The compound of paragraph 1, wherein the compound of Formula (I) has formula:

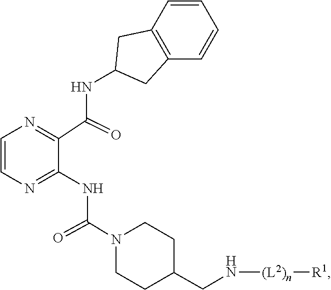

or a pharmaceutically acceptable salt thereof.

Paragraph 10. The compound of any one of paragraphs 1-9, wherein n is 0.

Paragraph 11. The compound of any one of paragraphs 1-9, wherein n is an integer from 1 to 12.

Paragraph 12. The compound of paragraph 11, wherein each L² is independently selected from O, S, NH, C═O, C═S, and C₁₋₆ alkylene.

Paragraph 13. The compound of paragraph 12, wherein the moiety (L²)ₙ comprises (C═O)O.

Paragraph 14. The compound of paragraph 12, wherein the moiety (L²)ₙ comprises NH(C═O)O.

Paragraph 15. The compound of paragraph 12, wherein the moiety (L²)ₙ comprises (C═O)—C₁₋₆ alkylene.

Paragraph 16. The compound of paragraph 12, wherein the moiety (L²)ₙ comprises NH(C═O).

Paragraph 17. The compound of paragraph 12, wherein the moiety (L²)ₙ comprises NH(C═O)NH.

Paragraph 18. The compound of paragraph 12, wherein the moiety (L²)ₙ comprises NH(C═S)NH.

Paragraph 19. The compound of paragraph 12, wherein the moiety (L²)ₙ comprises any one of the following fragments:

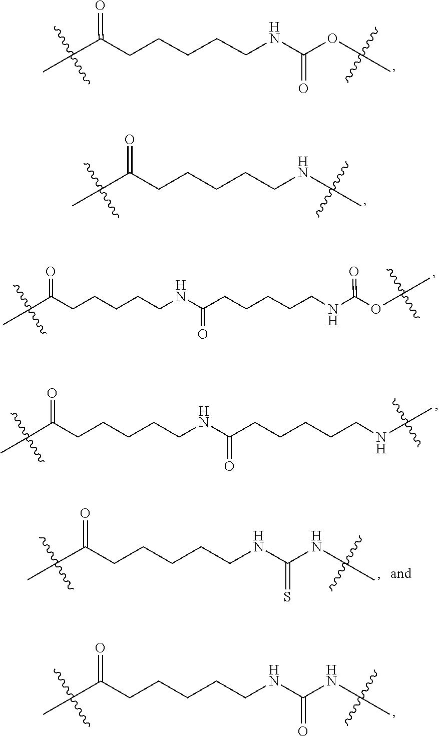 [SMILES: *OC(=O)NCCCCCC(*)=O ; *NCCCCCC(*)=O ; *OC(=O)NCCCCCC(=O)NCCCCCC(*)=O ; *NCCCCCC(=O)NCCCCCC(*)=O ; *NC(=S)NCCCCCC(*)=O ; *NC(=O)NCCCCCC(*)=O]

Paragraph 20. The compound of any one of paragraphs 1-19, R¹ is H.

Paragraph 21. The compound of any one of paragraphs 1-19, R¹ is C₁₋₆ alkyl.

Paragraph 22. The compound of any one of paragraphs 1-19, R¹ is C₃₋₆ cycloalkyl.

Paragraph 23. The compound of any one of paragraphs 1-19, R¹ is C₆₋₁₀ aryl.

Paragraph 24. The compound of paragraph 1, wherein the compound of Formula (I) is selected from any one of the following compounds:

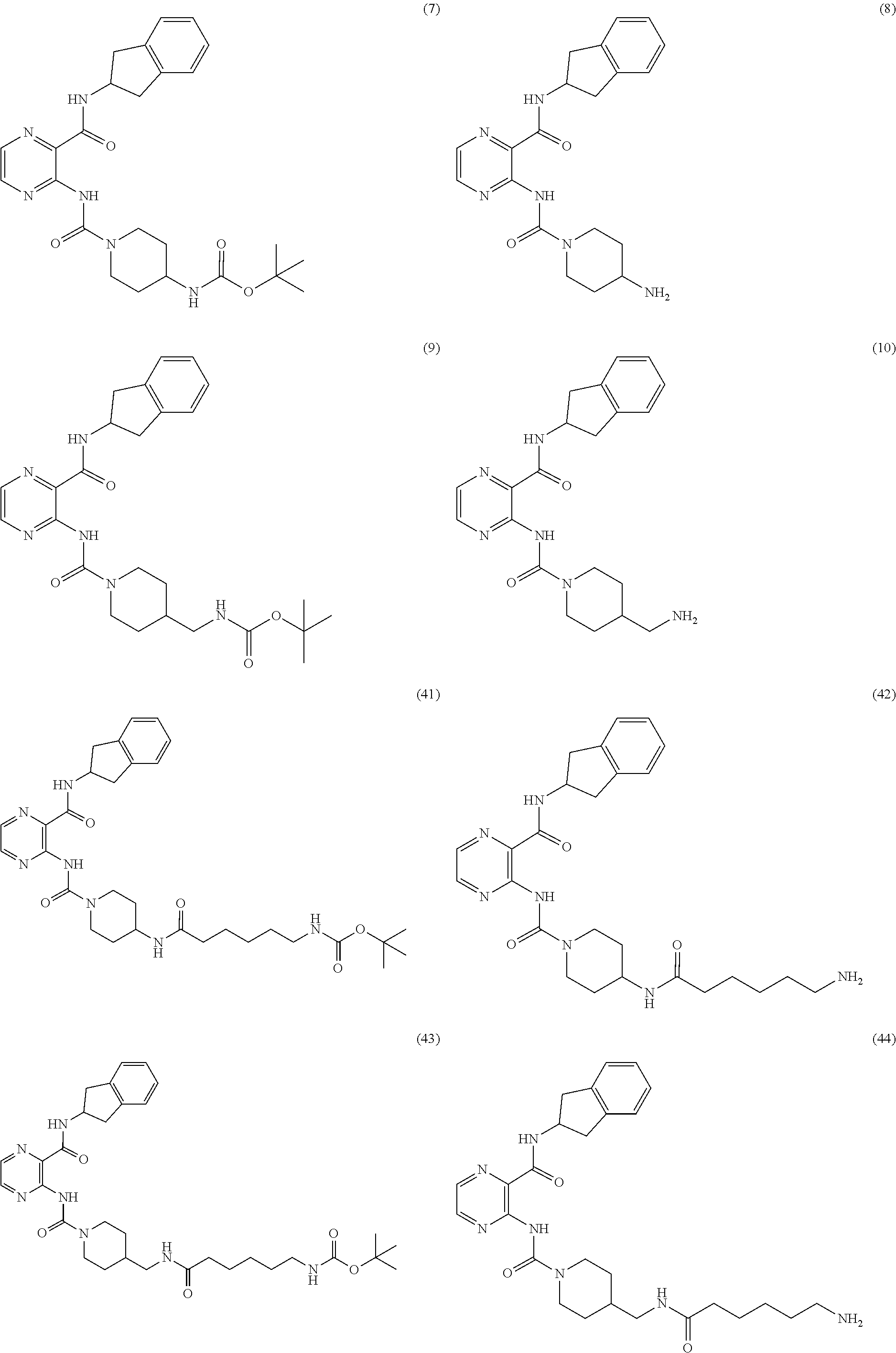

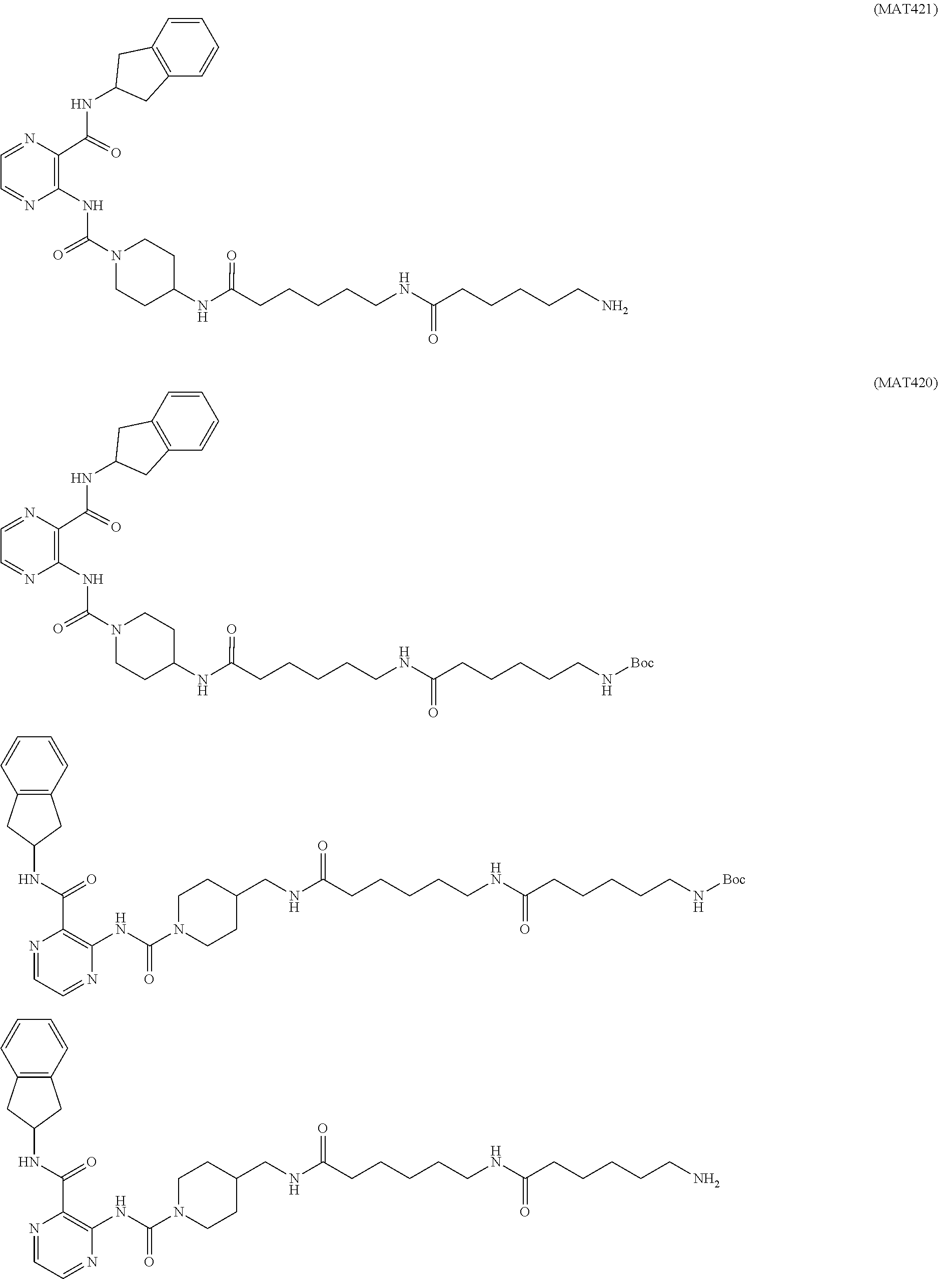

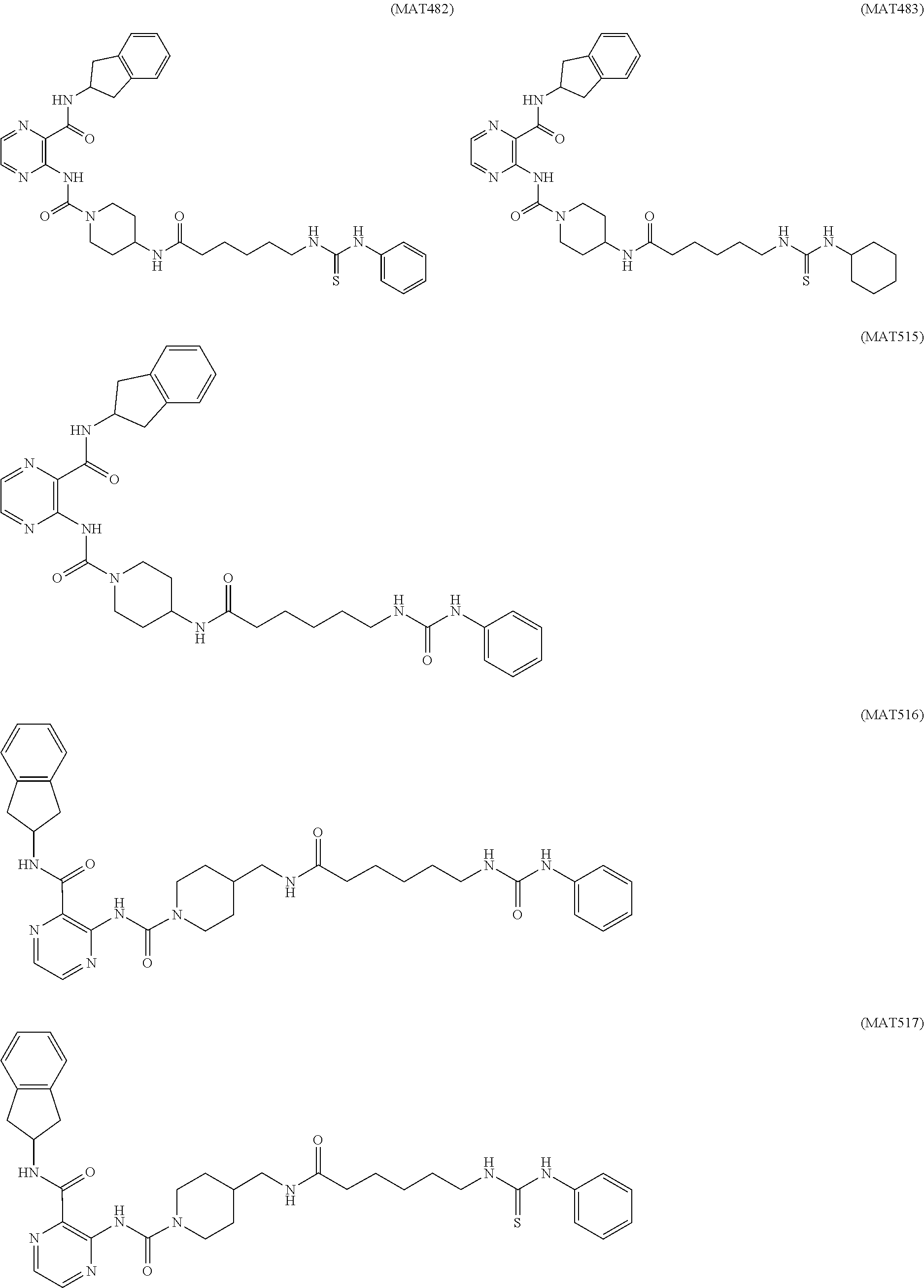 [SMILES: O=C(CCCCCNC(=S)Nc1ccccc1)NC1CCN(C(=O)Nc2nccnc2C(=O)NC2Cc3ccccc3C2)CC1 ; O=C(CCCCCNC(=S)NC1CCCCC1)NC1CCN(C(=O)Nc2nccnc2C(=O)NC2Cc3ccccc3C2)CC1 ; O=C(CCCCCNC(=O)Nc1ccccc1)NC1CCN(C(=O)Nc2nccnc2C(=O)NC2Cc3ccccc3C2)CC1 ; O=C(CCCCCNC(=O)Nc1ccccc1)NCC1CCN(C(=O)Nc2nccnc2C(=O)NC2Cc3ccccc3C2)CC1 ; O=C(CCCCCNC(=S)Nc1ccccc1)NCC1CCN(C(=O)Nc2nccnc2C(=O)NC2Cc3ccccc3C2)CC1]

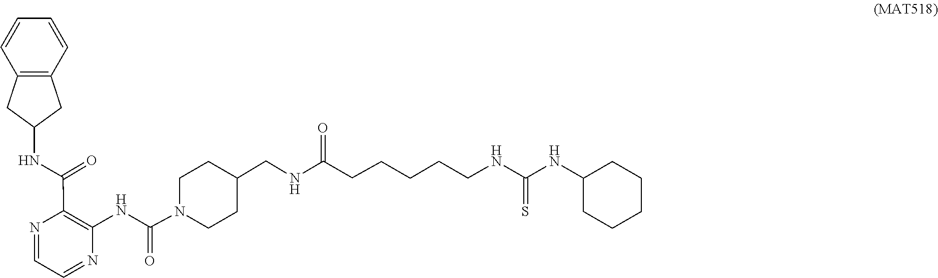 [SMILES: O=C(CCCCCNC(=S)NC1CCCCC1)NCC1CCN(C(=O)Nc2nccnc2C(=O)NC2Cc3ccccc3C2)CC1]

or a pharmaceutically acceptable salt thereof.

Paragraph 25. A compound of formula:

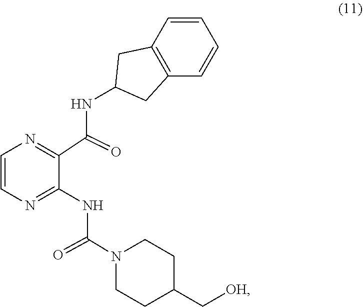 [SMILES: O=C(NC1Cc2ccccc2C1)c1nccnc1NC(=O)N1CCC(CO)CC1]

or a pharmaceutically acceptable salt thereof.

Paragraph 26. A compound of formula:

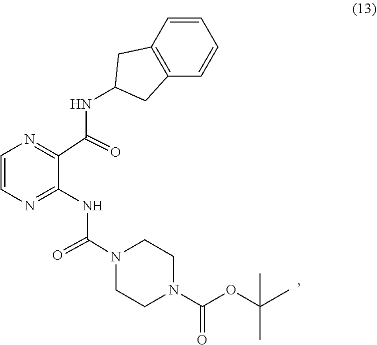 [SMILES: CC(C)(C)OC(=O)N1CCN(C(=O)Nc2nccnc2C(=O)NC2Cc3ccccc3C2)CC1]

or a pharmaceutically acceptable salt thereof.

Paragraph 27. A compound of Formula (II):

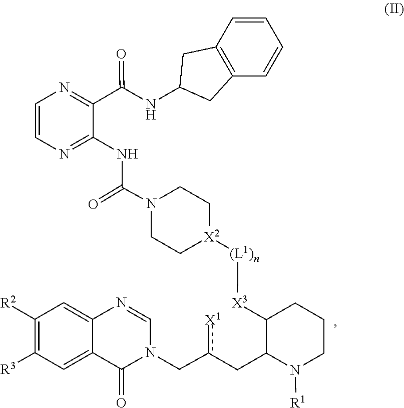

or a pharmaceutically acceptable salt thereof, wherein:
[symbol] is a single bond or a double bond;
when [symbol] is a double bond, X¹ is O or S;
when [symbol] is a single bond, X¹ is OH or SH;
R¹ is selected from H, C₁₋₃ alkyl, C₁₋₃ haloalkyl, —C(═O)R^{A1}, and —C(═O)OR^{A1};
each R^{A1} is selected from H, C₁₋₆ alkyl, and C₁₋₃ haloalkyl;
R² and R³ are each independently a halogen;
X² is selected from N and CH;
X³ is selected from O and NH;
n is an integer from 1 to 6; and
each L¹ is independently selected from O, S, S(═O)₂, NR^{N}, C═O, C═S, and C₁₋₆ alkylene.

Paragraph 28. The compound of paragraph 27, wherein the compound has formula:

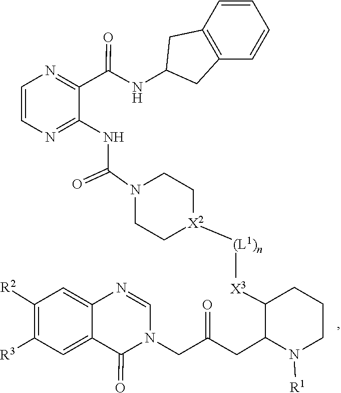

or a pharmaceutically acceptable salt thereof.

Paragraph 29. The compound of paragraph 27, wherein the compound has formula:

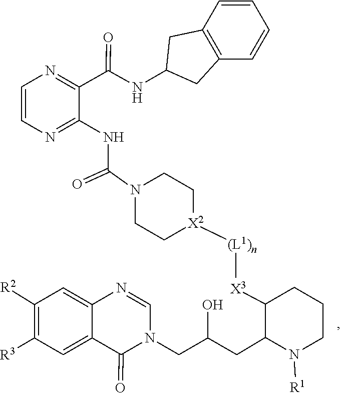

or a pharmaceutically acceptable salt thereof.

Paragraph 30. The compound of any one of paragraphs 27-29, wherein R¹ is H.

Paragraph 31. The compound of any one of paragraphs 27-29, wherein R¹ is C₁₋₃ alkyl or C₁₋₃ haloalkyl.

Paragraph 32. The compound of any one of paragraphs 27-29, wherein R¹ is —C(═O)R^{A1}.

Paragraph 33. The compound of any one of paragraphs 27-29, wherein R¹ is —C(═O)OR^{A1}.

Paragraph 34. The compound of any one of paragraphs 27-22, wherein R^{A1} is H.

Paragraph 35. The compound of any one of paragraphs 27-22, wherein R^{A1} is C₁₋₆ alkyl.

Paragraph 36. The compound of any one of paragraphs 27-35, wherein R² and R³ are each independently Br or Cl.

Paragraph 37. The compound of any one of paragraphs 27-36, wherein X³ is O.

Paragraph 38. The compound of any one of paragraphs 27-36, wherein X³ is NH.

Paragraph 39. The compound of any one of paragraphs 27-38, wherein n is selected from 3, 4, and 5.

Paragraph 40. The compound of any one of paragraphs 27-39, wherein each L¹ is independently selected from S(═O)₂, NH, C═O, and C₁₋₃ alkylene.

Paragraph 41. The compound of paragraph 42, wherein (L¹)ₙ comprises S(═O)₂NH.

Paragraph 42. The compound of paragraph 42, wherein (L¹)ₙ comprises NHC(═O).

Paragraph 43. The compound of paragraph 42, wherein (L¹)ₙ comprises NHS(═O)₂NH.

Paragraph 44. The compound of paragraph 42, wherein (L¹)ₙ comprises CH₂N.

Paragraph 45. The compound of any one of paragraphs 27-44, wherein X² is N.

Paragraph 46. The compound of paragraph 45, wherein (L¹)ₙ comprises:

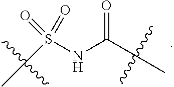 [SMILES: *C(=O)NS(*)(=O)=O]

Paragraph 47. The compound of any one of paragraphs 27-43, wherein X² is CH.

Paragraph 48. The compound of paragraph 47, wherein (L¹)ₙ comprises:

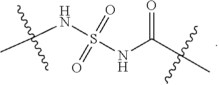 [SMILES: *NS(=O)(=O)NC(*)=O]

Paragraph 49. The compound of paragraph 47, wherein (L¹)ₙ comprises:

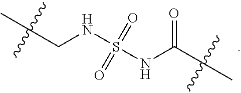 [SMILES: *CNS(=O)(=O)NC(*)=O]

Paragraph 50. The compound of paragraph 27, having formula:

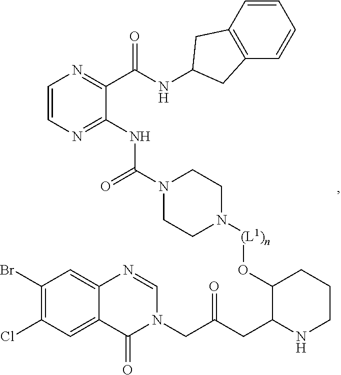

or a pharmaceutically acceptable salt thereof.

Paragraph 51. The compound of paragraph 27, having formula:

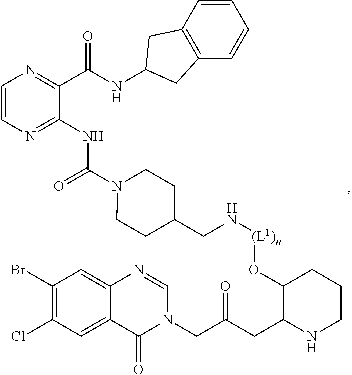

or a pharmaceutically acceptable salt thereof.

Paragraph 52. The compound of paragraph 27, having formula:

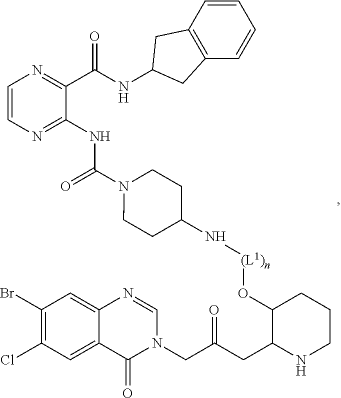

or a pharmaceutically acceptable salt thereof.

Paragraph 53. The compound of paragraph 27, wherein the compound of Formula (II) is selected from any one of the following compounds:

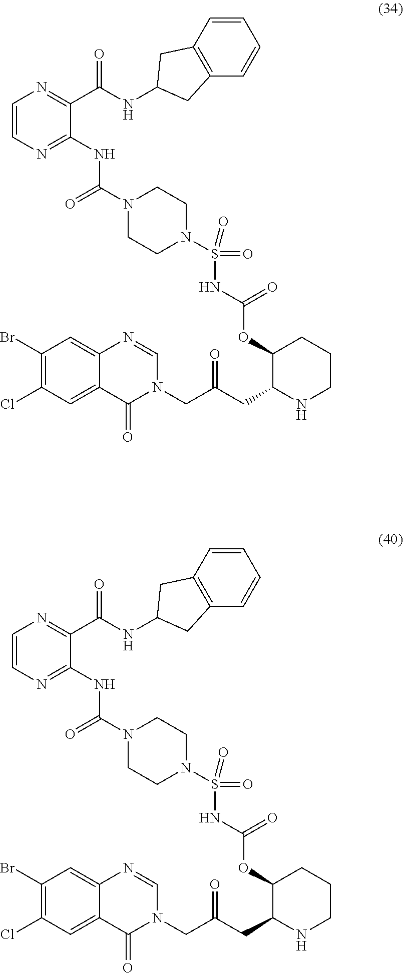 [SMILES: O=C(C[C@H]1NCCC[C@@H]1OC(=O)NS(=O)(=O)N1CCN(C(=O)Nc2nccnc2C(=O)NC2Cc3ccccc3C2)CC1)Cn1cnc2cc(Br)c(Cl)cc2c1=O ; O=C(C[C@@H]1NCCC[C@@H]1OC(=O)NS(=O)(=O)N1CCN(C(=O)Nc2nccnc2C(=O)NC2Cc3ccccc3C2)CC1)Cn1cnc2cc(Br)c(Cl)cc2c1=O]

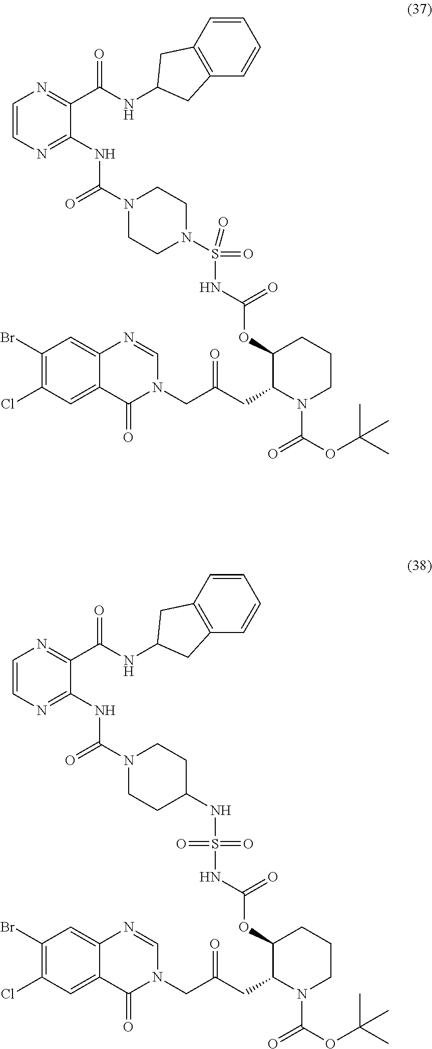 [SMILES: CC(C)(C)OC(=O)N1CCC[C@H](OC(=O)NS(=O)(=O)N2CCN(C(=O)Nc3nccnc3C(=O)NC3Cc4ccccc4C3)CC2)[C@H]1CC(=O)Cn1cnc2cc(Br)c(Cl)cc2c1=O ; CC(C)(C)OC(=O)N1CCC[C@H](OC(=O)NS(=O)(=O)NC2CCN(C(=O)Nc3nccnc3C(=O)NC3Cc4ccccc4C3)CC2)[C@H]1CC(=O)Cn1cnc2cc(Br)c(Cl)cc2c1=O]

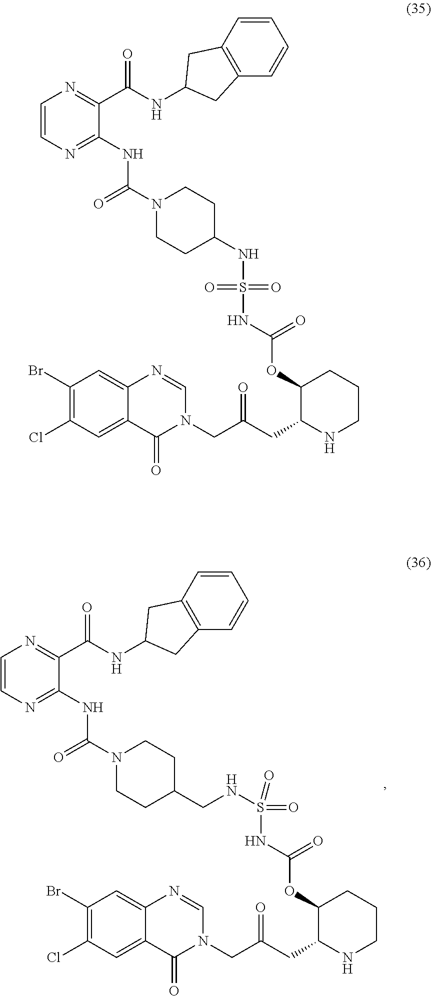 [SMILES: O=C(C[C@H]1NCCC[C@@H]1OC(=O)NS(=O)(=O)NC1CCN(C(=O)Nc2nccnc2C(=O)NC2Cc3ccccc3C2)CC1)Cn1cnc2cc(Br)c(Cl)cc2c1=O ; O=C(C[C@H]1NCCC[C@@H]1OC(=O)NS(=O)(=O)NCC1CCN(C(=O)Nc2nccnc2C(=O)NC2Cc3ccccc3C2)CC1)Cn1cnc2cc(Br)c(Cl)cc2c1=O]

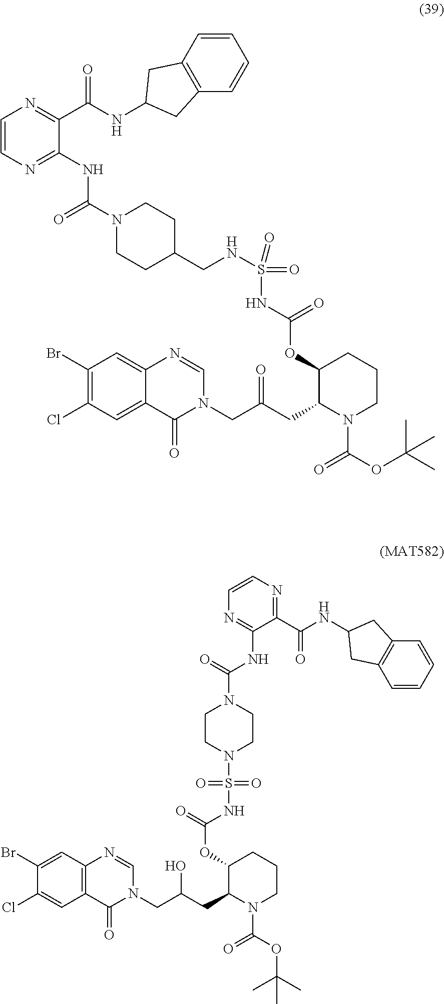 [SMILES: CC(C)(C)OC(=O)N1CCC[C@H](OC(=O)NS(=O)(=O)NCC2CCN(C(=O)Nc3nccnc3C(=O)NC3Cc4ccccc4C3)CC2)[C@H]1CC(=O)Cn1cnc2cc(Br)c(Cl)cc2c1=O ; CC(C)(C)OC(=O)N1CCC[C@@H](OC(=O)NS(=O)(=O)N2CCN(C(=O)Nc3nccnc3C(=O)NC3Cc4ccccc4C3)CC2)[C@@H]1CC(O)Cn1cnc2cc(Br)c(Cl)cc2c1=O]

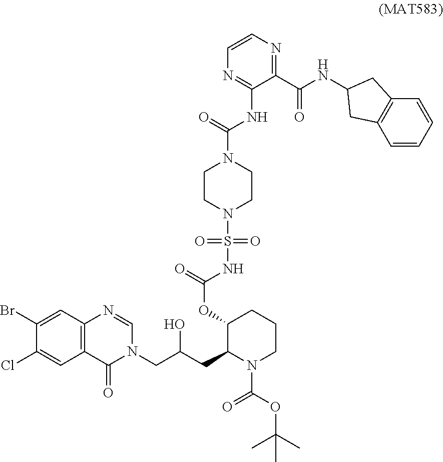 [SMILES: CC(C)(C)OC(=O)N1CCC[C@@H](OC(=O)NS(=O)(=O)N2CCN(C(=O)Nc3nccnc3C(=O)NC3Cc4ccccc4C3)CC2)[C@@H]1CC(O)Cn1cnc2cc(Br)c(Cl)cc2c1=O]

or a pharmaceutically acceptable salt thereof.

Paragraph 54. A compound of formula:

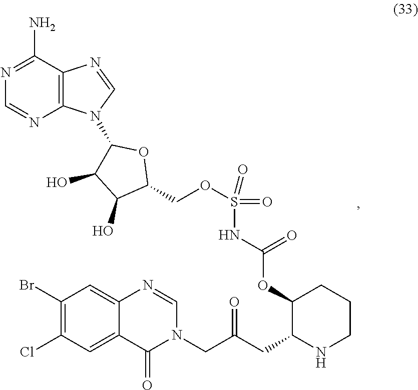 [SMILES: Nc1ncnc2c1ncn2[C@@H]1O[C@H](COS(=O)(=O)NC(=O)O[C@H]2CCCN[C@@H]2CC(=O)Cn2cnc3cc(Br)c(Cl)cc3c2=O)[C@@H](O)[C@H]1O]

or a pharmaceutically acceptable salt thereof.

Paragraph 55. A compound of formula:

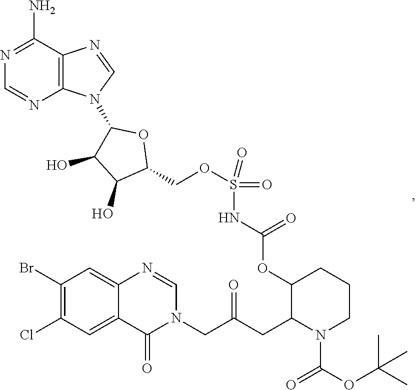 [SMILES: CC(C)(C)OC(=O)N1CCCC(OC(=O)NS(=O)(=O)OC[C@H]2O[C@@H](n3cnc4c(N)ncnc43)[C@H](O)[C@@H]2O)C1CC(=O)Cn1cnc2cc(Br)c(Cl)cc2c1=O]

or a pharmaceutically acceptable salt thereof.

Paragraph 56. A compound of Formula (III):

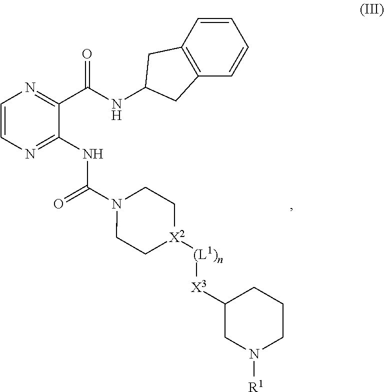

or a pharmaceutically acceptable salt thereof, wherein:
R¹ is selected from H, C₁₋₃ alkyl, C₁₋₃ haloalkyl, —C(═O)R^{A1}, and —C(═O)OR^{A1};
each R^{A1} is selected from H, C₁₋₆ alkyl, and C₁₋₃ haloalkyl;
X² is selected from N and CH;
X³ is selected from O and NH;
n is an integer from 1 to 6; and
each L¹ is independently selected from O, S, S(═O)₂, NR^{N}, C═O, C═S, and C₁₋₆ alkylene.

Paragraph 57. The compound of paragraph 56, wherein R¹ is H.

Paragraph 58. The compound of paragraph 56, wherein R¹ is C₁₋₃ alkyl or C₁₋₃ haloalkyl.

Paragraph 59. The compound of paragraph 56, wherein R¹ is —C(═O)R^{A1}.

Paragraph 60. The compound of paragraph 56, wherein R¹ is —C(═O)OR^{A1}.

Paragraph 61. The compound of any one of paragraphs 56-60, wherein R^{A1} is H.

Paragraph 62. The compound of any one of paragraphs 56-60, wherein R^{A1} is C₁₋₆ alkyl.

Paragraph 63. The compound of any one of paragraphs 56-62, wherein X³ is O.

Paragraph 64. The compound of any one of paragraphs 56-62, wherein X³ is NH.

Paragraph 65. The compound of any one of paragraphs 56-64, wherein n is selected from 3, 4, and 5.

Paragraph 66. The compound of any one of paragraphs 56-65, wherein each L¹ is independently selected from S(═O)₂, NH, C═O, and C₁₋₃ alkylene.

Paragraph 67. The compound of paragraph 66, wherein (L¹)ₙ comprises S(═O)₂NH.

Paragraph 68. The compound of paragraph 66, wherein (L¹)ₙ comprises NHC(═O).

Paragraph 69. The compound of paragraph 66, wherein (L¹)ₙ comprises NHS(═O)₂NH.

Paragraph 70. The compound of paragraph 66, wherein (L¹)ₙ comprises CH₂N.

Paragraph 71. The compound of any one of paragraphs 56-70, wherein X² is N.

Paragraph 72. The compound of paragraph 71, wherein (L¹)ₙ comprises:

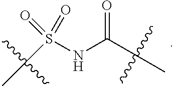 [SMILES: *C(=O)NS(*)(=O)=O]

Paragraph 73. The compound of any one of paragraphs 56-70, wherein X² is CH.

Paragraph 74. The compound of paragraph 73, wherein (L¹)ₙ comprises:

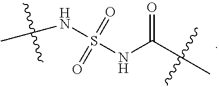 [SMILES: *NS(=O)(=O)NC(*)=O]

Paragraph 75. The compound of paragraph 73, wherein (L¹)ₙ comprises:

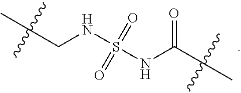 [SMILES: *CNS(=O)(=O)NC(*)=O]

Paragraph 76. The compound of paragraph 56, wherein the compound has formula:

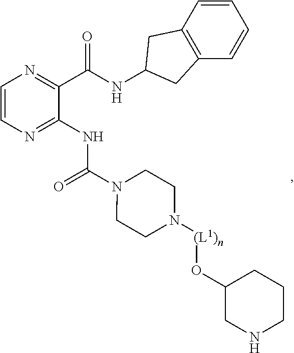

or a pharmaceutically acceptable salt thereof.

Paragraph 77. The compound of paragraph 56, wherein the compound has formula:

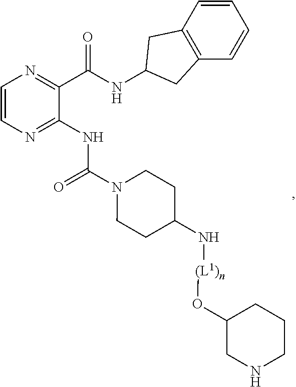

or a pharmaceutically acceptable salt thereof.

Paragraph 78. The compound of paragraph 56, wherein the compound has formula:

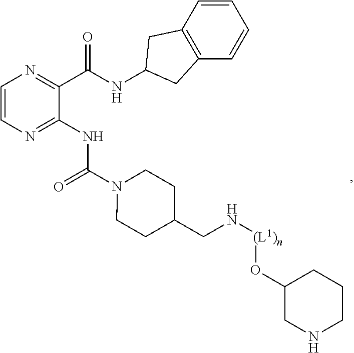

or a pharmaceutically acceptable salt thereof.

Paragraph 79. The compound of paragraph 56, wherein the compound of Formula (III) is selected from any one of the following compounds:

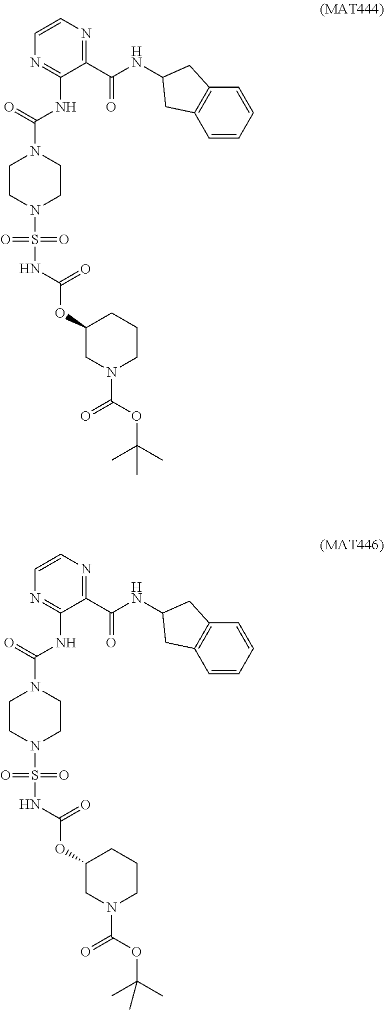 [SMILES: CC(C)(C)OC(=O)N1CCC[C@H](OC(=O)NS(=O)(=O)N2CCN(C(=O)Nc3nccnc3C(=O)NC3Cc4ccccc4C3)CC2)C1 ; CC(C)(C)OC(=O)N1CCC[C@@H](OC(=O)NS(=O)(=O)N2CCN(C(=O)Nc3nccnc3C(=O)NC3Cc4ccccc4C3)CC2)C1]

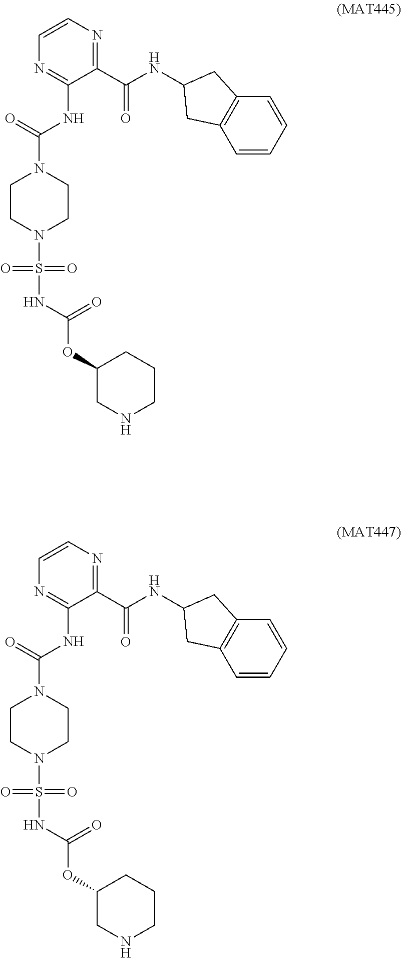 [SMILES: O=C(NS(=O)(=O)N1CCN(C(=O)Nc2nccnc2C(=O)NC2Cc3ccccc3C2)CC1)O[C@H]1CCCNC1 ; O=C(NS(=O)(=O)N1CCN(C(=O)Nc2nccnc2C(=O)NC2Cc3ccccc3C2)CC1)O[C@@H]1CCCNC1]

or a pharmaceutically acceptable salt thereof.

Paragraph 80. A compound of Formula (IV):

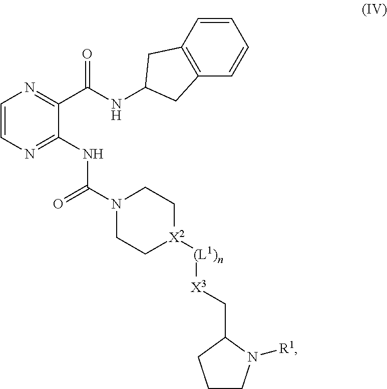

or a pharmaceutically acceptable salt thereof, wherein:
R¹ is selected from H, C₁₋₃ alkyl, C₁₋₃ haloalkyl, —C(═O)R^{A1}, and —C(═O)OR^{A1};
each R^{A1} is selected from H, C₁₋₆ alkyl, and C₁₋₃ haloalkyl;
X² is selected from N and CH;
X³ is selected from O and NH;
n is an integer from 1 to 6; and
each L¹ is independently selected from O, S, S(═O)₂, NR^{N}, C═O, C═S, and C₁₋₆ alkylene.

Paragraph 81. The compound of paragraph 80, wherein R¹ is H.

Paragraph 82. The compound of paragraph 80, wherein R¹ is C₁₋₃ alkyl or C₁₋₃ haloalkyl.

Paragraph 83. The compound of paragraph 80, wherein R¹ is —C(═O)R^{A1}.

Paragraph 84. The compound of paragraph 80, wherein R¹ is —C(═O)OR^{A1}.

Paragraph 85. The compound of any one of paragraphs 80-84, wherein R^{A1} is H.

Paragraph 86. The compound of any one of paragraphs 80-84, wherein R^{A1} is C₁₋₆ alkyl.

Paragraph 87. The compound of any one of paragraphs 80-86, wherein X³ is O.

Paragraph 88. The compound of any one of paragraphs 80-86, wherein X³ is NH.

Paragraph 89. The compound of any one of paragraphs 80-88, wherein n is selected from 3, 4, and 5.

Paragraph 90. The compound of any one of paragraphs 80-89, wherein each L¹ is independently selected from S(═O)₂, NH, C═O, and C₁₋₃ alkylene.

Paragraph 91. The compound of paragraph 90, wherein (L¹)ₙ comprises S(═O)₂NH.

Paragraph 92. The compound of paragraph 90, wherein (L¹)ₙ comprises NHC(═O).

Paragraph 93. The compound of paragraph 90, wherein (L¹)ₙ comprises NHS(═O)₂NH.

Paragraph 94. The compound of paragraph 90, wherein (L¹)ₙ comprises CH₂N.

Paragraph 95. The compound of any one of paragraphs 80-94, wherein X² is N.

Paragraph 96. The compound of paragraph 95, wherein (L¹)ₙ comprises:

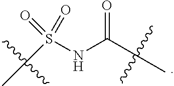 [SMILES: *C(=O)NS(*)(=O)=O]

Paragraph 97. The compound of any one of paragraphs 80-94, wherein X² is CH.

Paragraph 98. The compound of paragraph 97, wherein (L¹)ₙ comprises:

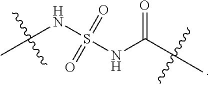 [SMILES: *NS(=O)(=O)NC(*)=O]

Paragraph 99. The compound of paragraph 97, wherein (L¹)ₙ comprises:

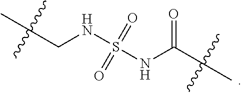 [SMILES: *CNS(=O)(=O)NC(*)=O]

Paragraph 100. The compound of paragraph 80, wherein the compound of Formula (IV) has formula:

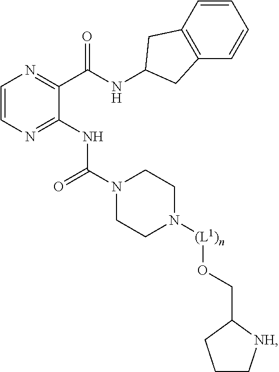

or a pharmaceutically acceptable salt thereof.

Paragraph 101. The compound of paragraph 80, wherein the compound of Formula (IV) has formula:

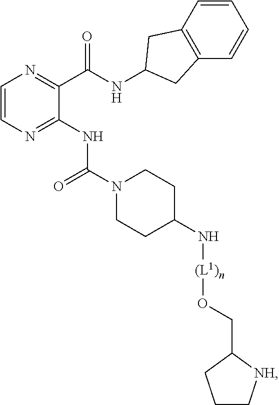

or a pharmaceutically acceptable salt thereof.

Paragraph 102. The compound of paragraph 80, wherein the compound of Formula (IV) has formula:

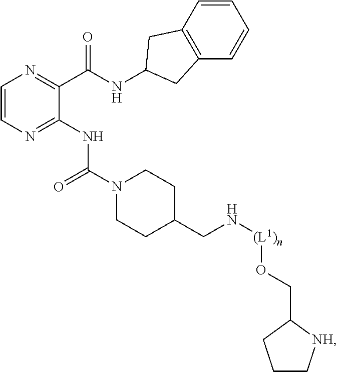

or a pharmaceutically acceptable salt thereof.

Paragraph 103. The compound of paragraph 80, wherein the compound of Formula (IV) is selected from any one of the following compounds:

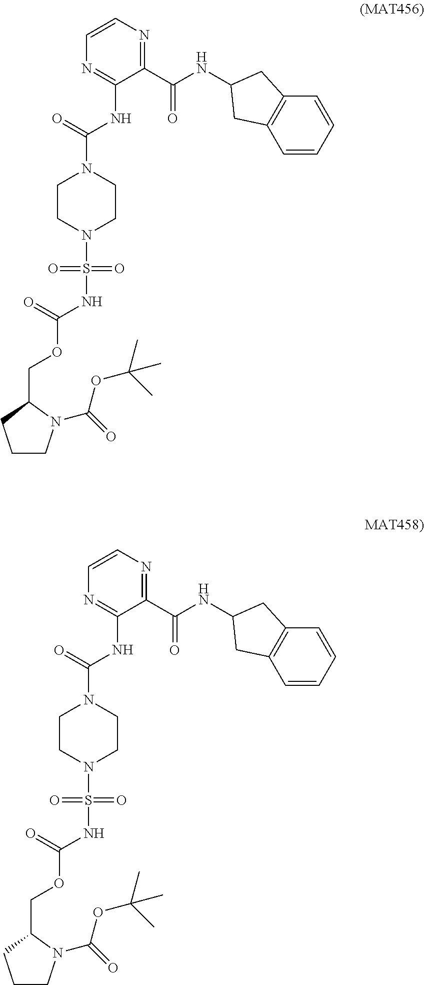 [SMILES: CC(C)(C)OC(=O)N1CCC[C@H]1COC(=O)NS(=O)(=O)N1CCN(C(=O)Nc2nccnc2C(=O)NC2Cc3ccccc3C2)CC1 ; CC(C)(C)OC(=O)N1CCC[C@@H]1COC(=O)NS(=O)(=O)N1CCN(C(=O)Nc2nccnc2C(=O)NC2Cc3ccccc3C2)CC1]

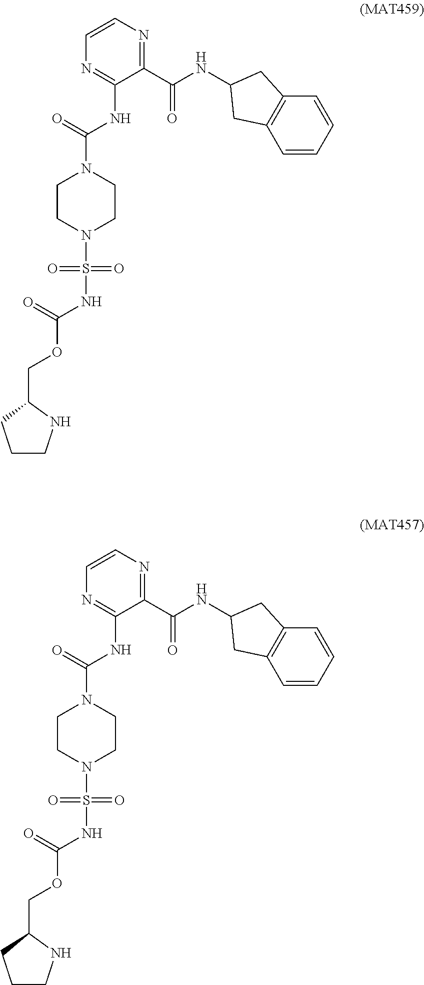 [SMILES: O=C(NS(=O)(=O)N1CCN(C(=O)Nc2nccnc2C(=O)NC2Cc3ccccc3C2)CC1)OC[C@H]1CCCN1 ; O=C(NS(=O)(=O)N1CCN(C(=O)Nc2nccnc2C(=O)NC2Cc3ccccc3C2)CC1)OC[C@@H]1CCCN1]

or a pharmaceutically acceptable salt thereof.

Paragraph 104. A compound of formula:

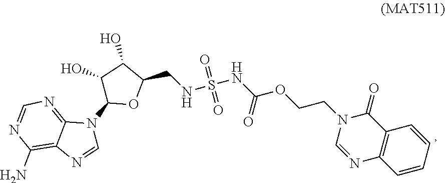 [SMILES: Nc1ncnc2c1ncn2[C@@H]1O[C@H](CNS(=O)(=O)NC(=O)OCCn2cnc3ccccc3c2=O)[C@@H](O)[C@H]1O]

or a pharmaceutically acceptable salt thereof.

Paragraph 105. A compound of Formula (V):

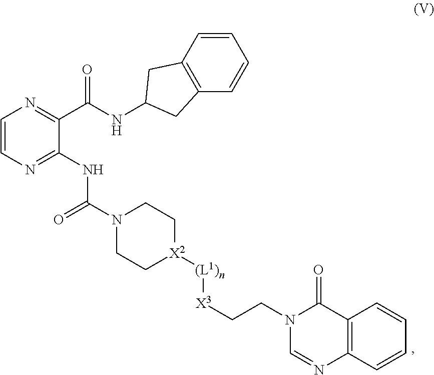

or a pharmaceutically acceptable salt thereof, wherein:
X² is selected from N and CH;
X³ is selected from O and NH;
n is an integer from 1 to 6; and
each L¹ is independently selected from O, S, S(═O)₂, NR^{N}, C═O, C═S, and C₁₋₆ alkylene.

Paragraph 106. The compound of paragraph 105, wherein X³ is O.

Paragraph 107. The compound of paragraph 105, wherein X³ is NH.

Paragraph 108. The compound of any one of paragraphs 105-107, wherein n is selected from 3, 4, and 5.

Paragraph 109. The compound of any one of paragraphs 105-108, wherein each L¹ is independently selected from S(═O)₂, NH, C═O, and C₁₋₃ alkylene.

Paragraph 110. The compound of paragraph 109, wherein (L¹)ₙ comprises S(═O)₂NH.

Paragraph 111. The compound of paragraph 109, wherein (L¹)ₙ comprises NHC(═O).

Paragraph 112. The compound of paragraph 109, wherein (L¹)ₙ comprises NHS(═O)₂NH.

Paragraph 113. The compound of paragraph 109, wherein (L¹)ₙ comprises CH₂N.

Paragraph 114. The compound of any one of paragraphs 105-113, wherein X² is N.

Paragraph 115. The compound of paragraph 114, wherein (L¹)ₙ comprises:

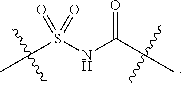 [SMILES: *C(=O)NS(*)(=O)=O]

Paragraph 116. The compound of any one of paragraphs 105-113, wherein X² is CH.

Paragraph 117. The compound of paragraph 116, wherein (L¹)ₙ comprises:

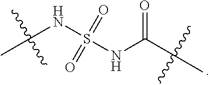 [SMILES: *NS(=O)(=O)NC(*)=O]

Paragraph 118. The compound of paragraph 116, wherein (L¹)ₙ comprises:

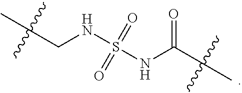 [SMILES: *CNS(=O)(=O)NC(*)=O]

Paragraph 119. The compound of paragraph 105, wherein the compound has

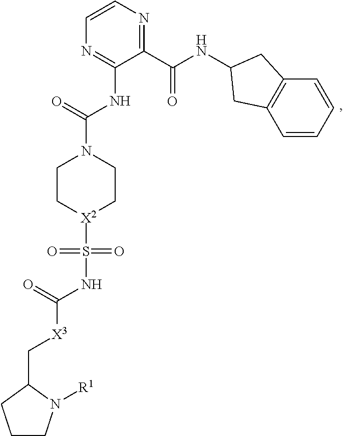

or a pharmaceutically acceptable salt thereof.

Paragraph 120. The compound of paragraph 105, wherein the compound has formula:

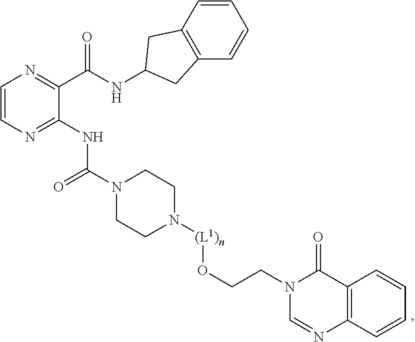

or a pharmaceutically acceptable salt thereof.

Paragraph 121. The compound of paragraph 105, wherein the compound has formula:

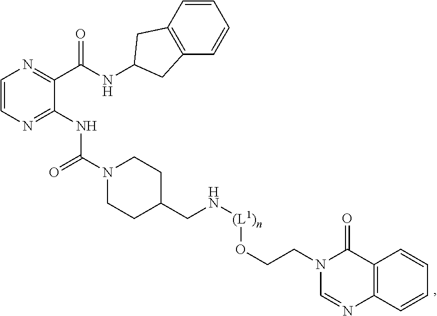

or a pharmaceutically acceptable salt thereof.

Paragraph 122. The compound of paragraph 105, wherein the compound has formula:

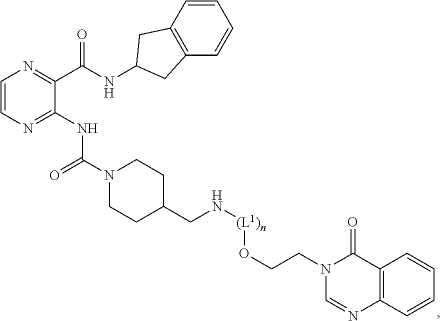

or a pharmaceutically acceptable salt thereof.

Paragraph 123. The compound of paragraph 105, wherein the compound of Formula (V) is selected from any one of the following compounds:

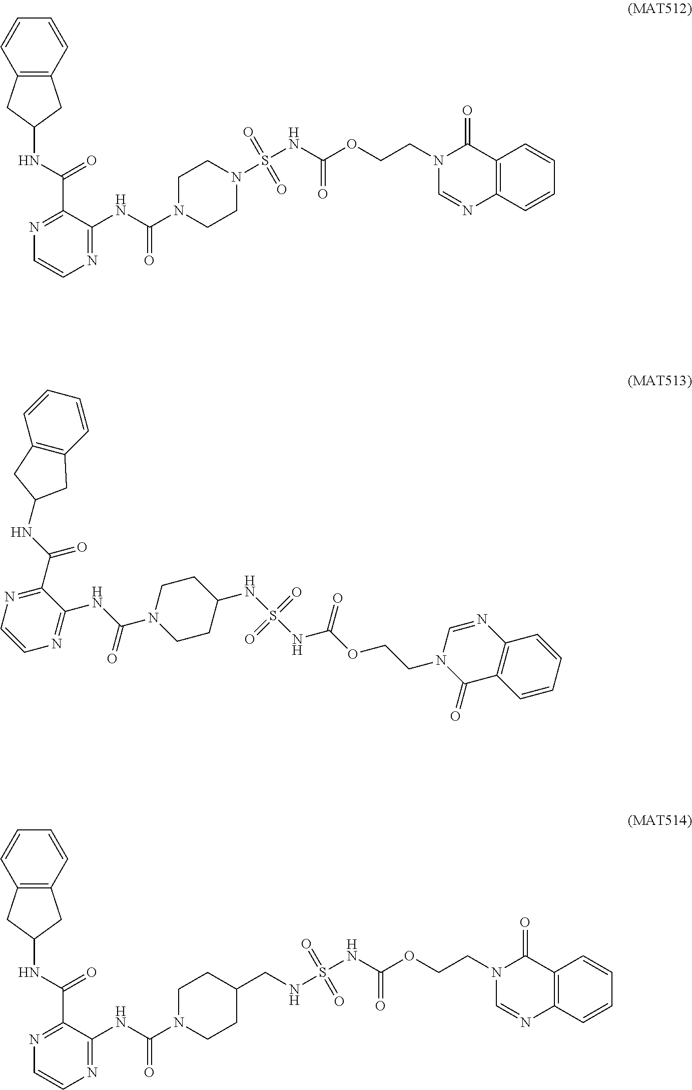 [SMILES: O=C(NS(=O)(=O)N1CCN(C(=O)Nc2nccnc2C(=O)NC2Cc3ccccc3C2)CC1)OCCn1cnc2ccccc2c1=O ; O=C(NS(=O)(=O)NC1CCN(C(=O)Nc2nccnc2C(=O)NC2Cc3ccccc3C2)CC1)OCCn1cnc2ccccc2c1=O ; O=C(NS(=O)(=O)NCC1CCN(C(=O)Nc2nccnc2C(=O)NC2Cc3ccccc3C2)CC1)OCCn1cnc2ccccc2c1=O]

or a pharmaceutically acceptable salt thereof.

Paragraph 124. A pharmaceutical composition comprising a compound of any one of paragraphs 1-123, or a pharmaceutically acceptable salt thereof, and a pharmaceutically acceptable carrier.

Paragraph 125. A method of inhibiting prolyl-tRNA-synthetase in a cell, comprising contacting the cell with a compound of any one of paragraphs 1-123, or a pharmaceutically acceptable salt thereof.

Paragraph 126. The method of paragraph 125, wherein the cell is a human cell or a protozoan parasitic cell.

Paragraph 127. The method of paragraph 126, the protozoan parasitic cell is selected from the group consisting of a Cryptosporidium, Babesia, Cyclospora, Cystoisospora, Toxoplasma, Giardia, and Plasmodia parasitic cell.

Paragraph 128. The method of paragraph 127, wherein the protozoan parasitic cell is selected a Plasmodia parasitic cell.

Paragraph 129. The method of paragraph 128, wherein the protozoan parasitic cell is selected from the group consisting of Plasmodium vivax, Plasmodium falciparum, Plasmodium malariae, Plasmodium ovale, and Plasmodium knowlesi.

Paragraph 130. The method of paragraph 128, wherein the protozoan parasitic cell is Plasmodium falciparum.

Paragraph 131. The method of paragraph 126, wherein the human cell is a cancer cell.

Paragraph 132. A method of inhibiting prolyl-tRNA-synthetase in a subject, comprising administering to the subject a compound of any one of paragraphs 1-123, or a pharmaceutically acceptable salt thereof.

Paragraph 133. A method of treating a disorder associated with glutamyl-prolyl-tRNA synthetase, prolyl-tRNA synthetase, or a combination thereof, the method comprising administering to a subject in need thereof a therapeutically effective amount of a compound of any one of paragraphs 1-123, or a pharmaceutically acceptable salt thereof, and a pharmaceutically acceptable carrier.

Paragraph 134. The method of paragraph 133, wherein the disorder is a parasitic infection.

Paragraph 135. The method of paragraph 134, wherein the parasitic infection is selected from malaria, toxoplasmosis, leishmaniasis, cryptosporidiosis, coccidiosis, Chagas disease, African sleeping sickness, giardiasis, and babesiosis.

Paragraph 136. The method of paragraph 135, wherein the disorder is malaria.

Paragraph 137. The method of paragraph 133, wherein the disorder is an autoimmune disease.

Paragraph 138. The method of paragraph 137, wherein the autoimmune disease is selected from multiple sclerosis, rheumatoid arthritis, lupus, psoriasis, scleroderma, dry eye syndrome, Crohn's Disease, inflammatory bowel disease, chronic obstructive pulmonary disease (COPD), asthma, fibrosis, scar formation, ischemic damage, and graft versus host disease.

Paragraph 139. The method of paragraph 133, wherein the disorder is a bacterial infection.

Paragraph 140. The method of paragraph 133, wherein the disorder is a fungal infection.

Paragraph 141. The method of paragraph 133, wherein the disorder is a viral infection.

Paragraph 142. The method of paragraph 141, wherein infection caused by corona virus, dengue virus and chikungunya virus.

Paragraph 143. The method of paragraph 133, wherein the disorder is selected from neurological disorder, a genetic disorder, a cardiovascular disorder, a protein aggregation disorder, a metabolic disorder, an inflammatory disorder, and a cosmetic disorder.

Paragraph 144. The method of paragraph 143, wherein the genetic disorder is Duchenne muscular dystrophy.

Paragraph 145. The method of paragraph 143, wherein the metabolic disorder is selected from diabetes and obesity.

Paragraph 146. The method of paragraph 143, wherein the cosmetic disorder is selected from the group consisting of cellulite and stretch marks.

Paragraph 147. The method of paragraph 143, wherein the inflammatory disorder is selected from restenosis, macular degeneration, choroidal neovascularization, chronic inflammation Paragraph 148. The method of paragraph 133, wherein the disorder is cancer.

Paragraph 149. The method of paragraph 148, wherein the cancer is a T-cell neoplasm selected from mature T-cell leukemia, nodal peripheral T-cell lymphoma (PTCL), extranodal PTCLs, and cutaneous T-cell lymphoma (CTCL).

Paragraph 150. The method of paragraph 148, wherein the cancer is selected from adrenocortical carcinoma, bladder urothelial carcinoma, breast invasive carcinoma, cervical squamous cell carcinoma and endocervical adenocarcinoma, cholangio carcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine corpus endometrial carcinoma, uterine carcinosarcoma, uveal melanoma, multiple myeloma, and chordoma.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A compound of Formula (I): 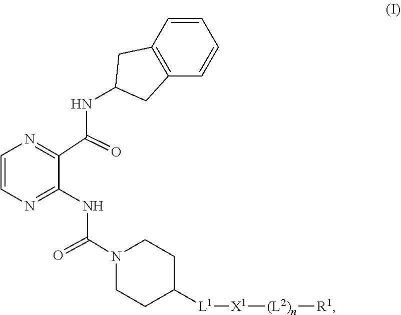
or a pharmaceutically acceptable salt thereof, wherein:
L¹ is C₁₋₃ alkylene, or L¹ is absent;
X¹ is selected from O and NR^{N};
R^{N} is selected from H, C₁₋₃ alkyl, and C₁₋₃ haloalkyl;
each L² is independently selected from O, S, S(═O)₂, NR^{N}, C═O, C═S, and C₁₋₆ alkylene;
n is an integer from 0 to 12; and
R¹ is selected from H, C₁₋₆ alkyl, C₃₋₆ cycloalkyl, C₆₋₁₀ aryl.

2. The compound of claim 1, wherein the compound of Formula (I) is selected from: 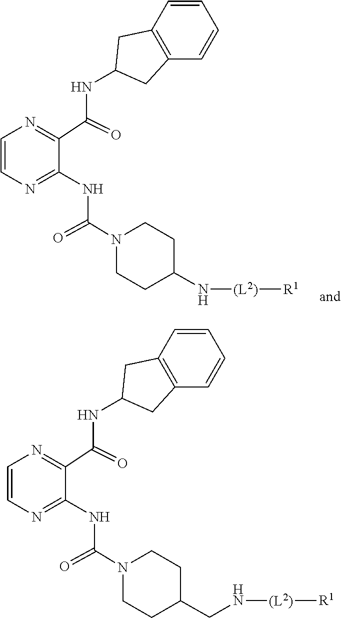
or a pharmaceutically acceptable salt thereof.

3. The compound of claim 1, wherein the moiety (L²)ₙ comprises any one of the following fragments:

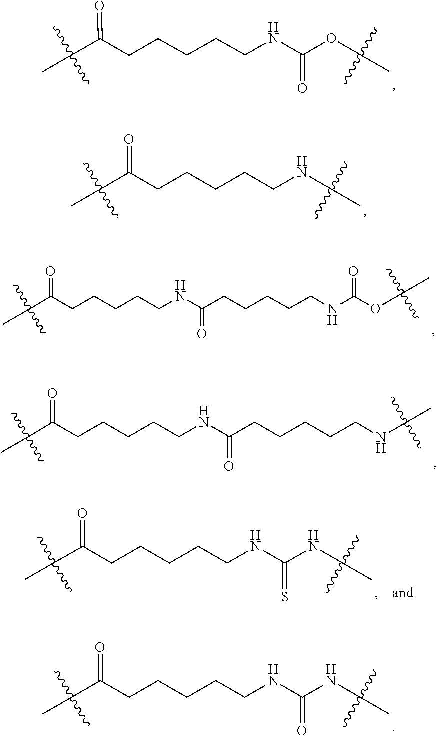 [SMILES: *OC(=O)NCCCCCC(*)=O ; *NCCCCCC(*)=O ; *OC(=O)NCCCCCC(=O)NCCCCCC(*)=O ; *NCCCCCC(=O)NCCCCCC(*)=O ; *NC(=S)NCCCCCC(*)=O ; *NC(=O)NCCCCCC(*)=O]

4. The compound of claim 1, wherein the compound of Formula (I) is selected from: 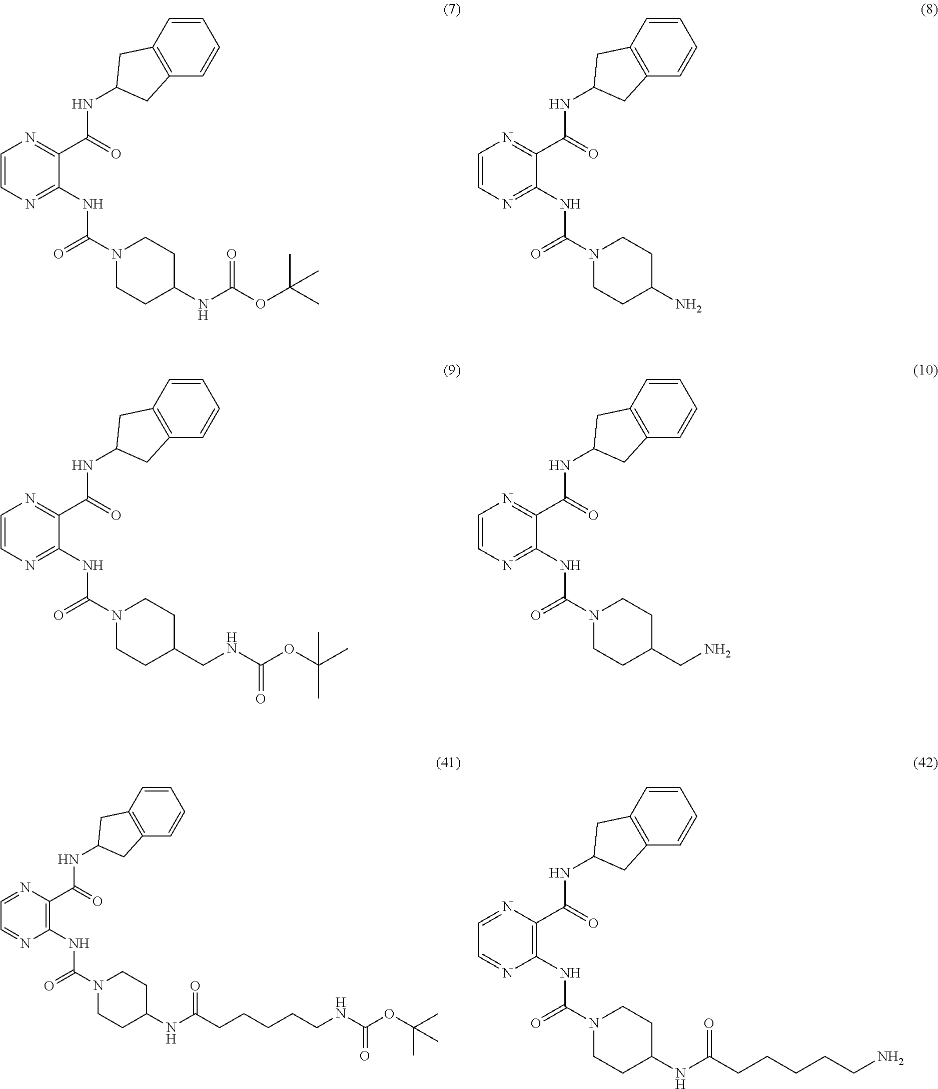 [SMILES: CC(C)(C)OC(=O)NC1CCN(C(=O)Nc2nccnc2C(=O)NC2Cc3ccccc3C2)CC1 ; NC1CCN(C(=O)Nc2nccnc2C(=O)NC2Cc3ccccc3C2)CC1 ; CC(C)(C)OC(=O)NCC1CCN(C(=O)Nc2nccnc2C(=O)NC2Cc3ccccc3C2)CC1 ; NCC1CCN(C(=O)Nc2nccnc2C(=O)NC2Cc3ccccc3C2)CC1 ; CC(C)(C)OC(=O)NCCCCCC(=O)NC1CCN(C(=O)Nc2nccnc2C(=O)NC2Cc3ccccc3C2)CC1 ; NCCCCCC(=O)NC1CCN(C(=O)Nc2nccnc2C(=O)NC2Cc3ccccc3C2)CC1] 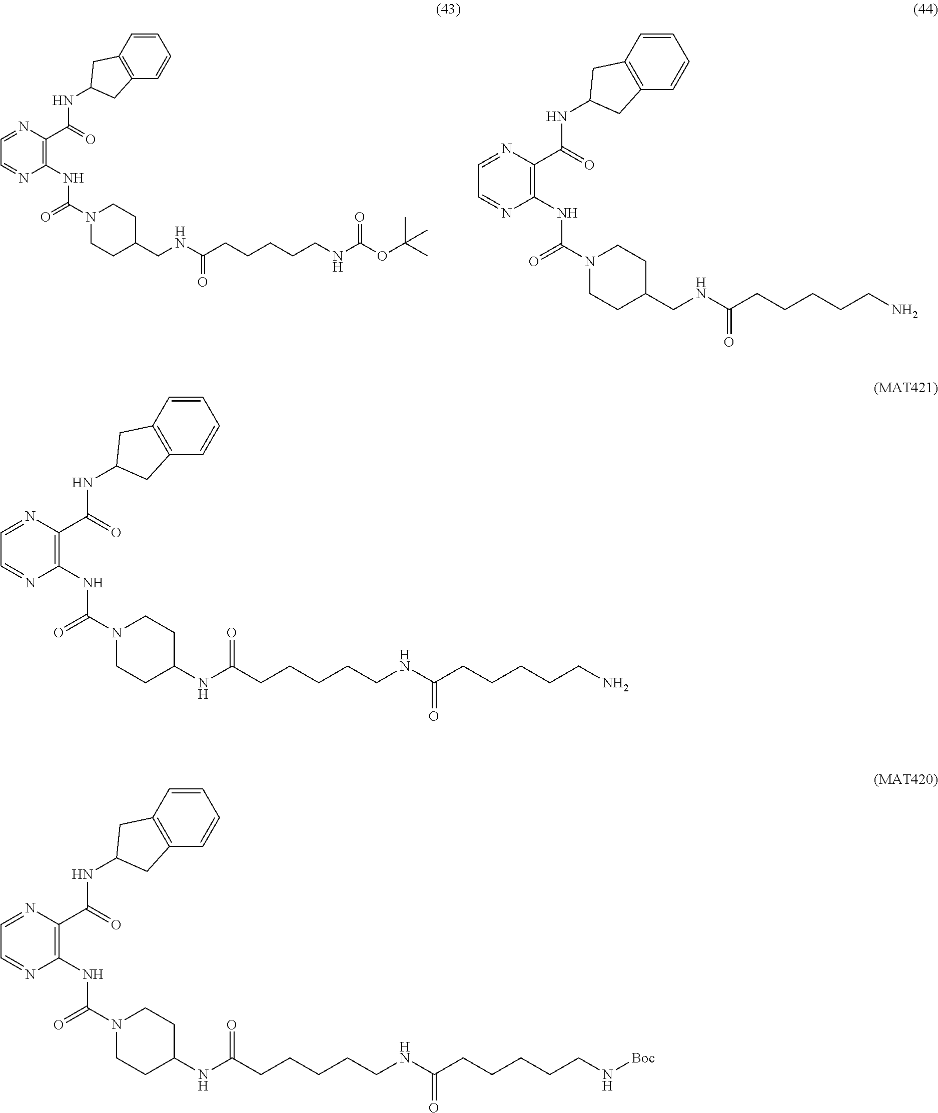 [SMILES: CC(C)(C)OC(=O)NCCCCCC(=O)NCC1CCN(C(=O)Nc2nccnc2C(=O)NC2Cc3ccccc3C2)CC1 ; NCCCCCC(=O)NCC1CCN(C(=O)Nc2nccnc2C(=O)NC2Cc3ccccc3C2)CC1 ; NCCCCCC(=O)NCCCCCC(=O)NC1CCN(C(=O)Nc2nccnc2C(=O)NC2Cc3ccccc3C2)CC1 ; CC(C)(C)OC(=O)NCCCCCC(=O)NCCCCCC(=O)NC1CCN(C(=O)Nc2nccnc2C(=O)NC2Cc3ccccc3C2)CC1] 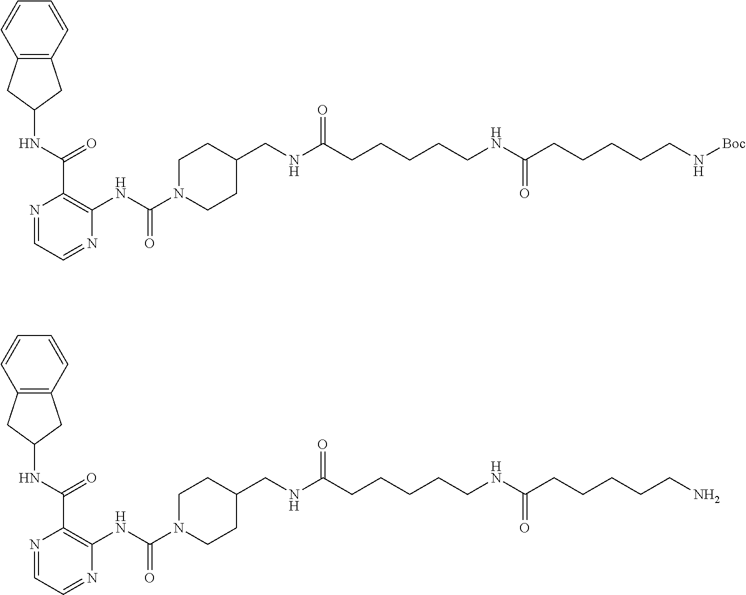 [SMILES: CC(C)(C)OC(=O)NCCCCCC(=O)NCCCCCC(=O)NCC1CCN(C(=O)Nc2nccnc2C(=O)NC2Cc3ccccc3C2)CC1 ; NCCCCCC(=O)NCCCCCC(=O)NCC1CCN(C(=O)Nc2nccnc2C(=O)NC2Cc3ccccc3C2)CC1] 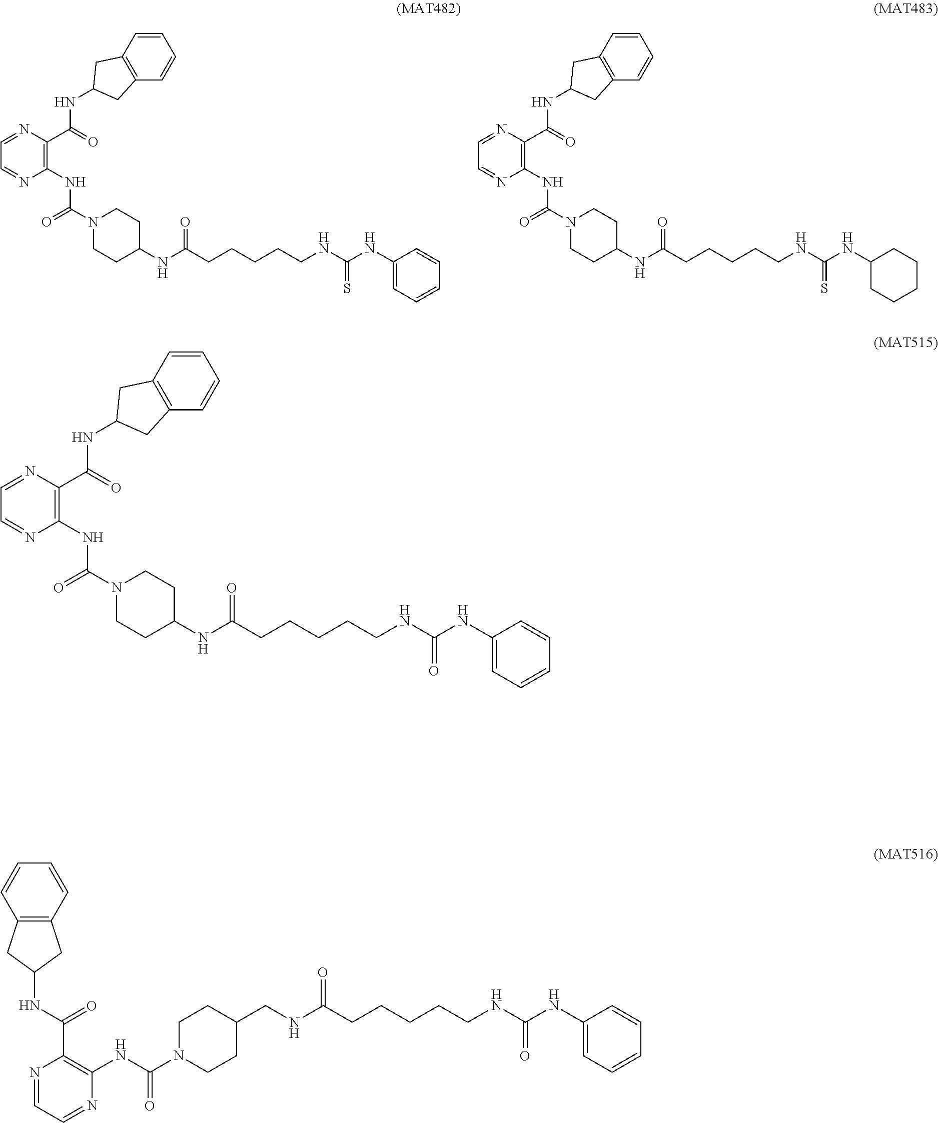 [SMILES: O=C(CCCCCNC(=S)Nc1ccccc1)NC1CCN(C(=O)Nc2nccnc2C(=O)NC2Cc3ccccc3C2)CC1 ; O=C(CCCCCNC(=S)NC1CCCCC1)NC1CCN(C(=O)Nc2nccnc2C(=O)NC2Cc3ccccc3C2)CC1 ; O=C(CCCCCNC(=O)Nc1ccccc1)NC1CCN(C(=O)Nc2nccnc2C(=O)NC2Cc3ccccc3C2)CC1 ; O=C(CCCCCNC(=O)Nc1ccccc1)NCC1CCN(C(=O)Nc2nccnc2C(=O)NC2Cc3ccccc3C2)CC1] 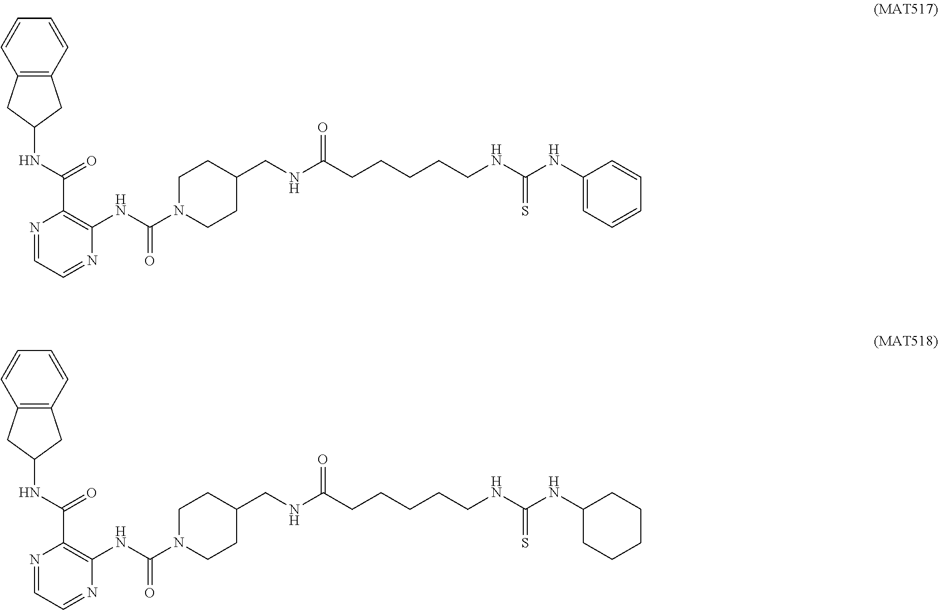 [SMILES: O=C(CCCCCNC(=S)Nc1ccccc1)NCC1CCN(C(=O)Nc2nccnc2C(=O)NC2Cc3ccccc3C2)CC1 ; O=C(CCCCCNC(=S)NC1CCCCC1)NCC1CCN(C(=O)Nc2nccnc2C(=O)NC2Cc3ccccc3C2)CC1]
or a pharmaceutically acceptable salt thereof.

5. A compound of Formula (II): 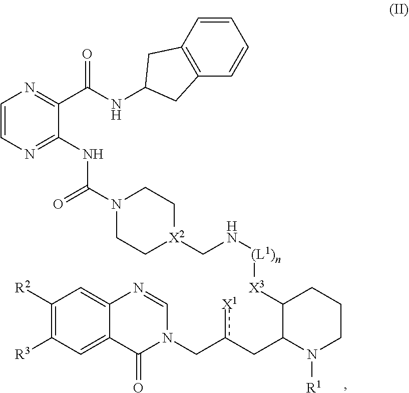
or a pharmaceutically acceptable salt thereof, wherein: [symbol]
is a single bond or a double bond;
when [symbol] is a double bond, X¹ is O or S;
when [symbol] is a single bond, X¹ is OH or SH;
R¹ is selected from H, C₁₋₃ alkyl, C₁₋₃ haloalkyl, —C(═O)R^{A1}, and —C(═O)OR^{A1};
each R^{A1} is selected from H, C₁₋₆ alkyl, and C₁₋₃ haloalkyl;
R² and R³ are each independently a halogen;
X² is selected from N and CH;
X³ is selected from O and NH;
n is an integer from 1 to 6; and
each L¹ is independently selected from O, S, S(═O)₂, NR^{N}, C═O, C═S, and C₁₋₆ alkylene.

6. The compound of claim 5, wherein the compound of Formula (II) is selected from any one of the following compounds: 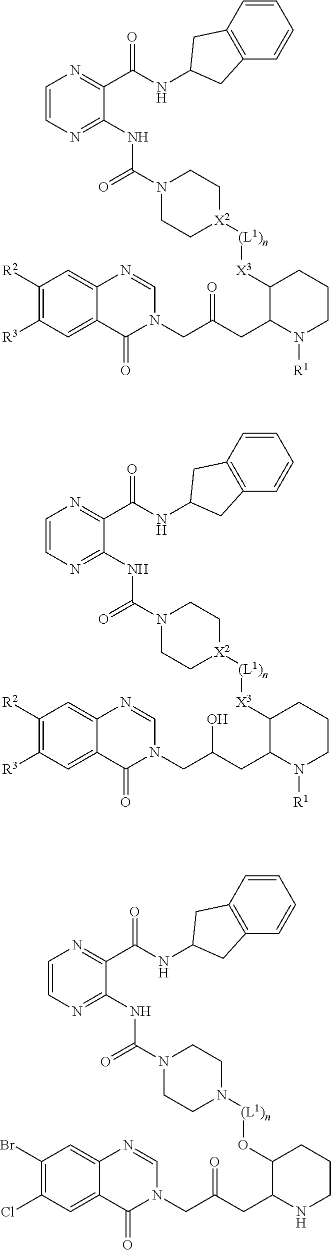 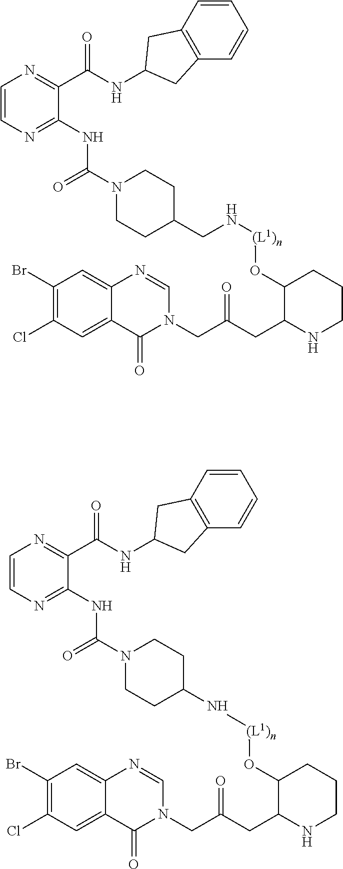
or a pharmaceutically acceptable salt thereof.

7. The compound of claim 5, wherein (L¹)ₙ comprises:

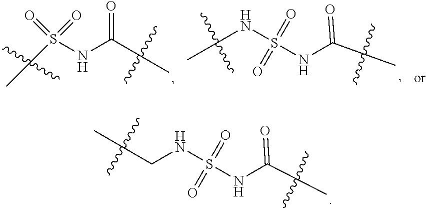 [SMILES: *C(=O)NS(*)(=O)=O ; *NS(=O)(=O)NC(*)=O ; *CNS(=O)(=O)NC(*)=O]

8. The compound of claim 5, wherein the compound of Formula (II) is selected from any one of the following compounds: 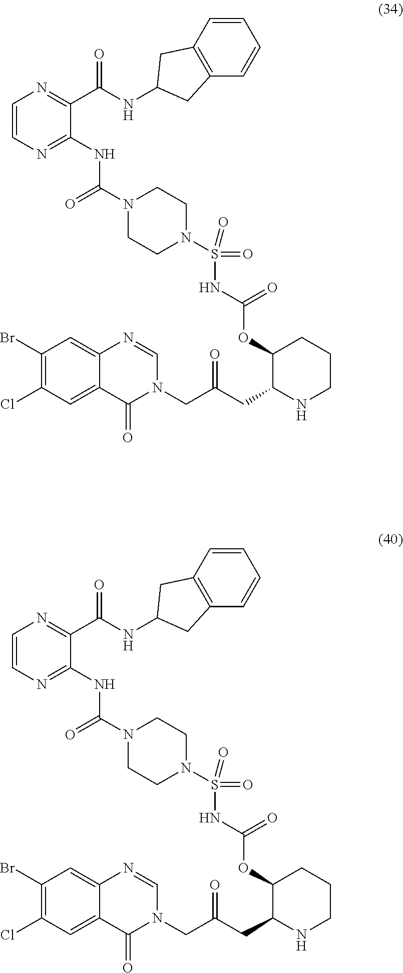 [SMILES: O=C(C[C@H]1NCCC[C@@H]1OC(=O)NS(=O)(=O)N1CCN(C(=O)Nc2nccnc2C(=O)NC2Cc3ccccc3C2)CC1)Cn1cnc2cc(Br)c(Cl)cc2c1=O ; O=C(C[C@@H]1NCCC[C@@H]1OC(=O)NS(=O)(=O)N1CCN(C(=O)Nc2nccnc2C(=O)NC2Cc3ccccc3C2)CC1)Cn1cnc2cc(Br)c(Cl)cc2c1=O] 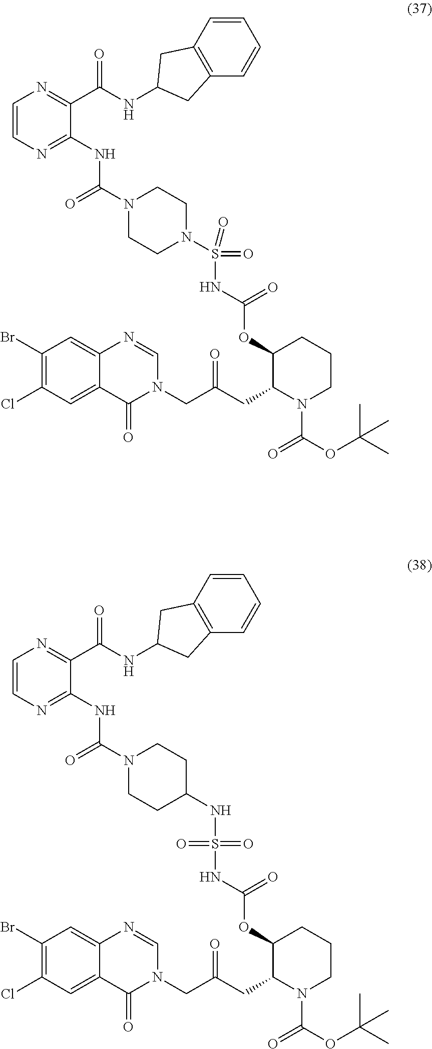 [SMILES: CC(C)(C)OC(=O)N1CCC[C@H](OC(=O)NS(=O)(=O)N2CCN(C(=O)Nc3nccnc3C(=O)NC3Cc4ccccc4C3)CC2)[C@H]1CC(=O)Cn1cnc2cc(Br)c(Cl)cc2c1=O ; CC(C)(C)OC(=O)N1CCC[C@H](OC(=O)NS(=O)(=O)NC2CCN(C(=O)Nc3nccnc3C(=O)NC3Cc4ccccc4C3)CC2)[C@H]1CC(=O)Cn1cnc2cc(Br)c(Cl)cc2c1=O] 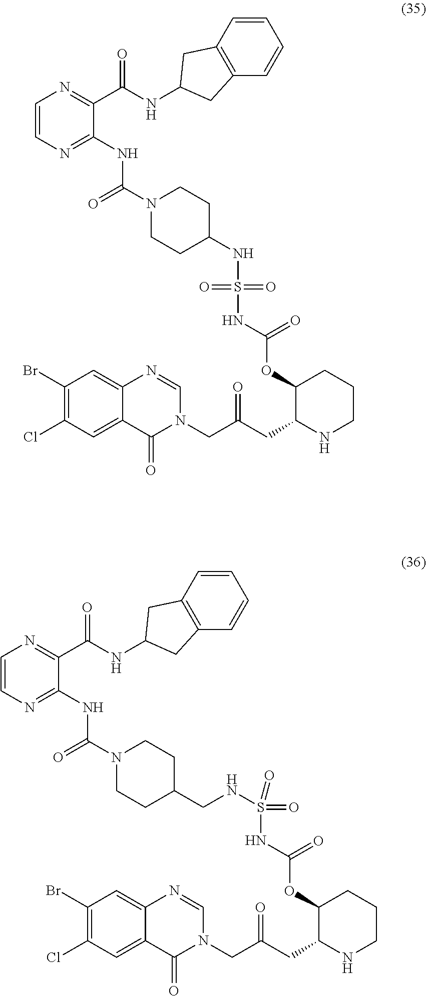 [SMILES: O=C(C[C@H]1NCCC[C@@H]1OC(=O)NS(=O)(=O)NC1CCN(C(=O)Nc2nccnc2C(=O)NC2Cc3ccccc3C2)CC1)Cn1cnc2cc(Br)c(Cl)cc2c1=O ; O=C(C[C@H]1NCCC[C@@H]1OC(=O)NS(=O)(=O)NCC1CCN(C(=O)Nc2nccnc2C(=O)NC2Cc3ccccc3C2)CC1)Cn1cnc2cc(Br)c(Cl)cc2c1=O] 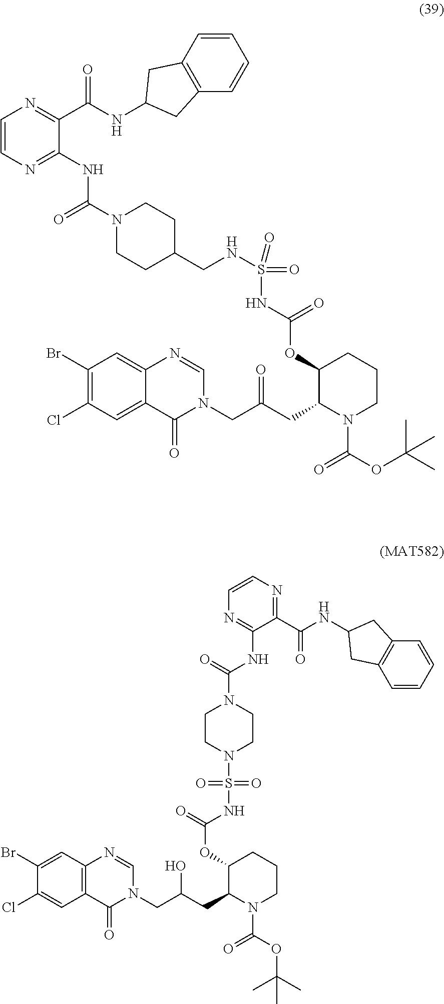 [SMILES: CC(C)(C)OC(=O)N1CCC[C@H](OC(=O)NS(=O)(=O)NCC2CCN(C(=O)Nc3nccnc3C(=O)NC3Cc4ccccc4C3)CC2)[C@H]1CC(=O)Cn1cnc2cc(Br)c(Cl)cc2c1=O ; CC(C)(C)OC(=O)N1CCC[C@@H](OC(=O)NS(=O)(=O)N2CCN(C(=O)Nc3nccnc3C(=O)NC3Cc4ccccc4C3)CC2)[C@@H]1CC(O)Cn1cnc2cc(Br)c(Cl)cc2c1=O] 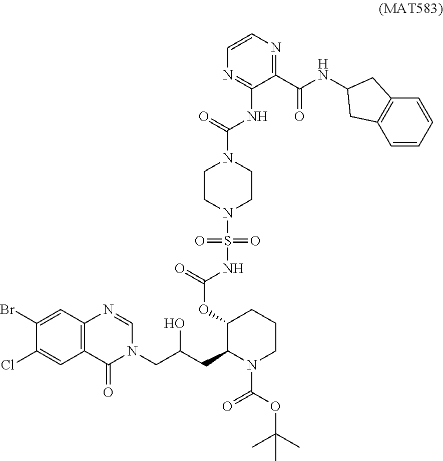 [SMILES: CC(C)(C)OC(=O)N1CCC[C@@H](OC(=O)NS(=O)(=O)N2CCN(C(=O)Nc3nccnc3C(=O)NC3Cc4ccccc4C3)CC2)[C@@H]1CC(O)Cn1cnc2cc(Br)c(Cl)cc2c1=O]
or a pharmaceutically acceptable salt thereof.

9. A compound of Formula (III): 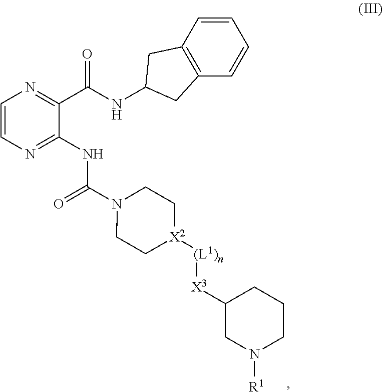
or a pharmaceutically acceptable salt thereof, wherein:
R¹ is selected from H, C₁₋₃ alkyl, C₁₋₃ haloalkyl, —C(═O)R^{A1}, and —C(═O)OR^{A1};
each R^{A1} is selected from H, C₁₋₆ alkyl, and C₁₋₃ haloalkyl;
X² is selected from N and CH;
X³ is selected from O and NH;
n is an integer from 1 to 6; and
each L¹ is independently selected from O, S, S(═O)₂, NR^{N}, C═O, C═S, and C₁₋₆ alkylene.

10. The compound of claim 9, wherein the compound of Formula (III) is selected from any one of the following compounds: 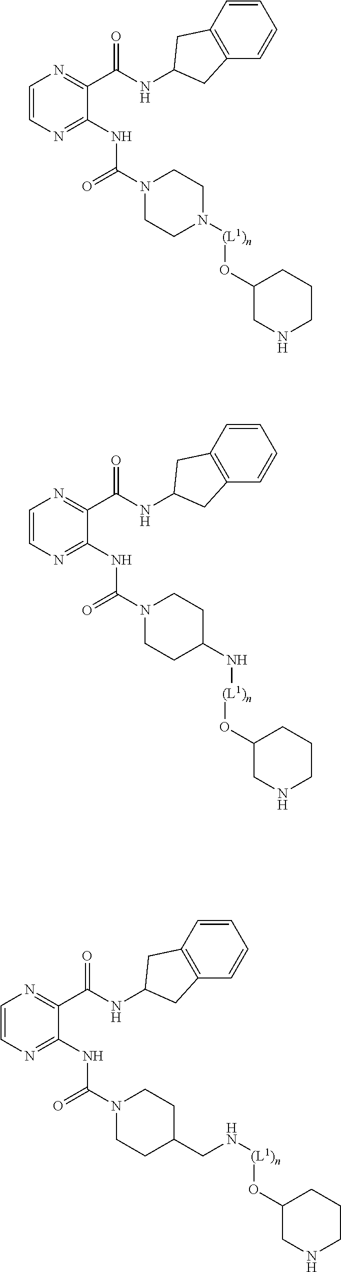
or a pharmaceutically acceptable salt thereof.

11. The compound of claim 9, wherein (L¹)ₙ comprises:

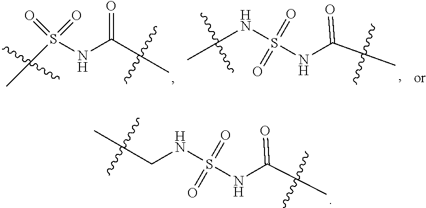 [SMILES: *C(=O)NS(*)(=O)=O ; *NS(=O)(=O)NC(*)=O ; *CNS(=O)(=O)NC(*)=O]

12. The compound of claim 9, wherein the compound of Formula (III) is selected from any one of the following compounds: 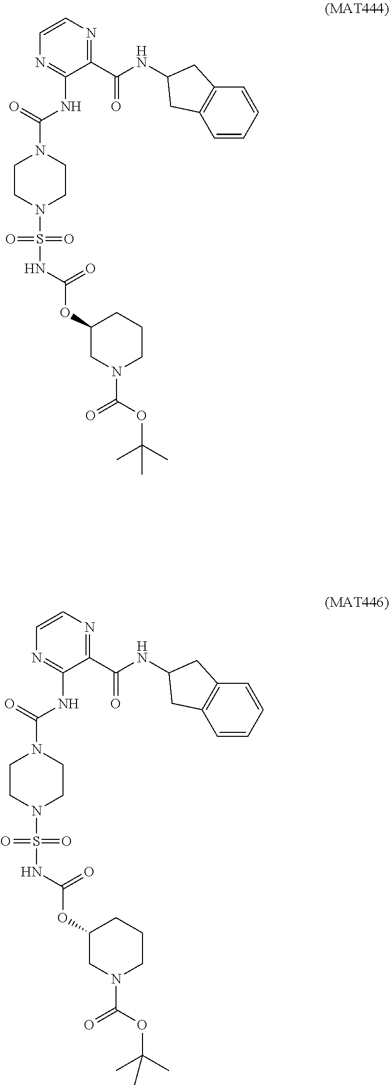 [SMILES: CC(C)(C)OC(=O)N1CCC[C@H](OC(=O)NS(=O)(=O)N2CCN(C(=O)Nc3nccnc3C(=O)NC3Cc4ccccc4C3)CC2)C1 ; CC(C)(C)OC(=O)N1CCC[C@@H](OC(=O)NS(=O)(=O)N2CCN(C(=O)Nc3nccnc3C(=O)NC3Cc4ccccc4C3)CC2)C1] 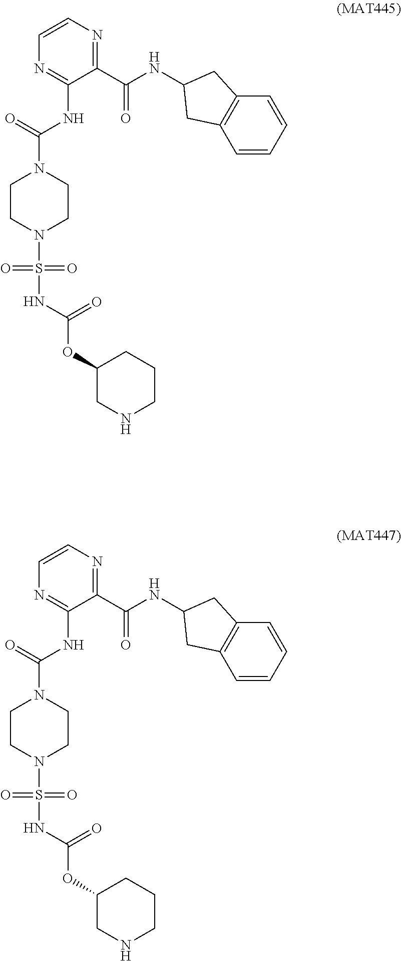 [SMILES: O=C(NS(=O)(=O)N1CCN(C(=O)Nc2nccnc2C(=O)NC2Cc3ccccc3C2)CC1)O[C@H]1CCCNC1 ; O=C(NS(=O)(=O)N1CCN(C(=O)Nc2nccnc2C(=O)NC2Cc3ccccc3C2)CC1)O[C@@H]1CCCNC1]
or a pharmaceutically acceptable salt thereof.

13. A compound of Formula (IV): 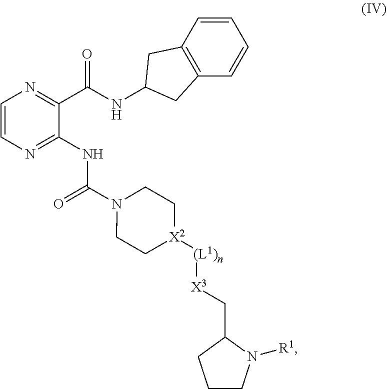
or a pharmaceutically acceptable salt thereof, wherein:
R¹ is selected from H, C₁₋₃ alkyl, C₁₋₃ haloalkyl, —C(═O)R^{A1}, and —C(═O)OR^{A1};
each R^{A1} is selected from H, C₁₋₆ alkyl, and C₁₋₃ haloalkyl;
X² is selected from N and CH;
X³ is selected from O and NH;
n is an integer from 1 to 6; and
each L¹ is independently selected from O, S, S(═O)₂, NR^{N}, C═O, C═S, and C₁₋₆ alkylene.

14. The compound of claim 9, wherein the compound of Formula (IV) is selected from any one of the following compounds: 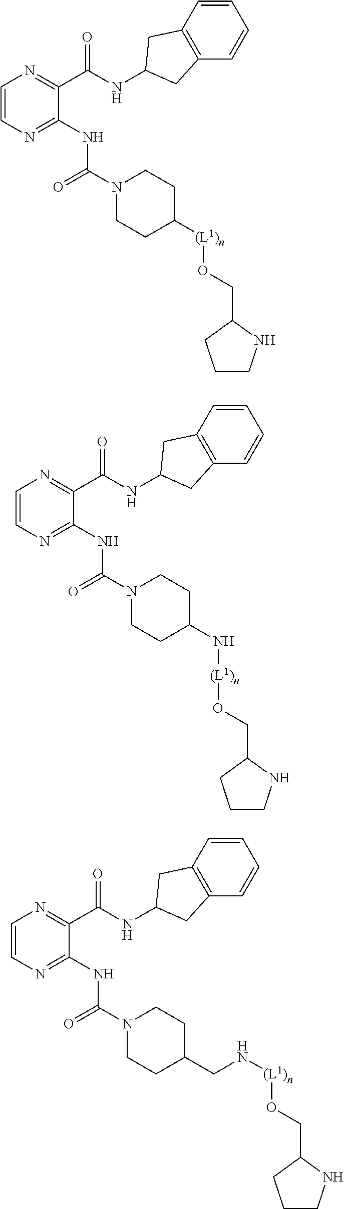
or a pharmaceutically acceptable salt thereof.

15. The compound of claim 9, wherein (L¹)ₙ comprises:

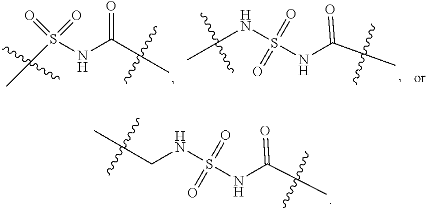 [SMILES: *C(=O)NS(*)(=O)=O ; *NS(=O)(=O)NC(*)=O ; *CNS(=O)(=O)NC(*)=O]

16. The compound of claim 9, wherein the compound of Formula (IV) is selected from any one of the following compounds: 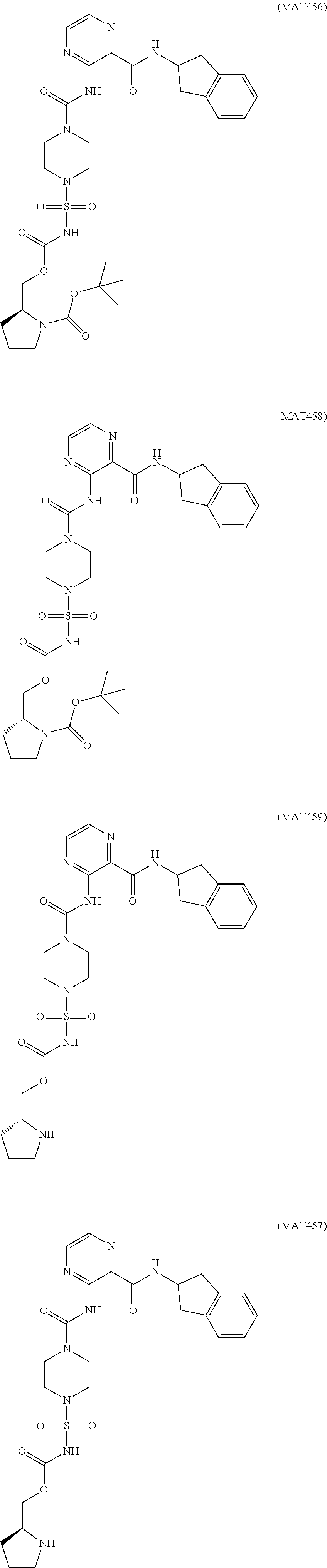 [SMILES: CC(C)(C)OC(=O)N1CCC[C@H]1COC(=O)NS(=O)(=O)N1CCN(C(=O)Nc2nccnc2C(=O)NC2Cc3ccccc3C2)CC1 ; CC(C)(C)OC(=O)N1CCC[C@@H]1COC(=O)NS(=O)(=O)N1CCN(C(=O)Nc2nccnc2C(=O)NC2Cc3ccccc3C2)CC1 ; O=C(NS(=O)(=O)N1CCN(C(=O)Nc2nccnc2C(=O)NC2Cc3ccccc3C2)CC1)OC[C@H]1CCCN1 ; O=C(NS(=O)(=O)N1CCN(C(=O)Nc2nccnc2C(=O)NC2Cc3ccccc3C2)CC1)OC[C@@H]1CCCN1] 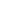
or a pharmaceutically acceptable salt thereof.

17. A compound of Formula (V): 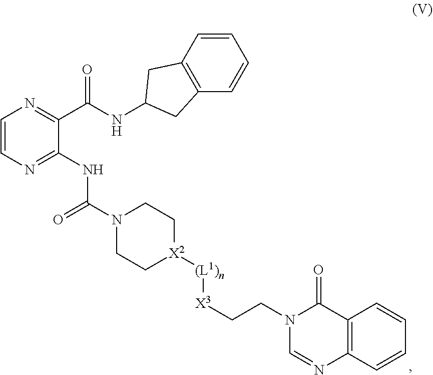
or a pharmaceutically acceptable salt thereof, wherein:
X² is selected from N and CH;
X³ is selected from O and NH;
n is an integer from 1 to 6; and
each L¹ is independently selected from O, S, S(═O)₂, NR^{N}, C═O, C═S, and C₁₋₆ alkylene.

18. The compound of claim 17, wherein the compound of Formula (IV) is selected from any one of the following compounds: 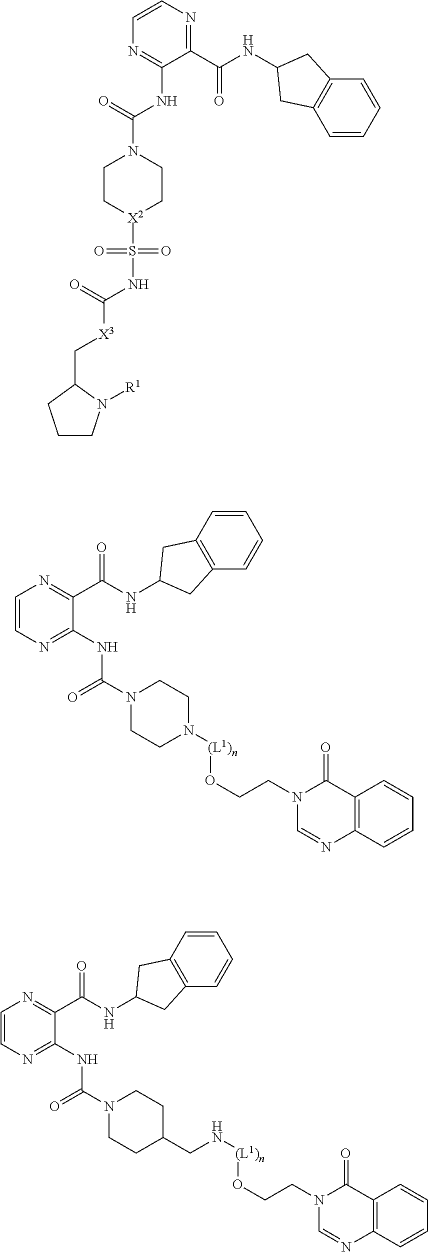 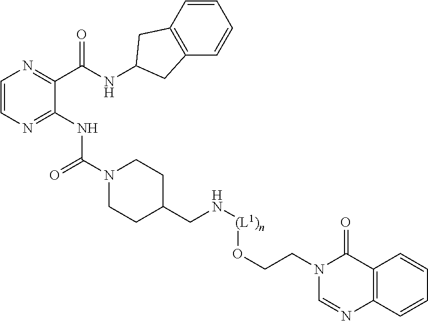
or a pharmaceutically acceptable salt thereof.

19. The compound of claim 17, wherein (L¹)ₙ comprises:

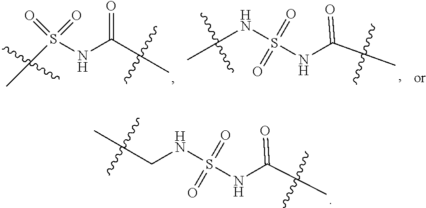 [SMILES: *C(=O)NS(*)(=O)=O ; *NS(=O)(=O)NC(*)=O ; *CNS(=O)(=O)NC(*)=O]

20. The compound of claim 17, wherein the compound of Formula (IV) is selected from any one of the following compounds: 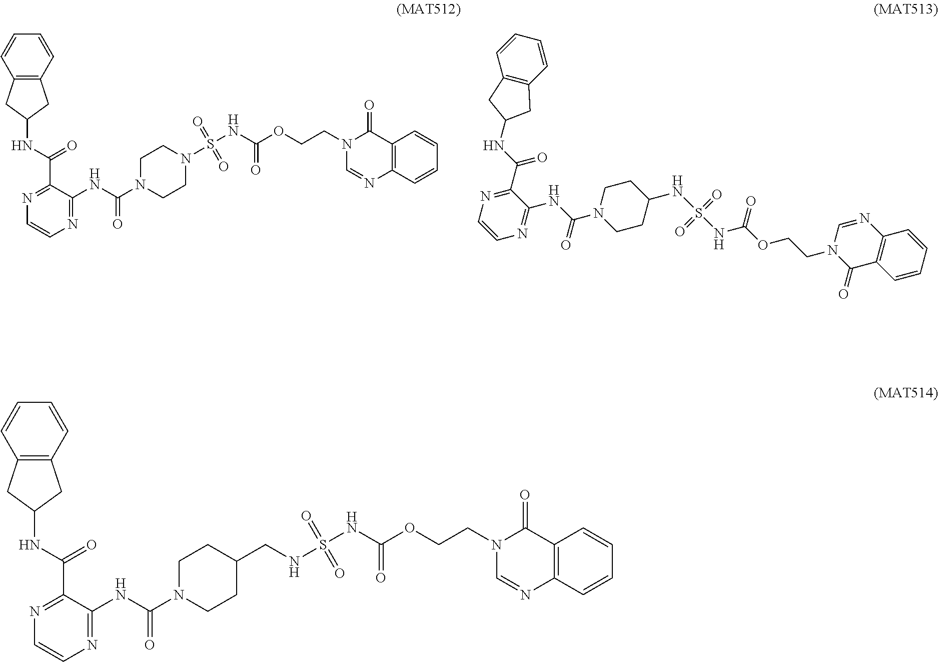 [SMILES: O=C(NS(=O)(=O)N1CCN(C(=O)Nc2nccnc2C(=O)NC2Cc3ccccc3C2)CC1)OCCn1cnc2ccccc2c1=O ; O=C(NS(=O)(=O)NC1CCN(C(=O)Nc2nccnc2C(=O)NC2Cc3ccccc3C2)CC1)OCCn1cnc2ccccc2c1=O ; O=C(NS(=O)(=O)NCC1CCN(C(=O)Nc2nccnc2C(=O)NC2Cc3ccccc3C2)CC1)OCCn1cnc2ccccc2c1=O]
or a pharmaceutically acceptable salt thereof.

21. A compound selected from: 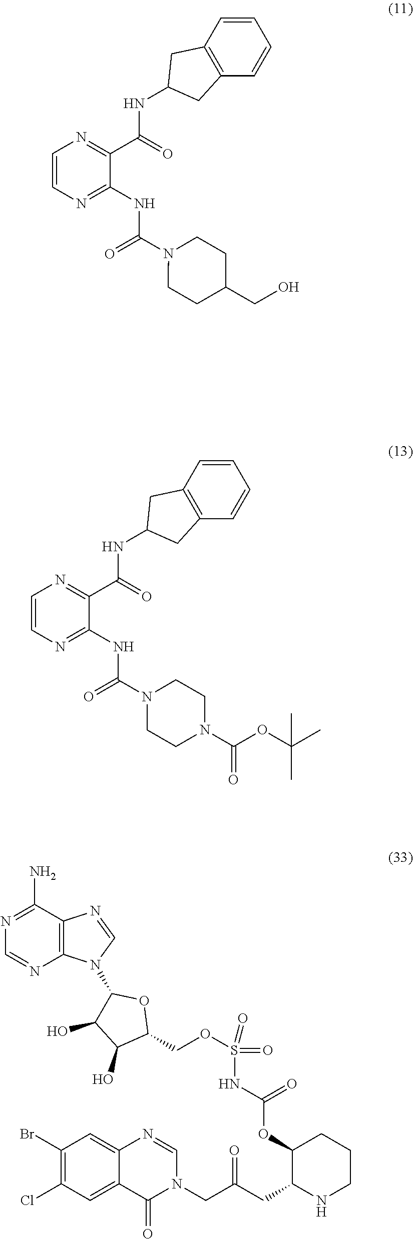 [SMILES: O=C(NC1Cc2ccccc2C1)c1nccnc1NC(=O)N1CCC(CO)CC1 ; CC(C)(C)OC(=O)N1CCN(C(=O)Nc2nccnc2C(=O)NC2Cc3ccccc3C2)CC1 ; Nc1ncnc2c1ncn2[C@@H]1O[C@H](COS(=O)(=O)NC(=O)O[C@H]2CCCN[C@@H]2CC(=O)Cn2cnc3cc(Br)c(Cl)cc3c2=O)[C@@H](O)[C@H]1O] 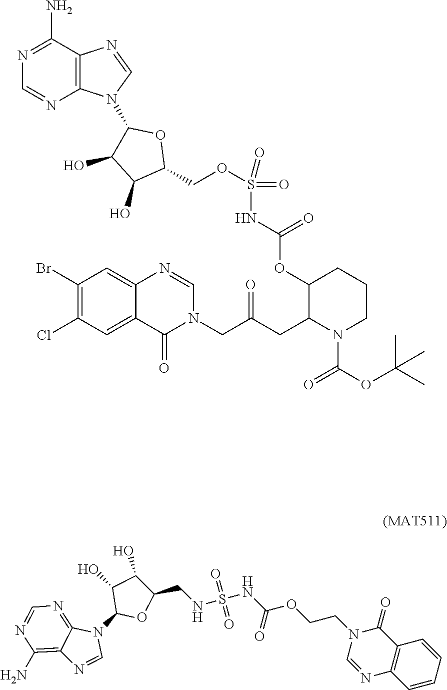 [SMILES: CC(C)(C)OC(=O)N1CCCC(OC(=O)NS(=O)(=O)OC[C@H]2O[C@@H](n3cnc4c(N)ncnc43)[C@H](O)[C@@H]2O)C1CC(=O)Cn1cnc2cc(Br)c(Cl)cc2c1=O ; Nc1ncnc2c1ncn2[C@@H]1O[C@H](CNS(=O)(=O)NC(=O)OCCn2cnc3ccccc3c2=O)[C@@H](O)[C@H]1O]
or a pharmaceutically acceptable salt thereof.

22. A pharmaceutical composition comprising a compound of claim 1, or a pharmaceutically acceptable salt thereof, and a pharmaceutically acceptable carrier.

23. A method of treating a disorder associated with glutamyl-prolyl-tRNA synthetase, prolyl-tRNA synthetase, or a combination thereof, the method comprising administering to a subject in need thereof a therapeutically effective amount of a compound of claim 1, or a pharmaceutically acceptable salt thereof.

24. The method of claim 23, wherein the disorder is selected from parasitic infection, autoimmune disease, bacterial infection, fungal infection, viral infection, a neurological disorder, a genetic disorder, a cardiovascular disorder, a protein aggregation disorder, a metabolic disorder, an inflammatory disorder, a cosmetic disorder, and cancer.

25. The method of claim 24, wherein the parasitic infection is malaria.
